(19) United States
(12) Patent Application Publication
(10) Pub. No.: US 2002/0012979 A1
(43) Pub. Date: Jan. 31, 2002

(54) VITAMIN C PRODUCTION IN MICROORGANISMS AND PLANTS

(76) Inventors: ALAN BERRY, BLOOMFIELD, NJ (US); JEFFREY A. RUNNING, MANITOWOC, WI (US); DAVID K. SEVERSON, TWO RIVERS, WI (US); RICHARD P. BURLINGGAME, MANITOWOC, WI (US)

Correspondence Address: SHERIDAN ROSS PC, 1560 BROADWAY, SUITE 1200, DENVER, CO, 80202

(21) Appl. No.: 09/318,271

(22) Filed: May 25, 1999

Related U.S. Application Data
(60) Provisional application No. 60/088,549, filed on Jun. 8, 1998.
(60) Provisional application No. 60/125,073, filed on Mar. 17, 1999.
(60) Provisional application No. 60/125,054, filed on Mar. 18, 1999.

(51) Int. Cl. C12P 7/40

(52) U.S. Cl. 435/136; 435/243; 435/233; 435/252.3; 435/252.31; 435/252.32; 435/252.33; 435/252.34; 435/252.35; 435/254.1; 435/254.11; 435/254.21

(57) ABSTRACT

A biosynthetic method for producing vitamin C (ascorbic acid, L-ascorbic acid, or AA) is disclosed. Such a method includes fermentation of a genetically modified microorganism or plant to produce L-ascorbic acid. In particular, the present invention relates to the use of microorganisms and plants having at least one genetic modification to increase the action of an enzyme involved in the ascorbic acid biosynthetic pathway. Included is the use of nucleotide sequences encoding epimerases, including the endogenous GDP-D-mannose:GDP-L-galactose epimerase from the L-ascorbic acid pathway and homologues thereof for the purposes of improving the biosynthetic production of ascorbic acid. The present invention also relates to genetically modified microorganisms, such as strains of microalgae, bacteria and yeast useful for producing L-ascorbic acid, and to genetically modified plants, useful for producing consumable plant food products.

### CROSS-REFERENCE TO RELATED APPLICATIONS

The present application claims priority under 35 U.S.C. §119(e) from U.S. Provisional Application Ser. No. 60/088,549, filed Jun. 8, 1998; from U.S. Provisional Application Ser. No. 60/125,073, filed Mar. 17, 1999; and from U.S. Provisional Application Ser. No. 60/125,054, filed Mar. 18, 1999. Each of U.S. Provisional Application Ser. Nos. 60/088,549, 60/125,073 and 60/125,054 is incorporated herein by reference in its entirety.

### FIELD OF THE INVENTION

The present invention relates to vitamin C (L-ascorbic acid) production using genetically modified microorganisms and plants. In particular, the present invention relates to the use of nucleotide sugar epimerase enzymes for the biological production of ascorbic acid in plants and microorganisms.

### BACKGROUND OF THE INVENTION

Nearly all forms of life, both plant and animal, either synthesize ascorbic acid (vitamin C) or require it as a nutrient. Ascorbic acid was first identified to be useful as a dietary supplement for humans and animals for the prevention of scurvy. Ascorbic acid, however, also affects human physiological functions such as the adsorption of iron, cold tolerance, the maintenance of the adrenal cortex, wound healing, the synthesis of polysaccharides and collagen, the formation of cartilage, dentine, bone and teeth, the maintenance of capillaries, and is useful as an antioxidant.

For use as a dietary supplement, ascorbic acid can be isolated from natural sources, such as rosehips, synthesized chemically through the oxidation of L-sorbose, or produced by the oxidative fermentation of calcium D-gluconate by Acetobacter suboxidans. Considine, “Ascorbic Acid,” Van Nostrand's Scientific Encyclopedia, Vol. 1, pp. 237-238, (1989). Ascorbic acid (predominantly intracellular) has also been obtained through the fermentation of strains of the microalga, Chlorella pyrenoidosa. See U.S. Pat. No. 5,001,059 by Skatrud, which is assigned to the assignee of the present application. It is believed that ascorbic acid is produced inside the chloroplasts of photosynthetic microorganisms and functions to neutralize energetic electrons produced during photosynthesis. Accordingly, ascorbic acid production is known in photosynthetic organisms as a protective mechanism.

Therefore, products and processes which improve the ability to biosynthetically produce ascorbic acid are desirable and beneficial for the improvement of human health.

### SUMMARY OF THE INVENTION

One embodiment of the present invention relates to a method for producing ascorbic acid or esters thereof in a microorganism. The method includes the steps of: (a) culturing a microorganism having a genetic modification to increase the action of an enzyme selected from the group of hexokinase, glucose phosphate isomerase, phosphomannose isomerase, phosphomannomutase, GDP-D-mannose pyrophosphorylase, GDP-D-mannose:GDP-L-galactose epimerase, GDP-L-galactose phosphorylase, L-galactose-1-P-phosphatase, L-galactose dehydrogenase, and/or L-galactono-γ-lactone dehydrogenase; and (b) recovering the ascorbic acid or esters produced by the microorganism. Preferably, the genetic modification is a genetic modification to increase the action of an enzyme selected from the group of GDP-D-mannose:GDP-L-galactose epimerase, GDP-L-galactose phosphorylase, L-galactose-1-P-phosphatase, L-galactose dehydrogenase, and/or L-galactono-γ-lactone dehydrogenase. In one embodiment of the method of the present invention, the microorganism further includes a genetic modification to decrease the action of an enzyme having GDP-D-mannose as a substrate, other than GDP-D-mannose:GDP-L-galactose epimerase. Such a genetic modification can include, for example, a genetic modification to decrease the action of GDP-D-mannose-dehydrogenase.

In one embodiment, the genetic modification is a genetic modification to increase the action of an epimerase that catalyzes conversion of GDP-D-mannose to GDP-L-galactose, which can include GDP-D-mannose:GDP-L-galactose epimerase. In one embodiment, the epimerase binds NADPH. In one embodiment of this method, the genetic modification includes transformation of the microorganism with a recombinant nucleic acid molecule that expresses the epimerase. Such an epimerase can have a tertiary structure that substantially conforms to the tertiary structure of a GDP-4-keto-6-deoxy-D-mannose epimerase/reductase represented by atomic coordinates having Brookhaven Protein Data Bank Accession Code 1bws. Preferably, the epimerase has a structure having an average root mean square deviation of less than about 2.5 Å, and more preferably less than about 1 Å, over at least about 25% of Ca positions of the tertiary structure of a GDP-4-keto-6-deoxy-D-mannose epimerase/reductase represented by atomic coordinates having Brookhaven Protein Data Bank Accession Code 1bws.

In one embodiment, the epimerase comprises a substrate binding site having a tertiary structure that substantially conforms to the tertiary structure of the substrate binding site of a GDP-4-keto-6-deoxy-D-mannose epimerase/reductase represented by atomic coordinates having Brookhaven Protein Data Bank Accession Code 1bws. Such a substrate binding site preferably has a tertiary structure with an average root mean square deviation of less than about 2.5 Å over at least about 25% of Cα positions of the tertiary structure of a substrate binding site of a GDP-4-keto-6-deoxy-D-mannose epimerase/reductase represented by atomic coordinates having Brookhaven Protein Data Bank Accession Code 1bws.

In another embodiment, the epimerase comprises a catalytic site having a tertiary structure that substantially conforms to the tertiary structure of the catalytic site of a GDP-4-keto-6-deoxy-D-mannose epimerase/reductase represented by atomic coordinates having Brookhaven Protein Data Bank Accession Code 1bws. Such a catalytic site preferably has a tertiary structure with an average root mean square deviation of less than about 1 Å over at least about 25% of Cα positions of the tertiary structure of a catalytic site of a GDP-4-keto-6-deoxy-D-mannose epimerase/reductase represented by atomic coordinates having Brookhaven Protein Data Bank Accession Code 1bws. The catalytic site preferably includes the amino acid residues serine, tyrosine and lysine and in one embodiment, the tertiary structure positions of the amino acid residues serine, tyrosine and lysine substantially conform to tertiary structure positions of residues Ser107, Tyr136 and Lys140, respectively, as represented by atomic coordinates in Brookhaven Protein Data Bank Accession Code 1bws.

In yet another embodiment of this method, the epimerase comprises an amino acid sequence that aligns with SEQ ID NO:11 using a CLUSTAL alignment program, wherein amino acid residues in the amino acid sequence align with 100% identity with at least about 50%, and in another embodiment with at least about 75%, and in yet another embodiment with at least about 90% of non-Xaa residues in SEQ ID NO:11. In another embodiment, the epimerase comprises an amino acid sequence having at least 4 contiguous amino acid residues that are 100% identical to at least 4 contiguous amino acid residues of an amino acid sequence selected from the group of SEQ ID NO:2, SEQ ID NO:4, SEQ ID NO:6, SEQ ID NO:8 and SEQ ID NO:10. In yet another embodiment, the recombinant nucleic acid molecule comprises a nucleic acid sequence comprising at least about 12 contiguous nucleotides having 100% identity with at least about 12 contiguous nucleotides of a nucleic acid sequence selected from the group of SEQ ID NO:1, SEQ ID NO:3, SEQ ID NO:5, SEQ ID NO:7 and SEQ ID NO:9.

In yet another embodiment of this method of the present invention, the epimerase comprises an amino acid sequence having a motif: Gly-Xaa-Xaa-Gly-Xaa-Xaa-Gly. In yet another embodiment, the recombinant nucleic acid molecule comprises a nucleic acid sequence that is at least about 15% identical, and in another embodiment, at least about 20% identical, and in another embodiment, at least about 25% identical, to a nucleic acid sequence selected from the group of SEQ ID NO:1, SEQ ID NO:3, SEQ ID NO:5, SEQ ID NO:7 and SEQ ID NO:9, as determined using a Lipman-Pearson method with Lipman-Pearson standard default parameters.

In yet another embodiment of this method of the present invention, the recombinant nucleic acid molecule comprises a nucleic acid sequence that hybridizes under stringent hybridization conditions to a nucleic acid sequence encoding a GDP-4-keto-6-deoxy-D-mannose epimerase/reductase. The nucleic acid sequence encoding the GDP-4-keto-6-deoxy-D-mannose epimerase/reductase includes nucleic acid sequences selected from the group of SEQ ID NO:1, SEQ ID NO:3 and SEQ ID NO:5, and the GDP-4-keto-6-deoxy-D-mannose epimerase/reductase can include an amino acid sequence selected from the group of SEQ ID NO:2, SEQ ID NO:4 and SEQ ID NO:6.

In one embodiment of the method of the present invention, the microorganism is selected from the group of bacteria, fungi and microalgae. In one embodiment, the microorganism is acid-tolerant. Preferred bacteria include, but are not limited to Azotobacter and Pseudomonas. Preferred fungi include, but are not limited to, yeast, including, but not limited to Saccharomyces yeast. Preferred microalgae include, but are not limited to, microalgae of the genera Prototheca and Chlorella, with microalgae of the genus Prototheca being particularly preferred.

In yet another embodiment of the method of the present invention, the microorganism is acid-tolerant and the step of culturing is conducted at a pH of less than about 6.0, and more preferably, at a pH of less than about 5.5, and even more preferably, at a pH of less than about 5.0. The step of culturing can be conducted in a fermentation medium that comprises a carbon source other than D-mannose in one embodiment, and in another embodiment, the step of culturing is conducted in a fermentation medium that comprises glucose as a carbon source.

In yet another embodiment of the present method, the step of culturing is conducted in a fermentation medium that is magnesium (Mg) limited. Preferably, the step of culturing is conducted in a fermentation medium that is Mg limited during a cell growth phase. In one embodiment, the fermentation medium includes less than about 0.5 g/L of Mg during a cell growth phase, and more preferably, less than about 0.2 g/L of Mg during a cell growth phase, and even more preferably, less than about 0.1 g/L of Mg during a cell growth phase.

Another embodiment of the present invention relates to a microorganism for producing ascorbic acid or esters thereof. The microorganism has a genetic modification to increase the action of an enzyme selected from the group of hexokinase, glucose phosphate isomerase, phosphomannose isomerase, phosphomannomutase, GDP-D-mannose pyrophosphorylase, GDP-D-mannose:GDP-L-galactose epimerase, GDP-L-galactose phosphorylase, L-galactose-1-P-phosphatase, L-galactose dehydrogenase, and/or L-galactono-γ-lactone dehydrogenase. Preferably, the genetic modification is a genetic modification to increase the action of an enzyme selected from the group of GDP-D-mannose:GDP-L-galactose epimerase, GDP-L-galactose phosphorylase, L-galactose-1-P-phosphatase, L-galactose dehydrogenase, and/or L-galactono-γ-lactone dehydrogenase, and even more preferably, to increase the action of GDP-D-mannose:GDP-L-galactose epimerase.

In one embodiment, the microorganism has been genetically modified to express a recombinant nucleic acid molecule encoding an epimerase that catalyzes conversion of GDP-D-mannose to GDP-L-galactose, wherein the epimerase has a tertiary structure having an average root mean square deviation of less than about 2.5 A over at least about 25% of Cα positions of the tertiary structure of a GDP-4-keto-6-deoxy-D-mannose epimerase/reductase represented by atomic coordinates having Brookhaven Protein Data Bank Accession Code 1bws. In another embodiment, the microorganism has been genetically modified to express a recombinant nucleic acid molecule encoding an epimerase that catalyzes conversion of GDP-D-mannose to GDP-L-galactose, wherein the epimerase comprises an amino acid sequence that aligns with SEQ ID NO:11 using a CLUSTAL alignment program, wherein amino acid residues in the amino acid sequence align with 100% identity with at least about 50% of non-Xaa residues in SEQ ID NO:11. Preferred microorganisms are disclosed as for the method discussed above.

Yet another embodiment of the present invention relates to a plant for producing ascorbic acid or esters thereof. Such a plant has a genetic modification to increase the action of an enzyme selected from the group of hexokinase, glucose phosphate isomerase, phosphomannose isomerase, phosphomannomutase, GDP-D-mannose pyrophosphorylase, GDP-D-mannose:GDP-L-galactose epimerase, GDP-L-galactose phosphorylase, L-galactose-1-P-phosphatase, L-galactose dehydrogenase, and/or L-galactono-γ-lactone dehydrogenase. In a preferred embodiment, the genetic modification is a genetic modification to increase the action of an enzyme selected from the group of GDP-D-mannose:GDP-L-galactose epimerase, GDP-L-galactose phosphorylase, L-galactose-1-P-phosphatase, L-galactose dehydrogenase, and/or L-galactono-γ-lactone dehydrogenase, and in a more preferred embodiment, the genetic modification is a genetic modification to increase the action of GDP-D-mannose:GDP-L-galactose epimerase.

In one embodiment, the plant further comprises a genetic modification to decrease the action of an enzyme having GDP-D-mannose as a substrate other than GDP-D-mannose:GDP-L-galactose epimerase. Such a genetic modification includes a genetic modification to decrease the action of GDP-D-mannose-dehydrogenase. Such a plant also includes a plant that has been genetically modified to express a recombinant nucleic acid molecule encoding an epimerase that catalyzes conversion of GDP-D-mannose to GDP-L-galactose, wherein the epimerase has a tertiary structure having an average root mean square deviation of less than about 2.5 Å over at least about 25% of Cα positions of the tertiary structure of a GDP-4-keto-6-deoxy-D-mannose epimerase/reductase represented by atomic coordinates having Brookhaven Protein Data Bank Accession Code 1bws. In another embodiment, such a plant has been genetically modified to express a recombinant nucleic acid molecule encoding an epimerase that catalyzes conversion of GDP-D-mannose to GDP-L-galactose, wherein the epimerase comprises an amino acid sequence that aligns with SEQ ID NO:11 using a CLUSTAL alignment program, wherein amino acid residues in the amino acid sequence align with 100% identity with at least about 50% of non-Xaa residues in SEQ ID NO:11.

In one embodiment, a plant for producing ascorbic acid or esters thereof according to the present invention is a microalga. Preferred microalgae include, but are not limited to microalgae of the genera Prototheca and Chlorella, with microalga of the genus Prototheca being particularly preferred. In another embodiment, the plant is a higher plant, with consumable higher plants being more preferred.

### BRIEF DESCRIPTION OF THE FIGURES

FIG. 1A is a schematic drawing of the pathway from glucose to GDP-D-mannose in plants.

FIG. 1B is a schematic drawing of the pathway from GDP-D-mannose to L-galactose-1-phosphate in plants.

FIG. 1C is a schematic drawing of the pathway from L-galactose to L-ascorbic acid in plants.

FIG. 2A is a schematic drawing of selected carbon flow from glucose in Prototheca.

FIG. 2B is a schematic drawing of selected carbon flow from glucose in Prototheca.

FIG. 3 is a schematic drawing that shows the lineage of mutants derived from Prototheca moriformis ATCC 75669, and their ability to produce L-ascorbic acid.

FIG. 4 is a bar graph illustrating the conversion of substrates by resting cells of strain NA45-3 following growth in media containing various magnesium concentrations and resuspension in media containing various magnesium concentrations.

FIG. 5 is a line graph showing the relationship between specific ascorbic acid formation in cultures of Prototheca strains and the specific activity of GDP-D-mannose:GDP-L-galactose epimerase in extracts prepared from cells harvested from the same cultures.

FIG. 6 is a line graph showing the relationship between specific epimerase activity and the degree of magnesium limitation in two strains, ATCC 75669 and EMS13-4.

FIG. 7 depicts the overall catalytic mechanism of GDP-D-mannose:GDP-L-galactose epimerase proposed by Barber (1979, J. Biol. Chem. 254:7600-7603).

FIG. 8A depicts the catalytic mechanism of GDP-D-mannose-4,6-dehydratase (converts GDP-D-mannose to GDP-4-keto-6-deoxy-D-mannose).

FIG. 8B depicts the catalytic mechanism of GDP-4-keto-6-deoxy-D-mannose epimerase/reductase (converts GDP-4-keto-6-deoxy-D-mannose to GDP-L-fucose) (Chang, et al., 1988, J. Biol. Chem. 263:1693-1697; Barber, 1980, Plant Physiol. 66: 326-329).

### DETAILED DESCRIPTION OF THE INVENTION

The present invention relates to a biosynthetic method and production microorganisms and plants for producing vitamin C (ascorbic acid, L-ascorbic acid, or AA). Such a method includes fermentation of a genetically modified microorganism to produce L-ascorbic acid. In particular, the present invention relates to the use of nucleotide sequences encoding epimerases, including the endogenous GDP-D-mannose:GDP-L-galactose epimerase from the L-ascorbic acid pathway, as well as epimerases having structural homology (e.g., by nucleotide/amino acid sequence and/or tertiary structure of the encoded protein) to GDP-4-keto-6-deoxy-D-mannose epimerase/reductases, or UDP-galactose 4-epimerases, for the purposes of improving the biosynthetic production of ascorbic acid. The present invention also relates to genetically modified microorganisms, such as strains of microalgae, bacteria and yeast useful for producing L-ascorbic acid, and to genetically modified plants, useful for producing consumable plant food products.

One embodiment of the present invention relates to a method to produce L-ascorbic acid by fermentation of a genetically modified microorganism. This method includes the steps of (a) culturing in a fermentation medium a microorganism having a genetic modification to increase the action of an enzyme selected from the group of hexokinase, glucose phosphate isomerase, phosphomannose isomerase, phosphomannomutase, GDP-mannose pyrophosphorylase, GDP-D-mannose:GDP-L-galactose epimerase, GDP-L-galactose phosphorylase, L-galactose-1-P-phosphatase, L-galactose dehydrogenase, and L-galactono-γ-lactone dehydrogenase; and (b) recovering L-ascorbic acid or esters thereof. The various enzymes in this list represent the enzymes involved in the vitamin C biosynthetic pathway in plants. It is uncertain at this time whether the enzyme represented by GDP-L-galactose phosphorylase is actually a phosphorylase or a pyrophosphorylase (i.e., GDP-L-galactose pyrophosphorylase). Therefore, use of the term “GDP-L-galactose phosphorylase” herein refers to either GDP-L-galactose phosphorylase or GDP-L-galactose pyrophosphorylase. In one aspect of the invention, this method includes the step of culturing in a fermentation medium a microorganism having a genetic modification to increase the action of an epimerase that catalyzes conversion of GDP-D-mannose to GDP-L-galactose. This aspect of the present invention is discussed in detail below.

Another embodiment of the present invention relates to a genetically modified microorganism for producing L-ascorbic acid or esters thereof. Another embodiment of the present invention relates to a genetically modified plant for producing L-ascorbic acid or esters thereof. Both genetically modified microorganisms (e.g., bacteria, yeast, microalgae) and plants (e.g., higher plants, microalgae) have a genetic modification to increase the action of an enzyme selected from the group of hexokinase, glucose phosphate isomerase, phosphomannose isomerase, phosphomannomutase, GDP-mannose pyrophosphorylase, GDP-D-mannose:GDP-L-galactose epimerase, GDP-L-galactose phosphorylase, L-galactose-1-P-phosphatase, L-galactose dehydrogenase, and/or L-galactono-γ-lactone dehydrogenase. In a preferred embodiment, both genetically modified microorganisms (e.g., bacteria, yeast, microalgae) and plants (e.g., higher plants, microalgae) have a genetic modification to increase the action of an epimerase that catalyzes conversion of GDP-D-mannose to GDP-L-galactose. In one embodiment, the genetic modification includes the transformation of the microorganism or plant with the epimerase as described above.

To produce significantly high yields of L-ascorbic acid by the method of the present invention, a plant and/or microorganism is genetically modified to enhance production of L-ascorbic acid. As used herein, a genetically modified plant (such as a higher plant or microalgae) or microorganism, such as a microalga (Prototheca, Chlorella), Escherichia coli, or a yeast, is modified (i.e., mutated or changed) within its genome and/or by recombinant technology (i.e., genetic engineering) from its normal (i.e., wild-type or naturally occurring) form. In a preferred embodiment, a genetically modified plant or microorganism according to the present invention has been modified by recombinant technology. Genetic modification of a plant or microorganism can be accomplished using classical strain development and/or molecular genetic techniques, include genetic engineering techniques. Such techniques are generally disclosed herein and are additionally disclosed, for example, in Sambrook et al., 1989, Molecular Cloning: A Laboratory Manual, Cold Spring Harbor Labs Press; Roessler, 1995, Plant Lipid Metabolism, pp. 46-48; and Roessler et al., 1994, in Bioconversion for Fuels, Himmel et al. eds., American Chemical Society, Washington D.C., pp 255-70). These references are incorporated by reference herein in their entirety.

In some embodiments, a genetically modified plant or microorganism can include a natural genetic variant as well as a plant or microorganism in which nucleic acid molecules have been inserted, deleted or modified, including by mutation of endogenous genes (e.g., by insertion, deletion, substitution, and/or inversion of nucleotides), in such a manner that the modifications provide the desired effect within the plant or microorganism. As discussed above, a genetically modified plant or microorganism includes a plant or microorganism that has been modified using recombinant technology.

As used herein, genetic modifications which result in a decrease in gene expression, an increase in inhibition of gene expression or inhibition of a gene product (i.e., the protein encoded by the gene), a decrease in the function of the gene, or a decrease in the function of the gene product can be referred to as inactivation (complete or partial), deletion, interruption, blockage, down-regulation, or decreased action of a gene. For example, a genetic modification in a gene which results in a decrease in the function of the protein encoded by such gene can be the result of a complete deletion of the gene encoding the protein (i.e., the gene does not exist, and therefore the protein does not exist), a mutation in the gene encoding the protein which results in incomplete or no translation of the protein (e.g., the protein is not expressed), or a mutation in the gene which decreases or abolishes the natural function of the protein (e.g., a protein is expressed which has decreased or no enzymatic activity).

Genetic modifications which result in an increase in gene expression or function can be referred to as amplification, overproduction, overexpression, activation, enhancement, addition, up-regulation or increased action of a gene. Additionally, a genetic modification to a gene which modifies the expression, function, or activity of the gene can have an impact on the action of other genes and their expression products within a given metabolic pathway (e.g., by inhibition or competition). In this embodiment, the action (e.g., activity) of a particular gene and/or its product can be affected (i.e., upregulated or downregulated) by a genetic modification to another gene within the same metabolic pathway, or to a gene within a different metabolic pathway which impacts the pathway of interest by competition, inhibition, substrate formation, etc.

In general, a plant or microorganism having a genetic modification that affects L-ascorbic acid production has at least one genetic modification, as discussed above, which results in a change in the L-ascorbic acid production pathway as compared to a wild-type plant or microorganism grown or cultured under the same conditions. Such a modification in an L-ascorbic acid production pathway changes the ability of the plant or microorganism to produce L-ascorbic acid. According to the present invention, a genetically modified plant or microorganism preferably has an enhanced ability to produce L-ascorbic acid compared to a wild-type plant or microorganism cultured under the same conditions.

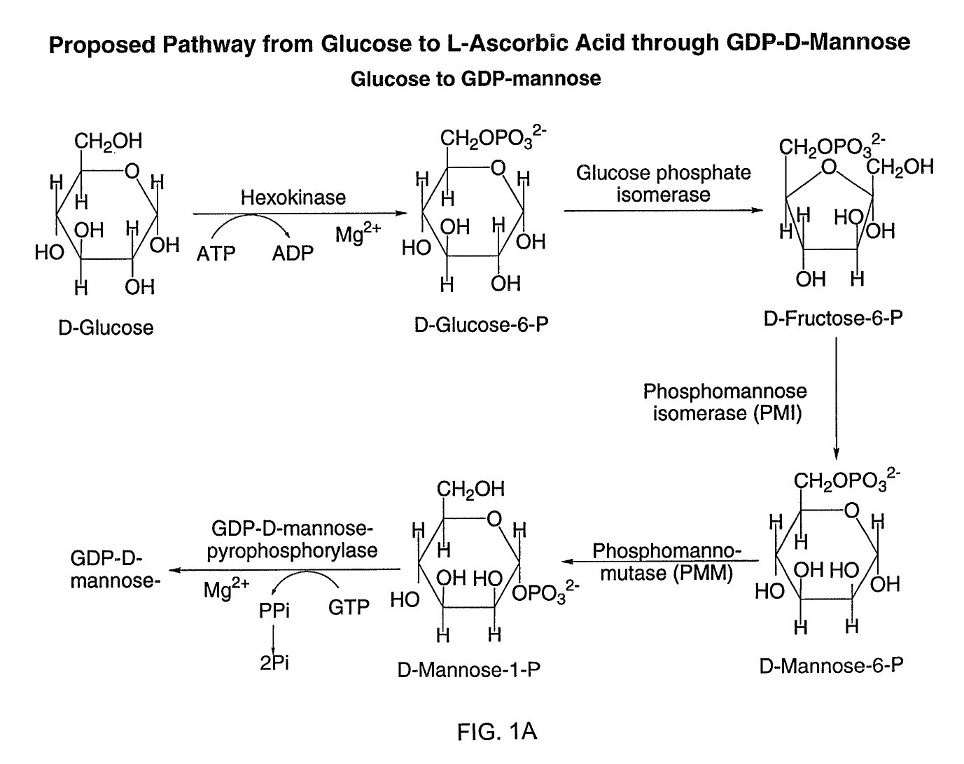

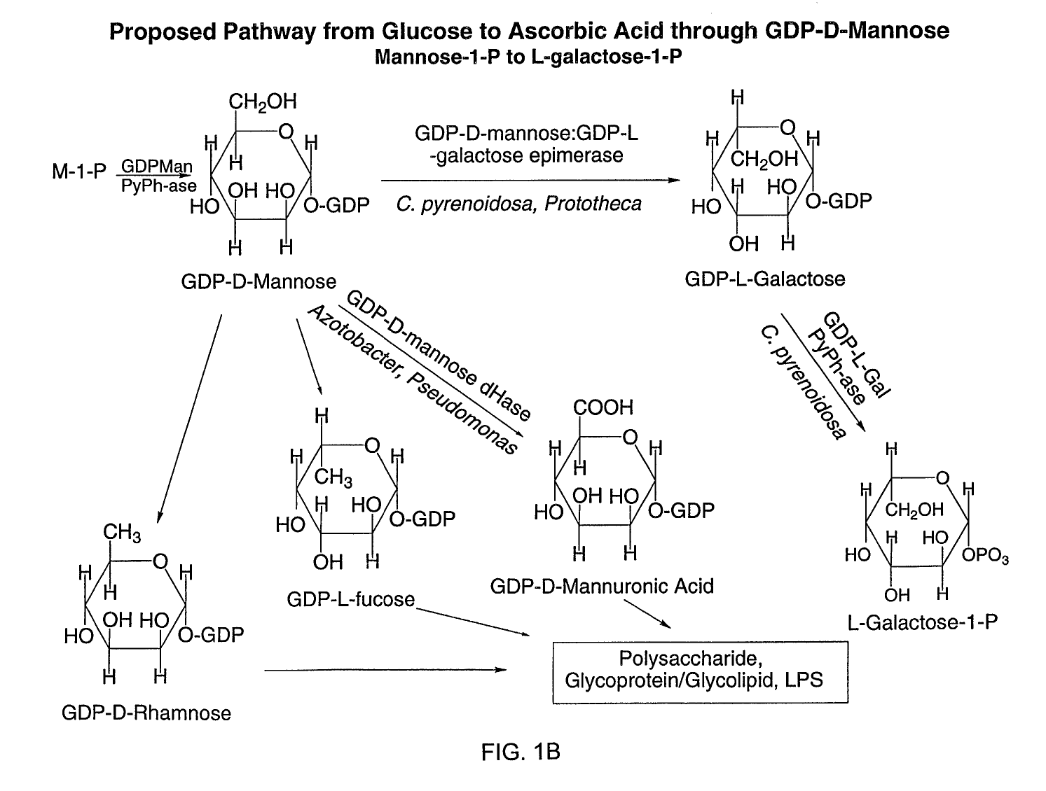

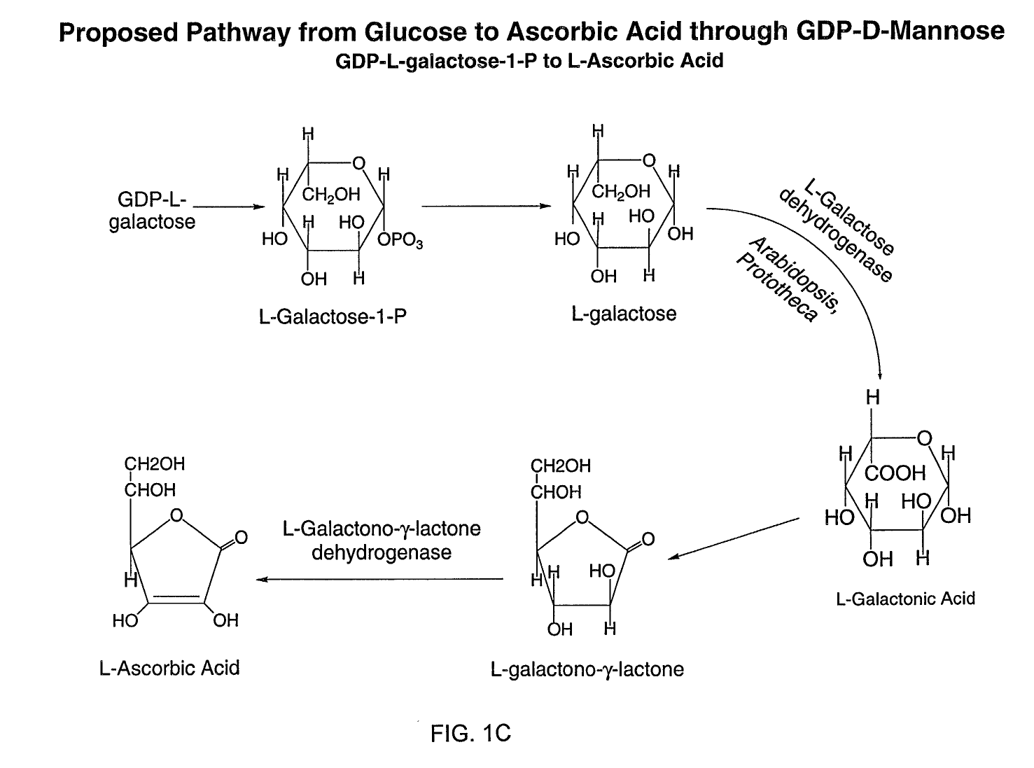

The present invention is based on the present inventors' discovery of the biosynthetic pathway for L-ascorbic acid (vitamin C) in plants and microorganisms. Prior to the present invention, the metabolic pathway by which plants produce L-ascorbic acid, was not completely elucidated. The present inventors have demonstrated that L-ascorbic acid production in plants, including L-ascorbic acid-producing microorganisms (e.g., microalgae), is a pathway which uses GDP-D-mannose and involves sugar phosphates and NDP-sugars. In addition, the present inventors have made the surprising discovery that both L-galactose and L-galactono-γ-lactone can be rapidly converted into L-ascorbic acid in L-ascorbic acid-producing microalgae, including Prototheca and Chlorella pyrenoidosa. The entire pathway for L-ascorbic acid production in plants is set forth in FIGS. 1A-1C. More particularly, FIG. 1A shows that the production of L-ascorbic acid in plants proceeds through the production of mannose intermediates to GDP-D-mannose, followed by the conversion of GDP-D-mannose to GDP-L-galactose by GDP-D-mannose:GDP-L-galactose epimerase (also known as GDP-D-mannose-3,5-epimerase) (FIG. 1B), and then by the subsequent progression to L-galactose-1-P, L-galactose, L-galactonic acid (optional), L-galactono-γ-lactone, and L-ascorbic acid (FIG. 1C). FIG. 1B also illustrates alternate pathways for the use of various intermediates, such as GDP-D-mannose. Certain aspects of this pathway have been independently described in a publication (Wheeler, et al., 1998, Nature 393:365-369), incorporated herein by reference in its entirety.

Points within the L-ascorbic acid production pathway which can be targeted by genetic modification to affect the production of L-ascorbic acid can generally be categorized into at least one of the following pathways: (a) pathways affecting the production of GDP-D-mannose (e.g., pathways for converting a carbon source into GDP-D-mannose); (b) pathways for converting GDP-D-mannose into other compounds, (c) pathways associated with or downstream of the action of GDP-D-mannose:GDP-L-galactose epimerase, (d) pathways which compete for substrates involved in the production of any of the intermediates within the L-ascorbic acid production pathway, and in particular, with GDP-D-mannose, GDP-L-galactose, L-galactose-1-phosphate, L-galactose, L-galactono-γ-lactone, and/or L-ascorbic acid; and (e) pathways which inhibit production of any of the intermediates within the L-ascorbic acid production pathway, and in particular, with GDP-D-mannose, GDP-L-galactose, L-galactose-1-phosphate, L-galactose, L-galactono-γ-lactone, and/or L-ascorbic acid.

A genetically modified plant or microorganism useful in a method of the present invention typically has at least one genetic modification in the L-ascorbic acid production pathway which results in an enhanced production of L-ascorbic acid. In one embodiment, a genetically modified plant or microorganism has at least one genetic modification that results in: (a) an enhanced production of GDP-D-mannose; (b) an inhibition of pathways which convert GDP-D-mannose into compounds other than GDP-L-galactose; (c) an enhancement of action of the GDP-D-mannose:GDP-L-galactose epimerase; (d) an enhancement of the action of enzymes downstream of the GDP-D-mannose:GDP-L-galactose epimerase; (e) an inhibition of pathways which compete for substrates involved in the production of any of the intermediates within the L-ascorbic acid production pathway, and in particular, with GDP-D-mannose, GDP-L-galactose, L-galactose-1-phosphate, L-galactose, L-galactono-γ-lactone, and/or L-ascorbic acid; and (e) an inhibition of pathways which inhibit production of any of the intermediates within the L-ascorbic acid production pathway, and in particular, with GDP-D-mannose, GDP-L-galactose, L-galactose-1-phosphate, L-galactose, L-galactono-γ-lactone, and/or L-ascorbic acid.

An enhanced production of GDP-D-mannose by genetic modification of the plant or microorganism can be achieved by, for example, overexpression of enzymes such as hexokinase, glucose phosphate isomerase, phosphomannose isomerase (PMI), phosphomannomutase (PMM) and/or GDP-D-mannose pyrophosphorylase (GMP). Inhibition of pathways which convert GDP-D-mannose to compounds other than GDP-L-galactose can be achieved, for example, by modifications which inhibit polysaccharide synthesis, GDP-D-rhamnose synthesis, GDP-L-fucose synthesis and/or GDP-D-mannuronic acid synthesis. An increase in the action of the GDP-D-mannose:GDP-L-galactose epimerase and of enzymes downstream of the epimerase in the L-ascorbic acid production pathway can be achieved by genetic modifications which include, but are not limited to: overexpression of the epimerase gene (i.e, by overexpression of a recombinant nucleic acid molecule encoding the epimerase gene or a homologue thereof (discussed in detail below), and/or by mutation of the endogenous or recombinant gene to enhance expression of the gene) and/or overexpression of genes downstream of the epimerase which encode subsequent enzymes in the L-ascorbic acid pathway. Finally, metabolic pathways which compete with or inhibit the L-ascorbic acid production pathway can be inhibited by deleting or mutating enzymes, substrates or products which either inhibit or compete for an enzyme, substrate or product in the L-ascorbic acid pathway.

As discussed above, a genetically modified plant or microorganism useful in the method of the present invention can have at least one genetic modification (e.g., mutation in the endogenous gene or addition of a recombinant gene) in a gene encoding an enzyme involved in the L-ascorbic acid production pathway. Such genetic modifications preferably increase (i.e., enhance) the action of such enzymes such that L-ascorbic acid is preferentially produced as compared to other possible end products in related metabolic pathways. Such genetic modifications include, but are not limited to, overexpression of the gene encoding such enzyme, and deletion, mutation, or downregulation of genes encoding competitors or inhibitors of such enzyme. Preferred enzymes for which the action of the gene encoding such enzyme can be genetically modified include: hexokinase, glucose phosphate isomerase, phosphomannose isomerase (PMI), phosphomannomutase (PMM), GDP-D-mannose pyrophosphorylase (GMP), GDP-D-mannose:GDP-L-galactose epimerase, GDP-L-galactose phosphorylase, L-galactose-1-P-phosphatase, L-galactose dehydrogenase, and/or L-galactono-γ-lactone dehydrogenase. More preferably, a genetically modified plant or microorganism useful in the present invention has a genetic modification which increases the action of an enzyme selected from the group of GDP-D-mannose:GDP-L-galactose epimerase, GDP-L-galactose phosphorylase, L-galactose-1-P-phosphatase, L-galactose dehydrogenase, and/or L-galactono-γ-lactone dehydrogenase. Even more preferably, a genetically modified plant or microorganism useful in the present invention has a genetic modification which increases the action of GDP-D-mannose:GDP-L-galactose epimerase. These enzymes and the reactions catalyzed by such enzymes are illustrated in FIGS. 1A-1C.

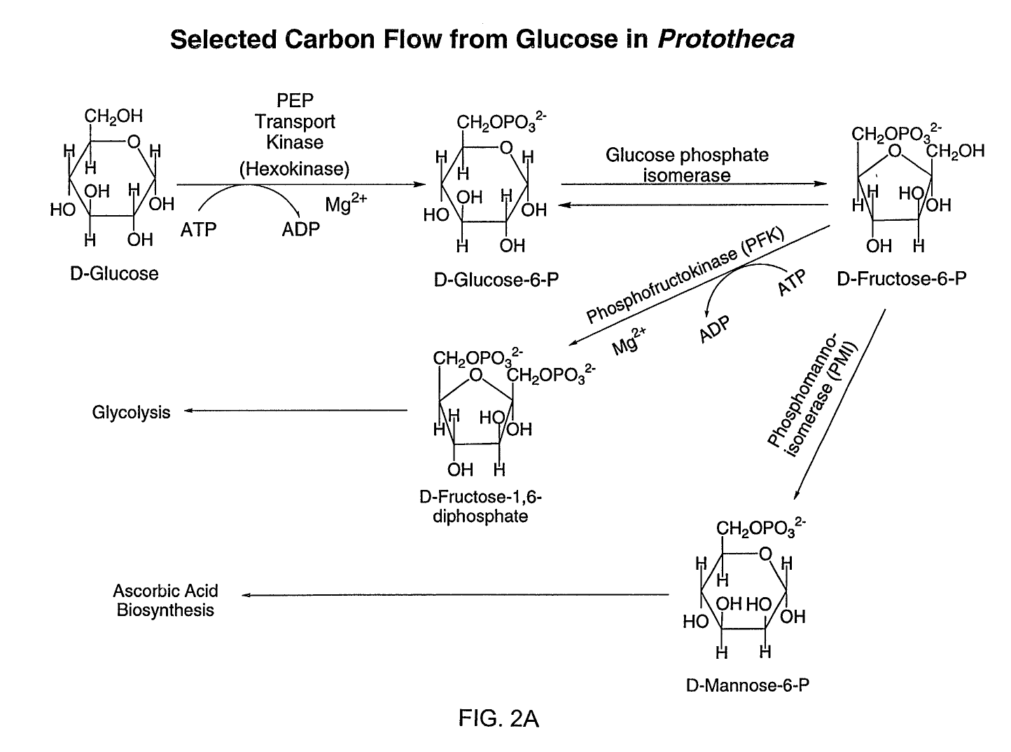

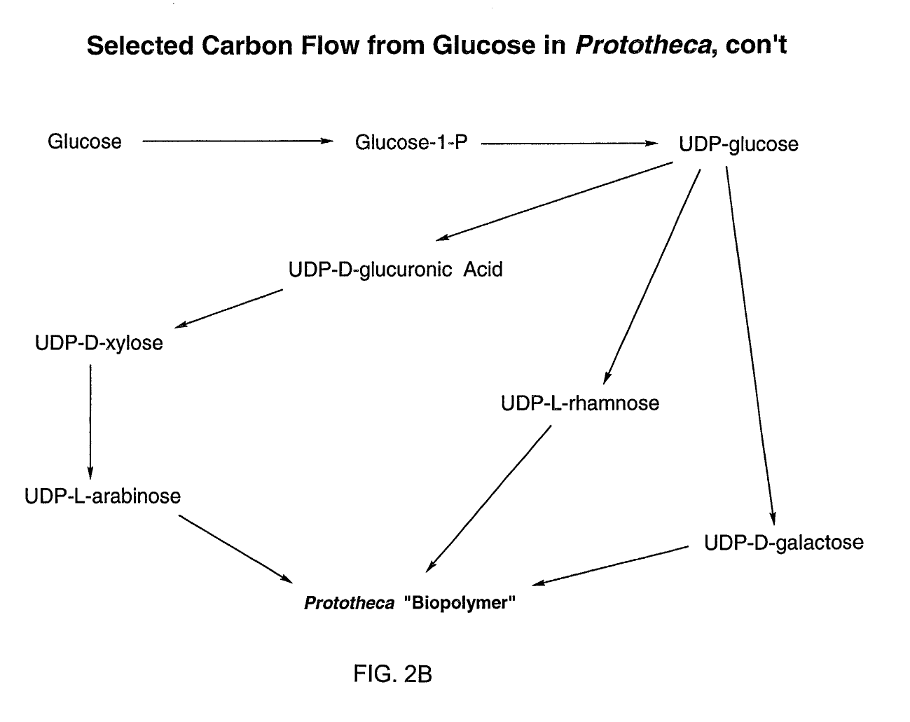

Prior to the present invention, without knowing the L-ascorbic acid biosynthetic (i.e., production) pathway, previous mutagenesis and screening efforts were limited in that only non-lethal mutations could be detected. One embodiment of the present invention relates to elimination of a key competing enzyme that diverts carbon flow from L-ascorbic acid synthesis. If such enzyme is absolutely required for growth on glucose, then mutants lacking the enzyme (and, therefore, having increased carbon flow to L-ascorbic acid) would have been nonviable and not have been detected during prior screening efforts. One such enzyme is phosphofructokinase (PFK) (See FIG. 2A). PFK is required for growth on glucose, and is the major step drawing carbon away from L-ascorbic acid biosynthesis (FIG. 2A). Elimination of PFK would render the cells nonviable on glucose-based media. Selection of a conditional mutant where PFK was inactivated by temperature shift, for example, may allow development of a L-ascorbic acid process where cell growth is achieved under permissive fermentation conditions, and L-ascorbic acid production (from glucose) is initiated by a shift to non-permissive condition. In this example, the temperature shift would eliminate carbon flow from glucose to glycolysis via PFK, thereby shunting carbon into the L-ascorbic acid branch of metabolism. This approach has application not only in natural L-ascorbic acid producing organisms, but also in L-ascorbic acid recombinant systems (genetically engineered plant or microorganisms) as discussed herein.

Knowing the identity and mechanism of the rate-limiting pathway enzymes in the L-ascorbic acid production pathway allows for design of specific inhibitors of the enzymes that are also growth inhibitory. Selection of mutants resistant to the inhibitors allows for the isolation of strains that contain L-ascorbic acid-pathway enzymes with more favorable kinetic properties. Therefore, one embodiment of the present invention is to identify inhibitors of the enzymes that are also growth inhibitory. These inhibitors are then used to select genetic mutants that overcome this inhibition and produce L-ascorbic acid at high levels. In this embodiment, the resultant plant or microorganism is a non-recombinant strain which can then be further modified by recombinant technology, if desired. In recombinant L-ascorbic acid producing strains, random mutagenesis and screening can be used as a final step to increase L-ascorbic acid production.

In yet another embodiment genetic modifications are made to an L-ascorbic acid producing organism directly. This allows one to build upon a base of data acquired during prior classical strain improvement efforts, and perhaps more importantly, allows one to take advantage of undefined beneficial mutations that occurred during classical strain improvement. Furthermore, fewer problems are encountered when expressing native, rather than heterologous, genes. The most advanced system for development of genetic systems for microalgae has been developed for Chlamydomonas reinhardtii. Preferably, development of such a genetically modified production organism would include: isolation of mutant(s) with a specific nutritional requirement for use with a cloned selectable marker gene (similar to the ura3 mutants used in yeast and fungal systems); a cloned selectable marker such as URA3 or alternatively, identification and cloning of a gene that specifies resistance to a toxic compound (this would be analogous to the use of antibiotic resistance genes in bacterial systems, and, as is the case in yeast and other fungi, a means of inserting/removing the marker gene repeatedly would be required, unless several different selectable markers were developed); a transformation system for introducing DNA into the production organism and achieving stable transformation and expression; and, a promoter system (preferably several) for high-level expression of cloned genes in the organism.

Another embodiment of the present invention, discussed in detail below, is to place key genes or allelic variants and homologues thereof from L-ascorbic acid producing organisms (i.e., higher plants and microalgae) into a plant or microorganism that is more amenable to molecular genetic manipulation, including endogenous L-ascorbic acid producing microorganisms and suitable plants. For example, it is possible to identify a suitable non-pathogenic organism based on the requirement of growth (on glucose) at low pH (i.e., acid-tolerant organisms, discussed in detail below).

One suitable candidate for recombinant production in any suitable host organism is the gene (nucleic acid molecule) encoding GDP-D-mannose:GDP-L-galactose epimerase and homologues of the GDP-D-mannose:GDP-L-galactose epimerase, as well as any other epimerase that has structural homology at the primary (i.e., sequence) or tertiary (i.e., three dimensional) level, to a GDP-4-keto-6-deoxy-D-mannose epimerase/reductase, or to a UDP-galactose 4-epimerase. Many microorganisms produce GDP-D-mannose as a precursor to exopolysaccharide and glycoprotein production, even though such organisms may not make L-ascorbic acid. This aspect of the present invention is discussed in detail below.

Referring to FIGS. 1A-1C, at least some of the enzymes from glucose-6-phosphate to GDP-D-mannose are present in many organisms. In fact, the entire sequence is present in bacteria such as Azotobacter vinelandii and Pseudomonas aeruginosa, and make up the early steps in the biosynthesis of the exopolysaccharide alginate. In this regard, it is possible that the only thing preventing these organisms from producing L-ascorbic acid could be the lack of GDP-D-mannose:GDP-L-galactose epimerase. The presence of PMI, PMM and GMP (see FIG. 1A) in so many organisms is important for two reasons. First, these organisms themselves could serve as alternate hosts for L-ascorbic acid production, by building on the existing early pathway enzymes and adding the required cloned genes (the epimerase and possibly others). Second, the genes encoding PMI, PMM and GMP can be cloned into a new organism where, together with the cloned epimerase, they would encode the overall pathway from glucose-6-phosphate to GDP-L-galactose.

In order to screen genomic DNA or cDNA libraries from different organisms and to isolate nucleic acid molecules encoding these enzymes such as the GDP-D-mannose:GDP-L-galactose epimerase, one can use any of a variety of standard molecular and biochemical techniques. For example, the GDP-D-mannose:GDP-L-galactose epimerase can be purified from an organism such as Prototheca, the N-terminal amino acid sequence can be determined (including, if necessary, the sequence of internal peptide fragments), and this information can be used to design degenerate primers for amplifying a gene fragment from the organism's DNA. This fragment would then be used to probe the library, and subsequently fragments that hybridize to the probe would be cloned in that organism or another suitable production organism. There is ample precedent for plant enzymes being expressed in an active form in bacteria, such as E. coli. Alternatively, yeast are also a suitable candidate for developing a heterologous system for L-ascorbic acid production.

It is to be understood that the present invention discloses a method comprising the use of a microorganism with an ability to produce commercially useful amounts of L-ascorbic acid in a fermentation process (i.e., preferably an enhanced ability to produce L-ascorbic acid compared to a wild-type microorganism cultured under the same conditions). This method is achieved by the genetic modification of one or more genes encoding a protein involved in an L-ascorbic acid pathway which results in the production (expression) of a protein having an altered (e.g., increased or decreased) function as compared to the corresponding wild-type protein. Preferably, such genetic modification is achieved by recombinant technology. It will be appreciated by those of skill in the art that production of genetically modified plants or microorganisms having a particular altered function as described elsewhere herein (e.g., an enhanced ability to produce GDP-D-mannose:GDP-L-galactose epimerase), such as by transformation of the plant or microorganism with a nucleic acid molecule which encodes a particular enzyme, can produce many organisms meeting the given functional requirement, albeit by virtue of a variety of different genetic modifications. For example, different random nucleotide deletions and/or substitutions in a given nucleic acid sequence may all give rise to the same phenotypic result (e.g., decreased enzymatic activity of the protein encoded by the sequence). The present invention contemplates any such genetic modification which results in the production of a plant or microorganism having the characteristics set forth herein.

A microorganism to be used in the fermentation method of the present invention is preferably a bacterium, a fungus, or a microalga which has been genetically modified according to the disclosure above. More preferably, a microorganism useful in the present invention is a microalga which is capable of producing L-ascorbic acid, although the present invention includes microorganisms which are genetically engineered to produce L-ascorbic acid using the knowledge of the key components of the pathway and the guidance provided herein. Even more preferably, a microorganism useful in the present invention is an acid-tolerant microorganism, such as microalgae of the genera Prototheca and Chlorella. Acid-tolerant yeast and bacteria are also known in the art. Acid-tolerant microorganisms are discussed in detail below. Particularly preferred microalgae include microalgae of the genera, Prototheca and Chlorella, with Prototheca being most preferred. All known species of Prototheca produce L-ascorbic acid. Production of ascorbic acid by microalgae of the genera Prototheca and Chlorella is described in detail in U.S. Pat. No. 5,792,631, issued Aug. 11, 1998, and in U.S. Pat. No. 5,900,370, issued May 4, 1999, both of which are incorporated herein by reference in their entirety. Preferred bacteria for use in the present invention include, but are not limited to, Azotobacter, Pseudomonas, and Escherichia, although acid-tolerant bacteria are more preferred. Preferred fungi for use in the present invention include yeast, and more preferably, yeast of the genus, Saccharomyces. A microorganism for use in the fermentation method of the present invention can also be referred to as a production organism. According to the present invention, microalgae can be referred to herein either as microorganisms or as plants.

A preferred plant to genetically modify according to the present invention is preferably a plant suitable for consumption by animals, including humans. More preferably, such a plant is a plant that naturally produces L-ascorbic acid, although other plants can be genetically modified to produce L-ascorbic acid using the guidance provided herein.

The L-ascorbic acid production pathways of the microalgae Prototheca and Chlorella pyrenoidosa will be addressed as specific embodiments of the present invention are described below. It will be appreciated that other plants and, in particular, other microorganisms, have similar L-ascorbic acid pathways and genes and proteins having similar structure and function within such pathways. It will also be appreciated that plants and microorganisms which do not naturally produce L-ascorbic acid can be modified according to the present invention to produce L-ascorbic acid. As such, the principles discussed below with regard to Prototheca and Chlorella pyrenoidosa are applicable to other plants and microorganisms, including genetically modified plants and microorganisms.

In one embodiment of the present invention, the action of an enzyme in the L-ascorbic acid production pathway is increased by amplification of the expression (i.e., overexpression) of an enzyme in the pathway, and particularly, the GDP-D-mannose:GDP-L-galactose epimerase, homologues of the epimerase, and/or enzymes downstream of the epimerase. Overexpression of an enzyme can be accomplished, for example, by introduction of a recombinant nucleic acid molecule encoding the enzyme. It is preferred that the gene encoding an enzyme in the L-ascorbic acid production pathway be cloned under control of an artificial promoter. The promoter can be any suitable promoter that will provide a level of enzyme expression required to maintain a sufficient level of L-ascorbic acid in the production organism. Preferred promoters are constitutive (rather than inducible) promoters, since the need for addition of expensive inducers is therefore obviated. The gene dosage (copy number) of a recombinant nucleic acid molecule according to the present invention can be varied according to the requirements for maximum product formation. In one embodiment, the recombinant nucleic acid molecule encoding a gene in the L-ascorbic acid production pathway is integrated into the chromosomes of the microorganism.

It is another embodiment of the present invention to provide a microorganism having one or more enzymes in the L-ascorbic acid production pathway with improved affinity for its substrates. An enzyme with improved affinity for its substrates can be produced by any suitable method of genetic modification or protein engineering. For example, computer-based protein engineering can be used to design an epimerase protein with greater stability and better affinity for its substrate. See for example, Maulik et al., 1997, Molecular Biotechnology: Therapeutic Applications and Strategies, Wiley-Liss, Inc., which is incorporated herein by reference in its entirety.

Recombinant nucleic acid molecules encoding proteins in the L-ascorbic acid production pathway can be modified to enhance or reduce the function (i.e., activity) of the protein, as desired to increase L-ascorbic acid production, by any suitable method of genetic modification. For example, a recombinant nucleic acid molecule encoding an enzyme can be modified by any method for inserting, deleting, and/or substituting nucleotides, such as by error-prone PCR. In this method, the gene is amplified under conditions that lead to a high frequency of misincorporation errors by the DNA polymerase used for the amplification. As a result, a high frequency of mutations are obtained in the PCR products. The resulting gene mutants can then be screened for enhanced substrate affinity, enhanced enzymatic activity, or reduced/increased inhibitory ability by testing the mutant genes for the ability to confer increased L-ascorbic acid production onto a test microorganism, as compared to a microorganism carrying the non-mutated recombinant nucleic acid molecule.

Another embodiment of the present invention includes a microorganism in which competitive side reactions are blocked, including all reactions for which GDP-D-mannose is a substrate other than the production of L-ascorbic acid. In a preferred embodiment, a microorganism having complete or partial inactivation (decrease in the action of) of genes encoding enzymes which compete with the GDP-D-mannose:GDP-L-galactose epimerase for the GDP-D-mannose substrate is provided. Such enzymes include GDP-D-mannase and/or GDP-D-mannose-dehydrogenase. As used herein, inactivation of a gene can refer to any modification of a gene which results in a decrease in the activity (i.e., expression or function) of such a gene, including attenuation of activity or complete deletion of activity.

As discussed above, a particularly preferred aspect of the method to produce L-ascorbic acid by fermentation of a genetically modified microorganism of the present invention includes the step of culturing in a fermentation medium a microorganism having a genetic modification to increase the action of an epimerase that catalyzes conversion of GDP-D-mannose to GDP-L-galactose. According to the present invention, such an epimerase can include the endogenous GDP-D-mannose:GDP-L-galactose epimerase from the L-ascorbic acid pathway, described above, as well as any other epimerase that has structural homology at the primary (i.e., sequence) or tertiary (i.e., three dimensional) level, to a GDP-4-keto-6-deoxy-D-mannose epimerase/reductase, or to a UDP-galactose 4-epimerase. Such structural homology is discussed in detail below. Preferably, such an epimerase is capable of catalyzing the conversion of GDP-D-mannose to GDP-L-galactose. In one embodiment, the genetic modification includes transformation of the microorganism with a recombinant nucleic acid molecule that expresses such an epimerase.

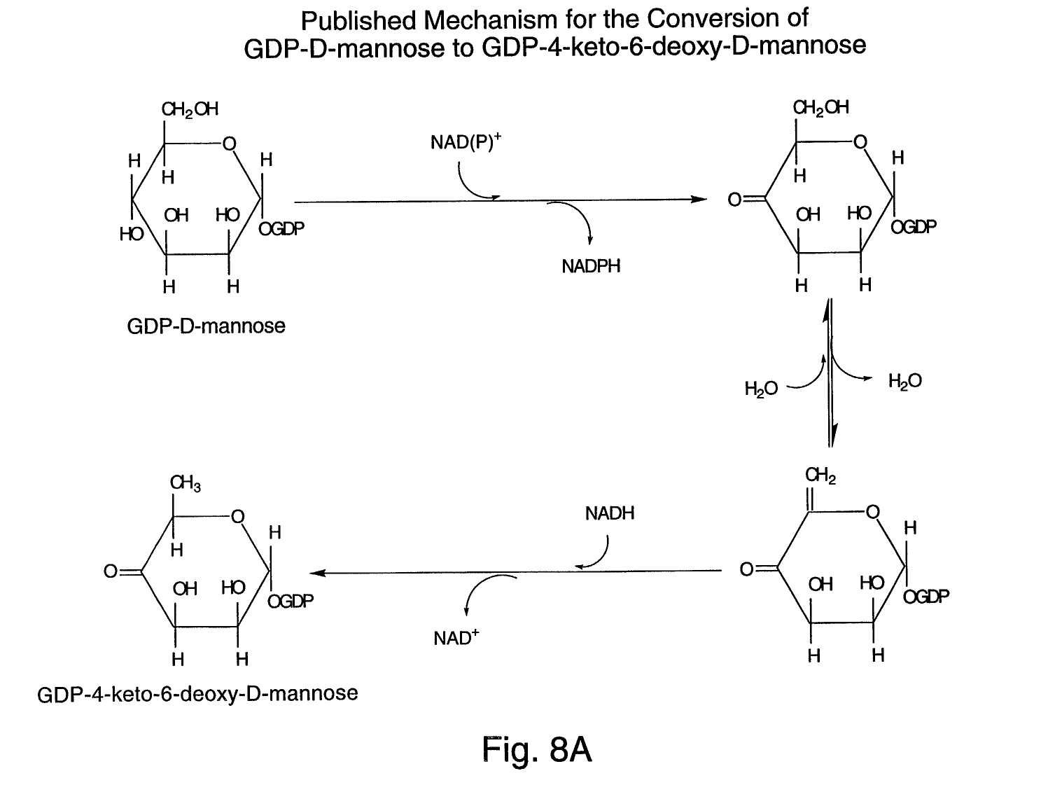

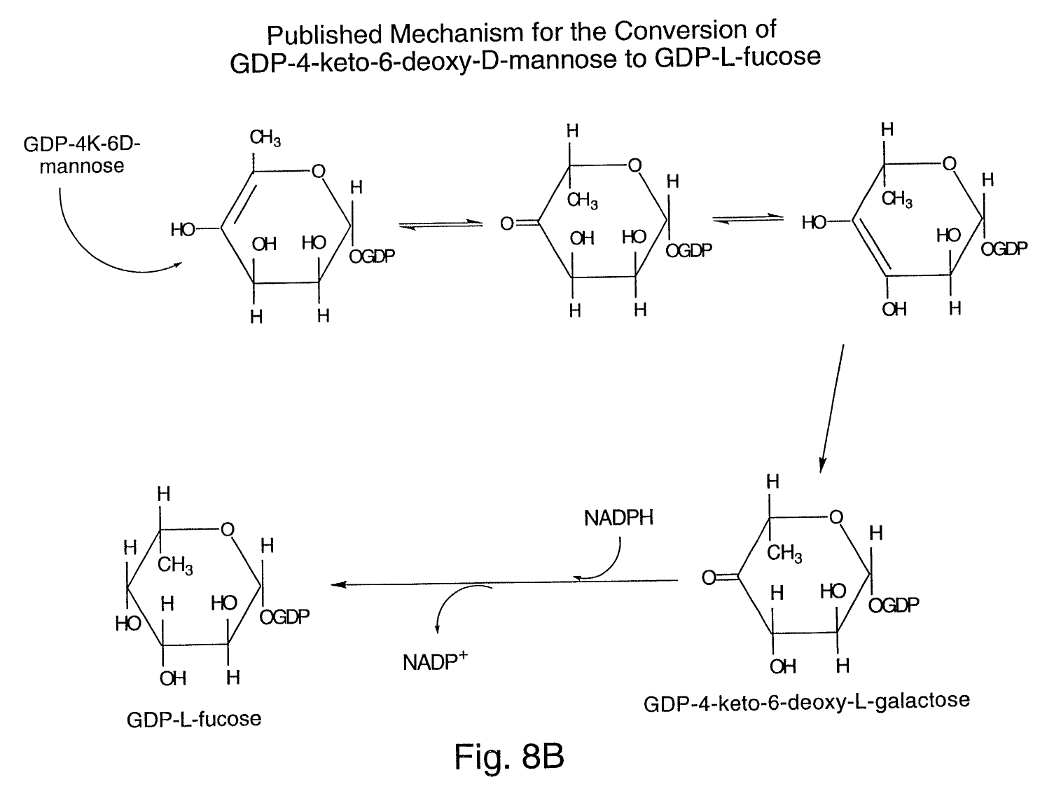

Therefore, the epimerase encompassed in the method and organisms of the present invention includes the endogenous epimerase which operates in the naturally occurring ascorbic acid biosynthetic pathway (referred to herein as GDP-D-mannose:GDP-L-galactose epimerase), GDP-4-keto-6-deoxy-D-mannose epimerase/reductases, and any other epimerase which is capable of catalyzing the conversion of GDP-D mannose to GDP-L-galactose and which is structurally homologous to a GDP-4-keto-6-deoxy-D-mannose epimerase/reductase or a UDP-galactose 4-epimerase. An epimerase that catalyzes conversion of GDP-D-mannose to GDP-L-galactose according the present invention can be identified by biochemical and functional characteristics as well as structural characteristics. For example, an epimerase according to the present invention is capable of acting on GDP-D-mannose as a substrate, and more particularly, such an epimerase is capable of catalyzing the conversion of GDP-D-mannose to GDP-L-galactose. It is to be understood that such capabilities need not necessarily be the normal or natural function of the epimerase as it acts in its endogenous (i.e., natural) environment. For example, GDP-4-keto-6-deoxy-D-mannose epimerase/reductase in its natural environment under normal conditions, catalyzes the conversion of GDP-D-mannose to GDP-L-fucose and does not act directly on GDP-D-mannose (See FIG. 8A, B), however, such an epimerase is encompassed by the present invention for use in catalyzing the conversion of GDP-D-mannose to GDP-L-galactose for production of ascorbic acid, to the extent that it is capable of, or can be modified to be capable of, catalyzing the conversion of GDP-D-mannose to GDP-L-galactose. Therefore, the present invention includes epimerases which have the desired enzyme activity for use in production of ascorbic acid, are capable of having such desired enzyme activity, and/or are capable of being modified or induced to have such desired enzyme activity.

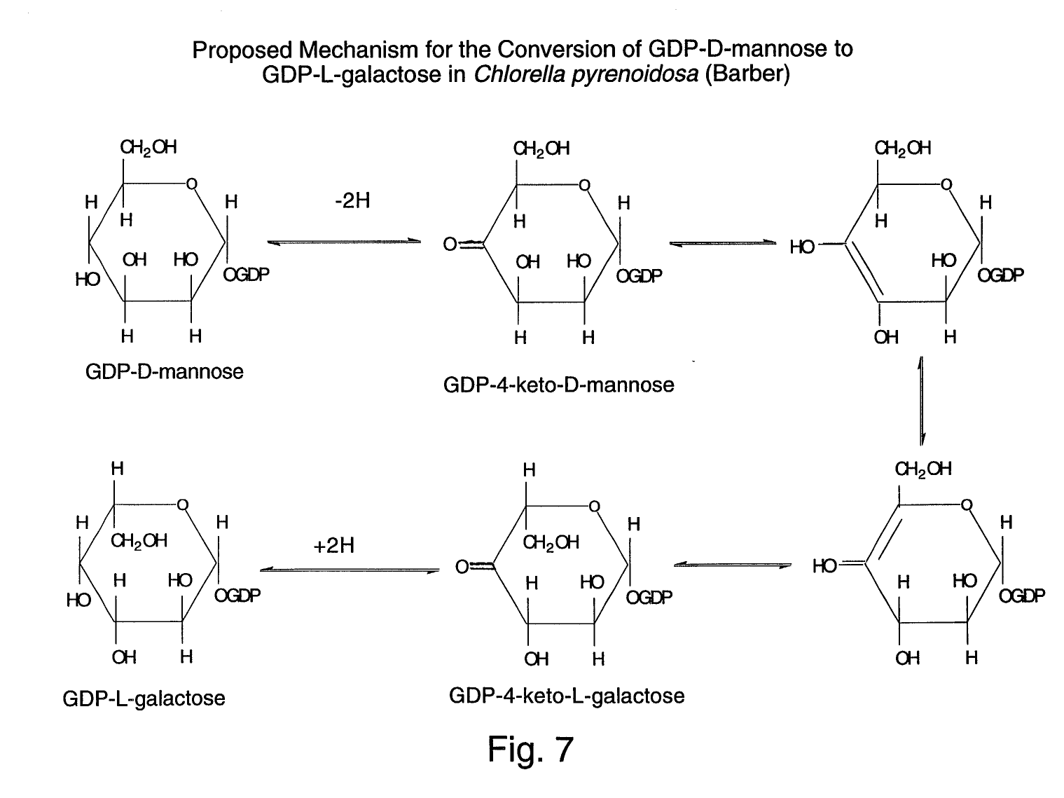

In one embodiment, an epimerase according to the present invention includes an epimerase that catalyzes the reaction depicted in FIG. 7. In another embodiment, an epimerase according to the present invention includes an epimerase that catalyzes the first of the reactions depicted in FIG. 8B. In one embodiment, an epimerase according to the present invention binds to NADPH. In another embodiment, an epimerase according to the present invention is NADPH-dependent for enzyme activity.

As discussed above, the present inventors have discovered that a key enzyme in L-ascorbic acid biosynthesis in plants and microorganisms is GDP-D-mannose:GDP-L-galactose epimerase (refer to FIGS. 1A-1C). One embodiment of the invention described herein is directed to the manipulation of this enzyme and structural homologues of this enzyme to increase L-ascorbic acid production in genetically engineered plants and/or microorganisms. More particularly, the GDP-D-mannose:GDP-L-galactose epimerase of the L-ascorbic acid pathway and GDP-4-keto-6-deoxy-D-mannose epimerase/reductases are believed to be structurally homologous at both the sequence and tertiary structure level; a GDP-4-keto-6-deoxy-D-mannose epimerase/reductase is believed to be capable of functioning in the L-ascorbic acid biosynthetic pathway; and a GDP-4-keto-6-deoxy-D-mannose epimerase/reductase or homologue thereof may be superior to a GDP-D-mannose-GDP-L-galactose epimerase for increasing L-ascorbic acid production in genetically engineered plants and/or microorganisms. Furthermore, the present inventors disclose the use of a nucleotide sequence encoding all or part of a GDP-4-keto-6-deoxy-D-mannose epimerase/reductase as a probe to identify the gene encoding GDP-D-mannose:GDP-L-galactose epimerase. Similarly, the present inventors disclose the use of a nucleotide sequence of the gene encoding GDP-4-keto-6-deoxy-D-mannose epimerase/reductase to design oligonucleotide primers for use in a PCR-based strategy for identifying and cloning a gene encoding GDP-D-mannose:GDP-L-galactose epimerase.

Without being bound by theory, the present inventors believe that the following evidence supports the novel concept that the GDP-D-mannose:GDP-L-galactose epimerase and GDP-4-keto-6-deoxy-D-mannose epimerase/reductases have significant structural homology at the level of sequence and/or tertiary structure, and that the GDP-4-keto-6-deoxy-D-mannose epimerase/reductases and/or homologues thereof would be useful for production of ascorbic acid and/or for isolating the endogenous GDP-D-mannose:GDP-L-galactose epimerase.

Although prior to the present invention, it was not known that the GDP-D-mannose:GDP-L-galactose epimerase enzyme (also known as GDP-D-mannose-3,5-epimerase) plays a critical role in L-ascorbic acid biosynthesis, this enzyme was previously described to catalyze the overall reversible reaction between GDP-D-mannose and GDP-L-galactose (Barber, 1971, Arch. Biochem. Biophys. 147:619-623; Barber, 1975, Arch. Biochem. Biophys. 167:718-722; Barber, 1979, J. Biol. Chem. 254:7600-7603; Hebda, et al., 1979, Arch. Biochem. Biophys. 194:496-502; Barber and Hebda, 1982, Meth. Enzymol., 83:522-525). Despite these studies, GDP-D-mannose:GDP-L-galactose epimerase has never been well characterized nor has the gene encoding this enzyme been cloned and sequenced. Since the original work by Barber, GDP-D-mannose:GDP-L-galactose epimerase activity has been detected in the colorless microalga Prototheca moriformis by the assignee of the present application, and in Arabidopsis thaliana and pea embryonic axes (Wheeler, et al., 1998, ibid.).

Barber (1979, J. Biol. Chem. 254:7600-7603) proposed a mechanism for GDP-D-mannose:GDP-L-galactose epimerase partially purified from the green microalga Chlorella pyrenoidosa. The overall conversion of GDP-D-mannose to GDP-L-galactose was proposed to proceed by oxidation of the hexosyl moiety at C-4 to a keto intermediate, ene-diol formation, and inversion of the configurations at C-3 and C-5 upon rehydration of the double bonds and stereospecific reduction of the keto group. The proposed mechanism is depicted in FIG. 7.

Based on Barber's work, Feingold and Avigad (1980, In The Biochemistry of Plants, Vol. 3: Carbohydrates; Structure and Function, P. K. Stompf and E. E. Conn, eds., Academic Press, NY) elaborated further on the proposed mechanism for GDP-D-mannose:GDP-L-galactose epimerase. This mechanism is based on the assumption that the epimerase contains tightly bound NAD⁺, and transfer of a hydride ion from C-4 of the substrate (GDP-D-mannose) to enzyme-associated NAD⁺ converts the enzyme to the reduced (NADH)form, generating enzyme-bound GDP-4-keto-D-mannose. The latter would then undergo epimerization by an ene-diol mechanism. The final product (GDP-L-galactose) would be released from the enzyme after stereospecific transfer of the hydride ion originally removed from C-4, simultaneously regenerating the oxidized form of the enzyme.

L-fucose (6-deoxy-L-galactose) is a component of bacterial lipopolysaccharides, mammalian and plant glycoproteins and polysaccharides of plant cell walls. L-fucose is synthesized de novo from GDP-D-mannose by the sequential action of GDP-D-mannose-4,6-dehydratase (an NAD(P)-dependent enzyme), and a bifunctional GDP-4-keto-6-deoxy-D-mannose epimerase/reductase (NADPH-dependent), also referred to in scientific literature as GDP-fucose synthetase (Rizzi, et al., 1998, Structure 6:1453-1465; Somers, et al., 1998, Structure 6:1601-1612). This pathway for L-fucose biosynthesis appears to be ubiquitous (Rizzi, et al., 1998, Structure 6:1453-1465). The mechanisms for GDP-D-mannose-4,6-dehydratase and GDP-4-keto-6-deoxy-D-mannose epimerase/reductase are shown in FIG. 8A, B (Chang, et al., 1988, J. Biol. Chem. 263:1693-1697; Barber, 1980, Plant Physiol. 66:326-329).

Comparison of FIGS. 7 and 8A, B reveals that Barber's proposed mechanism for GDP-D-mannose:GDP-L-galactose epimerase is analogous to the reaction mechanism for GDP-4-keto-6-deoxy-D-mannose epimerase/reductase. The same mechanism has also been demonstrated for the epimerization reaction that occurs in the biosynthesis of two TDP-6-deoxy hexoses, TDP-L-rhamnose and TDP-6-deoxy-L-talose, from TDP-D-glucose (Liu and Thorson, 1994, Ann. Rev. Microbiol. 48:223-256). In the latter cases, however, the final reduction at C-4 is catalyzed by NADPH-dependent reductases that are separate from the epimerase enzyme. These reductases have opposite stereospecificity, providing either TDP-L-rhamnose or TDP-6-deoxy-L-talose (Liu and Thorson, 1994, Ann. Rev. Microbiol. 48:223-256).

In all of the mechanisms described above, NAD(P)H is required for the final reduction at C-4 (refer to FIG. 8B). In the work of Hebda, et al. (1979, Arch. Biochem. Biophys. 194:496-502), it was reported that GDP-D-mannose:GDP-L-galactose epimerase from C. pyrenoidosa did not require NAD, NADP or NADH for activity. Strangely, NADPH was not tested. Based on the analogous mechanisms shown in FIGS. 7 and 8A, B, the present inventors believe that it is likely that GDP-D-mannose:GDP-L-galactose epimerase from C. pyrenoidosa requires NADPH for the final reduction step. Why activity was detected in vitro without NADPH addition is not known, but tight binding of NADPH to the enzyme could explain this observation. On the other hand, if the proposed mechanism of Feingold and Avigad (1980, in The Biochemistry of Plants, Vol. 3, p. 101-170: Carbohydrates; Structure and Function, P. K. Stompf and E. E. Conn, ed., Academic Press, NY) is correct, the reduced enzyme-bound cofactor generated in the first oxidation step of the epimerase reaction would serve as the source of electrons for the final reduction of the keto group at C-4 back to the alcohol. Thus no addition of exogenous reduced cofactor would be required for activity in vitro.

Recently, a human gene encoding the bifunctional GDP-4-keto-6-deoxy-D-mannose epimerase/reductase was cloned and sequenced (Tonetti, et al., 1996, J. Biol. Chem. 271-27274-27279). This amino acid sequence of the human GDP-4-keto-6-deoxy-D-mannose epimerase/reductase shows significant homology (29% identity) to the E. coli GDP-4-keto-6-deoxy-D-mannose epimerase/reductase (Tonetti, et al., 1998, Acta Cryst. D54:684-686; Somers, et al., 1998, Structure 6:1601-1612, both of which are incorporated herein by reference in their entireties). Tonetti et al. and Somers et al. additionally disclosed the tertiary (three dimensional) structure of the E. coli GDP-4-keto-6-deoxy-D-mannose epimerase/reductase (also known as GDP-fucose synthetase), and noted significant structural homology with another epimerase, UDP-galactose 4-epimerase (GalE). These epimerases also share significant homology at the sequence level. Since no gene encoding a GDP-D-mannose:GDP-L-galactose epimerase has been cloned and sequenced, homology with genes encoding GDP-4-keto-6-deoxy-D-mannose epimerase/reductases or with genes encoding a UDP-galactose 4-epimerase has not been demonstrated. However, based on the similarity of the reaction products for GDP-D-mannose:GDP-L-galactose epimerase and GDP-4-keto-6-deoxy-D-mannose epimerase/reductase (i.e., GDP-L-galactose and GDP-6-deoxy-L-galactose [i.e., GDP-L-fucose], respectively) and the common catalytic mechanisms (FIGS. 7 and 8A, B) the present inventors believe that the genes encoding the enzymes will have a high degree of sequence homology, as well as tertiary structural homology.

Significant structural homology between GDP-D-mannose:GDP-L-galactose epimerase and GDP-4-keto-6-deoxy-D-mannose epimerase/reductases may allow a GDP-4-keto-6-deoxy-D-mannose epimerase/reductase, or a homologue thereof, to function in the L-ascorbic acid biosynthetic pathway, and a GDP-4-keto-6-deoxy-D-mannose epimerase/reductase could potentially be even better than a GDP-D-mannose-GDP-L-galactose epimerase for increasing L-ascorbic acid production in genetically engineered plants and/or microorganisms. Furthermore, a nucleotide sequence encoding all or part of a GDP-4-keto-6-deoxy-D-mannose epimerase/reductase can be used as a probe to identify the gene encoding GDP-D-mannose:GDP-L-galactose epimerase. Likewise, the nucleotide sequence of the gene encoding GDP-4-keto-6-deoxy-D-mannose epimerase/reductase can be used to design oligonucleotide primers for use in a PCR-based strategy for identifying and cloning a gene encoding GDP-D-mannose:GDP-L-galactose epimerase.

The ability to substitute GDP-4-keto-6-D-mannose epimerase/reductase for GDP-D-mannose:GDP-L-galactose epimerase to enhance L-ascorbic acid biosynthesis in plants or microorganisms depends on the ability of GDP-4-keto-6-deoxy-D-mannose epimerase/reductase to act directly on GDP-D-mannose to form GDP-L-galactose. Evidence supporting this possibility already exists. Arabidopsis thaliana murl mutants are defective in GDP-D-mannose-4,6-dehydratase activity (Bonin, et al., 1997, Proc. Natl. Acad. Sci. 94:2085-2090). These mutants are thus blocked in GDP-L-fucose biosynthesis, and consequently have less than 2% of the normal amounts of L-fucose in the primary cell walls of aerial portions of the plant (Zablackis, et al., 1996, Science 272:1808-1810). The murl mutants are more brittle than wild-type plants, are slightly dwarfed and have an apparently normal life cycle (Zablackis, et al., 272:1808-1810). When murl mutants are grown in the presence of exogenous L-fucose, the L-fucose content in the plant is restored to the wild-type state (Bonin, et al., 1997, Proc. Natl. Acad. Sci. 94:2085-2090). It was discovered (Zablackis, et al., 1996, Science 272:1808-1810) that murl mutants contain, in the hemicellulose xyloglucan component of the primary cell wall, L-galactose in place of the normal L-fucose. L-galactose is not normally found in the xyloglucan component, but in murl mutants L-galactose partly replaces the terminal L-fucosyl residue. Bonin, et al. (1997, Proc. Natl. Acad. Sci. 94:2085-2090) hypothesized that in the absence of a functional GDP-D-mannose-4,6-dehydratase in the murl mutants, the GDP-4-keto-6-deoxy-D-mannose epimerase/reductase normally involved in L-fucose synthesis may be able to use GDP-D-mannose directly, forming GDP-L-galactose. Another possibility, however, is that the enzymes involved in L-ascorbic acid biosynthesis in A. thaliana are responsible for forming GDP-L-galactose in the murl mutant. If this were true, it would suggest that in the wild-type plant, some mechanism exists that prevents GDP-L-galactose formed in the L-ascorbic acid pathway from entering cell wall biosynthesis and substituting for (competing with) GDP-L-fucose for incorporation into the xyloglucan component (since L-galactose is not present in the primary cell wall of the wild-type plant).

Because of the similar reaction mechanisms of GDP-D-mannose:GDP-L-galactose epimerase and GDP-4-keto-6-deoxy-D-mannose epimerase/reductase, and because of the evidence that GDP-4-keto-6-deoxy-D-mannose epimerase/reductase can act directly on GDP-D-mannose to form GDP-L-galactose, the present inventors believe that genes encoding all epimerases and epimerase/reductases that act on GDP-D-mannose have high homology. As such, one aspect of the present invention relates to the use of any epimerase (and nucleic acid sequences encoding such epimerase) having significant homology (at the primary, secondary and/or tertiary structure level) to a GDP-4-keto-6-deoxy-D-mannose epimerase/reductase or to a UDP-galactose 4-epimerase for the purpose of improving the biosynthetic production of L-ascorbic acid.

Therefore, as described above, one embodiment of the present invention relates to a method for producing ascorbic acid or esters thereof in a microorganism, which includes culturing a microorganism having a genetic modification to increase the action of an epimerase that catalyzes conversion of GDP-D-mannose to GDP-L-galactose. Also included in the present invention are genetically modified microorganisms and plants in which the genetic modification increases the action of an epimerase that catalyzes conversion of GDP-D-mannose to GDP-L-galactose.

According to the present invention, an increase in the action of the GDP-D-mannose:GDP-L-galactose epimerase in the L-ascorbic acid production pathway can be achieved by genetic modifications which include, but are not limited to overexpression of the GDP-D-mannose:GDP-L-galactose epimerase gene, a homologue of such gene, or of any recombinant nucleic acid sequence encoding an epimerase that is homologous in primary (nucleic acid or amino acid sequence) or tertiary (three dimensional protein) structure to a GDP-4-keto-6-deoxy-D-mannose epimerase/reductase or a UDP-galactose 4-epimerase, such as by overexpression of a recombinant nucleic acid molecule encoding the epimerase gene or a homologue thereof, and/or by mutation of the endogenous or recombinant gene to enhance expression of the gene.

According to the present invention, an epimerase that has a tertiary structure that is homologous to the tertiary structure of a GDP-4-keto-6-deoxy-D-mannose epimerase/reductase is an epimerase that has a tertiary structure that substantially conforms to the tertiary structure of a GDP-4-keto-6-deoxy-D-mannose epimerase/reductase represented by the atomic coordinates having Brookhaven Protein Data Bank Accession Code 1bws (Table 12). In another embodiment, an epimerase that has a tertiary structure that is homologous to the tertiary structure of a GDP-4-keto-6-deoxy-D-mannose epimerase/reductase is an epimerase that has a tertiary structure that substantially conforms to the tertiary structure of a GDP-4-keto-6-deoxy-D-mannose epimerase/reductase represented by the atomic coordinates having Brookhaven Protein Data Bank Accession Code 1GFS. As used herein, a “tertiary structure” or “three dimensional structure” of a protein, such terms being interchangeable, refers to the components and the manner of arrangement of the components in three dimensional space to constitute the protein. The use of the term “substantially conforms” refers to at least a portion of a tertiary structure of an epimerase which is sufficiently spatially similar to at least a portion of a specified three dimensional configuration of a particular set of atomic coordinates (e.g., those represented by Brookhaven Protein Data Bank Accession Code 1bws) to allow the tertiary structure of at least said portion of the epimerase to be modeled or calculated (i.e., by molecular replacement) using the particular set of atomic coordinates as a basis for estimating the atomic coordinates defining the three dimensional configuration of the epimerase.

More particularly, a tertiary structure that substantially conforms to a given set of atomic coordinates is a structure having an average root-mean-square deviation (RMSD) of less than about 2.5 Å, and more preferably, less than about 2 Å, and, in increasing preference, less than about 1.5 Å, less than about 1 Å, less than about 0.5 Å, and most preferably, less than about 0.3 Å, over at least about 25% of the Cα positions as compared to the tertiary structure of a GDP-4-keto-6-deoxy-D-mannose epimerase/reductase represented by the atomic coordinates having Brookhaven Protein Data Bank Accession Code 1bws. In other embodiments, a structure that substantially conforms to a given set of atomic coordinates is a structure wherein such structure has the recited average root-mean-square deviation (RMSD) value over at least about 50% of the Cα positions as compared to the tertiary structure of a GDP-4-keto-6-deoxy-D-mannose epimerase/reductase represented by the atomic coordinates having Brookhaven Protein Data Bank Accession Code 1bws, and in another embodiment, such structure has the recited average root-mean-square deviation (RMSD) value over at least about 75% of the Cα positions as compared to the tertiary structure of a GDP-4-keto-6-deoxy-D-mannose epimerase/reductase represented by the atomic coordinates having Brookhaven Protein Data Bank Accession Code 1bws, and in another embodiment, such structure has the recited average root-mean-square deviation (RMSD) value over about 100% of the Cα positions as compared to the tertiary structure of a GDP-4-keto-6-deoxy-D-mannose epimerase/reductase represented by the atomic coordinates having Brookhaven Protein Data Bank Accession Code 1bws. Methods to calculate RMSD values are well known in the art. Various software programs for determining the tertiary structural homology between one or more proteins are known in the art and are publicly available, such as QUANTA (Molecular Simulations Inc.).

A preferred epimerase that catalyzes conversion of GDP-D-mannose to GDP-L-galactose according to the method and genetically modified organisms of the present invention includes an epimerase that comprises a substrate binding site having a tertiary structure that substantially conforms to the tertiary structure of the substrate binding site of a GDP-4-keto-6-deoxy-D-mannose epimerase/reductase represented by the atomic coordinates having Brookhaven Protein Data Bank Accession Code 1bws. Preferably, the tertiary structure of the substrate binding site of the epimerase has an average root-mean-square deviation (RMSD) of less than about 2.5 Å, and more preferably, less than about 2 Å, and, in increasing preference, less than about 1.5 Å, less than about 1 Å, less than about 0.5 Å, and most preferably, less than about 0.3 Å, over at least about 25% of the Cα positions as compared to the tertiary structure of the substrate binding site of a GDP-4-keto-6-deoxy-D-mannose epimerase/reductase represented by the atomic coordinates having Brookhaven Protein Data Bank Accession Code 1bws. In other embodiments, the tertiary structure of the substrate binding site of the epimerase has the recited average root-mean-square deviation (RMSD) value over at least about 50% of the Cα positions as compared to the tertiary structure of the substrate binding site of a GDP-4-keto-6-deoxy-D-mannose epimerase/reductase represented by the atomic coordinates having Brookhaven Protein Data Bank Accession Code 1bws, and in another embodiment, the tertiary structure of the substrate binding site of the epimerase has the recited average root-mean-square deviation (RMSD) value over at least about 75% of the Cα positions as compared to the tertiary structure of the substrate binding site of a GDP-4-keto-6-deoxy-D-mannose epimerase/reductase represented by the atomic coordinates having Brookhaven Protein Data Bank Accession Code 1bws, and in another embodiment, the tertiary structure of the substrate binding site of the epimerase has the recited average root-mean-square deviation (RMSD) value over about 100% of the Cα positions as compared to the tertiary structure of the substrate binding site of a GDP-4-keto-6-deoxy-D-mannose epimerase/reductase represented by the atomic coordinates having Brookhaven Protein Data Bank Accession Code 1bws. The tertiary structure of the substrate binding site of a GDP-4-keto-6-deoxy-D-mannose epimerase/reductase represented by the atomic coordinates having Brookhaven Protein Data Bank Accession Code 1bws is discussed in detail in Rizzi et al., 1998, ibid. Additionally, the tertiary structure of the substrate binding site of a GDP-4-keto-6-deoxy-D-mannose epimerase/reductase represented by the atomic coordinates having Brookhaven Protein Data Bank Accession Code 1GFS is discussed in detail in Somers et al., 1998, ibid.

Another preferred epimerase according to the present invention includes an epimerase that comprises a catalytic site having a tertiary structure that substantially conforms to the tertiary structure of the catalytic site of a GDP-4-keto-6-deoxy-D-mannose epimerase/reductase represented by the atomic coordinates having Brookhaven Protein Data Bank Accession Code 1bws. Preferably, the tertiary structure of the catalytic site of the epimerase has an average root-mean-square deviation (RMSD) of less than about 2.5 Å, and more preferably, less than about 2 Å, and, in increasing preference, less than about 1.5 Å, less than about 1 Å, less than about 0.5 Å, and most preferably, less than about 0.3 Å, over at least about 25% of the Cα positions as compared to the tertiary structure of the catalytic site of a GDP-4-keto-6-deoxy-D-mannose epimerase/reductase represented by the atomic coordinates having Brookhaven Protein Data Bank Accession Code 1bws. In other embodiments, the tertiary structure of the catalytic site of the epimerase has the recited average root-mean-square deviation (RMSD) value over at least about 50% of the Cα positions as compared to the tertiary structure of the catalytic site of a GDP-4-keto-6-deoxy-D-mannose epimerase/reductase represented by the atomic coordinates having Brookhaven Protein Data Bank Accession Code 1bws, and in another embodiment, the tertiary structure of the catalytic site of the epimerase has the recited average root-mean-square deviation (RMSD) value over at least about 75% of the Cα positions as compared to the tertiary structure of the catalytic site of a GDP-4-keto-6-deoxy-D-mannose epimerase/reductase represented by the atomic coordinates having Brookhaven Protein Data Bank Accession Code 1bws, and in another embodiment, the tertiary structure of the catalytic site of the epimerase has the recited average root-mean-square deviation (RMSD) value over 100% of the Cα positions as compared to the tertiary structure of the catalytic site of a GDP-4-keto-6-deoxy-D-mannose epimerase/reductase represented by the atomic coordinates having Brookhaven Protein Data Bank Accession Code 1bws.

In one embodiment, an epimerase encompassed by the present invention includes an epimerase that has a catalytic site which includes amino acid residues: serine, tyrosine and lysine. In a preferred embodiment, the tertiary structure positions of the amino acid residues serine, tyrosine and lysine substantially conform to the tertiary structure position of residues Ser107, Tyr136 and Lys140, respectively, as represented by atomic coordinates in Brookhaven Protein Data Bank Accession Code 1bws. The tertiary structure of the catalytic site of a GDP-4-keto-6-deoxy-D-mannose epimerase/reductase represented by the atomic coordinates having Brookhaven Protein Data Bank Accession Code 1bws is discussed in detail in Rizzi et al., 1998, ibid. Additionally, the tertiary structure of the catalytic site of a GDP-4-keto-6-deoxy-D-mannose epimerase/reductase represented by the atomic coordinates having Brookhaven Protein Data Bank Accession Code 1GFS is discussed in detail in Somers et al., 1998, ibid.

In an even more preferred embodiment, the above definition of “substantially conforms” can be further defined to include atoms of amino acid side chains. As used herein, the phrase “common amino acid side chains” refers to amino acid side chains that are common to both the structures which substantially conforms to a given set of atomic coordinates and the structure that is actually represented by such atomic coordinates. Preferably, a tertiary structure that substantially conforms to a given set of atomic coordinates is a structure having an average root-mean-square deviation (RMSD) of less than about 2.5 Å, and more preferably, less than about 2 Å, and, in increasing preference, less than about 1.5 Å, less than about 1 Å, less than about 0.5 Å, and most preferably, less than about 0.3 Å over at least about 25% of the common amino acid side chains as compared to the tertiary structure represented by the given set of atomic coordinates. In another embodiment, a structure that substantially conforms to a given set of atomic coordinates is a structure having the recited average root-mean-square deviation (RMSD) value over at least about 50% of the common amino acid side chains as compared to the tertiary structure represented by the given set of atomic coordinates, and in another embodiment, such structure has the recited average root-mean-square deviation (RMSD) value over at least about 75% of the common amino acid side chains as compared to the tertiary structure represented by the given set of atomic coordinates, and in another embodiment, such a structure has the recited average root-mean-square deviation (RMSD) value over 100% of the common amino acid side chains as compared to the tertiary structure represented by the given set of atomic coordinates.

A tertiary structure of an epimerase which substantially conforms to a specified set of atomic coordinates can be modeled by a suitable modeling computer program such as MODELER (A. Sali and T. L. Blundell, J. Mol. Biol., vol. 234:779-815, 1993 as implemented in the Insight II Homology software package (Insight II (97.0), MSI, San Diego)), using information, for example, derived from the following data: (1) the amino acid sequence of the epimerase; (2) the amino acid sequence of the related portion(s) of the protein represented by the specified set of atomic coordinates having a three dimensional configuration; and, (3) the atomic coordinates of the specified three dimensional configuration. Alternatively, a tertiary structure of an epimerase which substantially conforms to a specified set of atomic coordinates can be modeled using data generated from analysis of a crystallized structure of the epimerase. A tertiary structure of an epimerase which substantially conforms to a specified set of atomic coordinates can also be calculated by a method such as molecular replacement. Methods of molecular replacement are generally known by those of skill in the art (generally described in Brunger, Meth. Enzym., vol. 276, pp. 558-580, 1997; Navaza and Saludjian, Meth. Enzym., vol. 276, pp. 581-594, 1997; Tong and Rossmann, Meth. Enzym., vol. 276, pp. 594-611, 1997; and Bentley, Meth. Enzym., vol. 276, pp. 611-619, 1997, each of which are incorporated by this reference herein in their entirety) and are performed in a software program including, for example, XPLOR (Brunger, et al., Science, vol. 235, p. 458, 1987). In addition, a structure can be modeled using techniques generally described by, for example, Sali, Current opinions in Biotechnology, vol. 6, pp. 437-451, 1995, and algorithms can be implemented in program packages such as Homology 95.0 (in the program Insight II, available from Biosym/MSI, San Diego, Calif.). Use of Homology 95.0 requires an alignment of an amino acid sequence of a known structure having a known three dimensional structure with an amino acid sequence of a target structure to be modeled. The alignment can be a pairwise alignment or a multiple sequence alignment including other related sequences (for example, using the method generally described by Rost, Meth. Enzymol., vol. 266, pp. 525-539, 1996) to improve accuracy. Structurally conserved regions can be identified by comparing related structural features, or by examining the degree of sequence homology between the known structure and the target structure. Certain coordinates for the target structure are assigned using known structures from the known structure. Coordinates for other regions of the target structure can be generated from fragments obtained from known structures such as those found in the Protein Data Bank maintained by Brookhaven National Laboratory, Upton, N.Y. Conformation of side chains of the target structure can be assigned with reference to what is sterically allowable and using a library of rotamers and their frequency of occurrence (as generally described in Ponder and Richards, J. Mol. Biol., vol. 193, pp. 775-791, 1987). The resulting model of the target structure, can be refined by molecular mechanics (such as embodied in the program Discover, available from Biosym/MSI) to ensure that the model is chemically and conformationally reasonable.

According to the present invention, an epimerase that has a nucleic acid sequence that is homologous at the primary structure level (i.e., is a homologue of) to a nucleic acid sequence encoding a GDP-4-keto-6-deoxy-D-mannose epimerase/reductase or a UDP-galactose 4-epimerase includes any epimerase encoded by a nucleic acid sequence that is at least about 15%, and preferably at least about 20%, and more preferably at least about 25%, and even more preferably, at least about 30% identical to a nucleic acid sequence encoding a GDP-4-keto-6-deoxy-D-mannose epimerase/reductase or a UDP-galactose 4-epimerase, and preferably to a nucleic acid sequence selected from the group consisting of SEQ ID NO:1, SEQ ID NO:3, SEQ ID NO:5, SEQ ID NO:7 or SEQ ID NO:9. Similarly, an epimerase that has an amino acid sequence that is homologous to an amino acid sequence of a GDP-4-keto-6-deoxy-D-mannose epimerase/reductase or a UDP-galactose 4-epimerase includes any epimerase having an amino acid sequence that is at least about 15%, and preferably at least about 20%, and more preferably at least about 25%, and even more preferably, at least about 30% identical to an amino acid sequence of a GDP-4-keto-6-deoxy-D-mannose epimerase/reductase or a UDP-galactose 4-epimerase, and preferably to an amino acid sequence selected from the group consisting of SEQ ID NO:2, SEQ ID NO:4, SEQ ID NO:6, SEQ ID NO:8 or SEQ ID NO:10.

According to one embodiment of the present invention, homology or percent identity between two or more nucleic acid or amino acid sequences is performed using methods known in the art for aligning and/or calculating percentage identity. To compare the homology/percent identity between two or more sequences as set forth above, for example, a module contained within DNASTAR (DNASTAR, Inc., Madison, Wis.) can be used. In particular, to calculate the percent identity between two nucleic acid or amino acid sequences, the Lipman-Pearson method, provided by the MegAlign module within the DNASTAR program, is preferably used, with the following parameters, also referred to herein as the Lipman-Pearson standard default parameters:

(1) Ktuple=2;

(2) Gap penalty=4;

(3) Gap length penalty=12.

Using the Lipman-Pearson method with these parameters, for example, the percent identity between the amino acid sequence for E. coli GDP-4-keto-6-deoxy-D-mannose epimerase/reductase (SEQ ID NO:4) and human GDP-4-keto-6-deoxy-D-mannose epimerase/reductase (FX) (SEQ ID NO:6) is 27.7%, which is comparable to the 27% identity described for these enzymes in Tonetti et al., 1998, Acta Cryst. D54:684-686.

According to another embodiment of the present invention, to align two or more nucleic acid or amino acid sequences, for example to generate a consensus sequence or evaluate the similarity at various positions between such sequences, a CLUSTAL alignment program (e.g., CLUSTAL, CLUSTAL V, CLUSTAL W), also available as a module within the DNASTAR program, can be used using the following parameters, also referred to herein as the CLUSTAL standard default parameters:

Multiple Alignment Parameters (i.e., for more than 2 sequences):

(1) Gap penalty=10;

(2) Gap length penalty=10;

Pairwise Alignment Parameters (i.e., for two sequences):

(1) Ktuple=1;

(2) Gap penalty=3;

(3) Window=5;

(4) Diagonals saved=5.

According to the present invention, a GDP-4-keto-6-deoxy-D-mannose epimerase/reductase can be a GDP-4-keto-6-deoxy-D-mannose epimerase/reductase from any organism, including Arabidopsis thaliana, Escherichia coli, and human. A nucleic acid sequence encoding a GDP-4-keto-6-deoxy-D-mannose epimerase/reductase from Arabidopsis thaliana is represented herein by SEQ ID NO:1. SEQ ID NO:1 encodes a GDP-4-keto-6-deoxy-D-mannose epimerase/reductase having an amino acid sequence represented herein as SEQ ID NO:2. A nucleic acid sequence encoding a GDP-4-keto-6-deoxy-D-mannose epimerase/reductase from Escherichia coli is represented herein by SEQ ID NO:3. SEQ ID NO:3 encodes a GDP-4-keto-6-deoxy-D-mannose epimerase/reductase having an amino acid sequence represented herein as SEQ ID NO:4. A nucleic acid sequence encoding a GDP-4-keto-6-deoxy-D-mannose epimerase/reductase from homo sapiens is represented herein by SEQ ID NO:5. SEQ ID NO:5 encodes a GDP-4-keto-6-deoxy-D-mannose epimerase/reductase having an amino acid sequence represented herein as SEQ ID NO:6.

According to the present invention, a UDP-galactose 4-epimerase can be a UDP-galactose 4-epimerase from any organism, including Escherichia coli and human. A nucleic acid sequence encoding a UDP-galactose 4-epimerase from Escherichia coli is represented herein by SEQ ID NO:7. SEQ ID NO:7 encodes a UDP-galactose 4-epimerase having an amino acid sequence represented herein as SEQ ID NO:8. A nucleic acid sequence encoding a UDP-galactose 4-epimerase from homo sapiens is represented herein by SEQ ID NO:9. SEQ ID NO:9 encodes a UDP-galactose 4-epimerase having an amino acid sequence represented herein as SEQ ID NO:10.

In a preferred embodiment, an epimerase encompassed by the present invention has an amino acid sequence that aligns with the amino acid sequence of SEQ ID NO:11, for example using a CLUSTAL alignment program, wherein amino acid residues in the amino acid sequence of the epimerase align with 100% identity with at least about 50% of non-Xaa residues in SEQ ID NO:11, and preferably at least about 75% of non-Xaa residues in SEQ ID NO:11, and more preferably, at least about 90% of non-Xaa residues in SEQ ID NO:11, and even more preferably 100% of non-Xaa residues in SEQ ID NO:11. The percent identity of residues aligning with 100% identity with non-Xaa residues can be simply calculated by dividing the number of 100% identical matches at non-Xaa residues in SEQ ID NO:11 by the total number of non-Xaa residues in SEQ ID NO:11. A preferred nucleic acid sequence encoding an epimerase encompassed by the present invention include a nucleic acid sequence encoding an epimerase having an amino acid sequence with the above described identity to SEQ ID NO:11. Such an alignment using a CLUSTAL alignment program is based on the same parameters as previously disclosed herein. SEQ ID NO:11 represents a consensus amino acid sequence of an epimerase which was derived by aligning at least portions of amino acid sequences SEQ ID NO: 4, SEQ ID NO: 6 and SEQ ID NO: 8, as described in Somers et al., 1998, Structure 6:1601-1612, and can be approximately duplicated using CLUSTAL.

In another embodiment, an epimerase encompassed by the present invention includes an epimerase that has a catalytic site which includes amino acid residues: serine, tyrosine and lysine. Preferably, such serine, tyrosine and lysine residues are located at positions in the epimerase amino acid sequence which align using a CLUSTAL alignment program with positions Ser105, Tyr134 and Lys138 of consensus sequence SEQ ID NO:11, with positions Ser109, Tyr138 and Lys142 of sequence SEQ ID NO:2, with positions Ser107, Tyr136 and Lys140 of SEQ ID NO:4, with positions Ser114, Tyr143 and Lys147 of sequence SEQ ID NO:6, with positions Ser124, Tyr149 and Lys153 of sequence SEQ ID NO:8 or with positions Ser132, Tyr157 and Lys161 of sequence SEQ ID NO:10.

In another embodiment, an epimerase that has an amino acid sequence that is homologous to an amino acid sequence encoding a GDP-4-keto-6-deoxy-D-mannose epimerase/reductase includes any epimerase that has an amino acid motif: Gly-Xaa-Xaa-Gly-Xaa-Xaa-Gly, which is found, for example in positions 8 through 14 of the consensus sequence SEQ ID NO:11, in positions 12 through 18 of SEQ ID NO:2, in positions 10 through 16 of SEQ ID NO:4, in positions 14 through 20 of SEQ ID NO:6, in positions 7 through 13 of SEQ ID NO:8, and in positions 9 through 15 of SEQ ID NO:10. Such a motif can be identified by its alignment with the same motif in the above-identified amino acid sequences using a CLUSTAL alignment program. Preferably, such motif is located within the first 25 N-terminal amino acids of the amino acid sequence of the epimerase.

In yet another embodiment, an epimerase encompassed by the present invention includes an epimerase that has a substrate binding site which includes amino acid residues that align using a CLUSTAL alignment program with at least 50% of amino acid positions Asn177, Ser178, Arg187, Arg209, Lys283, Asn165, Ser107, Ser108, Cys109, Asn133, Tyr136 and His179 of SEQ ID NO:4. Alignment with positions Ser107, Tyr136, Asn165, Arg209, is preferably with 100% identity (i.e., exact match of residue, under parameters for alignment).

In another embodiment of the present invention, an epimerase encompassed by the present invention comprises at least 4 contiguous amino acid residues having 100% identity with at least 4 contiguous amino acid residues of an amino acid sequence selected from the group of SEQ ID NO:2, SEQ ID NO:4, SEQ ID NO:6, SEQ ID NO:8 or SEQ ID NO:10, as determined using a Lipman-Pearson method with Lipman-Pearson standard default parameters or by comparing an alignment using a CLUSTAL program with CLUSTAL standard default parameters. According to the present invention, the term “contiguous” means to be connected in an unbroken sequence. For a first sequence to have “100% identity” with a second sequence means that the first sequence exactly matches the second sequence with no gaps between nucleotides or amino acids.

In another embodiment of the present invention, an epimerase encompassed by the present invention is encoded by a nucleic acid sequence that comprises at least 12 contiguous nucleic acid residues having 100% identity with at least 12 contiguous nucleic acid residues of a nucleic acid sequence selected from the group of SEQ ID NO:1, SEQ ID NO:3, SEQ ID NO:5, SEQ ID NO:7 or SEQ ID NO:10, as determined using a Lipman-Pearson method with Lipman-Pearson standard default parameters or by comparing an alignment using a CLUSTAL program with CLUSTAL standard default parameters.

In another embodiment of the present invention, an epimerase encompassed by the present invention is encoded by a nucleic acid sequence that hybridizes under stringent hybridization conditions to a nucleic acid sequence selected from the group of SEQ ID NO:1, SEQ ID NO:3, SEQ ID NO:5, SEQ ID NO:7 or SEQ ID NO:9. As used herein, stringent hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, stringent hybridization and washing conditions, as referred to herein, refer to conditions which permit isolation of nucleic acid molecules having at least about 70% nucleic acid sequence identity with the nucleic acid molecule being used to probe in the hybridization reaction, more particularly at least about 75%, and most particularly at least about 80%. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6X SSC (0.9 M Na⁺) at a temperature of between about 20° C. and about 35° C., more preferably, between about 28° C. and about 40° C., and even more preferably, between about 35° C. and about 45° C. In particular embodiments, stringent hybridization conditions for DNA:RNA hybrids include hybridization at an ionic strength of 6X SSC (0.9 M Na⁺) at a temperature of between about 30° C. and about 45° C., more preferably, between about 38° C. and about 50° C., and even more preferably, between about 45° C. and about 55° C. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62.

In another embodiment of the present invention, an epimerase encompassed by the present invention is encoded by a nucleic acid sequence that comprises a nucleic acid sequence selected from the group of SEQ ID NO:1, SEQ ID NO:3, SEQ ID NO:5, SEQ ID NO:7 or SEQ ID NO:9 or a fragment thereof, wherein the fragment encodes a protein that is capable of catalyzing the conversion of GDP-D-mannose to GDP-L-galactose, such as under physiological conditions. In another embodiment, an epimerase encompassed by the present invention comprises an amino acid sequence selected from the group of SEQ ID NO:2, SEQ ID NO:4, SEQ ID NO:6, SEQ ID NO:8, SEQ ID NO:10 or a fragment thereof, wherein the fragment is capable of catalyzing the conversion of GDP-D-mannose to GDP-L-galactose. It is to be understood that the nucleic acid sequence encoding the amino acid sequences identified herein can vary due to degeneracies. As used herein, nucleotide degeneracies refers to the phenomenon that one amino acid can be encoded by different nucleotide codons.

One embodiment of the present invention relates to a method to identify an epimerase that catalyzes conversion of GDP-D-mannose to GDP-L-galactose. Preferably, such a method is useful for identifying the GDP-D-mannose:GDP-L-galactose epimerase which catalyzes the conversion of GDP-D-mannose to GDP-L-galactose in the endogenous (i.e., naturally occurring L-ascorbic acid biosynthetic pathway of microorganisms and/or plants). Such a method can include the steps of: (a) contacting a source of nucleic acid molecules with an oligonucleotide at least about 12 nucleotides in length under stringent hybridization conditions, wherein the oligonucleotide is identified by its ability to hybridize under stringent hybridization conditions to a nucleic acid sequence selected from the group consisting of SEQ ID NO:1, SEQ ID NO:3 and SEQ ID NO:5; and, (b) identifying nucleic acid molecules from the source of nucleic acid molecules which hybridize under stringent hybridization conditions to the oligonucleotide. Nucleic acid molecules identified by this method can then be isolated from the source using standard molecular biology techniques. Preferably, the source of nucleic acid molecules is obtained from a microorganism or plant that has an ascorbic acid production pathway. Such a source of nucleic acid molecules can be any source of nucleic acid molecules which can be isolated from an organism and/or which can be screened by hybridization with an oligonucleotide such as a probe or a PCR primer. Such sources include genomic and cDNA libraries and isolated RNA.

In order to screen cDNA libraries from different organisms and to isolate nucleic acid molecules encoding enzymes such as the GDP-D-mannose:GDP-L-galactose epimerase and related epimerases, one can use any of a variety of standard molecular and biochemical techniques. For example, oligonucleotide primers, preferably degenerate primers, can be designed using the most conserved regions of a GDP-4-keto-6-deoxy-D-mannose epimerase/reductase nucleic acid sequence, and such primers can be used in a polymerase chain reaction (PCR) protocol to amplify the same or related epimerases, including the GDP-D-mannose:GDP-L-galactose epimerase from the ascorbic acid pathway, from nucleic acids (e.g., genomic or cDNA libraries) isolated from a desired organism (e.g., a microorganism or plant having an L-ascorbic acid pathway). Similarly, oligonucleotide probes can be designed using the most conserved regions of a GDP-4-keto-6-deoxy-D-mannose epimerase/reductase nucleic acid sequence and such probe can be used to identify and isolate nucleic acid molecules, such as from a genomic or cDNA library, that hybridize under conditions of low, moderate, or high stringency with the probe.

Alternatively, the GDP-D-mannose:GDP-L-galactose epimerase can be purified from an organism such as Prototheca, the N-terminal amino acid sequence can be determined (including the sequence of internal peptide fragments), and this information can be used to design degenerate primers for amplifying a gene fragment from the organism cDNA. This fragment would then be used to probe the cDNA library, and subsequently fragments that hybridize to the probe would be cloned in that organism or another suitable production organism. There is ample precedent for plant enzymes being expressed in an active form in bacteria, such as E. coli. Alternatively, yeast are also a suitable candidate for developing a heterologous system for L-ascorbic acid production.

As discussed above in general for increasing the action of an enzyme in the L-ascorbic acid pathway according to the present invention, in one embodiment of the present invention, the action of an epimerase that catalyzes the conversion of GDP-D-mannose to GDP-L-galactose is increased by amplification of the expression (i.e., overexpression) of such an epimerase. Overexpression of an epimerase can be accomplished, for example, by introduction of a recombinant nucleic acid molecule encoding the epimerase. It is preferred that the gene encoding an epimerase according to the present invention be cloned under control of an artificial promoter. The promoter can be any suitable promoter that will provide a level of epimerase expression required to maintain a sufficient level of L-ascorbic acid in the production organism. Preferred promoters are constitutive (rather than inducible) promoters, since the need for addition of expensive inducers is therefore obviated. The gene dosage (copy number) of a recombinant nucleic acid molecule according to the present invention can be varied according to the requirements for maximum product formation. In one embodiment, the recombinant nucleic acid molecule encoding an epimerase according to the present invention is integrated into the chromosome of the microorganism.

It is another embodiment of the present invention to provide a microorganism having one or more epimerases according to the present invention with improved affinity for its substrate. An epimerase with improved affinity for its substrate can be produced by any suitable method of genetic modification or protein engineering. For example, computer-based protein engineering can be used to design an epimerase protein with greater stability and better affinity for its substrate. See for example, Maulik et al., 1997, Molecular Biotechnology: Therapeutic Applications and Strategies, Wiley-Liss, Inc., which is incorporated herein by reference in its entirety.

As noted above, in the method for production of L-ascorbic acid of the present invention, a microorganism having a genetically modified L-ascorbic acid production pathway is cultured in a fermentation medium for production of L-ascorbic acid. An appropriate, or effective, fermentation medium refers to any medium in which a genetically modified microorganism of the present invention, when cultured, is capable of producing L-ascorbic acid. Such a medium is typically an aqueous medium comprising assimilable carbon, nitrogen and phosphate sources. Such a medium can also include appropriate salts, minerals, metals and other nutrients. One advantage of genetically modifying a microorganism as described herein is that although such genetic modifications can significantly alter the production of L-ascorbic acid, they can be designed such that they do not create any nutritional requirements for the production organism. Thus, a minimal-salts medium containing glucose as the sole carbon source can be used as the fermentation medium. The use of a minimal-salts-glucose medium for the L-ascorbic acid fermentation will also facilitate recovery and purification of the L-ascorbic acid product.

In one mode of operation of the present invention, the carbon source concentration, such as the glucose concentration, of the fermentation medium is monitored during fermentation. Glucose concentration of the fermentation medium can be monitored using known techniques, such as, for example, use of the glucose oxidase enzyme test or high pressure liquid chromatography, which can be used to monitor glucose concentration in the supernatant, e.g., a cell-free component of the fermentation medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L, and can be determined readily by trial. Accordingly, when glucose is used as a carbon source the glucose concentration in the fermentation medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is preferred to maintain the carbon source concentration of the fermentation medium by addition of aliquots of the original fermentation medium. The use of aliquots of the original fermentation medium are desirable because the concentrations of other nutrients in the medium (e.g. the nitrogen and phosphate sources) can be maintained simultaneously. Likewise, the trace metals concentrations can be maintained in the fermentation medium by addition of aliquots of the trace metals solution.

In an embodiment of the fermentation process of the present invention, a fermentation medium is prepared as described above. This fermentation medium is inoculated with an actively growing culture of genetically modified microorganisms of the present invention in an amount sufficient to produce, after a reasonable growth period, a high cell density. Typical inoculation cell densities are within the range of from about 0.1 g/L to about 15 g/L, preferably from about 0.5 g/L to about 10 g/L and more preferably from about 1 g/L to about 5 g/L, based on the dry weight of the cells. The cells are then grown to a cell density in the range of from about 10 g/L to about 100 g/L preferably from about 20 g/L to about 80 g/L, and more preferably from about 50 g/L to about 70 g/L. The residence times for the microorganisms to reach the desired cell densities during fermentation are typically less than about 200 hours, preferably less than about 120 hours, and more preferably less than about 96 hours.

The microorganisms useful in the method of the present invention can be cultured in conventional fermentation modes, which include, but are not limited to, batch, fed-batch, and continuous. It is preferred, however, that the fermentation be carried out in fed-batch mode. In such a case, during fermentation some of the components of the medium are depleted. It is possible to initiate fermentation with relatively high concentrations of such components so that growth is supported for a period of time before additions are required. The preferred ranges of these components are maintained throughout the fermentation by making additions as levels are depleted by fermentation. Levels of components in the fermentation medium can be monitored by, for example, sampling the fermentation medium periodically and assaying for concentrations. Alternatively, once a standard fermentation procedure is developed, additions can be made at timed intervals corresponding to known levels at particular times throughout the fermentation. As will be recognized by those in the art, the rate of consumption of nutrient increases during fermentation as the cell density of the medium increases. Moreover, to avoid introduction of foreign microorganisms into the fermentation medium, addition is performed using aseptic addition methods, as are known in the art. In addition, a small amount of anti-foaming agent may be added during the fermentation.

The present inventors have determined that high levels of magnesium in the fermentation medium inhibits the production of L-ascorbic acid due to repression of enzymes early in the production pathway, although enzymes late in the pathway (i.e., from L-galactose to L-ascorbic acid) are not negatively affected (See Examples). Therefore, in a preferred embodiment of the method of the present invention, the step of culturing is carried out in a fermentation medium that is magnesium (Mg²⁺) limited. Even more preferably, the fermentation is magnesium limited during the cell growth phase. Preferably, the fermentation medium comprises less than about 0.5 g/L of Mg²⁺ during the cell growth phase of fermentation, and even more preferably, less than about 0.2 g/L of Mg²⁺, and even more preferably, less than about 0.1 g/L of Mg²⁺.

The temperature of the fermentation medium can be any temperature suitable for growth and ascorbic acid production, and may be modified according to the growth requirements of the production microorganism used. For example, prior to inoculation of the fermentation medium with an inoculum, the fermentation medium can be brought to and maintained at a temperature in the range of from about 20° C. to about 45° C., preferably to a temperature in the range of from about 25° C. to about 40° C., and more preferably in the range of from about 30° C. to about 38° C.

It is a further embodiment of the present invention to supplement and/or control other components and parameters of the fermentation medium, as necessary to maintain and/or enhance the production of L-ascorbic acid by a production organism. For example, in one embodiment, the pH of the fermentation medium is monitored for fluctuations in pH. In the fermentation method of the present invention, the pH is preferably maintained at a pH of from about pH 6.0 to about pH 8.0, and more preferably, at about pH 7.0. In the method of the present invention, if the starting pH of the fermentation medium is pH 7.0, the pH of the fermentation medium is monitored for significant variations from pH 7.0, and is adjusted accordingly, for example, by the addition of sodium hydroxide. In a preferred embodiment of the present invention, genetically modified microorganisms useful for production of L-ascorbic acid include acid-tolerant microorganisms. Such microorganisms include, for example, microalgae of the genera Prototheca and Chlorella (See U.S. Pat. No. 5,792,631, ibid. and U.S. Pat. No. 5,900,370, ibid.).

The production of ascorbic acid by culturing acid-tolerant microorganisms provides significant advantages over known ascorbic acid production methods. One such advantage is that such organisms are acidophilic, allowing fermentation to be carried out under low pH conditions, with the fermentation medium pH typically less than about 6. Below this pH, extracellular ascorbic acid produced by the microorganism during fermentation is relatively stable because the rate of oxidation of ascorbic acid in the fermentation medium by oxygen is reduced. Accordingly, high productivity levels can be obtained for producing L-ascorbic acid with acid-tolerant microorganisms according to the methods of the present invention. In addition, control of the dissolved oxygen content to very low levels to avoid oxidation of ascorbic acid is unnecessary. Moreover, this advantage allows for the use of continuous recovery methods because extracellular medium can be treated to recover the ascorbic acid product.

Thus, the present method can be conducted at low pH when acid-tolerant microorganisms are used as production organisms. The benefit of this process is that at low pH, extracellular ascorbic acid produced by the organism is degraded at a reduced rate than if the fermentation medium was at higher pH. For example, prior to inoculation of the fermentation medium with an inoculum, the pH of the fermentation medium can be adjusted, and further monitored during fermentation. Typically, the pH of the fermentation medium is brought to and maintained below about 6, preferably below 5.5, and more preferably below about 5. The pH of the fermentation medium can be controlled by the addition of ammonia to the fermentation medium. In such cases when ammonia is used to control pH, it also conveniently serves as a nitrogen source in the fermentation medium.

The fermentation medium can also be maintained to have a dissolved oxygen content during the course of fermentation to maintain cell growth and to maintain cell metabolism for L-ascorbic acid formation. The oxygen concentration of the fermentation medium can be monitored using known methods, such as through the use of an oxygen probe electrode. Oxygen can be added to the fermentation medium using methods known in the art, for example, through agitation and aeration of the medium by stirring or shaking. Preferably, the oxygen concentration in the fermentation medium is in the range of from about 20% to about 100% of the saturation value of oxygen in the medium based upon the solubility of oxygen in the fermentation medium at atmospheric pressure and at a temperature in the range of from about 30° C. to about 40° C. Periodic drops in the oxygen concentration below this range may occur during fermentation, however, without adversely affecting the fermentation.

The genetically modified microorganisms of the present invention are engineered to produce significant quantities of extracellular L-ascorbic acid. Extracellular L-ascorbic acid can be recovered from the fermentation medium using conventional separation and purification techniques. For example, the fermentation medium can be filtered or centrifuged to remove microorganisms, cell debris and other particulate matter, and L-ascorbic acid can be recovered from the cell-free supernate by conventional methods, such as, for example, ion exchange, chromatography, extraction, solvent extraction, membrane separation, electrodialysis, reverse osmosis, distillation, chemical derivatization and crystallization.

One such example of L-ascorbic acid recovery is provided in U.S. Pat. No. 4,595,659 by Cayle, incorporated herein in its entirety be reference, which discloses the isolation of L-ascorbic acid from an aqueous fermentation medium by ion exchange resin adsorption and elution, which is followed by decoloration, evaporation and crystallization. Further, isolation of the structurally similar isoascorbic acid from fermentation medium by a continuous multi-bed extraction system of anion-exchange resins is described by K. Shimizu, Agr. Biol. Chem. 31:346-353 (1967), which is incorporated herein in its entirety by reference.

Intracellular L-ascorbic acid produced in accordance with the present invention can also be recovered and used in a variety of applications. For example, cells from the microorganisms can be lysed and the ascorbic acid which is released can be recovered by a variety of known techniques. Alternatively, intracellular ascorbic acid can be recovered by washing the cells to extract the ascorbic acid, such as through diafiltration.

Development of a microorganism with enhanced ability to produce L-ascorbic acid by genetic modification can be accomplished using both classical strain development and molecular genetic techniques, and particularly, recombinant technology (genetic engineering). In general, the strategy for creating a microorganism with enhanced L-ascorbic acid production is to (1) inactivate or delete at least one, and preferably more than one of the competing or inhibitory pathways in which production of L-ascorbic acid is negatively affected (e.g., inhibited), and more significantly to (2) amplify the L-ascorbic acid production pathway by increasing the action of a gene(s) encoding an enzyme(s) involved in the pathway.

In one embodiment, the strategy for creating a microorganism with enhanced L-ascorbic acid production is to amplify the L-ascorbic acid production pathway by increasing the action of GDP-D-mannose:GDP-L-galactose epimerase, as discussed above. Such strategy includes genetically modifying the endogenous GDP-D-mannose:GDP-L-galactose epimerase such that L-ascorbic acid production is increased, and/or expressing/overexpressing a recombinant epimerase that catalyzes the conversion of GDP-D-mannose to GDP-L-galactose, which includes expression of recombinant GDP-D-mannose:GDP-L-galactose epimerase and/or homologues thereof, and of other recombinant epimerases such as GDP-4-keto-6-deoxy-D-mannose epimerase reductase and epimerases that share structural homology with such epimerase as discussed in detail above.

It is to be understood that a production organism can be genetically modified by recombinant technology in which a nucleic acid molecule encoding a protein involved in the L-ascorbic acid production pathway disclosed herein is transformed into a suitable host which is a different member of the plant kingdom from which the nucleic acid molecule was derived. For example, it is an embodiment of the present invention that a recombinant nucleic acid molecule encoding a GDP-D-mannose:GDP-L-galactose epimerase from a higher plant can be transformed into a microalgal host in order to overexpress the epimerase and enhance production of L-ascorbic acid in the microalgal production organism.

As previously discussed herein, in one embodiment, a genetically modified microorganism can be a microorganism in which nucleic acid molecules have been deleted, inserted or modified, such as by insertion, deletion, substitution, and/or inversion of nucleotides, in such a manner that such modifications provide the desired effect within the microorganism. A genetically modified microorganism is preferably modified by recombinant technology, such as by introduction of an isolated nucleic acid molecule into a microorganism. For example, a genetically modified microorganism can be transfected with a recombinant nucleic acid molecule encoding a protein of interest, such as a protein for which increased expression is desired. The transfected nucleic acid molecule can remain extrachromosomal or can integrate into one or more sites within a chromosome of the transfected (i.e., recombinant) host cell in such a manner that its ability to be expressed is retained. Preferably, once a host cell of the present invention is transfected with a nucleic acid molecule, the nucleic acid molecule is integrated into the host cell genome. A significant advantage of integration is that the nucleic acid molecule is stably maintained in the cell. In a preferred embodiment, the integrated nucleic acid molecule is operatively linked to a transcription control sequence (described below) which can be induced to control expression of the nucleic acid molecule.

A nucleic acid molecule can be integrated into the genome of the host cell either by random or targeted integration. Such methods of integration are known in the art. For example, an E. coli strain ATCC 47002 contains mutations that confer upon it an inability to maintain plasmids which contain a ColE1 origin of replication. When such plasmids are transferred to this strain, selection for genetic markers contained on the plasmid results in integration of the plasmid into the chromosome. This strain can be transformed, for example, with plasmids containing the gene of interest and a selectable marker flanked by the 5′- and 3′-termini of the E. coli lacZ gene. The lacZ sequences target the incoming DNA to the lacZ gene contained in the chromosome. Integration at the lacZ locus replaces the intact lacZ gene, which encodes the enzyme β-galactosidase, with a partial lacZ gene interrupted by the gene of interest. Successful integrants can be selected for β-galactosidase negativity.

A genetically modified microorganism can also be produced by introducing nucleic acid molecules into a recipient cell genome by a method such as by using a transducing bacteriophage. The use of recombinant technology and transducing bacteriophage technology to produce several different genetically modified microorganism of the present invention is known in the art.

According to the present invention, a gene, for example the GDP-D-mannose:GDP-L-galactose epimerase gene, includes all nucleic acid sequences related to a natural epimerase gene such as regulatory regions that control production of the epimerase protein encoded by that gene (such as, but not limited to, transcription, translation or post-translation control regions) as well as the coding region itself. In another embodiment, a gene, for example the GDP-D-mannose:GDP-L-galactose epimerase gene, can be an allelic variant that includes a similar but not identical sequence to the nucleic acid sequence encoding a given GDP-D-mannose:GDP-L-galactose epimerase gene. An allelic variant of a GDP-D-mannose:GDP-L-galactose epimerase gene which has a given nucleic acid sequence is a gene that occurs at essentially the same locus (or loci) in the genome as the gene having the given nucleic acid sequence, but which, due to natural variations caused by, for example, mutation or recombination, has a similar but not identical sequence. Allelic variants typically encode proteins having similar activity to that of the protein encoded by the gene to which they are being compared. Allelic variants can also comprise alterations in the 5′ or 3′ untranslated regions of the gene (e.g., in regulatory control regions). Allelic variants are well known to those skilled in the art and would be expected to be found within a given microorganism or plant and/or among a group of two or more microorganisms or plants.

In accordance with the present invention, an isolated nucleic acid molecule is a nucleic acid molecule that has been removed from its natural milieu (i.e., that has been subject to human manipulation). As such, “isolated” does not reflect the extent to which the nucleic acid molecule has been purified. An isolated nucleic acid molecule can include DNA, RNA, or derivatives of either DNA or RNA. There is no limit, other than a practical limit, on the maximal size of a nucleic acid molecule in that the nucleic acid molecule can include a portion of a gene, an entire gene, or multiple genes, or portions thereof.

An isolated nucleic acid molecule of the present invention can be obtained from its natural source either as an entire (i.e., complete) gene or a portion thereof capable of forming a stable hybrid with that gene. An isolated nucleic acid molecule can also be produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect within the microorganism. A structural homologue of a nucleic acid sequence has been described in detail above. Preferably, a homologue of a nucleic acid sequence encodes a protein which has an amino acid sequence that is sufficiently similar to the natural protein amino acid sequence that a nucleic acid sequence encoding the homologue is capable of hybridizing under stringent conditions to (i.e., with) a nucleic acid molecule encoding the natural protein (i.e., to the complement of the nucleic acid strand encoding the natural protein amino acid sequence). A nucleic acid molecule homologue encodes a protein homologue. As used herein, a homologue protein includes proteins in which amino acids have been deleted (e.g., a truncated version of the protein, such as a peptide), inserted, inverted, substituted and/or derivatized (e.g., by glycosylation, phosphorylation, acetylation, myristoylation, prenylation, palmitation, amidation and/or addition of glycosylphosphatidyl inositol) in such a manner that such modifications provide the desired effect on the protein and/or within the microorganism (e.g., increased or decreased action of the protein).

A nucleic acid molecule homologue can be produced using a number of methods known to those skilled in the art (see, for example, Sambrook et al., ibid.). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene.

Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a gene involved in an L-ascorbic acid production pathway.

Knowing the nucleic acid sequences of certain nucleic acid molecules of the present invention allows one skilled in the art to, for example, (a) make copies of those nucleic acid molecules and/or (b) obtain nucleic acid molecules including at least a portion of such nucleic acid molecules (e.g., nucleic acid molecules including full-length genes, full-length coding regions, regulatory control sequences, truncated coding regions). Such nucleic acid molecules can be obtained in a variety of ways including traditional cloning techniques using oligonucleotide probes to screen appropriate libraries or DNA and PCR amplification of appropriate libraries or DNA using oligonucleotide primers. Preferred libraries to screen or from which to amplify nucleic acid molecule include bacterial and yeast genomic DNA libraries, and in particular, microalgal genomic DNA libraries. Techniques to clone and amplify genes are disclosed, for example, in Sambrook et al., ibid.

The present invention includes a recombinant vector, which includes at least one isolated nucleic acid molecule of the present invention, inserted into any vector capable of delivering the nucleic acid molecule into a host microorganism of the present invention. Such a vector can contain nucleic acid sequences that are not naturally found adjacent to the isolated nucleic acid molecules to be inserted into the vector. The vector can be either RNA or DNA and typically is a plasmid. Recombinant vectors can be used in the cloning, sequencing, and/or otherwise manipulating of nucleic acid molecules. One type of recombinant vector, referred to herein as a recombinant molecule and described in more detail below, can be used in the expression of nucleic acid molecules. Preferred recombinant vectors are capable of replicating in a transformed bacterial cells, yeast cells, and in particular, in microalgal cells.

Transformation of a nucleic acid molecule into a cell can be accomplished by any method by which a nucleic acid molecule can be inserted into the cell. Transformation techniques include, but are not limited to, transfection, electroporation, microinjection and biolistics.

A recombinant cell is preferably produced by transforming a host cell with one or more recombinant molecules, each comprising one or more nucleic acid molecules operatively linked to an expression vector containing one or more transcription control sequences. The phrase, operatively linked, refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. In the present invention, expression vectors are typically plasmids. Expression vectors of the present invention include any vectors that function (i.e., direct gene expression) in a yeast host cell, a bacterial host cell, and preferably a microalgal host cell.

Nucleic acid molecules of the present invention can be operatively linked to expression vectors containing regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in yeast or bacterial cells or preferably, in microalgal cells. A variety of such transcription control sequences are known to those skilled in the art.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into the host cell chromosome, addition of vector stability sequences to plasmids, substitutions or modifications of transcription control signals (e.g., promoters, operators, enhancers), substitutions or modifications of translational control signals, modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein.

The following experimental results are provided for the purposes of illustration and are not intended to limit the scope of the invention.

### EXAMPLES

### Example 1

The present example describes the elucidation of the pathway from glucose to L-ascorbic acid through GDP-D-mannose in plants.

Since the present inventors have previously shown that Prototheca makes L-ascorbic acid (AA) from glucose, it was worthwhile to examine cultures for some of the early conversion products of glucose. In the past, the present inventors had concentrated on pathways from glucose to organic acids, based on the published pathway of L-ascorbic acid synthesis in animals and proposed pathways in plants. The present inventors demonstrate herein that the pathway from glucose to L-ascorbic acid involves not organic acids, but rather sugar phosphates and nucleotide diphosphate sugars (NDP-sugars).

Prior to the present invention, it was known that all cells synthesize polysaccharides by first forming NDP-sugars. The sugar moiety is then incorporated into polymer, while the cleaved NDP is recycled. A variety of polysaccharides are known, and are usually named based on the relative proportions of the sugar residues in the polymers. For example, a “galactomannan” contains mostly galactose, and to a lesser degree, mannose residues. The “biopolymer” from Prototheca strains isolated by the present inventors was analyzed and found to be 80% D-galactose, 18% rhamnose (D- or L-configuration not determined), and 2% L-arabinose. The present inventors provide evidence herein of how the respective NDP-sugars that make up the Prototheca biopolymer are formed, and what correlations exist between L-ascorbic acid synthesis and the formation of the NDP-sugar forms of the sugar residues found in the biopolymer.

The common NDP-sugar UDP-glucose is shown in FIG. 2B. This is formed in plants from glucose-1-P by the action of UDP-D-glucose pyrophosphorylase. UDP-glucose can be epimerized in plants to form UDP-D-galactose, using UDP-D-glucose-4-epimerase. UDP-D-galactose can also be formed by phosphorylation of D-galactose by galactokinase to form D-galactose-1-P, which can be converted to UDP-D-galactose by UDP-D-galactose pyrophosphorylase. These known routes were believed to account for the D-galactose in the Prototheca biopolymer. The UDP-L-arabinose can be formed by known reactions beginning with the oxidation of UDP-D-glucose to UDP-D-glucuronic acid (by UDP-D-glucose dehydrogenase), decarboxylation to UDP-D-xylose, and epimerization to UDP-L-arabinose. This accounts for the arabinose residues in the biopolymer. UDP-L-rhamnose is known to be formed from UDP-D-glucose, thus all three of the sugar moieties in the Prototheca biopolymer can be accounted for by a pathway through glucose-1-P and UDP-glucose. Alternatively, if the rhamnose in the biopolymer is D-rhamnose, it is not formed via UDP-D-glucose, but by oxidation of GDP-D-mannose (See FIG. 1).

GDP-D-rhamnose is formed by converting glucose, in turn, to D-glucose-6-P, D-fructose-6-P, D-mannose-6-P, D-mannose-1-P, GDP-D-mannose, and GDP-D-rhamnose. It was of interest to the present inventors that this route passes through GDP-D-mannose. Exogenous mannose is known to be converted to D-mannose-6-P in plants, and can enter the path above. D-mannose is converted to L-ascorbic acid by Prototheca cells cultured by the present inventors as well or better than glucose (see Example 4). The mechanism of conversion, in Chlorella pyrenoidosa, of GDP-D-mannose to GDP-L-galactose by GDP-D-mannose:GDP-L-galactose epimerase, has been known for years (See, Barber, 1971, Arch. Biochem. Biophys. 147:619-623, incorporated herein by reference in its entirety). The present inventors have discovered herein that L-galactose and L-galactono-γ-lactone are rapidly converted to L-ascorbic acid by strains of Prototheca and Chlorella pyrenoidosa. Prior to the present invention, it was known that L-galactono-γ-lactone is converted to L-ascorbic acid in several plant systems, but the synthesis steps prior to this step were unknown. Based on the published literature and the present experimental evidence, the present inventors have determined that the L-ascorbic acid biosynthetic pathway in plants passes through GDP-D-mannose and involves sugar phosphates and NDP-sugars. The proposed pathway is shown in FIG. 1. Salient points relevant to the design and production of genetically modified microorganisms useful in the present method include:

1. The enzymes leading from D-glucose to D-fructose-6-P are well known enzymes in the first, uncommitted steps of glycolysis.

2. The enzymes involved in the conversion of D-fructose-6-P to GDP-D-mannose have been well characterized in plants, yeast, and bacteria, particularly Azotobacter vinelandii and Pseudomonas aeruginosa, which convert GDP-D-mannose to GDP-D-mannuronic acid, which is the precursor for alginate (See for example, Sa-Correia et al., 1987, J. Bacteriol. 169:3224-3231; Koplin et al., 1992, J. Bacteriol. 174:191-199; Oesterhelt et al., 1996, Plant Science 121:19-27; Feingold et al., 1980, The Biochemistry of Plants: Vol 3: Carbohydrates, structure and function, P. K. Stampf & E. E. Conn, eds., Academic Press, New York, pp. 101-170; Smith et al., 1992, Mol. Cell Biol. 12:2924-2930; Boles et al., 1994, Eur. J. Med. 220:83-96; Hashimoto et al., 1997, J. Biol. Chem. 272:16308-16314, all of which are incorporated herein by reference in their entirety).

3. Barber (1971, supra, and 1975) identified in Chlorella pyrenoidosa the enzyme activities for the conversion of GDP-D-mannose to GDP-L-galactose and L-galactose-1-P.

4. The present inventors have shown herein the rapid conversion of L-galactose and L-galactono-γ-lactone to L-ascorbic acid by Prototheca cells.

5. L-galactono-y-lactone and L-galactonic acid can be interconverted in solution by changing the pH of the solution; addition of base shifts the equilibrium to L-galactonic acid, while addition of acid shifts the equilibrium to the lactone. Cells may have an enzymatic means for this conversion in addition to this non-enzymatic route.

6. In plants, GDP-L-fucose is also formed from GDP-D-mannose, presumably for incorporation into polysaccharide. Roberts (1971) fed labeled D-mannose to corn root tips and found the label in polysaccharide, specifically in the residues of D-mannose, L-galactose, and L-fucose. No label was detected in D-glucose, D-galactose, L-arabinose, or D-xylose. Prototheca and C. pyrenoidosa cells have the ability to convert L-fucose (6-deoxy-L-galactose) to a dipyridyl-positive product that was shown by HPLC not to be L-ascorbic acid. The present inventors believe that it is was the 6-deoxy analog of L-ascorbic acid.

### Example 2

This example shows that in Prototheca, like other plants (Loewus, F. A. 1988. In: J. Priess (ed.), The Biochemistry of Plants, 14:85-107. New York, Academic Press) and the green microalga Chlorella pyrenoidosa (Renstrom, et al., 1983. Plant Sci. Lett. 28:299-305), ascorbic acid (AA) production from glucose proceeds by a biosynthetic pathway that allows retention of the configuration of the carbon skeleton of glucose.

Cultures of the strain UV77-247 were grown to moderate cell density in shake flasks with 1-¹³C-labeled glucose as 10% of the total glucose (40 g/L) . Incubation was as per the standard Mg-limited screen (see Example 3). The culture supernates were clarified, deionized to remove salts, lyophilized, and subjected to nuclear magnetic resonance (nmr) analysis to determine where in the AA molecule the ¹³C was located. In each case, approximately 85% of the label was found at the C-1 position of AA, with most of the remaining label at the C-6 position. This strongly indicated that AA is synthesized from glucose by a pathway that retains the carbon chain configuration, i. e., C-1 of glucose becomes C-1 of AA. This has typically been observed in plants (Loewus, F. A. 1988. Ascorbic acid and its metabolic products. In: The Biochemistry of Plants, ed. J. Priess, 14:85-107. New York, Academic Press). Animals (Mapson, L. W. and F. A. Isherwood 1956. Biochem. J. 64:151-157; Loewus, F. A. 1960. J. Biol. Chem. 235(4) :937-939) and protists such as Euglena (Shigeoka, S., et al., 1979. J. Nutr. Sci. Vitaminol. 25:299-307), on the other hand, synthesize AA by a pathway that involves the inversion of configuration, i. e., C-1 of glucose becomes C-6 of AA. Demonstration of the inversion/non-inversion nature of the pathway was an important step in determining the pathway of AA biosynthesis since the two types of pathways require different types of enzymatic reactions. The label found at C-6 of AA is thought to be due to metabolism of glucose and subsequent gluconeogenesis. The metabolism of glucose in glycolysis proceeds through triose-phosphate intermediates. After this, the C-1 and C-6 carbons of glucose become biochemically equivalent. Hexose phosphates can be regenerated from the triose phosphates by gluconeogenesis, which is essentially a reversal of the degradative pathway. Consequently, metabolism of C-1-labeled glucose to triose phosphates with subsequent gluconeogenesis would result in the formation of hexose phosphate molecules labeled at either or both C-1 and C-6. If those hexose phosphates were precursors to AA, one would expect the AA to be similarly labeled. Consistent with this type of “isotopic mixing” is the observation that sucrose obtained from 1-¹³C-labeled glucose was labeled at positions 1, 6, 1′ and 6′.

Glucose can also be metabolized by the pentose phosphate pathway, the overall balanced equation for which is:

3Glucose-6-phosphate→2Fructose-6-phosphate+Glyceraldehyde-3-phosphate+3 CO₂

Based on the known biochemistry, it would then be expected that the label at each of the carbons in glucose (Table 1 left column) would appear at the positions for the other molecules shown, and that these patterns would be reflected in the AA formed from C-2- and C-3-labeled glucose.

TABLE 1
Predicted Carbon Labeling of Metabolites of Glucose in the Pentose
Phosphate Pathway

| Labeled Glucose | Position of Labeled Carbon in: | | | |
|---|---|---|---|---|
| Carbon | CO₂ | F6P(1) | F6P(2) | G3P |
| 1 | + | − | − | − |
| 2 | − | 1, 3 | 1 | − |
| 3 | − | 2 | 2, 3 | − |
| 4 | − | 4 | 4 | 1 |
| 5 | − | 5 | 5 | 2 |
| 6 | − | 6 | 6 | 3 |

AA recovered from cultures fed glucose labeled at C-2 or C-3 was also analyzed for its labeling patterns (Table 2).

TABLE 2
Labeling Pattern in AA after Cells were Fed 2-¹³C and 3-¹³C-glucose

| | |
|---|---|
| Carbon | Isotopic enhancement after growth on: |

| Position in AA | C-2 labeled glucose | C-3 labeled glucose |
|---|---|---|
| 1 | 1.0 | 0.4 |
| 2 | 10.0 | 0.9 |
| 3 | 0.5 | 9.9 |
| 4 | 0 | 2.8 |
| 5 | 2.2 | 0.2 |
| 6 | 0 | 0 |

The data above again suggest a pathway from glucose to AA that proceeds by retention of configuration. As in the experiments with C-1 labeled glucose, approximately one-fifth of the label is present in “mirror image” position to the glucose label (C-5 for C-2 labeled glucose and C-4 for C-3 labeled glucose), indicating levels of gluconeogenesis consistent with those previously observed.

The small, but significant amount of enhancement observed in other positions is consistent with flux through the pentose phosphate pathway. As predicted above, carbon flux through this pathway would result in isotopic enhancement at positions 1 and 3 when cells were grown on 2-¹³C glucose and enhancement at position 2 when cells were grown on 3-¹³C glucose. This is indeed observed. That there is twice as much enhancement at C-1 as there is at C-3 after growth on 2-¹³C glucose is also predicted. These data indicate a small but measurable amount of carbon flux through the pentose phosphate pathway.

### Example 3

This example shows the methods for generating, screening and isolating mutants of Prototheca with altered AA productivities compared to the starting strain ATCC 75669.

ATCC No. 75669, identified as Prototheca moriformis RSP1385 (unicellular green microalga), was deposited on Feb. 8, 1994, with the American Type Culture Collection (ATCC), Rockville, Md., 20852, U.S.A., under the terms of the Budapest Treaty on the International Recognition of the Deposit of Microorganisms for the Purpose of Patent Procedure. Initial screening of Prototheca species and strains was reported in U.S. Pat. No. 5,900,370, ibid. Table 3 lists the formulations of the media for growth and maintenance of the strains. Glucose for fermentors was supplied as glucose monohydrate and calculated on an anhydrous basis. The recipe for the trace metals solution is given in Table 4. The standard growth temperature was 35° C. All organisms were cultured axenically.

TABLE 3
Media for Growth and Maintenance of Prototheca Strains
All quantities are in g/L unless otherwise specified

| | | | Agar | | |
|---|---|---|---|---|---|
| | Liquid | | | Ferro- | Stan- |
| | Stand- | Mg- | | zine | dard |
| Ingredient | ard | limiting | Slants | Plates | Plates |
| Potassium phosphate | 1.3 | 1.3 | 2.0 | 0.27 | 2.0 |
| monobasic | | | | | |
| Potassium phosphate | 3.8 | 3.8 | 2.0 | 1.4 | 2.0 |
| dibasic | | | | | |
| Trisodium citrate | 7.7 | 7.7 | 2.6 | 1.3 | 2.6 |
| dihydrate | | | | | |
| Magnesium sulfate | 0.40 | 0.02 | 0.4 | 0.01 | 0.4 |
| heptahydrate | | | | | |
| Ammonium sulfate | 3.7 | 3.7 | 1.0 | 1.0 | 1.0 |
| Trace Metals Solution | 2 mL | 2 mL | 2 mL | 2 mL | 2 mL |
| Ferrous sulfate | 1.5 mg | 4.5 mg | 1.5 mg | — | 1.5 mg |
| heptahydrate | | | | | |
| Calcium chloride | — | 0.25 | — | — | — |
| dihydrate | | | | | |
| Manganous sulfate | — | 0.08 | — | — | — |
| monohydrate | | | | | |
| Yeast extract | — | — | 2.5 | — | — |
| Agar | — | — | 15 | 15 | 15 |
| | | | | (Noble) | |
| pH before autoclaving | 7.2 | 7.2 | 7.2 | 7.2 | 7.2 |

Autoclave, then add

| | | | | | |
|---|---|---|---|---|---|
| Copper sulfate, penta- | — | — | — | 2 mL | — |
| hydrate, 100 g/L | | | | | |
| 40 g/L Ferrozine | — | — | — | 8.8 mL | — |
| in 5 mM phosphate | | | | | |
| (pH 7.5 final) | | | | | |
| Ferric ammonium sulfate | — | — | — | 3.8 mL | — |
| dodecahydrate, 40 g/L | | | | | |
| 50% glucose with | 40 mL | 60 mL | 10 mL | 10 mL | 10 mL |
| 25 mg/L thiamine HCl | | | | | |

TABLE 4
Trace Metals Solution

| | | | mL Indiv. Stock |
|---|---|---|---|
| | Molecular | Conc. of Individ. | per liter |
| Compound | Weight | Solutions, g/L | of Working Stock |
| Distilled Water | — | — | 823 |
| Hydrochloric Acid | — | Conc. | 20 |
| Cobalt Chloride | 237.9 | 24.0 | 6.5 |
| hexahydrate | | | |
| Boric acid | 61.8 | 38.1 | 24 |
| Zinc sulfate | 287.5 | 35.3 | 50 |
| heptahydrate | | | |
| Manganous sulfate | 169.0 | 24.6 | 50 |
| monohydrate | | | |
| Sodium molybdate | 242.0 | 23.8 | 2.0 |
| dihydrate | | | |
| Calcium chloride | 147.0 | — | 11.4 g |
| dihydrate | | | |
| Vanadyl sulfate | 199.0 | 10.0 | 8.0 |
| dihydrate | | | |
| Nickel nitrate | 290.8 | 5.0 | 8.0 |
| hexahydrate | | | |
| Sodium selenite | 173.0 | 5.0 | 8.0 |

Mutant isolates were generated by treatment with one or more of the following agents: nitrous acid (NA); ethyl methane sulfonate (EMS); or ultraviolet light (UV). Typically, glucose-depleted cells grown in standard liquid medium were washed and resuspended in 25 mM phosphate buffer, pH 7.2, diluted to approximately 10⁷ colony-forming units per mL (cfu/mL), exposed to the mutagen to achieve about 99% kill, incubated 4-8 hours in the dark, and spread onto standard agar medium, or agar media containing differential agents.

Some mutant colonies on standard agar medium were picked randomly and subcultured to master plates. Other isolation plates were inverted over chloroform to lyse cells on the surface of the colonies and allow them to release AA. Released AA was detected by spraying the treated plates with a solution of 2,6-dichrorophenol-indophenol (1.25 g/L in 70% EtOH). The ability of AA to reduce this blue redox dye to its colorless form is the basis for a standard assay of AA (Omaye, et al., 1979. Meth. Enzymol. 62:3-11.). Colonies derived from mutagenized cells were saved to master plates for further evaluation if their clear halos were significantly larger than the halos typical of the other mutants in that group. Other mutagenized cells were spread onto plates containing an AA detection system incorporated directly into the agar. This system is based on the ability of AA to reduce ferric iron to ferrous iron. The compound ferrozine (3-(2-pyridyl)-5,6-bis(4-phenylsulfonic acid)-1,2,4-triazine) was present in the agar to complex with the ferrous iron and give a violet color reaction. The ferrozine agar formulation is shown in Table 3. Colonies giving the darkest color reactions were master-plated. When screening for non-AA-producing strains (blocked mutants), white colonies were chosen against a background of relatively dark colonies.

For primary screening of tube cultures, cells were inoculated from master plates into 4 mL of Mg-limiting medium in 16×125 mm test tubes, and tubes were shaken in a slanted position on a rotary shaker at 300 rpm for four days. After both three and four days of incubation aliquots were removed for AA assay and cell density determination. Those for AA assay were centrifuged at 1500×g for 5 min and the resulting supernates were removed for either calorimetric assay or high pressure liquid chromatography (HPLC). Promising isolates were retested in tube culture. Those passing the tube screen were tested in shake flasks.

For secondary screening of flask cultures, cells were inoculated into 50 mL of standard flask medium in 250 mL baffled shake flasks, and incubated on a rotary shaker at 180 rpm until glucose depletion (24-48 hours). A second series of flasks of Mg-sufficient standard medium was inoculated from the first set to a cell density of 0.15 A₆₂₀, and incubated for 24 hours. A third series of Mg-limiting flask medium was inoculated from the second set by a 1/50 dilution and incubated for 96 hours. Flasks were sampled for AA analysis and cell density measurements during this time as required. Aliquots for supernatant AA analysis were centrifuged at 5000×g for 5 min. Aliquots for total whole broth AA analysis were first extracted for 15 min with an equal volume of 5% trichloroacetic acid (TCA) before centrifugation. Aliquots of the resulting supernates were removed for either colorimetric assay or HPLC analysis.

For colorimetric assay of AA, a modification of the method of Omaye, et al. (1979. Meth. Enzymol. 62:3-11) was used. Twenty-five μL aliquots of culture supernates were added to wells of 96-well microplates, and 125 μL of color reagent was added. The color reagent consisted of four parts 0.5% aqueous 2,2′-dipyridyl and one part 8.3 mM ferric ammonium sulfate in 27% (v/v) o-phosphoric acid, the two components being mixed immediately before use. After one hour, the absorbance at 520 nm was read. AA concentration was calculated by comparison of the absorbances of AA standards.

HPLC analysis was based on that of Running, et al., (1994). Supernates were chromatographed on a Bio-Rad HPX-87H organic acid column (Bio-Rad Laboratories, Richmond, Calif.) with 13 mM nitric acid as solvent, at a flow rate of 0.7 mL/min at room temperature. Detection was at either 254 nm using a Waters 441 detector (Millipore Corp., Milford, Mass.), or at 245 nm using a Waters 481 detector. This system can distinguish between the L- and D-isomers of AA.

For dry weight determinations of cell density, 5 mL whole broth samples were centrifuged at 5000×g for 5 min, washed once with distilled water, and the pellet was washed into a tared aluminum weighing pan. Cells were dried for 8-24 h at 105° C. Cell weight was calculated by difference.

Table 5 shows the abilities of various mutants of Prototheca to synthesize AA.

TABLE 5
AA Synthesizing Ability of Various Prototheca Mutants in Flask Screen

| | Specific AA Formation, mg AA per L/Culture A₆₂₀, | |
|---|---|---|
| | during Mg-limited Incubation | |
| Strain | 2 Days Incubation | 4 Days Incubation |
| ATCC 75669 | 22 | 35 |
| EMS13-4 | 79 | 166 |
| UV213-1 | 0 | 0 |
| UV218-1 | 0 | 0 |
| UV244-1 | 0 | 0 |
| UV244-15 | 58 | 68 |
| UV77-247 | 56 | 83 |
| UV140-1 | 67 | 100 |
| UV164-6 | 91 | 131 |
| NA21-14 | 27 | 78 |
| UV82-21 | 0 | 0 |
| UV127-10 | 50 | 95 |
| SP2-3 | 3 | 4 |

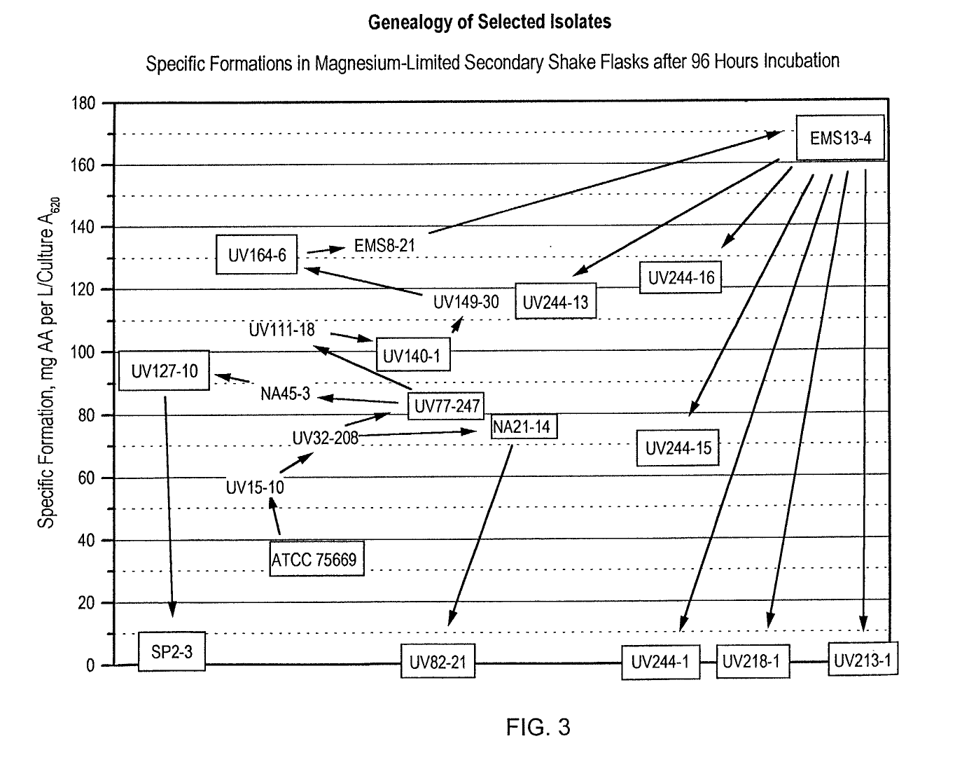

The genealogy of these isolates is presented graphically in the “family tree” of FIG. 3. ATCC No. ______, identified as Prototheca moriformis EMS13-4 (unicellular green microalga), was deposited on May 25, 1999, with the American Type Culture Collection (ATCC), 10801 University Boulevard, Manassas, Va. 20110, U.S.A., under the terms of the Budapest Treaty on the International Recognition of the Deposit of Microorganisms for the Purpose of Patent Procedure. ATCC No. ______, identified as Prototheca moriformis UV127-10 (unicellular green microalga), was deposited on May 25, 1999, with the American Type Culture Collection (ATCC), 10801 University Boulevard, Manassas, Va. 20110, U.S.A., under the terms of the Budapest Treaty on the International Recognition of the Deposit of Microorganisms for the Purpose of Patent Procedure. ATCC No. ______, identified as Prototheca moriformis SP2-3 (unicellular green microalga), was deposited on May 25, 1999, with the American Type Culture Collection (ATCC), 10801 University Boulevard, Manassas, Va. 20110, U.S.A., under the terms of the Budapest Treaty on the International Recognition of the Deposit of Microorganisms for the Purpose of Patent Procedure.

### Example 4

The following example shows that both growing and resting cells of Prototheca can rapidly convert L-galactose and L-galactono-γ-lactone to AA, and that conversion of D-mannose to AA by Prototheca is more rapid than conversion of D-glucose.

Shake flask cultures of the mutant strain UV77-247 were grown to glucose depletion in standard liquid medium (Table 3). Cells were washed twice and resuspended in complete medium with the glucose substituted by one of the compounds listed below. Cell suspensions were incubated for 24 hours at 35° C. with shaking, and the entire suspension was extracted with TCA as above and assayed for AA.

Tables 6-8 show that both growing and resting cells of strain UV77-247 can rapidly convert L-galactose and L-galactono-γ-lactone to AA. In these experiments, D-fructose and D-galactose were converted to AA at the same rate as D-glucose, suggesting that they are metabolized to AA through the same route as D-glucose. None of the organic acids suggested in the literature to be intermediates in the biosynthesis of AA were converted to AA, including sorbosone, which has been proposed as an intermediate by Saito et al. (1990 Plant Physiol. 94:1496-1500).

TABLE 6
Conversion of Compounds by Resting Cells of Strain UV77-247

| | | AA Relative to No |
|---|---|---|
| Substrate (50 mM) | Total AA, mg/L | Substrate Control |
| L-galactose | 965 | 623 |
| L-galactono-γ-lactone | 818 | 476 |
| D-fructose | 590 | 248 |
| D-glucosone | 589 | 247 |
| D-glucose | 584 | 242 |
| D-galactose | 542 | 200 |
| D-glucose (10 mM) | 388 | 46 |
| D-gluconolactone | 382 | 40 |
| D-gulono-γ-lactone | 366 | 24 |
| D-glucuronate | 364 | 22 |
| L-sorbosone | 342 | 0 |
| None | 342 | 0 |
| 2-keto-D-gluconic acid | 341 | −1 |
| D-isoascorbic acid (10 mM) | 330 | −12 |
| D-glucuronolactone | 329 | −13 |
| D-gluconic acid | 309 | −33 |
| D-galacturonic acid | 297 | −45 |
| L-idonate | 296 | −46 |

Since strain UV77-247 converted L-galactose and L-galactono-γ-lactone to AA much more rapidly than it did glucose, it suggests that these compounds are intermediates in the AA biosynthetic pathway and that they are “downstream” from glucose.

The data in Tables 7 and 8 also show that growing and resting cells of UV77-247 consistently convert D-mannose to AA at a rate greater than that of glucose.

TABLE 7
Conversion of Compounds to AA by Resting Cells of Strain UV77-247

| | Ascorbic Acid, mg/L | | |
|---|---|---|---|
| Compound | 25.5 h | 30 h | 47 h |
| L-galactose | 667 | 718 | 620 |
| L-galactono-γ-lactone | 644 | 681 | 749 |
| D-glucosone | 465 | 462 | 354 |
| D-mannose | 448 | 462 | 399 |
| D-fructose | 402 | 408 | 367 |
| d-glucose | 395 | 404 | 351 |
| D-galactose | 352 | 361 | 337 |
| none | 287 | 288 | 258 |

TABLE 8
Conversion of Compounds to AA by Growing Cells of Strain UV77-247

| | Ascorbic Acid, | | | |
|---|---|---|---|---|
| | mg/L | | A₆₂₀ | AA/A₆₂₀ |
| Compound | 25.5h | 44h | | |
| L-galactose | 249 | 506 | 4.5 | 112 |
| D-mannose | 228 | 488 | 5.6 | 87 |
| L-galactono-γ-lactone | 214 | 342 | 5.0 | 68 |
| D-glucose | 178 | 398 | 5.9 | 67 |
| D-fructose | 181 | 383 | 5.9 | 65 |
| D-glucosone | 176 | 362 | 5.7 | 64 |
| D-galactose | 185 | 380 | 5.9 | 64 |
| none | 182 | 249 | 4.4 | 57 |
| D-gluconic acid (K) | 178 | 262 | 5.0 | 52 |
| L-idonate (Na) | 182 | 232 | 4.7 | 49 |
| 2-keto-D-gluconic acid | 182 | 255 | 5.3 | 48 |
| 2-deoxy-D-glucose | 181 | 227 | 4.8 | 47 |
| D-glucuronic acid lactone | 165 | 218 | 5.0 | 44 |
| D-glucuronic acid (Na) | 173 | 241 | 5.6 | 43 |
| L-gulono-γ-lactone | 152 | 195 | 5.0 | 39 |
| L-sorbosone | 178 | 160 | 4.7 | 34 |
| D-glucono-δ-lactone | 130 | 190 | 5.7 | 33 |
| D-galacturonic acid | 130 | 180 | 6.0 | 30 |

These cells converted L-galactose, L-galactono-γ-lactone and D-mannose to AA more rapidly than they did glucose, suggesting that mannose exerts its effect in the biosynthetic pathway “downstream” from glucose.

### Example 5

Using the methods described above, a collection of mutants was assembled. The specific AA formation for representative mutants are shown in Table 5. The genealogy of these isolates is presented graphically in the “family tree” of FIG. 3.

These isolates were tested for their ability to convert compounds which could be converted to AA by strain UV77-247. Testing was done as in Example 4. Results are shown in Table 9.

TABLE 9
Conversion of Compounds to AA by Resting Cells
of Mutant Strains of Prototheca of Varying Abilities to Synthesize AA

| | Absolute AA, mg/L | | | | | |
|---|---|---|---|---|---|---|
| | | | L- | L-gal- | | Fruc- |
| Strain | Buffer | Glucose | galactose | γ-lact. | Mannose | tose |
| EMS13-4 | 53 | 97 | 191 | 173 | 139 | ND |
| UV127-10 | 45 | 140 | 213 | 140 | 128 | 143 |
| SP2-3 | 19 | 19 | 204 | 146 | 24 | 27 |
| NA21-14 | 61 | 80 | 147 | 158 | 118 | 115 |
| UV82-21 | 15 | 16 | 183 | 175 | 18 | 17 |
| UV213-1 | 16 | 15 | 170 | 135 | 17 | 16 |
| UV218-1 | 16 | 18 | 136 | 176 | 19 | 21 |
| UV244-1 | 16 | 16 | 164 | 162 | 16 | 16 |
| UV244-15 | 26 | 77 | 30 | 21 | 94 | 89 |
| UV244-16 | 28 | 64 | 53 | 53 | 53 | 66 |

ND = Not Determined

These data suggest that the mutational blocks in those strains which convert fructose and mannose to AA poorly are before (“upstream” from) L-galactose and L-galactono-γ-lactone in the pathway.

### Example 6

The following example shows that magnesium inhibits early steps in the production of AA.

To address the question of whether magnesium actually inhibits AA synthesis, strain NA45-3 (ATCC 209681) was grown in magnesium (Mg)-limited and Mg-sufficient medium. ATCC No. 209681, identified as Prototheca moriformis NA45-3 (Source: repeated mutagenesis of ATCC No. 75669; Eucaryotic alga. Division Chlorophyta, Class Chlorophyceae, Order Chlorococcales), was deposited on Mar. 13, 1998, with the American Type Culture Collection (ATCC), 10801 University Boulevard, Manassas, Va. 20110, U.S.A., under the terms of the Budapest Treaty on the International Recognition of the Deposit of Microorganisms for the Purpose of Patent Procedure. Cells from both cultures were harvested and resuspended in the cell-free supernate from the Mg-limited culture, and to half of each cell suspension additional magnesium was added in order to bring the level in the suspension to the Mg-sufficient level. The four conditions under which assays were run were as follows.

TABLE 10
Conditions Used to Test the Effect of Magnesium on AA Production

Magnesium concentration, g/L, during:

| Condition | Growth | Assay |
|---|---|---|
| 1 Mg > 1 Mg | 0.02 | 0.02 |
| 1 Mg > 10 Mg | 0.02 | 0.2 |
| 10 Mg > 1 Mg | 0.2 | 0.02 |
| 10 Mg > 10 Mg | 0.2 | 0.2 |

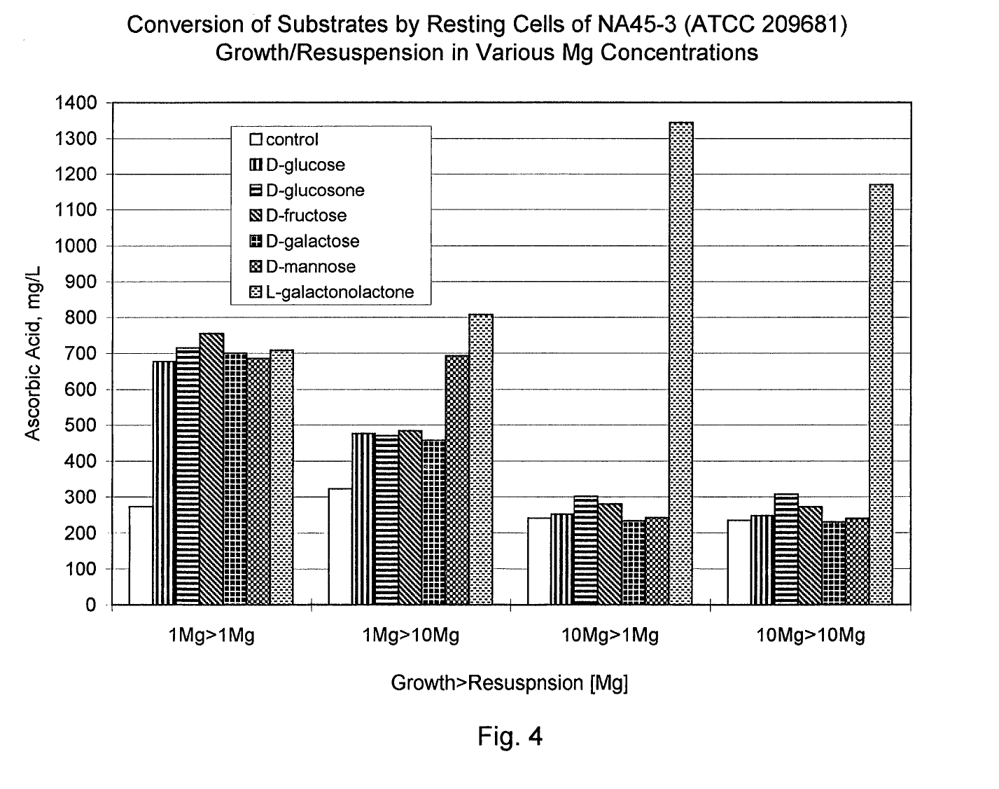

Substrates previously shown to lead to the formation of AA, namely D-glucose, D-glucosone, D-fructose. D-galactose, D-mannose, and L-galactono-γ-lactone, were added at 20 g/L to the four cell suspensions. Accumulation of AA after 24 hours was measured and compared to a control in which no substrate was added. The results of this study are shown graphically in FIG. 4.

When cells growing under magnesium-limited conditions were incubated with substrates in low-magnesium broth (1 Mg>1 Mg condition), all showed significant and similar accumulation of AA over the control condition. When the same cells were incubated in high magnesium broth (1 Mg>10 Mg condition), the accumulation of AA was reduced about 40% for all substrates except D-mannose and L-galactono-γ-lactone, suggesting that 1) the rate-limiting step in the conversion of D-glucose, D-glucosone, D-fructose, and D-galactose to AA is inhibited by magnesium or 2) magnesium stimulates an enzyme which results in the conversion of these compounds to some other compound(s), reducing the amount of substrate available for AA synthesis. On the other hand, conversion of D-mannose and L-galactono-γ-lactone appeared to be unaffected by the presence of magnesium in the resuspension buffer, indicating that either 1) magnesium-inhibited enzymes are not involved in the conversion of these substrates to AA or 2) D-mannose and L-galactono-γ-lactone enter the pathway far enough downstream from the point where they can be siphoned off by side reactions involving Mg-requiring enzymes.

When cells were grown under magnesium-sufficient conditions, very little AA accumulation from any of the D-sugars was observed, regardless of the level of magnesium in the resuspension broth. Accumulation of AA from L-galactono-γ-lactone, however, was enhanced over that observed when cells are grown in Mg-limited conditions. This suggests that enzymes early in the pathway are repressed under Mg-sufficient conditions. Thus, the D-substrates all behaved similarly, with the exception of the apparent lack of magnesium inhibition of D-mannose conversion to AA. This would suggest that D-mannose enters the AA biosynthetic pathway at a point other than the other D-sugars.

FIGS. 2A and 2B represent some of the fates of glucose in plants. The first enzymatic step in this scheme which commits carbon to glycolysis is the conversion of fructose-6-P to fructose-1,6-diP by phosphofructokinase (PFK). This reaction is essentially irreversible, and leads to the well known TCA cycle and oxidative phosphorylation, with concomitant ATP and NADH/NADPH generation. PFK has an absolute requirement for magnesium. If magnesium is limiting, this reaction could slow and eventually stop, blocking the flow of carbon through glycolysis and beyond, and would result in cessation of cell division even in the presence of excess glucose. One would expect fructose-6-P to accumulate under these conditions, fueling AA synthesis by the pathway shown in FIGS. 1 and 2.

### Example 7

The following example shows the correlation in Prototheca between AA production and the activity levels of the enzymes in the AA pathway.

### Phosphomannose Isomerase (PMI) Assay

PMI activity was first assayed (See FIG. 1). Ten strains representing a range of AA productivities were grown according to the standard protocol to measure AA-synthesizing ability. Cells were harvested 96 hours into magnesium-limited incubation, washed and resuspended in buffer containing 50 mM Tris/10 mM MgCl₂, pH 7.5. The suspended cells were broken in a French press, spun at 30,000×g for 30 minutes, and desalted through Sephadex G-25 (Pharmacia PD-10 columns). Reactions were carried out in the reverse direction by adding various volumes of extracts to solutions of Tris/Mg buffer containing 0.15 U phosphoglucose isomerase (EC 5.3.1.9), 0.5 U glucose-6-phosphate dehydrogenase (EC 1.1.1.49), and 1.0 mm NADP. Reactions were initiated by addition of 3 mM (final) mannose-6-phosphate. Final reaction volume was 1.0 mL. All components were dissolved in Tris/Mg buffer. Activities were taken as the change in A₃₄₀/min. From these activities was subtracted the activities measured in identical reaction mixtures lacking the M-6-P substrate. Specific activities were calculated by normalizing the activities for protein concentration in the reactions. Protein in the original extracts was determined by the method of Bradford, using a kit from Bio-Rad Laboratories (Hercules, Calif.). All enzymes and nucleotides were purchased from Sigma Chemical Co. (St. Louis, Mo.).

### Phosphomannomutase (PMM) Assay

Phosphomannomutase was measured in a similar manner in the same strains, but these assay reaction mixtures also contained 0.25 mM glucose-1,6-diphosphate, 0.5 U commercially available PMI, and the reactions were started with the addition of 3.0 mM (final) mannose-1-phosphate rather than mannose-6-phosphate.

### Phosphofructokinase (PFK) Assay

To shed light on the possibility that the enhancement of AA concentration in cultures which were limited for magnesium was due to a diversion of carbon from normal metabolism by a reduced activity of the first committed step in glycolysis (PFK) the strains were also assayed to confirm the presence of this enzyme activity. Cells were cultured, washed and broken as above. Extracts were centrifuged at 100,000×g for 90 min before desalting. Reactions were carried out in the forward direction by adding various volumes of extracts to solutions of Tris/Mg buffer containing 1.5 mM dithiothreitol, 0.86 U aldolase (EC 4.1.2.13), 1.4 U α-glycerophosphate dehydrogenase (EC 1.1.1.8), 14 U triosephosphate isomerase (EC 5.3.1.1), 0.11 mM NADH, and 1.0 mM ATP. Reactions were initiated by addition of 5 mM (final) fructose-6-phosphate. Final reaction volume was 1.0 mL. All components were dissolved in Tris/Mg buffer. Activities were taken as the change in A₃₄₀/min. From these activities were subtracted the activities measured in identical reaction mixtures lacking the F-6-P substrate. Specific activities were calculated by normalizing the activities for protein concentration in the reaction. Protein in the original extracts was determined as above.

### GDP-D-mannose Pyrophosphorylase (GMP) Assay

These same mutant strains were assayed for the next enzyme in the proposed pathway, GMP. Strains were grown both according to the standard Mg-limiting protocol (harvested 43-48 hours into magnesium-limited incubation) and in standard Mg-sufficient medium (harvesting all cells before glucose depletion). Washed cell pellets were resuspended in 50 mM phosphate buffer, pH 7.0, containing 20% (v/v) glycerol and 0.1 M sodium chloride (3 mL buffer/g wet cells), and broken in a French press. Crude extracts were spun at 15,000×g for 15 minutes. Reactions were carried out in the forward direction by adding various volumes of extracts to solutions of 50 mM phosphate/4 mM MgCl₂ buffer, pH 7.0, containing 1 mM GTP. Reactions were initiated by addition of 1 mM (final) mannose-1-phosphate. Final reaction volume was 0.1 mL. Reaction mixtures were incubated at 30° C. for 10 min, filtered through a 0.45 μm PVDF syringe filter, and analyzed for GDP-mannose by HPLC. A Supelcosil SAX1 column (4.6×250 mm) was used with a solvent gradient (1 mL/min) of: A—6 mM potassium phosphate, pH 3.6; B—500 mM potassium phosphate, pH 4.5. The gradient was: 0-3 min, 100% A; 3-10 min, 79% A; 10-15 min, 29% A. Column temperature was 30° C. Two assays that showed enzyme activity proportional to the amount of protein were averaged. Control no-substrate and no-extract reactions were also run. Specific activity was calculated by normalizing the activity for protein concentration in the reaction. Protein in the original extracts was determined as above.

### GDP-D-mannose:GDP-L-galactose Epimerase Assay

Further tests measured the activities of the next enzyme in the proposed pathway, GDP-D-mannose:GDP-L-galactose epimerase. Strains were grown according to the standard protocol, harvested 43-48 hours into magnesium-limited incubation, washed, and resuspended in buffer containing 50 mM MOPS/S mM EDTA, pH 7.2. Washed pellets were broken in a French press, and spun at 20,000×g for 20 min. Protein determinations were made as above and a dilution series of each was made, ranging from 0.4 to 2.2 mg protein/mL. 50 μL aliquots of these dilutions were added to 10 μL aliquots of 6.3 mM GDP-D-mannose in which a portion of this substrate was universally labeled with ¹⁴C in the mannose moiety. This substrate had an activity of 16 μCi/mL before dilution into the reaction mixture. Reactions were stopped after 10 min by transferring 20 μL of the mixture into microfuge tubes containing 20 μL of 250 mM trifluoroacetic acid (TFA) containing 1.0 g/L each D-mannose and L-galactose. These tubes were sealed and boiled for 10 min, cooled, spun for 60 sec in a Beckman Microfuge E, and 5 μL of each hydrolysate was spotted on 20×20 cm plastic-backed EM Science Silica gel 60 thin-layer chromatography plates (#5748/7), with 1 cm lanes created by scoring with a blunt stylus. After drying, plates were twice chromatographed for 2.5 hours in ethyl acetate:isopropanol:water, 65:22.3:12.7 (plates were dried between runs). Spots of free sugars were visualized by spraying dried plates with 0.5% p-anisaldehyde in a 62% ethanolic solution of 0.89 M sulfuric acid and 0.17 mM glacial acetic acid, and heating at 105° C. for about 15 min. Spots of L-galactose and D-mannose were cut from the plates and counted in a scintillation counter (Beckman model 2800). For time-zero control counts, 16.7 μL of each extract dilution was added to 23.3 μL of the labeled substrate above, which had been diluted 1:7 with the TFA/mannose/galactose solution.

Table 11 summarizes the results of the five enzyme assays for the strains tested, along with their specific AA formations.

TABLE 11
Specific Enzyme Activities (mU)* of Selected Mutant Prototheca Strains

| | | | | | GMP | | |
|---|---|---|---|---|---|---|---|
| | AA Specific | | | | | Mg- | |
| Strain | Form, mg/g | PMI | PMM | PFK | Mg-limited | sufficient | Epimerase |
| UVI 64-6 | 78.4 | | | | | | 0.79 |
| EMS13-4 | 73.7 | 10.8 | 69.6 | 13.5 | 2.6 | 6.8 | 0.78 |
| UV140-1 | 69.9 | | | | | | 0.78 |
| NA45-3 | 61.4 | | | | | | 0.58 |
| UV77-247 | 44.4 | | | | | | 0.52 |
| UV127-10 | 40.1 | 11.1 | 45.8 | 24.4 | 4.3 | 5.9 | 0.39 |
| UV244-15 | 24.5 | 14.3 | 41.5 | | 3.1 | 5.3 | 0.42 |
| NA21-14 | 23.6 | 12.1 | 60.3 | 47.4 | 2.4 | 7.6 | 0.27 |
| ATCC 75669 | 21.9 | | | | | | 0.28 |
| UV244-16 | 5.0 | 16.5 | 85.6 | 4.3 | 5.2 | | |
| SP2-3 | 2.0 | 17.7 | 47.0 | 64.5 | 2.0 | 7.5 | 0.03 |
| UV218-1 | 0.4 | 15.9 | 72.1 | | 2.7 | 7.0 | 0.83 |
| UV213-1 | 0.1 | 19.7 | 47.7 | 32.6 | 3.2 | 6.7 | 0.60 |
| UV82-21 | 0.0 | 14.6 | 70.6 | 30.4 | 4.1 | 7.5 | 0.15 |
| UV244-1 | 0.0 | 18.2 | 51.1 | | 5.5 | 12 | 0.15 |

Units: PMI and PMM, nmoles NADP reduced per mm/mg protein; PFK, nmoles NADH oxidized per min/mg protein; GMP, nmoles GDP-D-mannose formed per mm/mg protein; epimerase, nmoles GDP-L-galactose formed per min/mg protein.

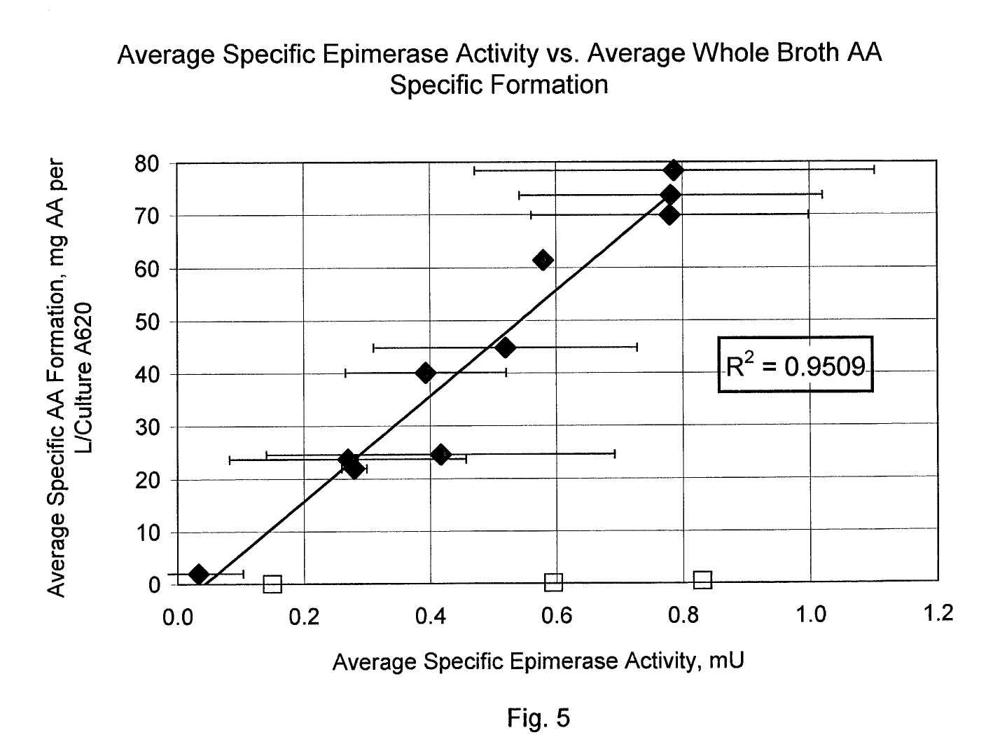

The only enzyme which showed a strong correlation between activity and the ability to synthesize AA was the GDP-D-mannose:GDP-L-galactose epimerase. This correlation is depicted in FIG. 5. All of the strains which produced measurable amounts of AA had measurable amounts of epimerase activity. The converse was not true: four of the strains which synthesize little or no AA had significant epimerase activities. These strains are candidates for having mutations which affect enzymatic steps downstream from the epimerase. Since all of the strains tested can synthesize AA from L-galactose and L-galactono-γ-lactone (see Examples 4 and 5), the genetic lesion(s) in these four mutants must lie between GDP-L-galactose and free L-galactose.

### Example 8

The next example shows the relationship between GDP-D-mannose:GDP-L-galactose epimerase activity and the degree of magnesium limitation in two strains, the original unmutagenized parent strain ATCC 75669, and one of the best AA producers, EMS13-4 (ATCC ______).

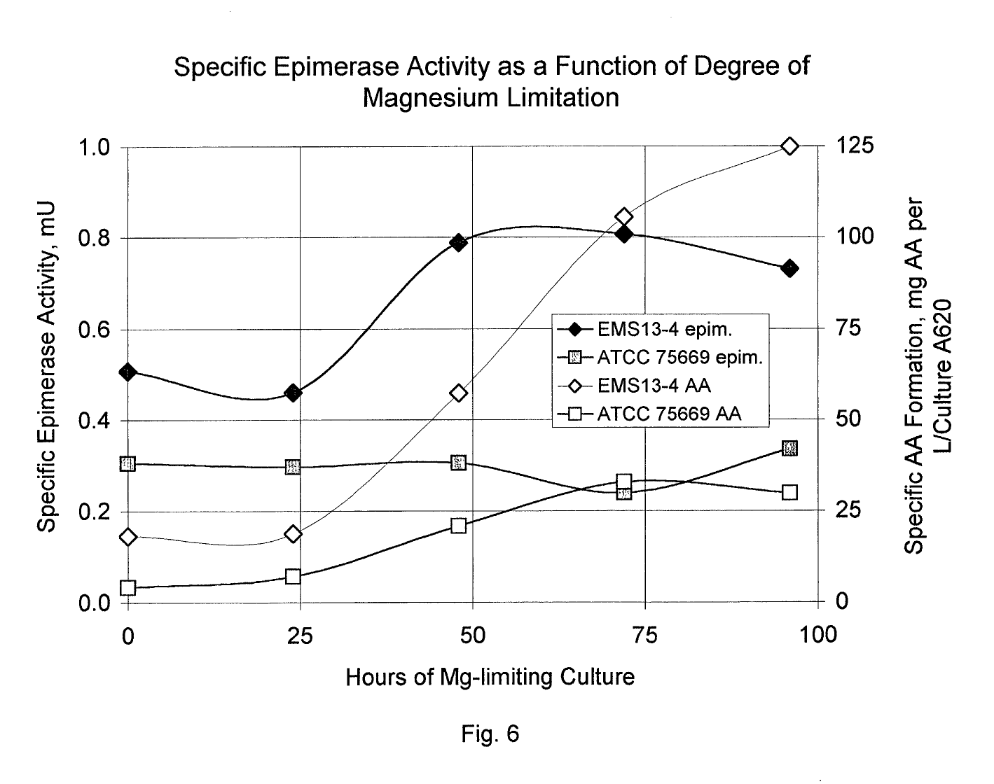

Four flasks of each strain were grown according to the standard protocol. One culture of each was harvested 24 hours into magnesium-limited incubation, and every 24 hours thereafter for a total of four days. One flask of each strain was also harvested 24 hours into magnesium sufficient incubation. All cultures had glucose remaining when harvested. FIG. 6 shows graphically the AA productivity and epimerase activity in EMS13-4 and ATCC 75669 as the cultures became Mg-limited. Epimerase activity in EMS13-4 was significantly greater than that in ATCC 75669 at all time points. There was also a concurrent rapid rise in both AA productivity and epimerase activity in EMS13-4 as the cultures became increasingly Mg-limited. While there was a moderate increase in AA productivity in ATCC 75669 as Mg became more limiting, there was no effect on epimerase activity.

### Example 9

The following example shows the results of epimerase assays performed with extracts of two E. coli strains into which were cloned the E. coli gene for GDP-4-keto-6-deoxy-D-mannose epimerase/reductase.

The E. coli K12 wca gene cluster is responsible for cholanic acid production; wcaG encodes a GDP-4-keto-6-deoxy-D-mannose epimerase/reductase.

The E. coli wcaG sequence (nucleotides 4 through 966 of SEQ ID NO:3) was amplified by PCR from E. coli W3110 genomic DNA using primers WG EcoRI 5 (5′ TAGAATTCAGTAAACAACGAGTTTTTATTGCTGG 3′; SEQ ID NO:12) and WG Xhol 3 (5′ AACTCGAGTTACCCCCAAAGCGGTCTTGATTC 3′; SEQ ID NO:13). The 973-bp PCR product was ligated into the vector pPCR-Script SK(+) (Stratagene, LaJolla, Calif.). The 973-bp ExoRII/XhoI fragment was moved from this plasmid into the ExoRII/XhoI sites of pGEX-5X-1 (Amersham Pharmacia Biotech, Piscataway, N.J.), creating plasmid pSW67-1. Plasmid pGEX-5X-1 is a GST gene fusion vector which adds a 26-kDa GST moiety onto the N-terminal end of the protein of interest. E. coli BL21(DE3) was transformed with pSW67-1 and pGEX-5X-1, resulting in strains BL21(DE3)/pSW67-1 and BL21(DE3)/pGEX-5X-1.

The E. coli wcaG sequence (nucleotides 1 through 966 of SEQ ID NO:3) was also amplified by PCR from E. coli W3110 genomic DNA using primers WG EcoRI 5-2 (5′ CTGGAGTCGAATTCATGAGTAAACAACGAG 3′; SEQ ID NO:14) and WG PstI 3 (5′ AACTGCAGTTACCCCCGAAAGCGGTCTTGATTC 3′; SEQ ID NO:15). The 976-bp PCR product was ligated into a pPCR-Script (Stratagene). The 976-bp ExoRII/PstI fragment was moved from this plasmid into the ExoRII/PstI sites of expression vector pKK223-3 (Amersham Pharmacia Biotech), creating plasmid pSW75-2. E. coli JM105 was transformed with pKK223-3 and pSW75-2, resulting in strains JM105/pKK223-3 and JM105/pSW75-2.

All six strains were grown in duplicate at 37° C. with shaking in 2X YTA medium until an optical density of 0.8-1.0 at 600 nm was reached (about three hours). 2X YTA contains 16 g/L tryptone, 10 g/L yeast extract, 5 g/L sodium chloride and 100 mg/L ampicillin. One of each culture was induced by adding isopropyl β-D-thiogalactopyranoside (IPTG) to 1 mM final concentration. All 12 cultures were incubated for an additional four hours, washed in 0.9% NaCl, and the cells were frozen at −80° C. Prior to pelleting the cells for preparation of extracts, a portion of each culture was used for a plasmid DNA miniprep to confirm the presence of the appropriate plasmids in these strains. A protein preparation of each culture was also run on SDS gels to confirm expression of a protein of the appropriate size where expected. Frozen pellets were thawed, resuspended in 2.5 mL MOPS/EDTA buffer, pH 7.2, broken in a French Press (10,000 psi), spun for 20 min at 20,000×g, assayed for protein as above and diluted to 0.01, 0.1, 1.0 and 3 mg/mL protein.

Induction of the strain BL21(DE3)/pGEX-5X-1 resulted in high-level expression of a 26-kDa protein indicating the synthesis of the native GST protein. Induction of strain BL21(DE3)/pSW67-1 resulted in high-level expression of a 62-kDa protein, indicating the synthesis of the native GST protein (26K) fused to the wcaG gene product (36K). An aliquot of the fusion protein was treated with the protease Factor Xa (New England Biolabs, Beverly, Mass.), which cleaves near the GST/wcaG junction. Induction of the strain JM105/pSW75-2 resulted in high level expression of a 36-kDa protein, indicating the synthesis of the wcaG gene product. No such protein was detected in JM105/pKK223-3 (vector only).

Next, it was of interest to test extracts in the standard epimerase assay described in Example 7 to determine if any of the extracts containing the wcaG product could bring about the conversion of GDP-D-mannose to GDP-L-galactose. The extracts to be assayed are:

BL21(DE3) Group

1. BL21(DE3) uninduced

2. BL21(DE3) induced with 1 mM IPTG

3. BL21(DE3)/pGEX-5X-1 uninduced

4. BL21(DE3)/pGEX-5X-1 induced with 1 mM IPTG

5. BL21(DE3)/pSW67-1 uninduced

6. BL21(DE3)/pSW67-1 induced with 1 mM IPTG; fusion protein intact

7. BL21(DE3)/pSW67-1 induced with 1 mM IPTG; GST moiety cleaved

JM105 Group

1. JM105 uninduced

2. JM105 induced with 1 mM IPTG

3. JM105/pKK223-3 uninduced

4. JM105/pKK223-3 induced with 1 mM IPTG

5. JM105/pSW75-2 uninduced

6. JM105/pSW75-2 induced with 1 mM IPTG

Extracts 1 and 7 from the BL21(DE3) group and extracts 1 and 6 from the JM105 group were tested for GDP-D-mannose:GDP-L-galactose epimerase-like activity in a pilot experiment. In this initial experiment, no epimerase activity was detected in any of the extracts. At this time, such a result can be attributed to a number of possibilities. First, it is possible that the wcaG gene product is incapable of catalyzing the conversion of GDP-D-mannose to GDP-L-galactose, although this conclusion can not be reached until several other parameters are tested. Second, it is possible that under the assay conditions which are satisfactory to measure activity for the endogenous GDP-D-mannose:GDP-L-galactose epimerase, the wcaG gene product does not have GDP-D-mannose:GDP-L-galactose epimerase-like activity. Therefore, alternate conditions should be tested. Additionally, confirmation experiments should be performed to confirm the accuracy of the pilot conditions. Third, although the BL21(DE3) and the JM105 clones produce proteins of the expected size, the constructs have not been sequenced to confirm the proper coding sequence for the wcaG gene product and thereby rule out PCR or cloning errors which may render the wcaG gene product inactive. Fourth, the protein formed from the cloned sequence is full-length, but inactive, for example, due to incorrect tertiary structure (folding). Fifth, the gene is overexpressed, resulting in accumulation of insoluble and inactive protein products (inclusion bodies). Future experiments will attempt to determine whether the constructs have or can be induced to have the ability to catalyze the conversion of GDP-D-mannose to GDP-L-galactose, and to use the sequences to isolate the endogenous GDP-D-mannose:GDP-L-galactose epimerase.

Table 12 provides the atomic coordinates for Brookhaven Protein Data Bank Accession Code 1bws:

TABLE 12

| | | | |
|---|---|---|---|
| HEADER | EPIMERASE/REDUCTASE | 27-SEP-98 | 1BWS |

| | | | |
|---|---|---|---|
| TITLE | | CRYSTAL STRUCTURE OF GDP-4-KETO-6-DEOXY-D-MANNOSE | |
| TITLE | 2 | EPIMERASE/REDUCTASE FROM ESCHERICHIA COLI A KEY ENZYME IN | |
| TITLE | 3 | THE BIOSYNTHESIS OF GDP-L-FUCOSE | |
| COMPND | | MOL ID: 1; | |
| COMPND | 2 | MOLECULE: GDP-4-KETO-6-DEOXY-D-MANNOSE EPIMERASE/REDUCTASE; | |
| COMPND | 3 | CHAIN: A; | |
| COMPND | 4 | ENGINEERED: YES; | |
| COMPND | 5 | BIOLOGICAL UNIT: HOMODIMER | |
| SOURCE | | MOL ID: 1; | |
| SOURCE | 2 | ORGANISM SCIENTIFIC: ESCHERICHIA COLI; | |
| SOURCE | 3 | EXPRESSION SYSTEM: ESCHERICHIA COLI | |
| KEYWDS | | EPIMERASE/REDUCTASE, GDP-L-FUCOSE BIOSYNTHESIS | |
| EXPDTA | | X-RAY DIFFRACTION | |
| AUTHOR | | DE M. RIZZITONETTIFLORA | |
| REVDAT | 1 | 13-JAN-99 1BWS 0 | |
| JRNL | | AUTH DE D .RIZZITONETTIVIGEVANISTURLABISSOFLORA | |
| JRNL | | TITL GDP-4-KETO-6-DEOXYD-MANNOSE EPIMERASE/REDUCTASE | |
| JRNL | | TITL 2 FROM ESCHERICHIA COLI, A KEY ENZYME IN THE | |
| JRNL | | TITL 3 BIOSYNTHESIS OF GDP-L-FUCOSE, DISPLAYS THE | |
| JRNL | | TITL 4 STRUCTURAL CHARACTERISTICS OF THE RED PROTEIN | |
| JRNL | | TITL 5 HOMOLOGY SUPERFAMILY | |
| JRNL | | REF STRUCTURE (LONDON) | 1998 |
| JRNL | | REFN | 9999 |
| REMARK | 1 | | |
| REMARK | 2 | | |
| REMARK | 2 | RESOLUTION. 2.2 ANGSTROMS | |
| REMARK | 3 | | |
| REMARK | 3 | REFINEMENT. | |
| REMARK | 3 | PROGRAM | : TNT |
| REMARK | 3 | AUTHORS | : TRONRUD, TEN EYCK, MATTHEWS |
| REMARK | 3 | | |
| REMARK | 3 | DATA USED IN REFINEMENT. | |

| | | | |
|---|---|---|---|
| REMARK | 3 | RESOLUTION RANGE HIGH (ANGSTROMS) | : 2.2 |
| REMARK | 3 | RESOLUTION RANGE LOW (ANGSTROMS) | : 15.0 |
| REMARK | 3 | DATA CUTOFF (SIGMA(F)) | : 0.0 |
| REMARK | 3 | COMPLETENESS FOR RANGE (%) | : 99.7 |
| REMARK | 3 | NUMBER OF REFLECTIONS | : 24481 |
| REMARK | 3 | | |
| REMARK | 3 | USING DATA ABOVE SIGMA CUTOFF. | |
| REMARK | 3 | CROSS-VALIDATION METHOD | : NONE |
| REMARK | 3 | FREE R VALUE TEST SET SELECTION | : NULL |
| REMARK | 3 | R VALUE (WORKING + TEST SET) | : NULL |
| REMARK | 3 | R VALUE (WORKING SET) | : NONE |
| REMARK | 3 | FREE R VALUE | : NULL |
| REMARK | 3 | FREE R VALUE TEST SET SIZE (%) | : NONE |
| REMARK | 3 | FREE R VALUE TEST SET COUNT | : NULL |
| REMARK | 3 | | |
| REMARK | 3 | USING ALL DATA, NO SIGMA CUTOFF. | |

| | | | |
|---|---|---|---|
| REMARK | 3 | R VALUE (WORKING + TEST SET, NO CUTOFF) | : NULL |
| REMARK | 3 | R VALUE (WORKING SET, NO CUTOFF) | : 0.202 |
| REMARK | 3 | FREE R VALUE (NO CUTOFF) | : 0.287 |
| REMARK | 3 | FREE R VALUE TEST SET SIZE (%, NO CUTOFF) | : NULL |
| REMARK | 3 | FREE R VALUE TEST SET COUNT (NO CUTOFF) | : NULL |
| REMARK | 3 | TOTAL NUMBER OF REFLECTIONS (NO CUTOFF) | : NULL |
| REMARK | 3 | | |

| REMARK | 3 | NUMBER OF NON-HYDROGEN ATOMS USED IN REFINEMENT. | |
|---|---|---|---|
| REMARK | 3 | PROTEIN ATOMS | : 2527 |
| REMARK | 3 | NUCLEIC ACID ATOMS | : NULL |
| REMARK | 3 | OTHER ATOMS | : 109 |
| REMARK | 3 | | |

| REMARK | 3 | WILSON B VALUE (FROM FCALC, A**2) | : NULL | | |
|---|---|---|---|---|---|
| REMARK | 3 | | | | |
| REMARK | 3 | EMS DEVIATIONS FROMIDEAL VALUES. | EMS | WEIGHT | COUNT |
| REMARK | 3 | BOND LENGTHS (A) | : 0.016 | ; NULL | ; NULL |
| REMARK | 3 | BOND ANGLES (DEGREES) | : 1.65 | ; NULL | ; NULL |
| REMARK | 3 | TORSION ANGLES (DEGREES) | : NULL | ; NULL | ; NULL |
| REMARK | 3 | PSEUDOROTATION ANGLES (DEGREES) | : NULL | ; NULL | ; NULL |
| REMARK | 3 | TRIGONAL CARBON PLANES (A) | : NULL | ; NULL | ; NULL |
| REMARK | 3 | GENERAL PLANES (A) | : NULL | ; NULL | ; NULL |
| REMARK | 3 | ISOTROPIC THERMAL FACTORS (A**2) | : NULL | ; NULL | ; NULL |
| REMARK | 3 | NON-BONDED CONTACTS (A) | : NULL | ; NULL | ; NULL |
| REMARK | 3 | | | | |
| REMARK | 3 | INCORRECT CHIRAL-CENTERS (COUNT) | : NULL | | |
| REMARK | 3 | | | | |
| REMARK | 3 | BULK SOLVENT MODELING. | | | |

| | | | |
|---|---|---|---|
| REMARK | 3 | METHOD USED | : NULL |
| REMARK | 3 | KSOL | : NULL |
| REMARK | 3 | ESOL | : NULL |
| REMARK | 3 | | |
| REMARK | 3 | RESTRAINT LIBRARIES. | |

| | | | |
|---|---|---|---|
| REMARK | 3 | STEREOCHEMISTRY : NULL | |
| REMARK | 3 | ISOTROPIC THERMAL FACTOR RESTRAINTS : NULL | |
| REMARK | 3 | | |
| REMARK | 3 | OTHER REFINEMENT REMARKS: NULL | |
| REMARK | 4 | | |
| REMARK | 4 | 1BWS COMPLIES WITH FORMAT V. 2.2, 16-DEC-1996 | |
| REMARK | 5 | | |
| REMARK | 5 | WARNING | |
| REMARK | 5 | 1BWS: THIS IS LAYER 1 RELFASE. | |
| REMARK | 5 | | |
| REMARK | 5 | PLEASE NOTE THAT THIS ENTRY WAS RELEASED AFTER DEPOSITOR | |
| REMARK | 5 | CHECKING AND APPROVAL BUT WITHOUT PDB STAFF INTERVENTION. | |
| REMARK | 5 | AN AUXILIARY FILE, AUX1BWS.RPT, IS AVAILABLE FROM THE | |
| REMARK | 5 | PDB FTP SERVER AND IS ACCESSIBLE THROUGH THE 3DB BROWSER. | |
| REMARK | 5 | THE FILE CONTAINS THE OUTPUT OF THE PROGRAM WHAT CHECK AND | |
| REMARK | 5 | OTHER DIAGNOSTICS. | |
| REMARK | 5 | | |
| REMARK | 5 | NOMENCLATURE IN THIS ENTRY, INCLUDING HET RESIDUE NAMES | |
| REMARK | 5 | AND HET ATOM NAMES, HAS NOT BEEN STANDARDIZED BY THE PDB | |
| REMARK | 5 | PROCESSING STAFF. | A LAYER 2 ENTRY WILL BE RELEASED SHORTLY |
| REMARK | 5 | AFTER THIS STANDARDIZATION IS COMPLETED AND APPROVED BY THE | |
| REMARK | 5 | DEPOSITOR. THE LAYER 2 ENTRY WILL BE TREATED AS A | |
| REMARK | 5 | CORRECTION TO THIS ONE, WITH THE APPROPRIATE REVDAT RECORD. | |
| REMARK | 5 | | |
| REMARK | 5 | FURTHER INFORMATION INCLUDING VALIDATION CRITERIA USED IN | |
| REMARK | 5 | CHECKING THIS ENTRY AND A LIST OF MANDATORY DATA FIELDS | |
| REMARK | 5 | ARE AVAILABLE FROM THE PDB WEB SITE AT | |
| REMARK | 5 | HTTP://WWW.PDB.BNL.GOV/. | |
| REMARK | 200 | | |
| REMARK | 200 | EXPERIMENTAL DETAILS | |

| REMARK | 200 | EXPERIMENT TYPE | : X-RAY DIFFRACTION |
|---|---|---|---|
| REMARK | 200 | DATE OF DATA COLLECTION | : AUG-1997 |
| REMARK | 200 | TEMPERATURE (KELVIN) | : 120 |
| REMARK | 200 | PH | : 6.5 |
| REMARK | 200 | NUMBER OF CRYSTALS USED | :1 |
| REMARK | 200 | | |
| REMARK | 200 | SYNCHROTRON (Y/N) | N |
| REMARK | 200 | RADIATION SOURCE | : NONE |
| REMARK | 200 | BEAMLINE | : NULL |
| REMARK | 200 | X-RAY GENERATOR MODEL | : RIGAKU RU200 |
| REMARK | 200 | MONOCHROMATIC OR LAUE (M/L) | : M |
| REMARK | 200 | WAVELENGTH OR RANGE (A) | : 1.5418 |
| REMARK | 200 | MONOCHROMATOR | : NULL |
| REMARK | 200 | OPTICS | : NULL |
| REMARK | 200 | | |
| REMARK | 200 | DETECTOR TYPE | : IMAGE PLATE |
| REMARK | 200 | DETECTOR MANUFACTURER | : RAXIS |
| REMARK | 200 | INTENSITY-INTEGRATION SOFTWARE | : MOSFLM |
| REMARK | 200 | DATA SCALING SOFTWARE | : SCALA |
| REMARK | 200 | | |
| REMARK | 200 | NUMBER OF UNIQUE REFLECTIONS | : 24481 |
| REMARK | 200 | RESOLUTION RANGE HIGH (A) | : 2.2 |
| REMARK | 200 | RESOLUTION RANGE LOW (A) | : 15.0 |
| REMARK | 200 | REJECTION CRITERIA (SIGMA(I)) | : NONE |
| REMARK | 200 | | |
| REMARK | 200 | OVERALL. | |
| REMARK | 200 | COMPLETENESS FOR RANGE (%) | : 99.7 |
| REMARK | 200 | DATA REDUNDANCY | : 4.3 |
| REMARK | 200 | R MERGE (I) | : 0.057 |
| REMARK | 200 | R SYM (I) | : NONE |
| REMARK | 200 | <I/SIGMA(I)> FOR THE DATA SET | : 13.6 |
| REMARK | 200 | | |
| REMARK | 200 | IN THE HIGHEST RESOLUTION SHELL. | |
| REMARK | 200 | HIGHEST RESOLUTION SHELL, RANGE HIGH (A) : NULL | |
| REMARK | 200 | HIGHEST RESOLUTION SHELL, RANGE LOW (A) : NULL | |
| REMARK | 200 | COMPLETENESS FOR SHELL (%) | : NULL |
| REMARK | 200 | DATA REDUNDANCY IN SHELL | : NULL |
| REMARK | 200 | R MERGE FOR SHELL (I) | : NULL |
| REMARK | 200 | R SYM FOR SHELL (I) | : NULL |
| REMARK | 200 | <I/SIGMA(I)> FOR SHELL | : NULL |
| REMARK | 200 | | |

| REMARK | 200 | DIFFRACTION PROTOCOL: NULL | | |
|---|---|---|---|---|
| REMARK | 200 | METHOD USED TO DETERMINE THE STRUCTURE: MIR | | |
| REMARK | 200 | SOFTWARE USED: NULL | | |
| REMARK | 200 | STARTING MODEL: NULL | | |
| REMARK | 200 | | | |
| REMARK | 200 | REMARK: NULL | | |
| REMARK | 280 | | | |
| REMARK | 280 | CRYSTAL | | |
| REMARK | 280 | SOLVENT CONTENT, VS (%): NULL | | |
| REMARK | 280 | MATTHEWS COEFFICIENT, VM (ANGSTROMS**3/DA): NULL | | |
| REMARK | 280 | | | |
| REMARK | 280 | CRYSTALLIZATION CONDITIONS: NULL | | |
| REMARK | 290 | | | |
| REMARK | 290 | CRYSTALLOGRAPHIC SYNMETRY | | |
| REMARK | 290 | SYMMETRY OPERATORS FOR SPACE GROUP: P 32 2 1 | | |
| REMARK | 290 | | | |
| REMARK | 290 | | SYMOP | SYMMETRY |
| REMARK | 290 | | NNWHMM | OPERATOR |
| REMARK | 290 | | 1555 | X,Y,Z |
| REMARK | 290 | | 2555 | −Y,X−Y,Z+2/3 |
| REMARK | 290 | | 3555 | Y−X,−X,Z+1/3 |
| REMARK | 290 | | 4555 | Y,X,−Z |
| REMARK | 290 | | 5555 | X−Y,−Y,1/3−Z |
| REMARK | 290 | | 6555 | −X,Y−X,2/3−Z |
| REMARK | 290 | | | |

| | | |
|---|---|---|
| REMARK | 290 | WHERE NNN —> OPERATOR NUMBER |

| | | |
|---|---|---|
| REMARK | 290 | MMM —> TRANSLATION VECTOR |
| REMARK | 290 | |

| REMARK | 290 | CRYSTALLOGRAPHIC SYMMETRY TRANSFORMATIONS | | | | | |
|---|---|---|---|---|---|---|---|
| REMARK | 290 | THE FOLLOWING TRANSFORMATIONS OPERATE ON THE ATOM/HETATM | | | | | |
| REMARK | 290 | RECORDS IN THIS ENTRY TO PRODUCE CRYSTALLOGRAPHICALLY | | | | | |
| REMARK | 290 | RELATED MOLECULES. | | | | | |
| REMARK | 290 | SMTRY1 | 1 | 1.000000 | 0.000000 | 0.000000 | 0.00000 |
| REMARK | 290 | SMTRY2 | 1 | 0.000000 | 1.000000 | 0.000000 | 0.00000 |
| REMARK | 290 | SMTRY3 | 1 | 0.000000 | 0.000000 | 1.000000 | 0.00000 |
| REMARK | 290 | SMTRY1 | 2 | −0.500045 | −0.865974 | 0.000000 | 0.00000 |
| REMARK | 290 | SMTRY2 | 2 | 0.866077 | −0.499955 | 0.000000 | 0.00000 |
| REMARK | 290 | SMTRY3 | 2 | 0.000000 | 0.000000 | 1.000000 | 50.58553 |
| REMARK | 290 | SMTRY1 | 3 | −0.499955 | 0.865974 | 0.000000 | 0.00000 |
| REMARK | 290 | SMTRY2 | 3 | −0.866077 | −0.500045 | 0.000000 | 0.00000 |
| REMARK | 290 | SMTRY3 | 3 | 0.000000 | 0.000000 | 1.000000 | 25.29276 |
| REMARK | 290 | SMTRY1 | 4 | −0.500045 | 0.865922 | 0.000000 | 0.00000 |
| REMARK | 290 | SMTRY2 | 4 | 0.866077 | 0.500045 | 0.000000 | 0.00000 |
| REMARK | 290 | SMTRY3 | 4 | 0.000000 | 0.000000 | 1.000000 | 0.00000 |
| REMARK | 290 | SMTRY1 | 5 | 1.000000 | 0.000104 | 0.000000 | 0.00000 |
| REMARK | 290 | SMTRY2 | 5 | 0.000000 | 1.000000 | 0.000000 | 0.00000 |
| REMARK | 290 | SMTRY3 | 5 | 0.000000 | 0.000000 | 1.000000 | 25.29276 |
| REMARK | 290 | SMTRY1 | 6 | −0.499955 | 0.866026 | 0.000000 | 0.00000 |
| REMARK | 290 | SMTRY2 | 6 | −0.866077 | 0.499955 | 0.000000 | 0.00000 |
| REMARK | 290 | SMTRY3 | 6 | 0.000000 | 0.000000 | 1.000000 | 50.58553 |
| REMARK | 290 | | | | | | |

| REMARK | 290 | REMARK: NULL | |
|---|---|---|---|
| REMARK | 465 | | |
| REMARK | 465 | MISSING RESIDUES | |
| REMARK | 465 | THE FOLLOWING RESIDUES WERE NOT LOCATED IN THE | |
| REMARK | 465 | EXPERIMENT. (M = MODEL NUMBER; RES = RESIDUE NAME; C = CHAIN | |
| REMARK | 465 | IDENTIFIER; SSSEQ = SEQUENCE NUMBER; I = INSERTION CODE): | |
| REMARK | 465 | | |
| REMARK | 465 | M RES C SSSEQI | |
| REMARK | 465 | MET A | 1 |
| REMARK | 465 | SER A | 2 |
| REMARK | 465 | ASP A | 317 |
| REMARK | 465 | ARG A | 318 |
| REMARK | 465 | PHE A | 319 |
| REMARK | 465 | ARG A | 320 |
| REMARK | 465 | GLY A | 321 |
| REMARK | 800 | | |

| | | |
|---|---|---|
| REMARK | 800 | SITE |
| REMARK | 800 | SITE IDENTIFIER: CAT |
| REMARK | 800 | SITE DESCRIPTION: |
| REMARK | 800 | CATALYTIC RESIDUE |
| REMARK | 800 | |
| REMARK | 800 | SITE IDENTIFIER: CAT |
| REMARK | 800 | SITE DESCRIPTION: |
| REMARK | 800 | CATALYTIC RESIDUE |
| REMARK | 800 | |
| REMARK | 800 | SITE IDENTIFIER: CAT |
| REMARK | 800 | SITE DESCRIPTION: |
| REMARK | 800 | CATALYTIC RESIDUE |
| REMARK | 800 | |

| | | | | | | |
|---|---|---|---|---|---|---|
| DBREF | 1BWS A | 3 | 316 | SWS | P32055 | FCL ECOLI |

| | | | | | | | | | | | | | | | | |
|---|---|---|---|---|---|---|---|---|---|---|---|---|---|---|---|---|
| SEQRES | 1 | A | 321 | MET | SER | LYS | GLN | ARG | VAL | PHE | ILE | ALA | GLY | HIS | ARG | GLY |
| SEQRES | 2 | A | 321 | MET | VAL | GLY | SER | ALA | ILE | ARG | ARG | GLN | LEU | GLU | GLN | ARS |
| SEQRES | 3 | A | 321 | GLY | ASP | VAL | GLU | LEU | VAL | LEU | ARS | THR | ARG | ASP | GLU | LEO |
| SEQRES | 4 | A | 321 | ASN | LEU | LEU | ASP | SER | ARG | ALA | VAL | HIS | ASP | PHE | PHE | ALA |
| SEQRES | 5 | A | 321 | SER | GLU | ARS | ILE | ASP | GLN | VAL | TYR | LEU | ALA | ALA | ALA | LYS |
| SEQRES | 6 | A | 321 | VAL | GLY | GLY | ILE | VAL | ALA | ASN | ASN | THR | TYR | PRO | ALA | ASP |
| SEQRES | 7 | A | 321 | PHE | ILE | TYR | GLN | ASN | MET | MET | ILE | GLU | SER | ASN | ILE | ILE |
| SEQRES | 8 | A | 321 | HIS | ALA | ALA | HIS | GLN | ASN | ASP | VAL | ASN | LYS | LEU | LEU | PHE |
| SEQRES | 9 | A | 321 | LEU | SLY | SER | SER | CYS | ILE | TYR | PRO | LYS | LEU | ALA | LYS | GLN |
| SEQRES | 10 | A | 321 | PRO | MET | ALA | GLU | SER | GLU | LEU | LEU | GLN | GLY | THR | LEU | GLU |
| SEQRES | 11 | A | 321 | PRO | TER | ASN | GLU | PRO | TYR | ALA | ILE | ALA | LYS | ILE | ALA | SLY |
| SEQRES | 12 | A | 321 | ILE | LYS | LEU | CYS | GLU | SER | TYR | ASN | ARG | GLN | TYR | GLY | ARG |
| SEQRES | 13 | A | 321 | ASP | TYR | ARS | SER | VAL | MET | PRO | THE | ASN | LEU | TYR | GLY | PRO |
| SEQRES | 14 | A | 321 | HIS | ASP | ASN | PHE | HIS | PRO | SER | ASN | SER | HIS | VAL | ILE | PRO |
| SEQRES | 15 | A | 321 | ALA | LEU | LEU | ARG | ARG | PHE | HIS | GLU | ALA | THR | ALA | GLN | ASN |
| SEQRES | 16 | A | 321 | ALA | PRO | ASP | VAL | VAL | VAL | TRP | GLY | SER | GLY | THR | PRO | MET |
| SEQRES | 17 | A | 321 | ARS | GLU | PHE | LEU | HIS | VAL | ASP | ASP | MET | ALA | ALA | ALA | SER |
| SEQRES | 18 | A | 321 | ILE | HIS | VAL | MET | GLU | LEU | ALA | HIS | GLU | VAL | TRP | LEU | GLU |
| SEQRES | 19 | A | 321 | ASN | THR | GLN | PRO | MET | LEU | SER | HIS | ILE | ASN | VAL | GLY | THR |
| SEQRES | 20 | A | 321 | SLY | VAL | ASP | CYS | THR | ILE | ARG | ASP | VAL | ALA | GLN | THR | ILE |
| SEQRES | 21 | A | 321 | ALA | LYS | VAL | VAL | GLY | TYR | LYS | GLY | ARG | VAL | VAL | PHE | ASP |
| SEQRES | 22 | A | 321 | ALA | SER | LYS | PRO | ASP | GLY | THR | PRO | ARG | LYS | LEU | LEU | ASP |
| SEQRES | 23 | A | 321 | VAL | THR | ARG | LEU | HIS | GLN | LEU | GLY | TRP | TYR | HIS | GLU | ILE |
| SEQRES | 24 | A | 321 | SER | LEU | GLU | ALA | GLY | LEU | ALA | SER | THR | TYR | GLN | TRP | PHE |
| SEQRES | 25 | A | 321 | LEU | GLU | ASN | GLN | ASP | ARG | PHE | ARG | GLY | | | | |

| | | | |
|---|---|---|---|
| HET | NDP | 1 | 0 |

| HETNAM | | NDP NICOTINAMIDE ADENINE DINUCLEOTIDE PHOSPHATE | |
|---|---|---|---|
| HETSYN | | NDP NADP | |
| FORMUL | 2 | NDP | C21 H23 N7 O17 P3 3- |
| FORMUL | 3 | HOH | *109(E2 O1) |

| | | | | | | | | | | | | | | | |
|---|---|---|---|---|---|---|---|---|---|---|---|---|---|---|---|
| HELIX | 1 | 1 | MET A | 14 | GLN A | 25 | 1 | | | | | | | 12 | |
| HELIX | 2 | 2 | SER A | 44 | GLU A | 54 | 1 | | | | | | | 11 | |
| HELIX | 3 | 3 | ILE A | 69 | THR A | 74 | 1 | | | | | | | 6 | |
| HELIX | 4 | 4 | PRO A | 76 | ASN A | 97 | 1 | | | | | | | 22 | |
| HELIX | 5 | 5 | SER A | 108 | ILE A | 110 | 5 | | | | | | | 3 | |
| HELIX | 6 | 6 | GLU A | 121 | GLU A | 123 | 5 | | | | | | | 3 | |
| HELIX | 7 | 7 | GLU A | 134 | TYR A | 154 | 1 | | | | | | | 21 | |
| HELIX | 8 | 8 | VAL A | 180 | ALA A | 193 | 1 | | | | | | | 14 | |
| HELIX | 9 | 9 | VAL A | 214 | GLU A | 226 | 1 | | | | | | | 13 | |
| HELIX | 10 | 10 | HIS A | 229 | GLU | A | 234 | 1 | | | | | | | 6 |
| HELIX | 11 | 11 | ILE A | 253 | VAL A | 264 | 1 | | | | | | | 12 | |
| HELIX | 12 | 12 | THR A | 288 | GLN A | 292 | 1 | | | | | | | 5 | |
| HELIX | 13 | 13 | LEU A | 301 | GLU A | 314 | 1 | | | | | | | 14 | |
| SHEET | 1 | A | 6 VAL A | 29 | VAL A | 32 | 0 | | | | | | | | |
| SHEET | 2 | A | 6 GLN A | 4 | ALA A | 9 | 1 | N | GLN A | 4 | O | GLU A | 30 | | |
| SHEET | 3 | A | 6 GLN A | 58 | LEU A | 61 | 1 | N | GLN A | 58 | O | PHE A | 7 | | |
| SHEET | 4 | A | 6 LYS A | 101 | LEU A | 105 | 1 | N | LYS A | 101 | O | VAL A | 59 | | |
| SHEET | 5 | A | 6 ASP A | 157 | PRO A | 163 | 1 | N | ASP A | 157 | O | LEU A | 102 | | |
| SHEET | 6 | A | 6 ILE A | 243 | VAL A | 245 | 1 | N | ILE A | 243 | O | MET A | 162 | | |
| SHEET | 1 | B | 2 ASN A | 165 | TYR A | 167 | 0 | | | | | | | | |
| SHEET | 2 | B | 2 PHE A | 211 | HIS A | 213 | 1 | N | LEU A | 212 | O | ASN A | 165 | | |
| SHEET | 1 | C | 2 ASP A | 198 | TRP A | 202 | 0 | | | | | | | | |
| SHEET | 2 | C | 2 ARG A | 269 | ASP A | 273 | 1 | N | ARG A | 269 | O | VAL A | 199 | | |

| | | | | |
|---|---|---|---|---|
| SITE | 1 | CAT | 1 TYR | 136 |
| SITE | 2 | CAT | 1 LYS | 140 |
| SITE | 3 | CAT | 1 SER | 107 |

| | | | | | | | | | | | |
|---|---|---|---|---|---|---|---|---|---|---|---|
| CRYST1 | 104.200 | 104.200 | 75.880 | 90.00 | 90.00 | 120.00 | P | 32 | 2 | 1 | 6 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.009597 | 0.005541 | 0.000000 | 0.00000 |
| SCALE2 | 0.000000 | 0.011081 | 0.000000 | 0.00000 |
| SCALE3 | 0.000000 | 0.000000 | 0.013179 | 0.00000 |

| | | | | | | | | | | | |
|---|---|---|---|---|---|---|---|---|---|---|---|
| HETATM | 1 | O | HOH | | 1 | 55.652 | −16.806 | 22.535 | 1.00 | 8.73 | O |
| HETATM | 2 | O | HOH | | 3 | 58.494 | −10.639 | 18.740 | 1.00 | 13.17 | O |
| HETATM | 3 | O | HOH | | 4 | 58.230 | −11.715 | 27.770 | 1.00 | 19.07 | O |
| HETATM | 4 | O | HOH | | 5 | 57.252 | −3.759 | 30.107 | 1.00 | 11.21 | O |
| HETATM | 5 | O | HOH | | 6 | 58.298 | −10.011 | 25.527 | 1.00 | 15.74 | O |
| HETATM | 6 | O | HOH | | 7 | 49.321 | 6.583 | 38.815 | 1.00 | 19.33 | O |
| HETATM | 7 | O | HOH | | 8 | 53.785 | −4.262 | 22.464 | 1.00 | 10.94 | O |
| HETATM | 8 | O | HOH | | 10 | 74.652 | 2.888 | 9.141 | 1.00 | 17.80 | O |
| HETATM | 9 | O | HOH | | 11 | 49.761 | 0.826 | 32.896 | 1.00 | 22.02 | O |
| HETATM | 10 | O | HOH | | 12 | 55.530 | −11.162 | 28.526 | 1.00 | 11.39 | O |
| HETATM | 11 | O | HOH | | 13 | 75.027 | 7.034 | 27.353 | 1.00 | 16.30 | O |
| HETATM | 12 | O | HOH | | 14 | 49.994 | −2.314 | 11.032 | 1.00 | 21.33 | O |
| HETATM | 13 | O | HOH | | 15 | 61.323 | −8.959 | 29.657 | 1.00 | 22.84 | O |
| HETATM | 14 | O | HOH | | 16 | 61.029 | −11.560 | 29.131 | 1.00 | 21.24 | O |
| HETATM | 15 | O | HOH | | 17 | 50.684 | 5.881 | 10.130 | 1.00 | 15.88 | O |
| HETATM | 16 | O | HOH | | 18 | 64.506 | −6.302 | 32.989 | 1.00 | 21.05 | O |
| HETATM | 17 | O | HOH | | 19 | 57.856 | −16.398 | 25.085 | 1.00 | 22.86 | O |
| HETATM | 18 | O | HOH | | 20 | 38.979 | 26.536 | 19.070 | 1.00 | 21.08 | O |
| HETATM | 19 | O | HOH | | 21 | 38.042 | 33.487 | 21.909 | 1.00 | 19.01 | O |
| HETATM | 20 | O | HOH | | 24 | 38.172 | 35.775 | 20.827 | 1.00 | 33.46 | O |
| HETATM | 21 | O | HOH | | 25 | 70.916 | −11.128 | 15.244 | 1.00 | 31.37 | O |
| HETATM | 22 | O | HOH | | 26 | 54.205 | 19.360 | 28.396 | 1.00 | 35.76 | O |
| HETATM | 23 | O | HOH | | 27 | 50.436 | 2.654 | 16.783 | 1.00 | 12.25 | O |
| HETATM | 24 | O | HOH | | 28 | 69.692 | 19.108 | 38.979 | 1.00 | 49.77 | O |
| HETATM | 25 | O | HOH | | 29 | 56.432 | −8.877 | 19.303 | 1.00 | 22.52 | O |
| HETATM | 26 | O | HOH | | 30 | 60.832 | 3.415 | 42.349 | 1.00 | 17.39 | O |
| HETATM | 27 | O | HOH | | 31 | 53.889 | −12.706 | 29.764 | 1.00 | 22.40 | O |
| HETATM | 28 | O | HOH | | 32 | 37.887 | 26.373 | 28.058 | 1.00 | 18.09 | O |
| HETATM | 29 | O | HOH | | 33 | 49.201 | 11.173 | 26.867 | 1.00 | 33.95 | O |
| HETATM | 30 | O | HOH | | 34 | 46.762 | −0.278 | 31.394 | 1.00 | 20.63 | O |
| HETATM | 31 | O | HOH | | 35 | 41.731 | 27.568 | 43.302 | 1.00 | 27.39 | O |
| HETATM | 32 | O | HOH | | 36 | 66.827 | 11.202 | 28.929 | 1.00 | 13.23 | O |
| HETATM | 33 | O | HOH | | 37 | 46.834 | 14.396 | 40.819 | 1.00 | 46.02 | O |
| HETATM | 34 | O | HOH | | 38 | 61.342 | 1.064 | 43.868 | 1.00 | 26.68 | O |
| HETATM | 35 | O | HOH | | 42 | 70.597 | 16.422 | 37.837 | 1.00 | 19.26 | O |
| HETATM | 36 | O | HOH | | 44 | 72.275 | −9.089 | 33.407 | 1.00 | 22.11 | O |
| HETATM | 37 | O | HOH | | 45 | 42.685 | 34.461 | 33.955 | 1.00 | 17.32 | O |
| HETATM | 38 | O | HOH | | 46 | 53.480 | 13.394 | 38.364 | 1.00 | 20.19 | O |
| HETATM | 39 | O | HOH | | 47 | 56.085 | 21.757 | 44.744 | 1.00 | 33.50 | O |
| HETATM | 40 | O | HOH | | 48 | 35.741 | 32.691 | 23.517 | 1.00 | 19.49 | O |
| HETATM | 41 | O | HOH | | 49 | 40.458 | 36.700 | 34.312 | 1.00 | 34.53 | O |
| HETATM | 42 | O | HOH | | 50 | 75.440 | 7.267 | 29.948 | 1.00 | 18.07 | O |
| HETATM | 43 | O | HOH | | 51 | 47.476 | 18.347 | 20.851 | 1.00 | 34.16 | O |
| HETATM | 44 | O | HOH | | 53 | 52.837 | −16.344 | 19.587 | 1.00 | 25.92 | O |
| HETATM | 45 | O | HOH | | 55 | 46.415 | 9.073 | 20.108 | 1.00 | 31.91 | O |
| HETATM | 46 | O | HOH | | 57 | 45.912 | 35.170 | 36.133 | 1.00 | 35.55 | O |
| HETATM | 47 | O | HOH | | 58 | 60.247 | −2.880 | 41.919 | 1.00 | 16.85 | O |
| HETATM | 48 | O | HOH | | 60 | 64.974 | 6.086 | 24.501 | 1.00 | 32.16 | O |
| HETATM | 49 | O | HOH | | 61 | 52.103 | 4.683 | 4.978 | 1.00 | 35.72 | O |
| HETATM | 50 | O | HOH | | 62 | 50.888 | 40.154 | 36.463 | 1.00 | 38.35 | O |
| HETATM | 51 | O | HOH | | 63 | 44.373 | 31.233 | 37.336 | 1.00 | 20.07 | O |
| HETATM | 52 | O | HOH | | 64 | 57.280 | 27.757 | 42.451 | 1.00 | 21.74 | O |
| HETATM | 53 | O | HOH | | 65 | 58.409 | 23.769 | 45.517 | 1.00 | 58.42 | O |
| HETATM | 54 | O | HOH | | 66 | 68.690 | −11.764 | 35.335 | 1.00 | 57.07 | O |
| HETATM | 55 | O | HOH | | 67 | 42.746 | 25.153 | 23.465 | 1.00 | 27.05 | O |
| HETATM | 56 | O | HOH | | 68 | 53.638 | −16.457 | 32.292 | 1.00 | 31.71 | O |
| HETATM | 57 | O | HOH | | 69 | 33.390 | 41.716 | 31.408 | 1.00 | 29.92 | O |
| HETATM | 58 | O | HOH | | 70 | 57.768 | 17.897 | 42.434 | 1.00 | 25.75 | O |
| HETATM | 59 | O | HOH | | 71 | 75.647 | 9.164 | 11.766 | 1.00 | 35.13 | O |
| HETATM | 60 | O | HOH | | 72 | 62.032 | 33.292 | 44.749 | 1.00 | 46.18 | O |
| HETATM | 61 | O | HOH | | 73 | 47.310 | 14.312 | 34.285 | 1.00 | 31.18 | O |
| HETATM | 62 | O | HOH | | 74 | 79.660 | −3.947 | 15.913 | 1.00 | 34.63 | O |
| HETATM | 63 | O | HOH | | 75 | 46.929 | 5.343 | 4.550 | 1.00 | 23.14 | O |
| HETATM | 64 | O | HOH | | 76 | 73.475 | 12.039 | 28.412 | 1.00 | 27.26 | O |
| HETATM | 65 | O | HOH | | 77 | 46.297 | −6.982 | 30.032 | 1.00 | 43.41 | O |
| HETATM | 66 | O | HOH | | 78 | 68.528 | −3.422 | 40.869 | 1.00 | 38.47 | O |
| HETATM | 67 | O | HOH | | 79 | 62.080 | −1.448 | 42.803 | 1.00 | 24.60 | O |
| HETATM | 68 | O | HOH | | 80 | 65.330 | 18.150 | 40.726 | 1.00 | 41.00 | O |
| HETATM | 69 | O | HOH | | 81 | 51.775 | 16.128 | 37.607 | 1.00 | 25.11 | O |
| HETATM | 70 | O | HOH | | 83 | 54.266 | 28.682 | 43.313 | 1.00 | 27.61 | O |
| HETATM | 71 | O | HOH | | 85 | 73.291 | −15.479 | 20.603 | 1.00 | 37.54 | O |
| HETATM | 72 | O | HOH | | 86 | 34.760 | 21.479 | 28.544 | 1.00 | 43.87 | O |
| HETATM | 73 | O | HOH | | 87 | 37.326 | 24.131 | 29.677 | 1.00 | 24.47 | O |
| HETATM | 74 | O | HOH | | 88 | 65.168 | 20.148 | 6.735 | 1.00 | 26.10 | O |
| HETATM | 75 | O | HOH | | 89 | 59.196 | 12.089 | 13.630 | 1.00 | 25.24 | O |
| HETATM | 76 | O | HOH | | 91 | 66.576 | −6.235 | 40.279 | 1.00 | 43.11 | O |
| HETATM | 77 | O | HOH | | 93 | 37.339 | 29.394 | 25.515 | 1.00 | 27.56 | O |
| HETATM | 78 | O | HOH | | 94 | 52.339 | -17.014 | 42.271 | 1.00 | 48.96 | O |
| HETATM | 79 | O | HOH | | 95 | 40.511 | 32.927 | 31.717 | 1.00 | 22.46 | O |
| HETATM | 80 | O | HOH | | 96 | 78.580 | 13.121 | 34.138 | 1.00 | 27.98 | O |
| HETATM | 81 | O | HOH | | 97 | 65.090 | 15.704 | 34.876 | 1.00 | 18.96 | O |
| HETATM | 82 | O | HOH | | 99 | 84.562 | 2.951 | 27.181 | 1.00 | 35.92 | O |
| HETATM | 83 | O | HOH | | 100 | 50.386 | 9.761 | 9.646 | 1.00 | 23.18 | O |
| HETATM | 84 | O | HOH | | 101 | 67.649 | −0.851 | 38.764 | 1.00 | 24.99 | O |
| HETATM | 85 | O | HOH | | 102 | 44.001 | 4.293 | 34.315 | 1.00 | 31.13 | O |
| HETATM | 86 | O | HOH | | 103 | 59.386 | −5.071 | 26.211 | 1.00 | 29.10 | O |
| HETATM | 87 | O | HOH | | 104 | 77.364 | 4.745 | 41.506 | 1.00 | 35.32 | O |
| HETATM | 88 | O | HOH | | 105 | 59.034 | 21.201 | 32.414 | 1.00 | 23.43 | O |
| HETATM | 89 | O | HOH | | 106 | 42.463 | 34.698 | 14.327 | 1.00 | 38.86 | O |
| HETATM | 90 | O | HOH | | 107 | 70.217 | 14.292 | 20.864 | 1.00 | 42.39 | O |
| HETATM | 91 | O | HOH | | 108 | 76.999 | 8.130 | 25.862 | 1.00 | 32.91 | O |
| HETATM | 92 | O | HOH | | 109 | 49.766 | 29.937 | 22.173 | 1.00 | 42.52 | O |
| HETATM | 93 | O | HOH | | 110 | 72.473 | 13.536 | 38.823 | 1.00 | 33.32 | O |
| HETATM | 94 | O | HOH | | 111 | 64.328 | −12.084 | 38.608 | 1.00 | 37.99 | O |
| HETATM | 95 | O | HOH | | 112 | 60.161 | 16.382 | 42.682 | 1.00 | 35.68 | O |
| HETATM | 96 | O | HOH | | 113 | 47.602 | 13.639 | 27.016 | 1.00 | 26.01 | O |
| HETATM | 97 | O | HOH | | 115 | 64.606 | 11.644 | 40.107 | 1.00 | 30.33 | O |
| HETATM | 98 | O | HOH | | 116 | 61.231 | −15.137 | 27.255 | 1.00 | 38.76 | O |
| HETATM | 99 | O | HOH | | 117 | 65.324 | −11.223 | 35.098 | 1.00 | 30.45 | O |
| HETATM | 100 | O | HOH | | 119 | 56.602 | 17.219 | 44.932 | 1.00 | 36.53 | O |
| HETATM | 101 | O | HOH | | 120 | 37.564 | 19.860 | 23.135 | 1.00 | 31.27 | O |
| HETATM | 102 | O | HOH | | 121 | 64.845 | 5.057 | 21.132 | 1.00 | 45.57 | O |
| HETATM | 103 | O | HOH | | 123 | 63.391 | 16.801 | 26.898 | 1.00 | 38.46 | O |
| HETATM | 104 | O | HOH | | 124 | 42.567 | 6.134 | 32.635 | 1.00 | 31.56 | O |
| HETATM | 105 | O | HOH | | 125 | 72.485 | 13.236 | 35.059 | 1.00 | 29.61 | O |
| HETATM | 106 | O | HOH | | 126 | 65.229 | 3.650 | 44.032 | 1.00 | 36.86 | O |
| HETATM | 107 | O | HOH | | 127 | 37.089 | 7.148 | 31.083 | 1.00 | 39.58 | O |
| HETATM | 108 | O | HOH | | 128 | 73.327 | 10.546 | 12.123 | 1.00 | 34.97 | O |
| HETATM | 109 | O | HOH | | 129 | 74.450 | 10.299 | 26.598 | 1.00 | 30.80 | O |
| HETATM | 110 | AO5* | NDP | A | 1 | 67.524 | 13.055 | 26.692 | 1.00 | 36.42 | O |
| HETATM | 111 | AC5* | NDP | A | 1 | 68.089 | 12.297 | 25.614 | 1.00 | 9.30 | C |
| HETATM | 112 | AC4* | NDP | A | 1 | 69.601 | 12.124 | 25.858 | 1.00 | 27.73 | C |
| HETATM | 113 | AO4* | NDP | A | 1 | 70.193 | 11.258 | 24.848 | 1.00 | 22.87 | O |
| HETATM | 114 | AC3* | NDP | A | 1 | 70.484 | 13.390 | 25.873 | 1.00 | 17.83 | C |
| HETATM | 115 | AO3* | NDP | A | 1 | 71.192 | 13.436 | 27.066 | 1.00 | 16.11 | O |
| HETATM | 116 | AC2* | NDP | A | 1 | 71.373 | 13.220 | 24.626 | 1.00 | 11.46 | C |
| HETATM | 117 | AO2* | NDP | A | 1 | 72.623 | 13.886 | 24.655 | 1.00 | 31.96 | O |
| HETATM | 118 | AC1* | NDP | A | 1 | 71.510 | 11.702 | 24.656 | 1.00 | 19.02 | C |
| HETATM | 119 | O3 | NDP | A | 1 | 65.336 | 13.590 | 26.129 | 1.00 | 20.59 | O |
| HETATM | 120 | NO5* | NDP | A | 1 | 63.536 | 11.943 | 26.448 | 1.00 | 28.99 | O |
| HETATM | 121 | NC5* | NDP | A | 1 | 64.328 | 10.843 | 25.957 | 1.00 | 24.89 | C |
| HETATM | 122 | NC4* | NDP | A | 1 | 63.467 | 9.646 | 25.686 | 1.00 | 31.79 | C |
| HETATM | 123 | NO4* | NDP | A | 1 | 62.837 | 9.337 | 26.908 | 1.00 | 28.82 | O |
| HETATM | 124 | NC3* | NDP | A | 1 | 62.340 | 9.837 | 24.665 | 1.00 | 11.50 | C |
| HHTATM | 125 | NO3* | NDP | A | 1 | 62.891 | 9.402 | 23.461 | 1.00 | 28.60 | O |
| HETATM | 126 | NC2* | NDP | A | 1 | 61.152 | 8.996 | 25.138 | 1.00 | 28.11 | C |
| HETATM | 127 | NO2* | NDP | A | 1 | 60.881 | 7.662 | 24.715 | 1.00 | 24.30 | O |
| HETATM | 128 | NC1* | NDP | A | 1 | 61.547 | 8.875 | 26.580 | 1.00 | 35.35 | C |
| HETATM | 129 | AP2* | NDP | A | 1 | 73.104 | 15.069 | 23.823 | 1.00 | 32.96 | P |
| HETATM | 130 | AOP1 | NDP | A | 1 | 74.500 | 15.308 | 24.308 | 1.00 | 37.84 | O |
| HETATM | 131 | AOP2 | NDP | A | 1 | 72.797 | 14.925 | 22.348 | 1.00 | 36.66 | O |
| HETATM | 132 | AOP3 | NDP | A | 1 | 72.163 | 16.217 | 23.958 | 1.00 | 31.97 | O |
| HETATM | 133 | AP | NDP | A | 1 | 66.660 | 14.257 | 26.393 | 1.00 | 26.17 | XX |
| HETATM | 134 | AO1 | NDP | A | 1 | 66.886 | 14.795 | 25.047 | 1.00 | 15.31 | XX |
| HETATM | 135 | AO2 | NDP | A | 1 | 66.439 | 15.207 | 27.521 | 1.00 | 34.39 | XX |
| HETATM | 136 | AN9 | NDP | A | 1 | 71.820 | 11.224 | 23.353 | 1.00 | 13.63 | XX |
| HETATM | 137 | AC8 | NDP | A | 1 | 71.104 | 11.316 | 22.200 | 1.00 | 12.41 | XX |
| HETATM | 138 | AN7 | NDP | A | 1 | 71.758 | 10.835 | 21.161 | 1.00 | 15.71 | XX |
| HETATM | 139 | AC5 | NDP | A | 1 | 72.933 | 10.313 | 21.710 | 1.00 | 16.17 | XX |
| HETATM | 140 | AC6 | NDP | A | 1 | 74.053 | 9.657 | 21.140 | 1.00 | 31.35 | XX |
| HETATM | 141 | AN6 | NDP | A | 1 | 74.165 | 9.464 | 19.819 | 1.00 | 12.59 | XX |
| HETATM | 142 | AN1 | NDP | A | 1 | 75.078 | 9.280 | 21.942 | 1.00 | 17.56 | XX |
| HETATM | 143 | AC2 | NDP | A | 1 | 74.971 | 9.578 | 23.251 | 1.00 | 15.44 | XX |
| HETATM | 144 | AN3 | NDP | A | 1 | 74.027 | 10.302 | 23.889 | 1.00 | 24.82 | XX |
| HETATM | 145 | AC4 | NDP | A | 1 | 73.036 | 10.653 | 23.047 | 1.00 | 17.48 | XX |
| HETATM | 146 | NP | NDP | A | 1 | 64.183 | 13.106 | 27.191 | 1.00 | 25.47 | N |
| HETATM | 147 | NO1 | NDP | A | 1 | 63.142 | 14.169 | 27.253 | 1.00 | 28.69 | N |
| HETATM | 148 | NO2 | NDP | A | 1 | 64.837 | 12.643 | 28.492 | 1.00 | 24.32 | N |
| HETATM | 149 | NN1 | NDP | A | 1 | 60.598 | 9.775 | 27.109 | 1.00 | 23.63 | N |
| HETATM | 150 | NC2 | NDP | A | 1 | 60.143 | 10.905 | 26.442 | −99.00 | 78.36 | N |
| HETATM | 151 | NC3 | NDP | A | 1 | 59.070 | 11.648 | 27.007 | −99.00 | 100.00 | N |
| HETATM | 152 | NC7 | NDP | A | 1 | 58.497 | 13.017 | 26.528 | −99.00 | 100.00 | N |
| HETATM | 153 | NO7 | NDP | A | 1 | 59.358 | 13.703 | 25.972 | −99.00 | 100.00 | N |
| HETATM | 154 | NN7 | NDP | A | 1 | 57.207 | 13.400 | 26.912 | −99.00 | 84.38 | N |
| HETATM | 155 | NC4 | NDP | A | 1 | 58.442 | 11.146 | 28.137 | −99.00 | 100.00 | N |
| HETATM | 156 | NC5 | NDP | A | 1 | 58.912 | 9.963 | 28.754 | −99.00 | 100.00 | N |
| HETATM | 157 | NC6 | NDP | A | 1 | 59.951 | 9.266 | 28.147 | −99.00 | 100.00 | N |
| ATOM | 158 | N | LYS | A | 3 | 76.227 | −5.632 | 44.315 | 1.00 | 61.49 | N |
| ATOM | 159 | CA | LYS | A | 3 | 76.152 | −4.302 | 43.684 | 1.00 | 58.00 | C |
| ATOM | 160 | C | LYS | A | 3 | 75.985 | −4.421 | 42.171 | 1.00 | 52.79 | C |
| ATOM | 161 | O | LYS | A | 3 | 76.921 | −4.737 | 41.419 | 1.00 | 44.76 | O |
| ATOM | 162 | CE | LYS | A | 3 | 77.359 | −3.417 | 44.030 | 1.00 | 59.74 | C |
| ATOM | 163 | CG | LYS | A | 3 | 77.011 | −1.944 | 44.314 | 1.00 | 50.87 | C |
| ATOM | 164 | CD | LYS | A | 3 | 78.208 | −1.161 | 44.894 | 1.00 | 61.21 | C |
| ATOM | 165 | CE | LYS | A | 3 | 77.855 | −0.377 | 46.186 | 1.00 | 100.00 | C |
| ATOM | 166 | NZ | LYS | A | 3 | 78.857 | −0.401 | 47.343 | 1.00 | 70.61 | N |
| ATOM | 167 | N | GLN | A | 4 | 74.746 | −4.242 | 41.747 | 1.00 | 45.15 | N |
| ATOM | 168 | CA | GLN | A | 4 | 74.408 | −4.326 | 40.347 | 1.00 | 37.18 | C |
| ATOM | 169 | C | GLN | A | 4 | 74.983 | −3.166 | 39.561 | 1.00 | 34.93 | C |
| ATOM | 170 | O | GLN | A | 4 | 75.127 | −2.050 | 40.087 | 1.00 | 28.48 | O |
| ATOM | 171 | CE | GLN | A | 4 | 72.915 | −4.445 | 40.221 | 1.00 | 34.65 | C |
| ATOM | 172 | CG | GLN | A | 4 | 72.456 | −5.854 | 40.584 | 1.00 | 31.82 | C |
| ATOM | 173 | CD | GLN | A | 4 | 72.570 | −6.788 | 39.405 | 1.00 | 79.25 | C |
| ATOM | 174 | OE1 | GLN | A | 4 | 72.165 | −6.452 | 38.286 | 1.00 | 100.00 | O |
| ATOM | 175 | NE2 | GLN | A | 4 | 73.206 | −7.925 | 39.623 | 1.00 | 80.24 | N |
| ATOM | 176 | N | ARG | A | 5 | 75.475 | −3.495 | 38.375 | 1.00 | 27.16 | N |
| ATOM | 177 | CA | ARG | A | 5 | 76.146 | −2.546 | 37.483 | 1.00 | 39.16 | C |
| ATOM | 178 | C | ARG | A | 5 | 75.191 | +321 2.018 | 36.433 | 1.00 | 38.22 | C |
| ATOM | 179 | O | ARG | A | 5 | 74.938 | −2.698 | 35.438 | 1.00 | 32.44 | O |
| ATOM | 180 | CB | ARG | A | 5 | 77.398 | −3.163 | 36.826 | 1.00 | 41.76 | C |
| ATOM | 181 | CG | ARG | A | 5 | 78.692 | −2.954 | 37.663 | 1.00 | 37.34 | C |
| ATOM | 182 | CD | ARG | A | 5 | 80.015 | −3.236 | 36.876 | 1.00 | 32.99 | C |
| ATOM | 183 | NE | ARG | A | 5 | 81.036 | −2.203 | 37.125 | 1.00 | 25.71 | N |
| ATOM | 184 | CZ | ARG | A | 5 | 81.617 | −1.488 | 36.169 | 1.00 | 32.53 | C |
| ATOM | 185 | NE1 | ARG | A | 5 | 81.293 | −1.704 | 34.904 | 1.00 | 40.07 | N |
| ATOM | 186 | NH2 | ARG | A | 5 | 82.516 | −0.551 | 36.474 | 1.00 | 100.00 | N |
| ATOM | 187 | N | VAL | A | 6 | 74.743 | −0.773 | 36.659 | 1.00 | 32.08 | N |
| ATOM | 188 | CA | VAL | A | 6 | 73.715 | −0.082 | 35.881 | 1.00 | 28.89 | C |
| ATOM | 189 | C | VAL | A | 6 | 74.161 | 1.021 | 34.897 | 1.00 | 29.37 | C |
| ATOM | 190 | O | VAL | A | 6 | 74.745 | 2.041 | 35.274 | 1.00 | 22.50 | O |
| ATOM | 191 | CB | VAL | A | 6 | 72.577 | 0.378 | 36.813 | 1.00 | 23.52 | C |
| ATOM | 192 | CG1 | VAL | A | 6 | 71.366 | 0.960 | 36.006 | 1.00 | 20.29 | C |
| ATOM | 193 | CG2 | VAL | A | 6 | 72.108 | −0.852 | 37.644 | 1.00 | 18.45 | C |
| ATOM | 194 | N | PHE | A | 7 | 73.948 | 0.749 | 33.615 | 1.00 | 22.92 | N |
| ATOM | 195 | CA | PHE | A | 7 | 74.267 | 1.710 | 32.573 | 1.00 | 27.15 | C |
| ATOM | 196 | C | PHE | A | 7 | 72.975 | 2.423 | 32.192 | 1.00 | 20.24 | C |
| ATOM | 197 | O | PHE | A | 7 | 71.994 | 1.788 | 31.815 | 1.00 | 20.71 | O |
| ATOM | 198 | CB | PHE | A | 7 | 74.864 | 1.004 | 31.374 | 1.00 | 18.98 | C |
| ATOM | 199 | CG | PHE | A | 7 | 74.916 | 1.836 | 30.115 | 1.00 | 21.83 | C |
| ATOM | 200 | CD1 | PHE | A | 7 | 75.521 | 3.087 | 30.108 | 1.00 | 19.36 | C |
| ATOM | 201 | CD2 | PHE | A | 7 | 74.483 | 1.284 | 28.886 | 1.00 | 23.50 | C |
| ATOM | 202 | CE1 | PHE | A | 7 | 75.614 | 3.828 | 28.902 | 1.00 | 27.52 | C |
| ATOM | 203 | CE2 | PHE | A | 7 | 74.548 | 1.996 | 27.685 | 1.00 | 19.33 | C |
| ATOM | 204 | CZ | PHE | A | 7 | 75.128 | 3.255 | 27.673 | 1.00 | 18.59 | C |
| ATOM | 205 | N | ILE | A | 8 | 72.959 | 3.727 | 32.454 | 1.00 | 18.75 | N |
| ATOM | 206 | CA | ILE | A | 8 | 71.844 | 4.588 | 32.112 | 1.00 | 14.25 | C |
| ATOM | 207 | C | ILE | A | 8 | 72.337 | 5.351 | 30.909 | 1.00 | 11.22 | C |
| ATOM | 208 | O | ILE | A | 8 | 73.259 | 6.165 | 30.998 | 1.00 | 17.76 | O |
| ATOM | 209 | CE | ILE | A | 8 | 71.507 | 5.605 | 33.212 | 1.00 | 14.15 | C |
| ATOM | 210 | CG1 | ILE | A | 8 | 71.356 | 4.949 | 34.582 | 1.00 | 8.24 | C |
| ATOM | 211 | CG2 | ILE | A | 8 | 70.183 | 6.342 | 32.874 | 1.00 | 16.85 | C |
| ATOM | 212 | CD1 | ILE | A | 8 | 71.091 | 5.961 | 35.707 | 1.00 | 10.32 | C |
| ATOM | 213 | N | ALA | A | 9 | 71.896 | 4.906 | 29.752 | 1.00 | 16.42 | N |
| ATOM | 214 | CA | ALA | A | 9 | 72.256 | 5.559 | 28.513 | 1.00 | 18.74 | C |
| ATOM | 215 | C | ALA | A | 9 | 71.530 | 6.913 | 28.511 | 1.00 | 28.45 | C |
| ATOM | 216 | O | ALA | A | 9 | 70.411 | 7.032 | 29.045 | 1.00 | 22.39 | O |
| ATOM | 217 | CB | ALA | A | 9 | 71.808 | 4.731 | 27.311 | 1.00 | 14.43 | C |
| ATOM | 218 | N | GLY | A | 10 | 72.199 | 7.922 | 27.940 | 1.00 | 20.06 | N |
| ATOM | 219 | CA | GLY | A | 10 | 71.706 | 9.284 | 27.911 | 1.00 | 18.62 | C |
| ATOM | 220 | C | GLY | A | 10 | 71.407 | 9.819 | 29.305 | 1.00 | 16.40 | C |
| ATOM | 221 | O | GLY | A | 10 | 70.379 | 10.448 | 29.481 | 1.00 | 17.36 | O |
| ATOM | 222 | N | HIS | A | 11 | 72.295 | 9.581 | 30.272 | 1.00 | 10.32 | N |
| ATOM | 223 | CA | HIS | A | 11 | 72.068 | 9.966 | 31.688 | 1.00 | 13.90 | C |
| ATOM | 224 | C | HIS | A | 11 | 72.008 | 11.504 | 31.916 | 1.00 | 21.52 | C |
| ATOM | 225 | O | HIS | A | 11 | 71.700 | 11.994 | 32.983 | 1.00 | 13.22 | O |
| ATOM | 226 | CE | HIS | A | 11 | 73.153 | 9.350 | 32.581 | 1.00 | 14.88 | C |
| ATOM | 227 | CG | HIS | A | 11 | 74.502 | 9.948 | 32.326 | 1.00 | 23.73 | C |
| ATOM | 228 | ND1 | HIS | A | 11 | 75.239 | 9.648 | 31.197 | 1.00 | 24.90 | N |
| ATOM | 229 | CD2 | HIS | A | 11 | 75.167 | 10.952 | 32.956 | 1.00 | 16.35 | C |
| ATOM | 230 | CE1 | HIS | A | 11 | 76.317 | 10.407 | 31.170 | 1.00 | 22.54 | C |
| ATOM | 231 | NE2 | HIS | A | 11 | 76.271 | 11.240 | 32.197 | 1.00 | 17.56 | N |
| ATOM | 232 | N | ARG | A | 12 | 72.310 | 12.288 | 30.908 | 1.00 | 22.31 | N |
| ATOM | 233 | CA | ARG | A | 12 | 72.147 | 13.693 | 31.122 | 1.00 | 18.90 | C |
| ATOM | 234 | C | ARG | A | 12 | 70.851 | 14.244 | 30.495 | 1.00 | 26.34 | C |
| ATOM | 235 | O | ARG | A | 12 | 70.572 | 15.426 | 30.604 | 1.00 | 25.37 | O |
| ATOM | 236 | CE | ARG | A | 12 | 73.352 | 14.418 | 30.587 | 1.00 | 25.93 | C |
| ATOM | 237 | CG | ARG | A | 12 | 74.582 | 13.943 | 31.279 | 1.00 | 53.87 | C |
| ATOM | 238 | CD | ARG | A | 12 | 75.757 | 14.619 | 30.699 | 1.00 | 32.53 | C |
| ATOM | 239 | NE | ARG | A | 12 | 76.359 | 15.576 | 31.605 | 1.00 | 69.90 | N |
| ATOM | 240 | CZ | ARG | A | 12 | 76.971 | 16.675 | 31.178 | 1.00 | 100.00 | C |
| ATOM | 241 | NH1 | ARG | A | 12 | 77.001 | 16.948 | 29.867 | 1.00 | 100.00 | N |
| ATOM | 242 | NH2 | ARG | A | 12 | 77.526 | 17.508 | 32.056 | 1.00 | 100.00 | N |
| ATOM | 243 | N | GLY | A | 13 | 70.078 | 13.420 | 29.800 | 1.00 | 18.25 | N |
| ATOM | 244 | CA | GLY | A | 13 | 68.802 | 13.904 | 29.258 | 1.00 | 16.50 | C |
| ATOM | 245 | C | GLY | A | 13 | 67.849 | 14.144 | 30.428 | 1.00 | 18.88 | C |
| ATOM | 246 | O | GLY | A | 13 | 68.202 | 13.902 | 31.624 | 1.00 | 14.04 | O |
| ATOM | 247 | N | MET | A | 14 | 66.653 | 14.632 | 30.103 | 1.00 | 16.00 | N |
| ATOM | 248 | CA | MET | A | 14 | 65.688 | 14.981 | 31.128 | 1.00 | 13.49 | C |
| ATOM | 249 | C | MET | A | 14 | 65.293 | 13.760 | 31.901 | 1.00 | 14.02 | C |
| ATOM | 250 | O | MET | A | 14 | 65.408 | 13.713 | 33.145 | 1.00 | 17.06 | O |
| ATOM | 251 | CE | MET | A | 14 | 64.442 | 15.605 | 30.524 | 1.00 | 11.57 | C |
| ATOM | 252 | CG | MET | A | 14 | 63.320 | 15.628 | 31.559 | 1.00 | 20.77 | C |
| ATOM | 253 | SD | MET | A | 14 | 61.926 | 16.766 | 31.110 | 1.00 | 29.16 | 5 |
| ATOM | 254 | CE | MET | A | 14 | 62.527 | 17.108 | 29.574 | 1.00 | 30.68 | C |
| ATOM | 255 | N | VAL | A | 15 | 64.798 | 12.769 | 31.158 | 1.00 | 25.23 | N |
| ATOM | 256 | CA | VAL | A | 15 | 64.439 | 11.468 | 31.738 | 1.00 | 20.90 | C |
| ATOM | 257 | C | VAL | A | 15 | 65.654 | 10.713 | 32.378 | 1.00 | 17.26 | C |
| ATOM | 258 | O | VAL | A | 15 | 65.590 | 10.239 | 33.524 | 1.00 | 18.41 | O |
| ATOM | 259 | CB | VAL | A | 15 | 63.752 | 10.550 | 30.680 | 1.00 | 23.25 | C |
| ATOM | 260 | CG1 | VAL | A | 15 | 63.330 | 9.253 | 31.310 | 1.00 | 15.71 | C |
| ATOM | 261 | CG2 | VAL | A | 15 | 62.528 | 11.193 | 30.183 | 1.00 | 13.40 | C |
| ATOM | 262 | N | GLY | A | 16 | 66.784 | 10.642 | 31.665 | 1.00 | 20.39 | N |
| ATOM | 263 | CA | GLY | A | 16 | 67.941 | 9.904 | 32.186 | 1.00 | 19.54 | C |
| ATOM | 264 | C | GLY | A | 16 | 68.522 | 10.432 | 33.492 | 1.00 | 29.29 | C |
| ATOM | 265 | O | GLY | A | 16 | 68.896 | 9.659 | 34.434 | 1.00 | 16.91 | O |
| ATOM | 266 | N | SER | A | 17 | 68.642 | 11.755 | 33.499 | 1.00 | 12.53 | N |
| ATOM | 267 | CA | SER | A | 17 | 69.154 | 12.460 | 34.650 | 1.00 | 21.93 | C |
| ATOM | 268 | C | SER | A | 17 | 68.209 | 12.214 | 35.818 | 1.00 | 13.35 | C |
| ATOM | 269 | O | SER | A | 17 | 68.677 | 11.957 | 36.915 | 1.00 | 24.19 | O |
| ATOM | 270 | CB | SER | A | 17 | 69.378 | 13.942 | 34.333 | 1.00 | 15.52 | C |
| ATOM | 271 | OG | SER | A | 17 | 68.153 | 14.619 | 34.372 | 1.00 | 22.95 | O |
| ATOM | 272 | N | ALA | A | 18 | 66.896 | 12.143 | 35.590 | 1.00 | 17.52 | N |
| ATOM | 273 | CA | ALA | A | 18 | 65.991 | 11.828 | 36.729 | 1.00 | 13.14 | C |
| ATOM | 274 | C | ALA | A | 18 | 66.220 | 10.393 | 37.307 | 1.00 | 19.29 | C |
| ATOM | 275 | O | ALA | A | 18 | 66.149 | 10.150 | 38.522 | 1.00 | 16.94 | O |
| ATOM | 276 | CB | ALA | A | 18 | 64.460 | 12.046 | 36.334 | 1.00 | 14.33 | C |
| ATOM | 277 | N | ILE | A | 19 | 66.484 | 9.432 | 36.430 | 1.00 | 20.80 | N |
| ATOM | 278 | CA | ILE | A | 19 | 66.705 | 8.078 | 36.900 | 1.00 | 18.08 | C |
| ATOM | 279 | C | ILE | A | 19 | 67.975 | 8.090 | 37.730 | 1.00 | 16.09 | C |
| ATOM | 280 | O | ILE | A | 19 | 68.018 | 7.530 | 38.820 | 1.00 | 20.73 | O |
| ATOM | 281 | CB | ILE | A | 19 | 66.804 | 7.079 | 35.710 | 1.00 | 17.58 | C |
| ATOM | 282 | CG1 | ILE | A | 19 | 65.444 | 6.812 | 35.162 | 1.00 | 10.09 | C |
| ATOM | 283 | CG2 | ILE | A | 19 | 67.309 | 5.666 | 36.133 | 1.00 | 21.60 | C |
| ATOM | 284 | CD1 | ILE | A | 19 | 65.528 | 6.361 | 33.741 | 1.00 | 19.05 | C |
| ATOM | 285 | N | ARG | A | 20 | 68.984 | 8.771 | 37.198 | 1.00 | 18.13 | N |
| ATOM | 286 | CA | ARG | A | 20 | 70.286 | 8.897 | 37.836 | 1.00 | 20.25 | C |
| ATOM | 287 | C | ARG | A | 20 | 70.231 | 9.491 | 39.242 | 1.00 | 30.62 | C |
| ATOM | 288 | O | ARG | A | 20 | 70.957 | 9.091 | 40.129 | 1.00 | 33.00 | O |
| ATOM | 289 | CB | ARG | A | 20 | 71.201 | 9.743 | 36.957 | 1.00 | 11.71 | C |
| ATOM | 290 | CG | ARG | A | 20 | 72.610 | 9.781 | 37.449 | 1.00 | 23.79 | C |
| ATOM | 291 | CD | ARG | A | 20 | 72.881 | 11.107 | 38.060 | 1.00 | 36.76 | C |
| ATOM | 292 | NE | ARG | A | 20 | 74.297 | 11.443 | 38.062 | 1.00 | 48.34 | N |
| ATOM | 293 | CZ | ARG | A | 20 | 74.990 | 11.841 | 36.988 | 1.00 | 100.00 | C |
| ATOM | 294 | NH1 | ARG | A | 20 | 74.393 | 11.931 | 35.808 | 1.00 | 100.00 | N |
| ATOM | 295 | NH2 | ARG | A | 20 | 76.289 | 12.139 | 37.076 | 1.00 | 100.00 | N |
| ATOM | 296 | N | ARG | A | 21 | 69.368 | 10.461 | 39.439 | 1.00 | 22.10 | N |
| ATOM | 297 | CA | ARG | A | 21 | 69.216 | 11.052 | 40.750 | 1.00 | 17.45 | C |
| ATOM | 298 | C | ARG | A | 21 | 68.721 | 10.007 | 41.730 | 1.00 | 26.71 | C |
| ATOM | 299 | O | ARG | A | 21 | 69.147 | 10.001 | 42.885 | 1.00 | 30.27 | O |
| ATOM | 300 | CB | ARG | A | 21 | 68.142 | 12.144 | 40.708 | 1.00 | 17.93 | C |
| ATOM | 301 | CG | ARG | A | 21 | 68.682 | 13.522 | 40.321 | 1.00 | 27.57 | C |
| ATOM | 302 | CD | ARG | A | 21 | 67.586 | 14.599 | 40.130 | 1.00 | 23.02 | C |
| ATOM | 303 | NE | ARG | A | 21 | 67.619 | 15.000 | 38.743 | 1.00 | 55.12 | N |
| ATOM | 304 | CZ | ARG | A | 21 | 66.538 | 15.103 | 37.995 | 1.00 | 10.55 | C |
| ATOM | 305 | NH1 | ARG | A | 21 | 65.343 | 14.974 | 38.552 | 1.00 | 29.80 | N |
| ATOM | 306 | NH2 | ARG | A | 21 | 66.665 | 15.435 | 36.715 | 1.00 | 61.45 | N |
| ATOM | 307 | N | GLN | A | 22 | 67.713 | 9.223 | 41.345 | 1.00 | 27.48 | N |
| ATOM | 308 | CA | GLN | A | 22 | 67.167 | 8.257 | 42.313 | 1.00 | 24.79 | C |
| ATOM | 309 | C | GLN | A | 22 | 68.137 | 7.127 | 42.547 | 1.00 | 31.37 | C |
| ATOM | 310 | O | GLN | A | 22 | 68.394 | 6.724 | 43.685 | 1.00 | 27.47 | O |
| ATOM | 311 | CB | GLN | A | 22 | 65.818 | 7.706 | 41.894 | 1.00 | 17.11 | C |
| ATOM | 312 | CG | GLN | A | 22 | 64.921 | 8.745 | 41.243 | 1.00 | 66.14 | C |
| ATOM | 313 | CD | GLN | A | 22 | 63.425 | 8.456 | 41.397 | 1.00 | 41.27 | C |
| ATOM | 314 | OE1 | GLN | A | 22 | 63.002 | 7.329 | 41.762 | 1.00 | 29.34 | O |
| ATOM | 315 | NE2 | GLN | A | 22 | 62.610 | 9.464 | 41.046 | 1.00 | 20.12 | N |
| ATOM | 316 | N | LEU | A | 23 | 68.697 | 6.652 | 41.448 | 1.00 | 27.99 | N |
| ATOM | 317 | CA | LEU | A | 23 | 69.649 | 5.575 | 41.500 | 1.00 | 24.48 | C |
| ATOM | 318 | C | LEU | A | 23 | 70.828 | 5.971 | 42.334 | 1.00 | 28.87 | C |
| ATOM | 319 | O | LEU | A | 23 | 71.288 | 5.218 | 43.165 | 1.00 | 30.79 | O |
| ATOM | 320 | CB | LEU | A | 23 | 70.036 | 5.107 | 40.089 | 1.00 | 22.72 | C |
| ATOM | 321 | CG | LEU | A | 23 | 68.966 | 4.0723 | 9.658 | 1.00 | 26.16 | C |
| ATOM | 322 | CD1 | LEU | A | 23 | 69.271 | 3.083 | 38.481 | 1.00 | 24.80 | C |
| ATOM | 323 | CD2 | LEU | A | 23 | 68.427 | 3.284 | 40.835 | 1.00 | 22.91 | C |
| ATOM | 324 | N | GLU | A | 24 | 71.279 | 7.192 | 42.153 | 1.00 | 28.77 | N |
| ATOM | 325 | CA | GLU | A | 24 | 72.419 | 7.675 | 42.909 | 1.00 | 33.79 | C |
| ATOM | 326 | C | GLU | A | 24 | 72.363 | 7.388 | 44.412 | 1.00 | 35.94 | C |
| ATOM | 327 | O | GLU | A | 24 | 73.381 | 7.140 | 45.031 | 1.00 | 39.07 | O |
| ATOM | 328 | CB | GLU | A | 24 | 72.647 | 9.165 | 42.653 | 1.00 | 36.21 | C |
| ATOM | 329 | CG | GLU | A | 24 | 74.068 | 9.482 | 42.243 | 1.00 | 42.54 | C |
| ATOM | 330 | CD | GLU | A | 24 | 74.158 | 10.689 | 41.333 | 1.00 | 89.51 | C |
| ATOM | 331 | OE1 | GLU | A | 24 | 73.386 | 11.663 | 41.549 | 1.00 | 43.21 | O |
| ATOM | 332 | OE2 | GLU | A | 24 | 74.994 | 10.646 | 40.398 | 1.00 | 66.28 | O |
| ATOM | 333 | N | GLN | A | 25 | 71.182 | 7.422 | 45.000 | 1.00 | 45.70 | N |
| ATOM | 334 | CA | GLN | A | 25 | 71.039 | 7.152 | 46.432 | 1.00 | 47.57 | C |
| ATOM | 335 | C | GLN | A | 25 | 70.887 | 5.669 | 46.740 | 1.00 | 67.34 | C |
| ATOM | 336 | O | GLN | A | 25 | 70.285 | 5.286 | 47.726 | 1.00 | 74.06 | O |
| ATOM | 337 | CB | GLN | A | 25 | 69.783 | 7.842 | 46.905 | 1.00 | 51.85 | C |
| ATOM | 338 | CG | GLN | A | 25 | 69.500 | 9.084 | 46.109 | 1.00 | 44.91 | C |
| ATOM | 339 | CD | GLN | A | 25 | 68.419 | 9.913 | 46.742 | 1.00 | 100.00 | C |
| ATOM | 340 | OE1 | GLN | A | 25 | 68.271 | 9.947 | 47.972 | 1.00 | 100.00 | O |
| ATOM | 341 | NE2 | GLN | A | 25 | 67.624 | 10.602 | 45.911 | 1.00 | 100.00 | N |
| ATOM | 342 | N | ARG | A | 26 | 71.322 | 4.831 | 45.825 | 1.00 | 75.37 | N |
| ATOM | 343 | CA | ARG | A | 26 | 71.182 | 3.4074 | 6.026 | 1.00 | 74.87 | C |
| ATOM | 344 | C | ARG | A | 26 | 72.568 | 2.791 | 46.147 | 1.00 | 74.08 | C |
| ATOM | 345 | O | ARG | A | 26 | 73.440 | 2.997 | 45.289 | 1.00 | 77.00 | O |
| ATOM | 346 | CB | ARG | A | 26 | 70.390 | 2.790 | 44.885 | 1.00 | 52.44 | C |
| ATOM | 347 | CG | ARG | A | 26 | 68.916 | 2.927 | 45.070 | 1.00 | 43.51 | C |
| ATOM | 348 | CD | ARG | A | 26 | 68.428 | 1.752 | 45.864 | 1.00 | 40.70 | C |
| ATOM | 349 | NE | ARG | A | 26 | 67.200 | 1.176 | 45.338 | 1.00 | 42.33 | N |
| ATOM | 350 | CZ | ARG | A | 26 | 67.126 | 0.508 | 44.196 | 1.00 | 32.07 | C |
| ATOM | 351 | NH1 | ARG | A | 26 | 68.215 | 0.324 | 43.486 | 1.00 | 44.02 | N |
| ATOM | 352 | NH2 | ARG | A | 26 | 65.968 | 0.017 | 43.771 | 1.00 | 77.32 | N |
| ATOM | 353 | N | GLY | A | 27 | 72.778 | 2.114 | 47.266 | 1.00 | 46.30 | N |
| ATOM | 354 | CA | GLY | A | 27 | 74.060 | 1.531 | 47.549 | 1.00 | 46.82 | C |
| ATOM | 355 | C | GLY | A | 27 | 74.140 | 0.165 | 46.923 | 1.00 | 55.45 | C |
| ATOM | 356 | O | GLY | A | 27 | 75.204 | −0.453 | 46.877 | 1.00 | 64.43 | O |
| ATOM | 357 | N | ASP | A | 28 | 73.017 | −0.315 | 46.428 | 1.00 | 40.98 | N |
| ATOM | 358 | CA | ASP | A | 28 | 73.016 | −1.647 | 45.861 | 1.00 | 40.35 | C |
| ATOM | 359 | C | ASP | A | 28 | 73.266 | −1.536 | 44.400 | 1.00 | 39.55 | C |
| ATOM | 360 | O | ASP | A | 28 | 73.109 | −2.518 | 43.654 | 1.00 | 48.80 | O |
| ATOM | 361 | CB | ASP | A | 28 | 71.680 | −2.335 | 46.127 | 1.00 | 47.80 | C |
| ATOM | 362 | CG | ASP | A | 28 | 70.503 | −1.373 | 46.064 | 1.00 | 35.34 | C |
| ATOM | 363 | OD1 | ASP | A | 28 | 70.705 | −0.140 | 46.095 | 1.00 | 39.23 | O |
| ATOM | 364 | OD2 | ASP | A | 28 | 69.383 | −1.870 | 45.872 | 1.00 | 69.86 | O |
| ATOM | 365 | N | VAL | A | 29 | 73.651 | −0.329 | 43.996 | 1.00 | 31.03 | N |
| ATOM | 366 | CA | VAL | A | 29 | 73.881 | −0.050 | 42.591 | 1.00 | 28.44 | C |
| ATOM | 367 | C | VAL | A | 29 | 75.166 | 0.676 | 42.281 | 1.00 | 28.00 | C |
| ATOM | 368 | O | VAL | A | 29 | 75.505 | 1.699 | 42.892 | 1.00 | 34.83 | O |
| ATOM | 369 | CB | VAL | A | 29 | 72.696 | 0.760 | 42.000 | 1.00 | 30.68 | C |
| ATOM | 370 | CG1 | VAL | A | 29 | 72.935 | 1.088 | 40.549 | 1.00 | 23.65 | C |
| ATOM | 371 | CG2 | VAL | A | 29 | 71.416 | −0.028 | 42.156 | 1.00 | 27.95 | C |
| ATOM | 372 | N | GLU | A | 30 | 75.824 | 0.219 | 41.230 | 1.00 | 30.76 | N |
| ATOM | 373 | CA | GLU | A | 30 | 76.995 | 0.924 | 40.736 | 1.00 | 28.38 | C |
| ATOM | 374 | C | GLU | A | 30 | 76.678 | 1.471 | 39.332 | 1.00 | 31.03 | C |
| ATOM | 375 | O | GLU | A | 30 | 76.368 | 0.720 | 38.397 | 1.00 | 26.64 | O |
| ATOM | 376 | CB | GLU | A | 30 | 78.199 | 0.006 | 40.722 | 1.00 | 31.84 | C |
| ATOM | 377 | CG | GLU | A | 30 | 79.355 | 0.539 | 41.533 | 1.00 | 89.26 | C |
| ATOM | 378 | CD | GLU | A | 30 | 80.667 | 0.264 | 40.858 | 1.00 | 100.00 | C |
| ATOM | 379 | OE1 | GLU | A | 30 | 81.082 | −0.922 | 40.872 | 1.00 | 88.94 | O |
| ATOM | 380 | OE2 | GLU | A | 30 | 81.202 | 1.206 | 40.219 | 1.00 | 100.00 | O |
| ATOM | 381 | N | LEU | A | 31 | 76.665 | 2.789 | 39.207 | 1.00 | 22.24 | N |
| ATOM | 382 | CA | LEU | A | 31 | 76.269 | 3.391 | 37.945 | 1.00 | 29.37 | C |
| ATOM | 383 | C | LEU | A | 31 | 77.404 | 3.507 | 36.941 | 1.00 | 25.79 | C |
| ATOM | 384 | O | LEU | A | 31 | 78.485 | 3.969 | 37.256 | 1.00 | 29.41 | O |
| ATOM | 385 | CB | LEU | A | 31 | 75.632 | 4.760 | 38.191 | 1.00 | 30.20 | C |
| ATOM | 386 | CG | LEU | A | 31 | 74.329 | 4.763 | 38.994 | 1.00 | 29.37 | C |
| ATOM | 387 | CD1 | LEU | A | 31 | 73.841 | 6.143 | 39.240 | 1.00 | 23.43 | C |
| ATOM | 388 | CD2 | LEU | A | 31 | 73.275 | 3.962 | 38.281 | 1.00 | 23.04 | C |
| ATOM | 389 | N | VAL | A | 32 | 77.146 | 3.100 | 35.711 | 1.00 | 21.94 | N |
| ATOM | 390 | CA | VAL | A | 32 | 78.143 | 3.265 | 34.685 | 1.00 | 25.48 | C |
| ATOM | 391 | C | VAL | A | 32 | 77.535 | 4.242 | 33.669 | 1.00 | 38.76 | C |
| ATOM | 392 | O | VAL | A | 32 | 76.429 | 3.999 | 33.180 | 1.00 | 29.70 | O |
| ATOM | 393 | CB | VAL | A | 32 | 78.517 | 1.902 | 34.055 | 1.00 | 34.25 | C |
| ATOM | 394 | CG1 | VAL | A | 32 | 79.587 | 2.079 | 32.970 | 1.00 | 30.56 | C |
| ATOM | 395 | CG2 | VAL | A | 32 | 79.003 | 0.950 | 35.139 | 1.00 | 25.27 | C |
| ATOM | 396 | N | LEU | A | 33 | 78.219 | 5.375 | 33.457 | 1.00 | 30.19 | N |
| ATOM | 397 | CA | LEU | A | 33 | 77.732 | 6.463 | 32.621 | 1.00 | 22.71 | C |
| ATOM | 398 | C | LEU | A | 33 | 78.727 | 6.979 | 31.645 | 1.00 | 29.55 | C |
| ATOM | 399 | O | LEU | A | 33 | 79.896 | 7.152 | 31.988 | 1.00 | 30.09 | O |
| ATOM | 400 | CB | LEU | A | 33 | 77.423 | 7.635 | 33.514 | 1.00 | 19.75 | C |
| ATOM | 401 | CG | LEU | A | 33 | 76.729 | 7.200 | 34.779 | 1.00 | 19.38 | C |
| ATOM | 402 | CD1 | LEU | A | 33 | 76.814 | 8.344 | 35.762 | 1.00 | 27.24 | C |
| ATOM | 403 | CD2 | LEU | A | 33 | 75.271 | 6.913 | 34.444 | 1.00 | 22.07 | C |
| ATOM | 404 | N | ARG | A | 34 | 78.239 | 7.4213 | 0.496 | 1.00 | 15.09 | N |
| ATOM | 405 | CA | ARG | A | 34 | 79.154 | 8.008 | 29.541 | 1.00 | 26.04 | C |
| ATOM | 406 | CA | ARG | A | 34 | 78.469 | 9.173 | 28.916 | 1.00 | 36.57 | C |
| ATOM | 407 | O | ARG | A | 34 | 77.288 | 9.130 | 28.651 | 1.00 | 38.59 | O |
| ATOM | 408 | CB | ARG | A | 34 | 79.486 | 7.048 | 28.398 | 1.00 | 22.89 | C |
| ATOM | 409 | CG | ARG | A | 34 | 80.579 | 6.081 | 28.706 | 1.00 | 23.29 | C |
| ATOM | 410 | CD | ARG | A | 34 | 81.370 | 6.575 | 29.860 | 1.00 | 52.06 | C |
| ATOM | 411 | NE | ARG | A | 34 | 81.783 | 5.458 | 30.711 | 1.00 | 80.25 | N |
| ATOM | 412 | CZ | ARG | A | 34 | 82.646 | 4.530 | 30.323 | 1.00 | 41.94 | C |
| ATOM | 413 | NH1 | ARG | A | 34 | 83.173 | 4.596 | 29.104 | 1.00 | 53.02 | N |
| ATOM | 414 | NH2 | ARG | A | 34 | 82.983 | 3.547 | 31.148 | 1.00 | 25.56 | N |
| ATOM | 415 | N | THR | A | 35 | 79.248 | 10.156 | 28.539 | 1.00 | 31.58 | N |
| ATOM | 416 | CA | THR | A | 35 | 78.703 | 11.282 | 27.833 | 1.00 | 29.33 | C |
| ATOM | 417 | C | THR | A | 35 | 78.719 | 10.951 | 26.340 | 1.00 | 32.53 | C |
| ATOM | 418 | O | THR | A | 35 | 79.350 | 9.944 | 25.962 | 1.00 | 28.08 | O |
| ATOM | 419 | CB | THR | A | 35 | 79.527 | 12.527 | 28.145 | 1.00 | 37.49 | C |
| ATOM | 420 | OG1 | THR | A | 35 | 80.644 | 12.429 | 27.560 | 1.00 | 31.91 | O |
| ATOM | 421 | CG2 | THR | A | 35 | 79.627 | 12.642 | 29.651 | 1.00 | 19.38 | C |
| ATOM | 422 | N | ARG | A | 36 | 78.032 | 11.780 | 25.529 | 1.00 | 30.02 | N |
| ATOM | 423 | CA | ARG | A | 36 | 78.002 | 11.639 | 24.056 | 1.00 | 29.37 | C |
| ATOM | 424 | C | ARG | A | 36 | 79.406 | 11.765 | 23.503 | 1.00 | 31.46 | C |
| ATOM | 425 | O | ARG | A | 36 | 79.772 | 11.012 | 22.591 | 1.00 | 36.56 | O |
| ATOM | 426 | CB | ARG | A | 36 | 77.054 | 12.650 | 23.354 | 1.00 | 37.34 | C |
| ATOM | 427 | CG | ARG | A | 36 | 76.937 | 12.465 | 21.846 | −99.00 | 49.47 | C |
| ATOM | 428 | CD | ARG | A | 36 | 76.020 | 13.515 | 21.232 | −99.00 | 63.09 | C |
| ATOM | 429 | NE | ARG | A | 36 | 75.528 | 13.124 | 19.915 | −99.00 | 75.23 | N |
| ATOM | 430 | CZ | ARG | A | 36 | 74.381 | 13.549 | 19.391 | −99.00 | 91.44 | C |
| ATOM | 431 | NH1 | ARG | A | 36 | 73.605 | 14.375 | 20.079 | −99.00 | 79.32 | N |
| ATOM | 432 | NH2 | ARG | A | 36 | 74.009 | 13.144 | 18.185 | −99.00 | 78.73 | N |
| ATOM | 433 | N | ASP | A | 37 | 80.217 | 12.677 | 24.063 | 1.00 | 41.30 | N |
| ATOM | 434 | CA | ASP | A | 37 | 81.606 | 12.710 | 23.601 | 1.00 | 44.91 | C |
| ATOM | 435 | C | ASP | A | 37 | 82.410 | 11.481 | 24.043 | 1.00 | 24.99 | C |
| ATOM | 436 | O | ASP | A | 37 | 83.211 | 10.978 | 23.261 | 1.00 | 42.22 | O |
| ATOM | 437 | CB | ASP | A | 37 | 82.347 | 14.048 | 23.718 | −99.00 | 47.07 | C |
| ATOM | 438 | CG | ASP | A | 37 | 81.881 | 14.887 | 24.876 | −99.00 | 62.99 | C |
| ATOM | 439 | OD1 | ASP | A | 37 | 80.679 | 14.839 | 25.204 | −99.00 | 64.45 | O |
| ATOM | 440 | OD2 | ASP | A | 37 | 82.711 | 15.638 | 25.429 | −99.00 | 69.84 | O |
| ATOM | 441 | N | GLU | A | 38 | 82.129 | 10.950 | 25.235 | 1.00 | 19.39 | N |
| ATOM | 442 | CA | GLU | A | 38 | 82.790 | 9.717 | 25.682 | 1.00 | 27.84 | C |
| ATOM | 443 | C | GLU | A | 38 | 82.203 | 8.527 | 24.901 | 1.00 | 37.14 | C |
| ATOM | 444 | O | GLU | A | 38 | 82.873 | 7.511 | 24.699 | 1.00 | 35.04 | O |
| ATOM | 445 | CB | GLU | A | 38 | 82.691 | 9.435 | 27.207 | 1.00 | 25.18 | C |
| ATOM | 446 | CG | GLU | A | 38 | 83.116 | 10.549 | 28.183 | 1.00 | 37.45 | C |
| ATOM | 447 | CD | GLU | A | 38 | 82.807 | 10.212 | 29.655 | 1.00 | 21.13 | C |
| ATOM | 448 | OE1 | GLU | A | 38 | 81.623 | 9.997 | 30.014 | 1.00 | 55.97 | O |
| ATOM | 449 | OE2 | GLU | A | 38 | 83.757 | 9.978 | 30.419 | 1.00 | 98.78 | O |
| ATOM | 450 | N | LEU | A | 39 | 80.948 | 8.610 | 24.478 | 1.00 | 25.52 | N |
| ATOM | 451 | CA | LEU | A | 39 | 80.440 | 7.483 | 23.739 | 1.00 | 18.17 | C |
| ATOM | 452 | C | LEU | A | 39 | 79.291 | 7.764 | 22.825 | 1.00 | 20.34 | C |
| ATOM | 453 | O | LEU | A | 39 | 78.152 | 7.810 | 23.259 | 1.00 | 26.35 | O |
| ATOM | 454 | CB | LEU | A | 39 | 80.123 | 6.313 | 24.657 | 1.00 | 14.56 | C |
| ATOM | 455 | CG | LEU | A | 39 | 79.410 | 5.075 | 24.058 | 1.00 | 19.52 | C |
| ATOM | 456 | CD1 | LEU | A | 39 | 80.205 | 4.392 | 22.994 | 1.00 | 18.84 | C |
| ATOM | 457 | CD2 | LEU | A | 39 | 78.890 | 4.051 | 25.084 | 1.00 | 17.41 | C |
| ATOM | 458 | N | ASN | A | 40 | 79.598 | 7.860 | 21.543 | 1.00 | 16.73 | N |
| ATOM | 459 | CA | ASN | A | 40 | 78.548 | 7.971 | 20.540 | 1.00 | 21.55 | C |
| ATOM | 460 | C | ASN | A | 40 | 77.798 | 6.649 | 20.308 | 1.00 | 24.53 | C |
| ATOM | 461 | O | ASN | A | 40 | 78.328 | 5.720 | 19.688 | 1.00 | 19.96 | O |
| ATOM | 462 | CB | ASN | A | 40 | 79.130 | 8.367 | 19.216 | 1.00 | 18.45 | C |
| ATOM | 463 | CG | ASN | A | 40 | 78.054 | 8.727 | 18.225 | 1.00 | 42.19 | C |
| ATOM | 464 | OD1 | ASN | A | 40 | 78.327 | 9.093 | 17.080 | 1.00 | 38.89 | O |
| ATOM | 465 | ND2 | ASN | A | 40 | 76.827 | 8.730 | 18.697 | 1.00 | 23.71 | N |
| ATOM | 466 | N | LEU | A | 41 | 76.543 | 6.622 | 20.754 | 1.00 | 21.08 | N |
| ATOM | 467 | CA | LEU | A | 41 | 75.649 | 5.465 | 20.650 | 1.00 | 15.03 | C |
| ATOM | 468 | C | LEU | A | 41 | 75.225 | 5.068 | 19.213 | 1.00 | 18.22 | C |
| ATOM | 469 | O | LEU | A | 41 | 74.681 | 3.971 | 18.980 | 1.00 | 25.72 | O |
| ATOM | 470 | CE | LEU | A | 41 | 74.426 | 5.705 | 21.532 | 1.00 | 15.65 | C |
| ATOM | 471 | CG | LEU | A | 41 | 74.822 | 6.029 | 22.974 | 1.00 | 21.90 | C |
| ATOM | 472 | CD1 | LEU | A | 41 | 73.604 | 6.413 | 23.749 | 1.00 | 20.59 | C |
| ATOM | 473 | CD2 | LEU | A | 41 | 75.481 | 4.796 | 23.609 | 1.00 | 17.97 | C |
| ATOM | 474 | N | LEU | A | 42 | 75.542 | 5.916 | 18.238 | 1.00 | 12.45 | N |
| ATOM | 475 | CA | LEU | A | 42 | 75.256 | 5.607 | 16.831 | 1.00 | 15.99 | C |
| ATOM | 476 | C | LEU | A | 42 | 76.290 | 4.680 | 16.280 | 1.00 | 26.18 | C |
| ATOM | 477 | O | LEU | A | 42 | 76.066 | 4.039 | 15.257 | 1.00 | 22.41 | O |
| ATOM | 478 | CE | LEU | A | 42 | 75.282 | 6.873 | 15.984 | 1.00 | 17.85 | C |
| ATOM | 479 | CG | LEU | A | 42 | 74.180 | 7.854 | 16.399 | 1.00 | 30.70 | C |
| ATOM | 480 | CD1 | LEU | A | 42 | 74.318 | 9.184 | 15.704 | 1.00 | 24.31 | C |
| ATOM | 481 | CD2 | LEU | A | 42 | 72.764 | 7.241 | 16.208 | 1.00 | 31.13 | C |
| ATOM | 482 | N | ASP | A | 43 | 77.462 | 4.705 | 16.911 | 1.00 | 26.87 | N |
| ATOM | 483 | CA | ASP | A | 43 | 78.579 | 3.875 | 16.486 | 1.00 | 19.29 | C |
| ATOM | 484 | C | ASP | A | 43 | 78.583 | 2.519 | 17.163 | 1.00 | 13.33 | C |
| ATOM | 485 | O | ASP | A | 43 | 79.051 | 2.348 | 18.297 | 1.00 | 18.75 | O |
| ATOM | 486 | CB | ASP | A | 43 | 79.870 | 4.580 | 16.776 | 1.00 | 31.06 | C |
| ATOM | 487 | CG | ASP | A | 43 | 81.083 | 3.758 | 16.380 | 1.00 | 30.68 | C |
| ATOM | 488 | OD1 | ASP | A | 43 | 80.971 | 2.551 | 16.082 | 1.00 | 32.36 | O |
| ATOM | 489 | OD2 | ASP | A | 43 | 82.187 | 4.308 | 16.499 | 1.00 | 37.83 | O |
| ATOM | 490 | N | SER | A | 44 | 78.139 | 1.544 | 16.377 | 1.00 | 16.89 | N |
| ATOM | 491 | CA | SER | A | 44 | 77.978 | 0.173 | 16.789 | 1.00 | 17.67 | C |
| ATOM | 492 | C | SER | A | 44 | 79.237 | −0.463 | 17.392 | 1.00 | 20.40 | C |
| ATOM | 493 | O | SER | A | 44 | 79.206 | −1.126 | 18.444 | 1.00 | 26.27 | O |
| ATOM | 494 | CE | SER | A | 44 | 77.504 | −0.617 | 15.581 | 1.00 | 13.85 | C |
| ATOM | 495 | OG | SER | A | 44 | 76.800 | −1.740 | 16.063 | 1.00 | 43.83 | O |
| ATOM | 496 | N | ARG | A | 45 | 80.335 | −0.301 | 16.682 | 1.00 | 15.63 | N |
| ATOM | 497 | CA | ARG | A | 45 | 81.616 | −0.788 | 17.154 | 1.00 | 19.94 | C |
| ATOM | 498 | C | ARG | A | 45 | 81.910 | −0.225 | 18.521 | 1.00 | 29.48 | C |
| ATOM | 499 | O | ARG | A | 45 | 82.244 | −0.937 | 19.457 | 1.00 | 27.65 | O |
| ATOM | 500 | CB | ARG | A | 45 | 82.684 | −0.261 | 16.203 | 1.00 | 27.46 | C |
| ATOM | 501 | CG | ARG | A | 45 | 83.463 | −1.338 | 15.495 | 1.00 | 92.03 | C |
| ATOM | 502 | CD | ARG | A | 45 | 84.854 | −1.418 | 16.077 | 1.00 | 100.00 | C |
| ATOM | 503 | NE | ARG | A | 45 | 85.636 | −2.533 | 15.527 | 1.00 | 100.00 | N |
| ATOM | 504 | CZ | ARG | A | 45 | 86.092 | −3.570 | 16.236 | 1.00 | 100.00 | C |
| ATOM | 505 | NH1 | ARG | A | 45 | 85.791 | −3.695 | 17.547 | 1.00 | 100.00 | N |
| ATOM | 506 | NH2 | ARG | A | 45 | 86.773 | −4.544 | 15.642 | 1.00 | 100.00 | N |
| ATOM | 507 | N | ALA | A | 46 | 81.772 | 1.090 | 18.629 | 1.00 | 31.04 | N |
| ATOM | 508 | CA | ALA | A | 46 | 82.045 | 1.743 | 19.881 | 1.00 | 24.72 | C |
| ATOM | 509 | C | ALA | A | 46 | 81.111 | 1.176 | 20.899 | 1.00 | 17.73 | C |
| ATOM | 510 | O | ALA | A | 46 | 81.512 | 0.825 | 22.027 | 1.00 | 22.73 | O |
| ATOM | 511 | CB | ALA | A | 46 | 81.839 | 3.221 | 19.751 | 1.00 | 27.16 | C |
| ATOM | 512 | N | VAL | A | 47 | 79.835 | 1.119 | 20.531 | 1.00 | 17.54 | N |
| ATOM | 513 | CA | VAL | A | 47 | 78.878 | 0.608 | 21.508 | 1.00 | 21.41 | C |
| ATOM | 514 | C | VAL | A | 47 | 79.262 | −0.812 | 21.914 | 1.00 | 30.25 | C |
| ATOM | 515 | C | VAL | A | 47 | 79.192 | −1.202 | 23.097 | 1.00 | 15.85 | O |
| ATOM | 516 | CB | VAL | A | 47 | 77.470 | 0.668 | 20.989 | 1.00 | 18.59 | C |
| ATOM | 517 | CG1 | VAL | A | 47 | 76.503 | 0.042 | 22.012 | 1.00 | 16.88 | C |
| ATOM | 518 | CG2 | VAL | A | 47 | 77.115 | 2.096 | 20.756 | 1.00 | 16.28 | C |
| ATOM | 519 | N | HIS | A | 48 | 79.692 | −1.585 | 20.920 | 1.00 | 21.00 | N |
| ATOM | 520 | CA | HIS | A | 48 | 80.028 | −2.969 | 21.192 | 1.00 | 20.17 | C |
| ATOM | 521 | C | HIS | A | 48 | 81.268 | −3.079 | 22.117 | 1.00 | 32.98 | C |
| ATOM | 522 | O | HIS | A | 48 | 81.289 | −3.850 | 23.102 | 1.00 | 28.20 | O |
| ATOM | 523 | CB | HIS | A | 48 | 80.063 | −3.801 | 19.855 | 1.00 | 14.93 | C |
| ATOM | 524 | CG | HIS | A | 48 | 78.686 | −4.172 | 19.338 | 1.00 | 26.67 | C |
| ATOM | 525 | ND1 | HIS | A | 48 | 78.085 | −5.394 | 19.600 | 1.00 | 28.83 | N |
| ATOM | 526 | CD2 | HIS | A | 48 | 77.758 | −3.448 | 18.659 | 1.00 | 25.56 | C |
| ATOM | 527 | CE1 | HIS | A | 48 | 76.887 | −5.430 | 19.043 | 1.00 | 20.08 | C |
| ATOM | 528 | NE2 | HIS | A | 48 | 76.660 | −4.260 | 18.475 | 1.00 | 25.22 | N |
| ATOM | 529 | N | ASP | A | 49 | 82.217 | −2.170 | 21.902 | 1.00 | 22.62 | N |
| ATOM | 530 | CA | ASP | A | 49 | 83.455 | −2.169 | 22.674 | 1.00 | 24.23 | C |
| ATOM | 531 | C | ASP | A | 49 | 83.171 | −1.899 | 24.122 | 1.00 | 38.72 | C |
| ATOM | 532 | O | ASP | A | 49 | 83.708 | −2.551 | 25.027 | 1.00 | 35.44 | O |
| ATOM | 533 | CB | ASP | A | 49 | 84.396 | −1.112 | 22.127 | 1.00 | 30.29 | C |
| ATOM | 534 | CG | ASP | A | 49 | 84.991 | −1.503 | 20.775 | 1.00 | 52.45 | C |
| ATOM | 535 | OD1 | ASP | A | 49 | 85.007 | −2.726 | 20.449 | 1.00 | 42.67 | O |
| ATOM | 536 | OD2 | ASP | A | 49 | 85.416 | −0.587 | 20.029 | 1.00 | 73.76 | O |
| ATOM | 537 | N | PHE | A | 50 | 82.294 | −0.929 | 24.324 | 1.00 | 32.19 | N |
| ATOM | 538 | CA | PHE | A | 50 | 81.902 | −0.550 | 25.649 | 1.00 | 29.76 | C |
| ATOM | 539 | C | PHE | A | 50 | 81.299 | −1.765 | 26.359 | 1.00 | 30.31 | C |
| ATOM | 540 | O | PHE | A | 50 | 81.715 | −2.124 | 27.449 | 1.00 | 29.22 | O |
| ATOM | 541 | CB | PHE | A | 50 | 80.892 | 0.610 | 25.576 | 1.00 | 23.82 | C |
| ATOM | 542 | CG | PHE | A | 50 | 80.137 | 0.843 | 26.859 | 1.00 | 19.13 | C |
| ATOM | 543 | CD1 | PHE | A | 50 | 80.740 | 1.515 | 27.931 | 1.00 | 20.14 | C |
| ATOM | 544 | CD2 | PHE | A | 50 | 78.835 | 0.360 | 27.018 | 1.00 | 13.99 | C |
| ATOM | 545 | CE1 | PHE | A | 50 | 80.034 | 1.742 | 29.129 | 1.00 | 25.81 | C |
| ATOM | 546 | CE2 | PHE | A | 50 | 78.114 | 0.553 | 28.212 | 1.00 | 22.84 | C |
| ATOM | 547 | CZ | PHE | A | 50 | 78.698 | 1.276 | 29.259 | 1.00 | 23.40 | C |
| ATOM | 548 | N | PHE | A | 51 | 80.280 | −2.367 | 25.768 | 1.00 | 21.75 | N |
| ATOM | 549 | CA | PHE | A | 51 | 79.655 | −3.451 | 26.457 | 1.00 | 22.61 | C |
| ATOM | 550 | C | PHE | A | 51 | 80.646 | −4.603 | 26.612 | 1.00 | 34.01 | C |
| ATOM | 551 | O | PHE | A | 51 | 80.550 | −5.401 | 27.590 | 1.00 | 25.28 | O |
| ATOM | 552 | CB | PHE | A | 51 | 78.389 | −3.898 | 25.751 | 1.00 | 22.63 | C |
| ATOM | 553 | CG | PHE | A | 51 | 77.158 | −3.140 | 26.170 | 1.00 | 27.58 | C |
| ATOM | 554 | CD1 | PHE | A | 51 | 76.426 | −3.525 | 27.280 | 1.00 | 21.78 | C |
| ATOM | 555 | CD2 | PHE | A | 51 | 76.663 | −2.100 | 25.380 | 1.00 | 19.55 | C |
| ATOM | 556 | CE1 | PHE | A | 51 | 75.267 | −2.796 | 27.662 | 1.00 | 28.34 | C |
| ATOM | 557 | CE2 | PHE | A | 51 | 75.492 | −1.403 | 25.734 | 1.00 | 14.47 | C |
| ATOM | 558 | CZ | PHE | A | 51 | 74.797 | −1.744 | 26.878 | 1.00 | 14.55 | C |
| ATOM | 559 | N | ALA | A | 52 | 81.576 | −4.706 | 25.659 | 1.00 | 26.43 | N |
| ATOM | 560 | CA | ALA | A | 52 | 82.587 | −5.793 | 25.714 | 1.00 | 29.44 | C |
| ATOM | 561 | C | ALA | A | 52 | 83.687 | −5.560 | 26.768 | 1.00 | 43.76 | C |
| ATOM | 562 | O | ALA | A | 52 | 84.502 | −6.446 | 27.022 | 1.00 | 40.33 | O |
| ATOM | 563 | CB | ALA | A | 52 | 83.228 | −6.049 | 24.344 | 1.00 | 24.25 | C |
| ATOM | 564 | N | SER | A | 53 | 83.702 | −4.382 | 27.385 | 1.00 | 31.96 | N |
| ATOM | 565 | CA | SER | A | 53 | 84.705 | −4.090 | 28.377 | 1.00 | 21.06 | C |
| ATOM | 566 | C | SER | A | 53 | 84.196 | −3.625 | 29.709 | 1.00 | 26.41 | C |
| ATOM | 567 | O | SER | A | 53 | 84.985 | −3.492 | 30.611 | 1.00 | 36.12 | O |
| ATOM | 568 | CB | SER | A | 53 | 85.709 | −3.088 | 27.843 | 1.00 | 14.22 | C |
| ATOM | 569 | OG | SER | A | 53 | 85.140 | −1.807 | 27.790 | 1.00 | 56.90 | O |
| ATOM | 570 | N | GLU | A | 54 | 82.892 | −3.431 | 29.874 | 1.00 | 22.38 | N |
| ATOM | 571 | CA | GLU | A | 54 | 82.380 | −2.893 | 31.139 | 1.00 | 17.27 | C |
| ATOM | 572 | C | GLU | A | 54 | 81.584 | −3.735 | 32.118 | 1.00 | 26.32 | C |
| ATOM | 573 | O | GLU | A | 54 | 81.229 | −3.281 | 33.191 | 1.00 | 37.43 | O |
| ATOM | 574 | CB | GLU | A | 54 | 81.677 | −1.563 | 30.906 | 1.00 | 27.30 | C |
| ATOM | 575 | CG | GLU | A | 54 | 82.573 | −0.543 | 30.262 | 1.00 | 44.77 | C |
| ATOM | 576 | CD | GLU | A | 54 | 83.669 | −0.142 | 31.194 | 1.00 | 86.31 | C |
| ATOM | 577 | OE1 | GLU | A | 54 | 83.392 | −0.232 | 32.428 | 1.00 | 50.11 | O |
| ATOM | 578 | OE2 | GLU | A | 54 | 84.785 | 0.198 | 30.692 | 1.00 | 50.99 | O |
| ATOM | 579 | N | ARG | A | 55 | 81.268 | −4.971 | 31.804 | 1.00 | 29.63 | N |
| ATOM | 580 | CA | ARG | A | 55 | 80.636 | −5.748 | 32.854 | 1.00 | 33.32 | C |
| ATOM | 581 | C | ARG | A | 55 | 79.347 | −5.149 | 33.378 | 1.00 | 38.45 | C |
| ATOM | 582 | O | ARG | A | 55 | 79.214 | −4.897 | 34.576 | 1.00 | 40.18 | O |
| ATOM | 583 | CB | ARG | A | 55 | 81.621 | −5.875 | 34.045 | 1.00 | 57.61 | C |
| ATOM | 584 | CG | ARG | A | 55 | 82.666 | −7.028 | 33.960 | 1.00 | 100.00 | C |
| ATOM | 585 | CD | ARG | A | 55 | 82.805 | −7.805 | 35.305 | 1.00 | 100.00 | C |
| ATOM | 586 | NE | ARG | A | 55 | 82.838 | −9.270 | 35.146 | 1.00 | 100.00 | N |
| ATOM | 587 | CZ | ARG | A | 55 | 83.206 | −10.129 | 36.102 | 1.00 | 100.00 | C |
| ATOM | 588 | NH1 | ARG | A | 55 | 83.583 | −9.681 | 37.301 | 1.00 | 100.00 | N |
| ATOM | 589 | NH2 | ARG | A | 55 | 83.208 | −11.440 | 35.855 | 1.00 | 100.00 | N |
| ATOM | 590 | N | ILE | A | 56 | 78.367 | −5.029 | 32.491 | 1.00 | 42.25 | N |
| ATOM | 591 | CA | ILE | A | 56 | 77.064 | −4.434 | 32.794 | 1.00 | 25.49 | C |
| ATOM | 592 | C | ILE | A | 56 | 75.982 | −5.474 | 33.244 | 1.00 | 20.18 | C |
| ATOM | 593 | O | ILE | A | 56 | 75.897 | −6.579 | 32.704 | 1.00 | 24.74 | O |
| ATOM | 594 | CB | ILE | A | 56 | 76.672 | −3.512 | 31.531 | 1.00 | 26.89 | C |
| ATOM | 595 | CG1 | ILE | A | 56 | 77.643 | −2.301 | 31.442 | 1.00 | 18.30 | C |
| ATOM | 596 | CG2 | ILE | A | 56 | 75.214 | −3.016 | 31.549 | 1.00 | 19.84 | C |
| ATOM | 597 | CD1 | ILE | A | 56 | 77.998 | −1.936 | 30.026 | 1.00 | 60.42 | C |
| ATOM | 598 | N | ASP | A | 57 | 75.166 | −5.133 | 34.237 | 1.00 | 16.84 | N |
| ATOM | 599 | CA | ASP | A | 57 | 74.040 | −5.999 | 34.630 | 1.00 | 16.33 | C |
| ATOM | 600 | C | ASP | A | 57 | 72.676 | −5.451 | 34.123 | 1.00 | 28.40 | C |
| ATOM | 601 | O | ASP | A | 57 | 71.836 | −6.198 | 33.657 | 1.00 | 25.50 | O |
| ATOM | 602 | CB | ASP | A | 57 | 74.009 | −6.194 | 36.164 | 1.00 | 16.94 | C |
| ATOM | 603 | CG | ASP | A | 57 | 75.369 | −6.720 | 36.703 | 1.00 | 34.27 | C |
| ATOM | 604 | OD1 | ASP | A | 57 | 75.875 | −7.729 | 36.141 | 1.00 | 31.76 | O |
| ATOM | 605 | OD2 | ASP | A | 57 | 76.040 | −6.007 | 37.499 | 1.00 | 28.36 | O |
| ATOM | 606 | N | GLN | A | 58 | 72.443 | −4.152 | 34.220 | 1.00 | 28.91 | N |
| ATOM | 607 | CA | GLN | A | 58 | 71.183 | −3.590 | 33.755 | 1.00 | 25.68 | C |
| ATOM | 608 | C | GLN | A | 58 | 71.425 | −2.364 | 32.881 | 1.00 | 23.21 | C |
| ATOM | 609 | O | GLN | A | 58 | 72.403 | −1.620 | 33.067 | 1.00 | 18.16 | O |
| ATOM | 610 | CB | GLN | A | 58 | 70.342 | −3.151 | 34.946 | 1.00 | 33.14 | C |
| ATOM | 611 | CG | GLN | A | 58 | 69.798 | −4.241 | 35.807 | 1.00 | 30.00 | C |
| ATOM | 612 | CD | GLN | A | 58 | 69.226 | −3.712 | 37.105 | 1.00 | 27.18 | C |
| ATOM | 613 | OE1 | GLN | A | 58 | 68.722 | −2.601 | 37.161 | 1.00 | 31.20 | O |
| ATOM | 614 | NE2 | GLN | A | 58 | 69.455 | −4.436 | 38.186 | 1.00 | 16.89 | N |
| ATOM | 615 | N | VAL | A | 59 | 70.496 | −2.138 | 31.961 | 1.00 | 18.35 | N |
| ATOM | 616 | CA | VAL | A | 59 | 70.562 | −0.998 | 31.045 | 1.00 | 15.59 | C |
| ATOM | 617 | C | VAL | A | 59 | 69.238 | −0.240 | 31.039 | 1.00 | 26.28 | C |
| ATOM | 618 | O | VAL | A | 59 | 68.178 | −0.820 | 30.762 | 1.00 | 19.51 | O |
| ATOM | 619 | CB | VAL | A | 59 | 70.707 | ‘1.456 | 29.601 | 1.00 | 15.32 | C |
| ATOM | 620 | CG1 | VAL | A | 59 | 70.477 | −0.274 | 28.649 | 1.00 | 11.93 | C |
| ATOM | 621 | CG2 | VAL | A | 59 | 72.080 | −2.111 | 29.364 | 1.00 | 15.83 | C |
| ATOM | 622 | N | TYR | A | 60 | 69.306 | 1.064 | 31.293 | 1.00 | 21.71 | N |
| ATOM | 623 | CA | TYR | A | 60 | 68.113 | 1.927 | 31.197 | 1.00 | 21.40 | C |
| ATOM | 624 | C | TYR | A | 60 | 68.289 | 2.756 | 29.928 | 1.00 | 18.69 | C |
| ATOM | 625 | O | TYR | A | 60 | 69.250 | 3.532 | 29.796 | 1.00 | 15.51 | O |
| ATOM | 626 | CB | TYR | A | 60 | 68.021 | 2.817 | 32.413 | 1.00 | 17.24 | C |
| ATOM | 627 | CG | TYR | A | 60 | 67.493 | 2.131 | 33.658 | 1.00 | 19.71 | C |
| ATOM | 628 | CD1 | TYR | A | 60 | 68.345 | 1.583 | 34.586 | 1.00 | 21.14 | C |
| ATOM | 629 | CD2 | TYR | A | 60 | 66.154 | 2.223 | 33.991 | 1.00 | 20.16 | C |
| ATOM | 630 | CE1 | TYR | A | 60 | 67.835 | 1.080 | 35.794 | 1.00 | 19.11 | C |
| ATOM | 631 | CE2 | TYR | A | 60 | 65.648 | 1.698 | 35.163 | 1.00 | 10.77 | C |
| ATOM | 632 | CZ | TYR | A | 60 | 66.476 | 1.094 | 36.054 | 1.00 | 20.07 | C |
| ATOM | 633 | OH | TYR | A | 60 | 65.921 | 0.585 | 37.248 | 1.00 | 16.04 | O |
| ATOM | 634 | N | LEU | A | 61 | 67.491 | 2.452 | 28.916 | 1.00 | 17.46 | N |
| ATOM | 635 | CA | LEU | A | 61 | 67.685 | 3.053 | 27.585 | 1.00 | 20.17 | C |
| ATOM | 636 | C | LEU | A | 61 | 67.003 | 4.412 | 27.409 | 1.00 | 23.36 | C |
| ATOM | 637 | O | LEU | A | 61 | 65.925 | 4.526 | 26.799 | 1.00 | 14.86 | O |
| ATOM | 638 | CB | LEU | A | 61 | 67.267 | 2.060 | 26.485 | 1.00 | 14.78 | C |
| ATOM | 639 | CG | LEU | A | 61 | 68.117 | 2.142 | 25.208 | 1.00 | 15.52 | C |
| ATOM | 640 | CD1 | LEU | A | 61 | 67.815 | 1.010 | 24.109 | 1.00 | 7.75 | C |
| ATOM | 641 | CD2 | LEU | A | 61 | 68.087 | 3.541 | 24.580 | 1.00 | 15.20 | C |
| ATOM | 642 | N | ALA | A | 62 | 67.656 | 5.434 | 27.956 | 1.00 | 20.35 | N |
| ATOM | 643 | CA | ALA | A | 62 | 67.120 | 6.784 | 27.963 | 1.00 | 18.55 | C |
| ATOM | 644 | C | ALA | A | 62 | 67.779 | 7.739 | 26.949 | 1.00 | 18.57 | C |
| ATOM | 645 | O | ALA | A | 62 | 67.455 | 8.924 | 26.920 | 1.00 | 24.31 | O |
| ATOM | 646 | CB | ALA | A | 62 | 67.071 | 7.377 | 29.439 | 1.00 | 11.69 | C |
| ATOM | 647 | N | ALA | A | 63 | 68.681 | 7.231 | 26.101 | 1.00 | 14.09 | N |
| ATOM | 648 | CA | ALA | A | 63 | 69.249 | 8.095 | 25.052 | 1.00 | 12.84 | C |
| ATOM | 649 | C | ALA | A | 63 | 68.310 | 8.005 | 23.877 | 1.00 | 27.00 | C |
| ATOM | 650 | O | ALA | A | 63 | 67.845 | 6.916 | 23.511 | 1.00 | 24.51 | O |
| ATOM | 651 | CB | ALA | A | 63 | 70.665 | 7.660 | 24.634 | 1.00 | 4.89 | C |
| ATOM | 652 | N | ALA | A | 64 | 68.076 | 9.148 | 23.262 | 1.00 | 21.05 | N |
| ATOM | 653 | CA | ALA | A | 64 | 67.202 | 9.286 | 22.086 | 1.00 | 13.50 | C |
| ATOM | 654 | C | ALA | A | 64 | 67.435 | 10.664 | 21.416 | 1.00 | 28.08 | C |
| ATOM | 655 | O | ALA | A | 64 | 67.987 | 11.600 | 22.021 | 1.00 | 26.63 | O |
| ATOM | 656 | CB | ALA | A | 64 | 65.642 | 9.171 | 22.518 | 1.00 | 7.63 | C |
| ATOM | 657 | N | LYS | A | 65 | 66.953 | 10.781 | 20.182 | 1.00 | 23.98 | N |
| ATOM | 658 | CA | LYS | A | 65 | 66.966 | 12.012 | 19.409 | 1.00 | 20.47 | C |
| ATOM | 659 | C | LYS | A | 65 | 65.488 | 12.443 | 19.551 | 1.00 | 24.37 | C |
| ATOM | 660 | O | LYS | A | 65 | 64.594 | 11.807 | 18.976 | 1.00 | 20.29 | O |
| ATOM | 661 | CE | LYS | A | 65 | 67.317 | 11.658 | 17.951 | 1.00 | 25.59 | C |
| ATOM | 662 | CG | LYS | A | 65 | 66.808 | 12.630 | 16.923 | 1.00 | 27.54 | C |
| ATOM | 663 | CD | LYS | A | 65 | 67.518 | 13.926 | 17.169 | 1.00 | 21.08 | C |
| ATOM | 664 | CE | LYS | A | 65 | 67.316 | 14.905 | 16.029 | 1.00 | 55.15 | C |
| ATOM | 665 | NZ | LYS | A | 65 | 67.876 | 16.263 | 16.392 | 1.00 | 81.63 | N |
| ATOM | 666 | N | VAL | A | 66 | 65.228 | 13.362 | 20.485 | 1.00 | 22.47 | N |
| ATOM | 667 | CA | VAL | A | 66 | 63.873 | 13.850 | 20.755 | 1.00 | 18.99 | C |
| ATOM | 668 | C | VAL | A | 66 | 63.711 | 15.343 | 20.394 | 1.00 | 31.44 | C |
| ATOM | 669 | O | VAL | A | 66 | 64.665 | 16.107 | 20.460 | 1.00 | 34.61 | O |
| ATOM | 670 | CE | VAL | A | 66 | 63.440 | 13.623 | 22.204 | 1.00 | 16.66 | C |
| ATOM | 671 | CG1 | VAL | A | 66 | 64.269 | 12.623 | 22.869 | 1.00 | 15.01 | C |
| ATOM | 672 | CG2 | VAL | A | 66 | 63.379 | 14.904 | 22.950 | 1.00 | 19.21 | C |
| ATOM | 673 | N | GLY | A | 67 | 62.514 | 15.755 | 19.994 | 1.00 | 18.03 | N |
| ATOM | 674 | CA | GLY | A | 67 | 62.298 | 17.149 | 19.614 | 1.00 | 14.90 | C |
| ATOM | 675 | C | GLY | A | 67 | 60.792 | 17.518 | 19.585 | 1.00 | 32.35 | C |
| ATOM | 676 | O | GLY | A | 67 | 59.922 | 16.666 | 19.888 | 1.00 | 18.88 | O |
| ATOM | 677 | N | GLY | A | 68 | 60.503 | 18.787 | 19.256 | 1.00 | 23.21 | N |
| ATOM | 678 | CA | GLY | A | 68 | 59.132 | 19.288 | 19.183 | 1.00 | 23.83 | C |
| ATOM | 679 | C | GLY | A | 68 | 58.540 | 19.137 | 17.771 | 1.00 | 19.31 | C |
| ATOM | 680 | O | GLY | A | 68 | 59.165 | 18.550 | 16.870 | 1.00 | 30.64 | O |
| ATOM | 681 | N | ILE | A | 69 | 57.343 | 19.684 | 17.588 | 1.00 | 15.20 | N |
| ATOM | 682 | CA | ILE | A | 69 | 56.595 | 19.632 | 16.317 | 1.00 | 16.80 | C |
| ATOM | 683 | C | ILE | A | 69 | 57.387 | 20.153 | 15.112 | 1.00 | 19.33 | C |
| ATOM | 684 | O | ILE | A | 69 | 57.425 | 19.519 | 14.061 | 1.00 | 14.66 | O |
| ATOM | 685 | CE | ILE | A | 69 | 55.257 | 20.432 | 16.480 | 1.00 | 30.11 | C |
| ATOM | 686 | CG1 | ILE | A | 69 | 54.271 | 19.683 | 17.385 | 1.00 | 24.27 | C |
| ATOM | 687 | CG2 | ILE | A | 69 | 54.610 | 20.749 | 15.181 | 1.00 | 47.53 | C |
| ATOM | 688 | CD1 | ILE | A | 69 | 53.259 | 20.608 | 18.056 | 1.00 | 85.71 | C |
| ATOM | 689 | N | VAL | A | 70 | 58.010 | 21.327 | 15.269 | 1.00 | 23.03 | N |
| ATOM | 690 | CA | VAL | A | 70 | 58.797 | 21.913 | 14.183 | 1.00 | 19.34 | C |
| ATOM | 691 | C | VAL | A | 70 | 59.983 | 21.011 | 13.840 | 1.00 | 24.42 | C |
| ATOM | 692 | O | VAL | A | 70 | 60.335 | 20.829 | 12.662 | 1.00 | 24.14 | O |
| ATOM | 693 | CB | VAL | A | 70 | 59.304 | 23.404 | 14.467 | 1.00 | 21.37 | C |
| ATOM | 694 | CG1 | VAL | A | 70 | 60.137 | 23.907 | 13.281 | 1.00 | 17.79 | C |
| ATOM | 695 | CG2 | VAL | A | 70 | 58.136 | 24.410 | 14.678 | 1.00 | 15.74 | C |
| ATOM | 696 | N | ALA | A | 71 | 60.621 | 20.450 | 14.861 | 1.00 | 19.68 | N |
| ATOM | 697 | CA | ALA | A | 71 | 61.782 | 19.617 | 14.572 | 1.00 | 16.57 | C |
| ATOM | 698 | C | ALA | A | 71 | 61.427 | 18.289 | 13.910 | 1.00 | 23.36 | C |
| ATOM | 699 | O | ALA | A | 71 | 61.980 | 17.923 | 12.849 | 1.00 | 21.84 | O |
| ATOM | 700 | CB | ALA | A | 71 | 62.685 | 19.439 | 15.805 | 1.00 | 9.36 | C |
| ATOM | 701 | N | ASN | A | 72 | 60.463 | 17.598 | 14.511 | 1.00 | 16.80 | N |
| ATOM | 702 | CA | ASN | A | 72 | 59.998 | 16.357 | 13.923 | 1.00 | 18.84 | C |
| ATOM | 703 | C | ASN | A | 72 | 59.608 | 16.539 | 12.440 | 1.00 | 23.87 | C |
| ATOM | 704 | O | ASN | A | 72 | 59.919 | 15.696 | 11.593 | 1.00 | 21.52 | O |
| ATOM | 705 | CB | ASN | A | 72 | 58.835 | 15.806 | 14.738 | 1.00 | 8.60 | C |
| ATOM | 706 | CG | ASN | A | 72 | 59.309 | 15.013 | 15.911 | 1.00 | 23.75 | C |
| ATOM | 707 | OD1 | ASN | A | 72 | 59.558 | 13.809 | 15.810 | 1.00 | 23.98 | O |
| ATOM | 708 | ND2 | ASN | A | 72 | 59.572 | 15.701 | 16.996 | 1.00 | 9.96 | N |
| ATOM | 709 | N | ASN | A | 73 | 58.931 | 17.647 | 12.138 | 1.00 | 23.07 | N |
| ATOM | 710 | CA | ASN | A | 73 | 58.521 | 17.971 | 10.761 | 1.00 | 26.05 | C |
| ATOM | 711 | C | ASN | A | 73 | 59.665 | 18.454 | 9.817 | 1.00 | 26.95 | C |
| ATOM | 712 | O | ASN | A | 73 | 59.613 | 18.276 | 8.569 | 1.00 | 22.13 | O |
| ATOM | 713 | CB | ASN | A | 73 | 57.383 | 19.001 | 10.800 | 1.00 | 14.86 | C |
| ATOM | 714 | CG | ASN | A | 73 | 56.015 | 18.349 | 10.987 | 1.00 | 19.88 | C |
| ATOM | 715 | OD1 | ASN | A | 73 | 55.620 | 17.468 | 10.217 | 1.00 | 27.02 | O |
| ATOM | 716 | ND2 | ASN | A | 73 | 55.322 | 18.732 | 12.051 | 1.00 | 20.78 | N |
| ATOM | 717 | N | THR | A | 74 | 60.710 | 19.029 | 10.419 | 1.00 | 18.69 | N |
| ATOM | 718 | CA | THR | A | 74 | 61.845 | 19.540 | 9.657 | 1.00 | 10.07 | C |
| ATOM | 719 | C | THR | A | 74 | 62.968 | 18.548 | 9.375 | 1.00 | 21.00 | C |
| ATOM | 720 | O | THR | A | 74 | 63.537 | 18.561 | 8.289 | 1.00 | 11.75 | O |
| ATOM | 721 | CB | THR | A | 74 | 62.411 | 20.746 | 10.306 | 1.00 | 29.10 | C |
| ATOM | 722 | OG1 | THR | A | 74 | 61.370 | 21.714 | 10.457 | 1.00 | 23.24 | O |
| ATOM | 723 | CG2 | THR | A | 74 | 63.541 | 21.299 | 9.452 | 1.00 | 21.63 | C |
| ATOM | 724 | N | TYR | A | 75 | 63.230 | 17.636 | 10.310 | 1.00 | 17.10 | N |
| ATOM | 725 | CA | TYR | A | 75 | 64.267 | 16.620 | 10.112 | 1.00 | 9.07 | C |
| ATOM | 726 | C | TYR | A | 75 | 63.733 | 15.203 | 10.318 | 1.00 | 6.17 | C |
| ATOM | 727 | O | TYR | A | 75 | 64.143 | 14.542 | 11.267 | 1.00 | 15.58 | O |
| ATOM | 728 | CB | TYR | A | 75 | 65.302 | 16.825 | 11.188 | 1.00 | 11.89 | C |
| ATOM | 729 | CG | TYR | A | 75 | 65.779 | 18.234 | 11.252 | 1.00 | 27.12 | C |
| ATOM | 730 | CD1 | TYR | A | 75 | 66.712 | 18.696 | 10.321 | 1.00 | 28.46 | C |
| ATOM | 731 | CD2 | TYR | A | 75 | 65.234 | 19.151 | 12.173 | 1.00 | 24.83 | C |
| ATOM | 732 | CE1 | TYR | A | 75 | 67.117 | 20.045 | 10.305 | 1.00 | 28.34 | C |
| ATOM | 733 | CE2 | TYR | A | 75 | 65.652 | 20.523 | 12.180 | 1.00 | 21.00 | C |
| ATOM | 734 | CZ | TYR | A | 75 | 66.593 | 20.940 | 11.234 | 1.00 | 45.42 | C |
| ATOM | 735 | OH | TYR | A | 75 | 67.066 | 22.230 | 11.215 | 1.00 | 35.37 | O |
| ATOM | 736 | N | PRO | A | 76 | 62.759 | 14.775 | 9.532 | 1.00 | 13.30 | N |
| ATOM | 737 | CA | PRO | A | 76 | 62.185 | 13.438 | 9.742 | 1.00 | 14.64 | C |
| ATOM | 738 | C | PRO | A | 76 | 63.209 | 12.264 | 9.618 | 1.00 | 14.40 | C |
| ATOM | 739 | O | PRO | A | 76 | 63.157 | 11.335 | 10.409 | 1.00 | 20.54 | O |
| ATOM | 740 | CB | PRO | A | 76 | 61.055 | 13.366 | 8.709 | 1.00 | 7.83 | C |
| ATOM | 741 | CG | PRO | A | 76 | 61.447 | 14.388 | 7.617 | 1.00 | 12.61 | C |
| ATOM | 742 | CD | PRO | A | 76 | 62.068 | 15.504 | 8.455 | 1.00 | 11.18 | C |
| ATOM | 743 | N | ALA | A | 77 | 64.163 | 12.339 | 8.681 | 1.00 | 15.25 | N |
| ATOM | 744 | CA | ALA | A | 77 | 65.206 | 11.312 | 8.538 | 1.00 | 6.79 | C |
| ATOM | 745 | C | ALA | A | 77 | 66.053 | 11.166 | 9.820 | 1.00 | 17.22 | C |
| ATOM | 746 | O | ALA | A | 77 | 66.306 | 10.069 | 10.292 | 1.00 | 18.74 | O |
| ATOM | 747 | CE | ALA | A | 77 | 66.097 | 11.601 | 7.330 | 1.00 | 9.04 | C |
| ATOM | 748 | N | ASP | A | 78 | 66.466 | 12.267 | 10.424 | 1.00 | 10.92 | N |
| ATOM | 749 | CA | ASP | A | 78 | 67.256 | 12.191 | 11.659 | 1.00 | 11.87 | C |
| ATOM | 750 | C | ASP | A | 78 | 66.572 | 11.486 | 12.827 | 1.00 | 16.09 | C |
| ATOM | 751 | O | ASP | A | 78 | 67.212 | 10.741 | 13.601 | 1.00 | 18.07 | O |
| ATOM | 752 | CB | ASP | A | 78 | 67.578 | 13.609 | 12.088 | 1.00 | 19.16 | C |
| ATOM | 753 | CG | ASP | A | 78 | 68.424 | 14.325 | 11.068 | 1.00 | 26.82 | C |
| ATOM | 754 | OD1 | ASP | A | 78 | 68.836 | 13.694 | 10.044 | 1.00 | 33.93 | O |
| ATOM | 755 | OD2 | ASP | A | 78 | 68.673 | 15.514 | 11.316 | 1.00 | 32.06 | O |
| ATOM | 756 | N | PHE | A | 79 | 65.279 | 11.771 | 12.975 | 1.00 | 14.70 | N |
| ATOM | 757 | CA | PHE | A | 79 | 64.471 | 11.192 | 14.044 | 1.00 | 20.69 | C |
| ATOM | 758 | C | PHE | A | 79 | 64.224 | 9.707 | 13.876 | 1.00 | 20.22 | C |
| ATOM | 759 | O | PHE | A | 79 | 64.269 | 8.987 | 14.862 | 1.00 | 22.37 | O |
| ATOM | 760 | CB | PHE | A | 79 | 63.144 | 11.933 | 14.219 | 1.00 | 27.38 | C |
| ATOM | 761 | CG | PHE | A | 79 | 63.264 | 13.218 | 14.990 | 1.00 | 28.59 | C |
| ATOM | 762 | CD1 | PHE | A | 79 | 63.137 | 13.230 | 16.386 | 1.00 | 27.49 | C |
| ATOM | 763 | CD2 | PHE | A | 79 | 63.509 | 14.415 | 14.325 | 1.00 | 28.20 | C |
| ATOM | 764 | CE1 | PHE | A | 79 | 63.281 | 14.413 | 17.109 | 1.00 | 21.76 | C |
| ATOM | 765 | CE2 | PHE | A | 79 | 63.625 | 15.593 | 15.037 | 1.00 | 31.48 | C |
| ATOM | 766 | CZ | PHE | A | 79 | 63.509 | 15.582 | 16.439 | 1.00 | 26.31 | C |
| ATOM | 767 | N | ILE | A | 80 | 63.942 | 9.249 | 12.650 | 1.00 | 10.79 | N |
| ATOM | 768 | CA | ILE | A | 80 | 63.828 | 7.795 | 12.410 | 1.00 | 18.12 | C |
| ATOM | 769 | C | ILE | A | 80 | 65.197 | 7.052 | 12.432 | 1.00 | 10.97 | C |
| ATOM | 770 | O | ILE | A | 80 | 65.406 | 6.090 | 13.195 | 1.00 | 8.92 | O |
| ATOM | 771 | CB | ILE | A | 80 | 62.944 | 7.408 | 11.148 | 1.00 | 17.41 | C |
| ATOM | 772 | CG1 | ILE | A | 80 | 62.651 | 5.886 | 11.105 | 1.00 | 10.16 | C |
| ATOM | 773 | CG2 | ILE | A | 80 | 63.583 | 7.888 | 9.901 | 1.00 | 17.46 | C |
| ATOM | 774 | CD1 | ILE | A | 80 | 61.722 | 5.410 | 9.980 | 1.00 | 7.30 | C |
| ATOM | 775 | N | TYR | A | 81 | 66.151 | 7.539 | 11.658 | 1.00 | 11.18 | N |
| ATOM | 776 | CA | TYR | A | 81 | 67.488 | 6.902 | 11.630 | 1.00 | 15.06 | C |
| ATOM | 777 | C | TYR | A | 81 | 68.237 | 6.782 | 12.959 | 1.00 | 16.83 | C |
| ATOM | 778 | O | TYR | A | 81 | 68.714 | 5.702 | 13.383 | 1.00 | 16.74 | O |
| ATOM | 779 | CB | TYR | A | 81 | 68.384 | 7.599 | 10.616 | 1.00 | 9.43 | C |
| ATOM | 780 | CG | TYR | A | 81 | 69.749 | 6.966 | 10.541 | 1.00 | 22.54 | C |
| ATOM | 781 | CD1 | TYR | A | 81 | 69.963 | 5.824 | 9.747 | 1.00 | 22.37 | C |
| ATOM | 782 | CD2 | TYR | A | 81 | 70.818 | 7.466 | 11.299 | 1.00 | 18.07 | C |
| ATOM | 783 | CE1 | TYR | A | 81 | 71.202 | 5.163 | 9.746 | 1.00 | 15.02 | C |
| ATOM | 784 | CE2 | TYR | A | 81 | 72.080 | 6.893 | 11.201 | 1.00 | 17.37 | C |
| ATOM | 785 | CZ | TYR | A | 81 | 72.255 | 5.698 | 10.472 | 1.00 | 24.27 | C |
| ATOM | 786 | OH | TYR | A | 81 | 73.491 | 5.063 | 10.409 | 1.00 | 19.57 | O |
| ATOM | 787 | N | GLN | A | 82 | 68.385 | 7.918 | 13.612 | 1.00 | 11.39 | N |
| ATOM | 788 | CA | GLN | A | 82 | 69.193 | 7.930 | 14.810 | 1.00 | 12.23 | C |
| ATOM | 789 | CG | GLN | A | 82 | 68.544 | 7.089 | 15.834 | 1.00 | 14.18 | C |
| ATOM | 790 | O | GLN | A | 82 | 69.180 | 6.415 | 16.631 | 1.00 | 11.35 | O |
| ATOM | 791 | CB | GLN | A | 82 | 69.280 | 9.354 | 15.291 | 1.00 | 18.73 | C |
| ATOM | 792 | CG | GLN | A | 82 | 69.986 | 10.209 | 14.250 | 1.00 | 13.54 | C |
| ATOM | 793 | CD | GLN | A | 82 | 70.285 | 11.617 | 14.736 | 1.00 | 26.00 | C |
| ATOM | 794 | OE1 | GLN | A | 82 | 70.410 | 11.850 | 15.927 | 1.00 | 22.99 | O |
| ATOM | 795 | NE2 | GLN | A | 82 | 70.404 | 12.561 | 13.808 | 1.00 | 16.59 | N |
| ATOM | 796 | N | ASN | A | 83 | 67.235 | 7.181 | 15.869 | 1.00 | 11.35 | N |
| ATOM | 797 | CA | ASN | A | 83 | 66.549 | 6.408 | 16.860 | 1.00 | 13.71 | C |
| ATOM | 798 | C | ASN | A | 83 | 66.623 | 4.902 | 16.557 | 1.00 | 21.43 | C |
| ATOM | 799 | O | ASN | A | 83 | 66.831 | 4.101 | 17.463 | 1.00 | 12.10 | O |
| ATOM | 800 | CB | ASN | A | 83 | 65.132 | 6.945 | 17.074 | 1.00 | 13.51 | C |
| ATOM | 801 | CG | ASN | A | 83 | 65.131 | 8.245 | 17.871 | 1.00 | 28.91 | C |
| ATOM | 802 | OD1 | ASN | A | 83 | 65.628 | 8.263 | 18.990 | 1.00 | 22.28 | O |
| ATOM | 803 | ND2 | ASN | A | 83 | 64.756 | 9.354 | 17.237 | 1.00 | 20.17 | N |
| ATOM | 804 | N | MET | A | 84 | 66.592 | 4.517 | 15.290 | 1.00 | 15.63 | N |
| ATOM | 805 | CA | MET | A | 84 | 66.704 | 3.101 | 15.007 | 1.00 | 15.66 | C |
| ATOM | 806 | C | MET | A | 84 | 68.054 | 2.588 | 15.348 | 1.00 | 14.66 | C |
| ATOM | 807 | O | MET | A | 84 | 68.148 | 1.514 | 15.902 | 1.00 | 11.45 | O |
| ATOM | 808 | CB | MET | A | 84 | 66.418 | 2.815 | 13.563 | 1.00 | 17.59 | C |
| ATOM | 809 | CG | MET | A | 84 | 64.911 | 2.894 | 13.220 | 1.00 | 14.40 | C |
| ATOM | 810 | SD | MET | A | 84 | 64.638 | 2.811 | 11.387 | 1.00 | 15.99 | S |
| ATOM | 811 | CE | MET | A | 84 | 65.164 | 1.105 | 10.952 | 1.00 | 8.90 | C |
| ATOM | 812 | N | MET | A | 85 | 69.098 | 3.338 | 15.024 | 1.00 | 11.20 | N |
| ATOM | 813 | CA | MET | A | 85 | 70.468 | 2.879 | 15.321 | 1.00 | 11.67 | C |
| ATOM | 814 | C | MET | A | 85 | 70.779 | 2.831 | 16.774 | 1.00 | 13.04 | C |
| ATOM | 815 | O | MET | A | 85 | 71.359 | 1.893 | 17.265 | 1.00 | 15.26 | O |
| ATOM | 816 | CB | MET | A | 85 | 71.525 | 3.798 | 14.693 | 1.00 | 15.07 | C |
| ATOM | 817 | CG | MET | A | 85 | 71.530 | 3.726 | 13.173 | 1.00 | 32.01 | C |
| ATOM | 818 | SD | MET | A | 85 | 71.918 | 2.027 | 12.487 | 1.00 | 37.79 | S |
| ATOM | 819 | CE | MET | A | 85 | 73.379 | 1.801 | 13.320 | 1.00 | 15.94 | C |
| ATOM | 820 | N | ILE | A | 86 | 70.471 | 3.892 | 17.481 | 1.00 | 13.92 | N |
| ATOM | 821 | CA | ILE | A | 86 | 70.760 | 3.893 | 18.912 | 1.00 | 12.58 | C |
| ATOM | 822 | C | ILE | A | 86 | 70.159 | 2.662 | 19.591 | 1.00 | 21.61 | C |
| ATOM | 823 | O | ILE | A | 86 | 70.813 | 1.981 | 20.362 | 1.00 | 18.68 | O |
| ATOM | 824 | CB | ILE | A | 86 | 70.225 | 5.189 | 19.606 | 1.00 | 11.84 | C |
| ATOM | 825 | CG1 | ILE | A | 86 | 70.978 | 6.429 | 19.119 | 1.00 | 19.78 | C |
| ATOM | 826 | CG2 | ILE | A | 86 | 70.435 | 5.132 | 21.112 | 1.00 | 6.59 | C |
| ATOM | 827 | CD1 | ILE | A | 86 | 70.505 | 7.694 | 19.772 | 1.00 | 20.37 | C |
| ATOM | 828 | N | GLU | A | 87 | 68.893 | 2.383 | 19.316 | 1.00 | 18.78 | N |
| ATOM | 829 | CA | GLU | A | 87 | 68.263 | 1.237 | 19.930 | 1.00 | 14.00 | C |
| ATOM | 830 | C | GLU | A | 87 | 68.797 | −0.116 | 19.454 | 1.00 | 15.93 | C |
| ATOM | 831 | O | GLU | A | 87 | 69.017 | −0.991 | 20.268 | 1.00 | 11.04 | O |
| ATOM | 832 | CB | GLU | A | 87 | 66.734 | 1.324 | 19.900 | 1.00 | 14.89 | C |
| ATOM | 833 | CG | GLU | A | 87 | 66.085 | 1.327 | 18.538 | 1.00 | 28.96 | C |
| ATOM | 834 | CD | GLU | A | 87 | 64.635 | 1.922 | 18.544 | 1.00 | 11.12 | C |
| ATOM | 835 | OE1 | GLU | A | 87 | 64.307 | 2.801 | 19.376 | 1.00 | 25.46 | O |
| ATOM | 836 | OE2 | GLU | A | 87 | 63.845 | 1.547 | 17.663 | 1.00 | 29.87 | O |
| ATOM | 837 | N | SER | A | 88 | 69.054 | −0.259 | 18.155 | 1.00 | 16.18 | N |
| ATOM | 838 | CA | SER | A | 88 | 69.650 | −1.482 | 17.569 | 1.00 | 19.52 | C |
| ATOM | 839 | C | SER | A | 88 | 71.029 | −1.792 | 18.160 | 1.00 | 22.54 | C |
| ATOM | 840 | O | SER | A | 88 | 71.313 | −2.929 | 18.592 | 1.00 | 13.80 | O |
| ATOM | 841 | CB | SER | A | 88 | 69.815 | −1.326 | 16.023 | 1.00 | 14.61 | C |
| ATOM | 842 | OG | SER | A | 88 | 68.551 | −1.201 | 15.355 | 1.00 | 15.41 | O |
| ATOM | 843 | N | ASN | A | 89 | 71.884 | −0.773 | 18.143 | 1.00 | 22.63 | N |
| ATOM | 844 | CA | ASN | A | 89 | 73.227 | −0.869 | 18.693 | 1.00 | 27.23 | C |
| ATOM | 845 | C | ASN | A | 89 | 73.195 | −1.363 | 20.134 | 1.00 | 21.34 | C |
| ATOM | 846 | O | ASN | A | 89 | 73.795 | −2.384 | 20.476 | 1.00 | 23.68 | O |
| ATOM | 847 | CB | ASN | A | 89 | 73.980 | 0.487 | 18.597 | 1.00 | 13.71 | C |
| ATOM | 848 | CG | ASN | A | 89 | 74.440 | 0.825 | 17.168 | 1.00 | 20.40 | C |
| ATOM | 849 | OD1 | ASN | A | 89 | 74.305 | −0.006 | 16.255 | 1.00 | 14.93 | O |
| ATOM | 850 | ND2 | ASN | A | 89 | 74.937 | 2.067 | 16.960 | 1.00 | 13.32 | N |
| ATOM | 851 | N | ILE | A | 90 | 72.488 | −0.646 | 20.979 | 1.00 | 16.55 | N |
| ATOM | 852 | CA | ILE | A | 90 | 72.437 | −1.014 | 22.398 | 1.00 | 21.51 | C |
| ATOM | 853 | C | ILE | A | 90 | 71.876 | −2.421 | 22.729 | 1.00 | 26.50 | C |
| ATOM | 854 | O | ILE | A | 90 | 72.384 | −3.159 | 23.590 | 1.00 | 19.71 | O |
| ATOM | 855 | CB | ILE | A | 90 | 71.670 | 0.070 | 23.233 | 1.00 | 13.32 | C |
| ATOM | 856 | CG1 | ILE | A | 90 | 72.539 | 1.299 | 23.401 | 1.00 | 11.05 | C |
| ATOM | 857 | CG2 | ILE | A | 90 | 71.371 | −0.445 | 24.637 | 1.00 | 7.54 | C |
| ATOM | 858 | CD1 | ILE | A | 90 | 71.749 | 2.597 | 23.668 | 1.00 | 20.71 | C |
| ATOM | 859 | N | ILE | A | 91 | 70.755 | −2.733 | 22.114 | 1.00 | 14.98 | N |
| ATOM | 860 | CA | ILE | A | 91 | 70.047 | −3.953 | 22.442 | 1.00 | 21.33 | C |
| ATOM | 861 | C | ILE | A | 91 | 70.927 | −5.098 | 21.994 | 1.00 | 26.27 | C |
| ATOM | 862 | O | ILE | A | 91 | 71.211 | −6.011 | 22.751 | 1.00 | 26.56 | O |
| ATOM | 863 | CB | ILE | A | 91 | 68.556 | −3.930 | 21.814 | 1.00 | 20.39 | C |
| ATOM | 864 | CG1 | ILE | A | 91 | 67.692 | −2.886 | 22.552 | 1.00 | 13.51 | C |
| ATOM | 865 | CG2 | ILE | A | 91 | 67.841 | −5.316 | 21.845 | 1.00 | 11.31 | C |
| ATOM | 866 | CD1 | ILE | A | 91 | 66.320 | −2.648 | 21.907 | 1.00 | 16.23 | C |
| ATOM | 867 | N | HIS | A | 92 | 71.446 | −4.983 | 20.785 | 1.00 | 24.12 | N |
| ATOM | 868 | CA | HIS | A | 92 | 72.293 | −6.015 | 20.243 | 1.00 | 26.71 | C |
| ATOM | 869 | C | HIS | A | 92 | 73.609 | −6.251 | 21.071 | 1.00 | 29.30 | C |
| ATOM | 870 | O | HIS | A | 92 | 73.983 | −7.366 | 21.443 | 1.00 | 18.58 | O |
| ATOM | 871 | CB | HIS | A | 92 | 72.561 | −5.682 | 18.775 | 1.00 | 22.23 | C |
| ATOM | 872 | CG | HIS | A | 92 | 73.366 | −6.720 | 18.077 | 1.00 | 26.32 | C |
| ATOM | 873 | ND1 | HIS | A | 92 | 72.798 | −7.711 | 17.307 | 1.00 | 27.19 | N |
| ATOM | 874 | CD2 | HIS | A | 92 | 74.699 | −6.978 | 18.106 | 1.00 | 21.95 | C |
| ATOM | 875 | CE1 | HIS | A | 92 | 73.755 | −8.487 | 16.826 | 1.00 | 23.66 | C |
| ATOM | 876 | NE2 | HIS | A | 92 | 74.918 | −8.062 | 17.296 | 1.00 | 17.36 | N |
| ATOM | 877 | N | ALA | A | 93 | 74.328 | −5.187 | 21.333 | 1.00 | 15.66 | N |
| ATOM | 878 | CA | ALA | A | 93 | 75.530 | −5.301 | 22.110 | 1.00 | 11.88 | C |
| ATOM | 879 | C | ALA | A | 93 | 75.222 | −5.900 | 23.512 | 1.00 | 28.78 | C |
| ATOM | 880 | O | ALA | A | 93 | 75.912 | −6.790 | 24.037 | 1.00 | 25.23 | O |
| ATOM | 881 | CB | ALA | A | 93 | 76.139 | −3.959 | 22.221 | 1.00 | 6.30 | C |
| ATOM | 882 | N | ALA | A | 94 | 74.142 | −5.442 | 24.113 | 1.00 | 18.82 | N |
| ATOM | 883 | CA | ALA | A | 94 | 73.777 | −5.971 | 25.399 | 1.00 | 15.61 | C |
| ATOM | 884 | C | ALA | A | 94 | 73.593 | −7.503 | 25.301 | 1.00 | 28.39 | C |
| ATOM | 885 | O | ALA | A | 94 | 74.133 | −8.263 | 26.099 | 1.00 | 21.67 | O |
| ATOM | 886 | CB | ALA | A | 94 | 72.449 | −5.279 | 25.911 | 1.00 | 18.46 | C |
| ATOM | 887 | N | HIS | A | 95 | 72.814 | −7.966 | 24.329 | 1.00 | 26.35 | N |
| ATOM | 888 | CA | HIS | A | 95 | 72.551 | −9.396 | 24.271 | 1.00 | 24.89 | C |
| ATOM | 889 | C | HIS | A | 95 | 73.845 | −10.176 | 24.140 | 1.00 | 22.81 | C |
| ATOM | 890 | O | HIS | A | 95 | 74.077 | −11.136 | 24.865 | 1.00 | 21.44 | O |
| ATOM | 891 | CB | HIS | A | 95 | 71.571 | −9.778 | 23.129 | 1.00 | 22.39 | C |
| ATOM | 892 | CG | HIS | A | 95 | 71.554 | −11.250 | 22.831 | 1.00 | 28.73 | C |
| ATOM | 893 | ND1 | HIS | A | 95 | 70.979 | −12.182 | 23.682 | 1.00 | 22.83 | N |
| ATOM | 894 | CD2 | HIS | A | 95 | 72.159 | −11.964 | 21.845 | 1.00 | 25.22 | C |
| ATOM | 895 | CE1 | HIS | A | 95 | 71.171 | −13.397 | 23.196 | 1.00 | 22.72 | C |
| ATOM | 896 | NE2 | HIS | A | 95 | 71.911 | −13.296 | 22.101 | 1.00 | 24.80 | N |
| ATOM | 897 | N | GLN | A | 96 | 74.709 | −9.658 | 23.281 | 1.00 | 19.97 | N |
| ATOM | 898 | CA | GLN | A | 96 | 75.960 | −10.299 | 22.917 | 1.00 | 22.27 | C |
| ATOM | 899 | C | GLN | A | 96 | 76.877 | −10.353 | 24.086 | 1.00 | 26.58 | C |
| ATOM | 900 | O | GLN | A | 96 | 77.836 | −11.093 | 24.088 | 1.00 | 24.17 | O |
| ATOM | 901 | CS | GLN | A | 96 | 76.642 | −9.492 | 21.818 | 1.00 | 23.38 | C |
| ATOM | 902 | CG | GLN | A | 96 | 77.043 | −10.299 | 20.596 | 1.00 | 61.06 | C |
| ATOM | 903 | CD | GLN | A | 96 | 78.033 | −9.557 | 19.675 | 1.00 | 75.83 | C |
| ATOM | 904 | OE1 | GLN | A | 96 | 78.999 | −8.941 | 20.131 | 1.00 | 56.89 | O |
| ATOM | 905 | NE2 | GLN | A | 96 | 77.815 | −9.668 | 18.366 | 1.00 | 100.00 | N |
| ATOM | 906 | N | ASN | A | 97 | 76.652 | −9.500 | 25.060 | 1.00 | 22.15 | N |
| ATOM | 907 | CA | ASN | A | 97 | 77.537 | −9.536 | 26.208 | 1.00 | 14.74 | C |
| ATOM | 908 | C | ASN | A | 97 | 76.732 | −10.022 | 27.387 | 1.00 | 29.78 | C |
| ATOM | 909 | O | ASN | A | 97 | 77.049 | −9.762 | 28.564 | 1.00 | 27.09 | O |
| ATOM | 910 | CB | ASN | A | 97 | 78.241 | −8.201 | 26.462 | 1.00 | 12.93 | C |
| ATOM | 911 | CG | ASN | A | 97 | 79.260 | −7.897 | 25.407 | 1.00 | 24.91 | C |
| ATOM | 912 | OD1 | ASN | A | 97 | 80.331 | −8.518 | 25.375 | 1.00 | 57.17 | O |
| ATOM | 913 | ND2 | ASN | A | 97 | 78.839 | −7.135 | 24.392 | 1.00 | 34.88 | N |
| ATOM | 914 | N | ASP | A | 98 | 75.666 | −10.732 | 27.055 | 1.00 | 27.98 | N |
| ATOM | 915 | CA | ASP | A | 98 | 74.907 | −11.361 | 28.089 | 1.00 | 29.25 | C |
| ATOM | 916 | C | ASP | A | 98 | 74.400 | −10.379 | 29.164 | 1.00 | 37.53 | C |
| ATOM | 917 | O | ASP | A | 98 | 74.505 | −10.634 | 30.367 | 1.00 | 36.42 | O |
| ATOM | 918 | CB | ASP | A | 98 | 75.791 | −12.450 | 28.700 | 1.00 | 36.37 | C |
| ATOM | 919 | CG | ASP | A | 98 | 75.016 | −13.712 | 29.053 | 1.00 | 88.62 | C |
| ATOM | 920 | OD1 | ASP | A | 98 | 73.775 | −13.749 | 28.877 | 1.00 | 82.53 | O |
| ATOM | 921 | OD2 | ASP | A | 98 | 75.656 | −14.670 | 29.542 | 1.00 | 100.00 | O |
| ATOM | 922 | N | VAL | A | 99 | 73.879 | −9.235 | 28.730 | 1.00 | 27.13 | N |
| ATOM | 923 | CA | VAL | A | 99 | 73.157 | −8.351 | 29.635 | 1.00 | 21.57 | C |
| ATOM | 924 | C | VAL | A | 99 | 71.706 | −8.868 | 29.530 | 1.00 | 16.15 | C |
| ATOM | 925 | O | VAL | A | 99 | 71.159 | −9.088 | 28.422 | 1.00 | 19.47 | O |
| ATOM | 926 | CB | VAL | A | 99 | 73.264 | −6.900 | 29.206 | 1.00 | 24.18 | C |
| ATOM | 927 | CG1 | VAL | A | 99 | 72.517 | −6.015 | 30.198 | 1.00 | 14.58 | C |
| ATOM | 928 | CG2 | VAL | A | 99 | 74.720 | −6.515 | 29.225 | 1.00 | 30.10 | C |
| ATOM | 929 | N | ASN | A | 100 | 71.149 | −9.262 | 30.662 | 1.00 | 17.39 | N |
| ATOM | 930 | CA | ASN | A | 100 | 69.852 | −9.925 | 30.613 | 1.00 | 25.77 | C |
| ATOM | 931 | C | ASN | A | 100 | 68.648 | −9.034 | 30.910 | 1.00 | 24.95 | C |
| ATOM | 932 | O | ASN | A | 100 | 67.498 | −9.377 | 30.582 | 1.00 | 20.88 | O |
| ATOM | 933 | CB | ASN | A | 100 | 69.846 | −11.157 | 31.527 | 1.00 | 14.98 | C |
| ATOM | 934 | CG | ASN | A | 100 | 68.724 | −12.112 | 31.180 | 1.00 | 20.38 | C |
| ATOM | 935 | OD1 | ASN | A | 100 | 68.737 | −12.709 | 30.100 | 1.00 | 29.59 | O |
| ATOM | 936 | ND2 | ASN | A | 100 | 67.716 | −12.240 | 32.076 | 1.00 | 16.35 | N |
| ATOM | 937 | N | LYS | A | 101 | 68.941 | −7.923 | 31.584 | 1.00 | 17.91 | N |
| ATOM | 938 | CA | LYS | A | 101 | 67.970 | −6.916 | 31.994 | 1.00 | 25.43 | C |
| ATOM | 939 | C | LYS | A | 101 | 68.107 | −5.510 | 31.323 | 1.00 | 25.29 | C |
| ATOM | 940 | O | LYS | A | 101 | 69.151 | −4.850 | 31.377 | 1.00 | 19.88 | O |
| ATOM | 941 | CB | LYS | A | 101 | 67.996 | −6.807 | 33.521 | 1.00 | 29.28 | C |
| ATOM | 942 | CG | LYS | A | 101 | 67.464 | −8.054 | 34.205 | 1.00 | 9.31 | C |
| ATOM | 943 | CD | LYS | A | 101 | 67.218 | −7.719 | 35.668 | 1.00 | 38.93 | C |
| ATOM | 944 | CE | LYS | A | 101 | 66.206 | −6.569 | 35.885 | 1.00 | 13.38 | C |
| ATOM | 945 | NZ | LYS | A | 101 | 64.750 | −7.006 | 35.825 | 1.00 | 15.26 | N |
| ATOM | 946 | N | LEU | A | 102 | 67.013 | −5.043 | 30.732 | 1.00 | 22.22 | N |
| ATOM | 947 | CA | LEU | A | 102 | 67.003 | −3.744 | 30.092 | 1.00 | 15.40 | C |
| ATOM | 948 | C | LEU | A | 102 | 65.612 | −3.115 | 30.156 | 1.00 | 18.55 | C |
| ATOM | 949 | O | LEU | A | 102 | 64.590 | −3.811 | 30.102 | 1.00 | 18.92 | O |
| ATOM | 950 | CB | LEU | A | 102 | 67.465 | −3.898 | 28.636 | 1.00 | 11.23 | C |
| ATOM | 951 | CG | LEU | A | 102 | 67.553 | −2.711 | 27.651 | 1.00 | 15.51 | C |
| ATOM | 952 | CD1 | LEU | A | 102 | 68.628 | −2.985 | 26.559 | 1.00 | 9.65 | C |
| ATOM | 953 | CD2 | LEU | A | 102 | 66.162 | −2.407 | 26.995 | 1.00 | 13.10 | C |
| ATOM | 954 | N | LEU | A | 103 | 65.595 | −1.798 | 30.318 | 1.00 | 17.05 | N |
| ATOM | 955 | CA | LEU | A | 103 | 64.356 | −1.036 | 30.265 | 1.00 | 16.23 | C |
| ATOM | 956 | C | LEU | A | 103 | 64.346 | −0.072 | 29.046 | 1.00 | 19.65 | C |
| ATOM | 957 | O | LEU | A | 103 | 65.215 | 0.789 | 28.875 | 1.00 | 19.68 | O |
| ATOM | 958 | CB | LEU | A | 103 | 64.099 | −0.289 | 31.562 | 1.00 | 12.28 | C |
| ATOM | 959 | CG | LEU | A | 103 | 62.686 | 0.259 | 31.594 | 1.00 | 14.13 | C |
| ATOM | 960 | CD1 | LEU | A | 103 | 61.645 | −0.822 | 31.902 | 1.00 | 10.31 | C |
| ATOM | 961 | CD2 | LEU | A | 103 | 62.646 | 1.360 | 32.601 | 1.00 | 12.30 | C |
| ATOM | 962 | N | PHE | A | 104 | 63.417 | −0.333 | 28.140 | 1.00 | 16.41 | N |
| ATOM | 963 | CA | PHE | A | 104 | 63.215 | 0.486 | 26.956 | 1.00 | 18.32 | C |
| ATOM | 964 | C | PHE | A | 104 | 62.126 | 1.546 | 27.249 | 1.00 | 21.85 | C |
| ATOM | 965 | O | PHE | A | 104 | 61.168 | 1.271 | 27.992 | 1.00 | 18.36 | O |
| ATOM | 966 | CE | PHE | A | 104 | 62.796 | −0.386 | 25.793 | 1.00 | 9.86 | C |
| ATOM | 967 | CG | PHE | A | 104 | 62.732 | 0.348 | 24.508 | 1.00 | 16.81 | C |
| ATOM | 968 | CD1 | PHE | A | 104 | 63.894 | 0.714 | 23.840 | 1.00 | 25.04 | C |
| ATOM | 969 | CD2 | PHE | A | 104 | 61.511 | 0.795 | 24.005 | 1.00 | 22.59 | C |
| ATOM | 970 | CE1 | PHE | A | 104 | 63.836 | 1.448 | 22.619 | 1.00 | 31.26 | C |
| ATOM | 971 | CE2 | PHE | A | 104 | 61.449 | 1.535 | 22.814 | 1.00 | 15.59 | C |
| ATOM | 972 | CZ | PHE | A | 104 | 62.625 | 1.895 | 22.139 | 1.00 | 11.67 | C |
| ATOM | 973 | N | LEU | A | 105 | 62.341 | 2.762 | 26.734 | 1.00 | 20.33 | N |
| ATOM | 974 | CA | LEU | A | 105 | 61.416 | 3.897 | 26.904 | 1.00 | 18.10 | C |
| ATOM | 975 | C | LEU | A | 105 | 60.711 | 4.237 | 25.634 | 1.00 | 17.04 | C |
| ATOM | 976 | O | LEU | A | 105 | 61.315 | 4.680 | 24.665 | 1.00 | 18.83 | O |
| ATOM | 977 | CB | LEU | A | 105 | 62.178 | 5.146 | 27.214 | 1.00 | 17.49 | C |
| ATOM | 978 | CG | LEU | A | 105 | 62.434 | 5.544 | 28.644 | 1.00 | 27.17 | C |
| ATOM | 979 | CD1 | LEU | A | 105 | 62.630 | 4.349 | 29.574 | 1.00 | 19.16 | C |
| ATOM | 980 | CD2 | LEU | A | 105 | 63.688 | 6.347 | 28.529 | 1.00 | 23.59 | C |
| ATOM | 981 | N | GLY | A | 106 | 59.407 | 4.153 | 25.652 | 1.00 | 20.66 | N |
| ATOM | 982 | CA | GLY | A | 106 | 58.679 | 4.536 | 24.455 | 1.00 | 21.03 | C |
| ATOM | 983 | C | GLY | A | 106 | 58.080 | 5.935 | 24.597 | 1.00 | 17.32 | C |
| ATOM | 984 | O | GLY | A | 106 | 58.690 | 6.858 | 25.113 | 1.00 | 26.89 | O |
| ATOM | 985 | N | SER | A | 107 | 56.831 | 6.047 | 24.219 | 1.00 | 22.05 | N |
| ATOM | 986 | CA | SER | A | 107 | 56.177 | 7.317 | 24.288 | 1.00 | 22.12 | C |
| ATOM | 987 | C | SER | A | 107 | 54.686 | 7.212 | 23.923 | 1.00 | 19.06 | C |
| ATOM | 988 | O | SER | A | 107 | 54.314 | 6.545 | 22.963 | 1.00 | 27.42 | O |
| ATOM | 989 | CB | SER | A | 107 | 56.882 | 8.232 | 23.300 | 1.00 | 20.99 | C |
| ATOM | 990 | OG | SER | A | 107 | 55.947 | 9.133 | 22.776 | 1.00 | 42.85 | O |
| ATOM | 991 | N | SER | A | 108 | 53.826 | 7.890 | 24.671 | 1.00 | 27.42 | N |
| ATOM | 992 | CA | SER | A | 108 | 52.382 | 7.947 | 24.339 | 1.00 | 26.43 | C |
| ATOM | 993 | C | SER | A | 108 | 52.144 | 8.259 | 22.842 | 1.00 | 30.97 | C |
| ATOM | 994 | O | SER | A | 108 | 51.242 | 7.709 | 22.217 | 1.00 | 33.46 | O |
| ATOM | 995 | CB | SER | A | 108 | 51.710 | 9.072 | 25.144 | 1.00 | 19.87 | C |
| ATOM | 996 | OG | SER | A | 108 | 52.495 | 10.266 | 25.071 | 1.00 | 70.88 | O |
| ATOM | 997 | N | CYS | A | 109 | 52.927 | 9.180 | 22.278 | 1.00 | 24.73 | N |
| ATOM | 998 | CA | CYS | A | 109 | 52.728 | 9.549 | 20.880 | 1.00 | 25.61 | C |
| ATOM | 999 | C | CYS | A | 109 | 52.970 | 8.482 | 19.815 | 1.00 | 21.29 | C |
| ATOM | 1000 | O | CYS | A | 109 | 52.967 | 8.737 | 18.623 | 1.00 | 31.31 | O |
| ATOM | 1001 | CB | CYS | A | 109 | 53.369 | 10.899 | 20.544 | 1.00 | 39.55 | C |
| ATOM | 1002 | SG | CYS | A | 109 | 55.153 | 11.077 | 20.847 | 1.00 | 49.24 | S |
| ATOM | 1003 | N | ILE | A | 110 | 53.101 | 7.264 | 20.258 | 1.00 | 18.31 | N |
| ATOM | 1004 | CA | ILE | A | 110 | 53.329 | 6.150 | 19.379 | 1.00 | 28.10 | C |
| ATOM | 1005 | C | ILE | A | 110 | 51.977 | 5.489 | 19.082 | 1.00 | 15.38 | C |
| ATOM | 1006 | O | ILE | A | 110 | 51.895 | 4.592 | 18.268 | 1.00 | 16.52 | O |
| ATOM | 1007 | CB | ILE | A | 110 | 54.154 | 5.153 | 20.206 | 1.00 | 40.45 | C |
| ATOM | 1008 | CG1 | ILE | A | 110 | 55.604 | 5.510 | 20.136 | 1.00 | 39.02 | C |
| ATOM | 1009 | CG2 | ILE | A | 110 | 53.879 | 3.715 | 19.875 | 1.00 | 61.33 | C |
| ATOM | 1010 | CD1 | ILE | A | 110 | 56.429 | 4.338 | 20.549 | 1.00 | 82.74 | C |
| ATOM | 1011 | N | TYR | A | 111 | 50.951 | 5.842 | 19.854 | 1.00 | 14.91 | N |
| ATOM | 1012 | CA | TYR | A | 111 | 49.630 | 5.227 | 19.678 | 1.00 | 13.96 | C |
| ATOM | 1013 | C | TYR | A | 111 | 48.956 | 5.831 | 18.459 | 1.00 | 20.40 | C |
| ATOM | 1014 | O | TYR | A | 111 | 49.302 | 6.933 | 18.056 | 1.00 | 11.71 | O |
| ATOM | 1015 | CB | TYR | A | 111 | 48.763 | 5.468 | 20.921 | 1.00 | 9.63 | C |
| ATOM | 1016 | CG | TYR | A | 111 | 49.117 | 4.550 | 22.065 | 1.00 | 14.94 | C |
| ATOM | 1017 | CD1 | TYR | A | 111 | 48.985 | 3.159 | 21.938 | 1.00 | 9.73 | C |
| ATOM | 1018 | CD2 | TYR | A | 111 | 49.755 | 5.038 | 23.216 | 1.00 | 14.96 | C |
| ATOM | 1019 | CE1 | TYR | A | 111 | 49.344 | 2.273 | 23.014 | 1.00 | 6.53 | C |
| ATOM | 1020 | CE2 | TYR | A | 111 | 50.146 | 4.155 | 24.272 | 1.00 | 13.66 | C |
| ATOM | 1021 | CZ | TYR | A | 111 | 49.873 | 2.787 | 24.171 | 1.00 | 17.86 | C |
| ATOM | 1022 | OH | TYR | A | 111 | 50.266 | 1.927 | 25.157 | 1.00 | 11.37 | O |
| ATOM | 1023 | N | PRO | A | 112 | 47.974 | 5.145 | 17.872 | 1.00 | 22.56 | N |
| ATOM | 1024 | CA | PRO | A | 112 | 47.279 | 5.743 | 16.721 | 1.00 | 23.44 | C |
| ATOM | 1025 | C | PRO | A | 112 | 46.589 | 7.111 | 16.988 | 1.00 | 17.82 | C |
| ATOM | 1026 | O | PRO | A | 112 | 46.197 | 7.453 | 18.115 | 1.00 | 19.72 | O |
| ATOM | 1027 | CB | PRO | A | 112 | 46.290 | 4.644 | 16.252 | 1.00 | 15.69 | C |
| ATOM | 1028 | CG | PRO | A | 112 | 46.895 | 3.343 | 16.769 | 1.00 | 22.83 | C |
| ATOM | 1029 | CD | PRO | A | 112 | 47.593 | 3.733 | 18.086 | 1.00 | 16.10 | C |
| ATOM | 1030 | N | LYS | A | 113 | 46.418 | 7.866 | 15.915 | 1.00 | 19.48 | N |
| ATOM | 1031 | CA | LYS | A | 113 | 45.793 | 9.167 | 15.994 | 1.00 | 23.50 | C |
| ATOM | 1032 | C | LYS | A | 113 | 44.396 | 9.077 | 16.655 | 1.00 | 34.28 | C |
| ATOM | 1033 | O | LYS | A | 113 | 44.046 | 9.887 | 17.524 | 1.00 | 46.14 | O |
| ATOM | 1034 | CB | LYS | A | 113 | 45.675 | 9.735 | 14.593 | 1.00 | 30.04 | C |
| ATOM | 1035 | CG | LYS | A | 113 | 46.219 | 11.124 | 14.477 | 1.00 | 43.78 | C |
| ATOM | 1036 | CD | LYS | A | 113 | 45.381 | 11.941 | 13.515 | 1.00 | 100.00 | C |
| ATOM | 1037 | CE | LYS | A | 113 | 44.361 | 12.836 | 14.250 | 1.00 | 100.00 | C |
| ATOM | 1038 | NZ | LYS | A | 113 | 43.480 | 13.625 | 13.304 | 1.00 | 100.00 | N |
| ATOM | 1039 | N | LEU | A | 114 | 43.591 | 8.103 | 16.250 | 1.00 | 26.33 | N |
| ATOM | 1040 | CA | LEU | A | 114 | 42.267 | 7.957 | 16.833 | 1.00 | 20.65 | C |
| ATOM | 1041 | C | LEU | A | 114 | 42.083 | 6.792 | 17.760 | 1.00 | 18.44 | C |
| ATOM | 1042 | O | LEU | A | 114 | 41.002 | 6.278 | 17.918 | 1.00 | 34.04 | O |
| ATOM | 1043 | CB | LEU | A | 114 | 41.194 | 8.002 | 15.780 | 1.00 | 24.37 | C |
| ATOM | 1044 | CG | LEU | A | 114 | 41.587 | 9.122 | 14.830 | 1.00 | 40.86 | C |
| ATOM | 1045 | CD1 | LEU | A | 114 | 40.991 | 8.797 | 13.504 | 1.00 | 49.29 | C |
| ATOM | 1046 | CD2 | LEU | A | 114 | 41.139 | 10.512 | 15.300 | 1.00 | 26.85 | C |
| ATOM | 1047 | N | ALA | A | 115 | 43.103 | 6.473 | 18.527 | 1.00 | 29.00 | N |
| ATOM | 1048 | CA | ALA | A | 115 | 42.920 | 5.446 | 19.528 | 1.00 | 25.66 | C |
| ATOM | 1049 | C | ALA | A | 115 | 41.722 | 5.727 | 20.454 | 1.00 | 28.76 | C |
| ATOM | 1050 | O | ALA | A | 115 | 41.364 | 6.855 | 20.682 | 1.00 | 24.12 | O |
| ATOM | 1051 | CE | ALA | A | 115 | 44.177 | 5.272 | 20.326 | 1.00 | 16.86 | C |
| ATOM | 1052 | N | LYS | A | 116 | 41.137 | 4.675 | 20.998 | 1.00 | 30.21 | N |
| ATOM | 1053 | CA | LYS | A | 116 | 40.036 | 4.792 | 21.928 | 1.00 | 25.85 | C |
| ATOM | 1054 | C | LYS | A | 116 | 40.668 | 5.248 | 23.195 | 1.00 | 14.18 | C |
| ATOM | 1055 | O | LYS | A | 116 | 41.750 | 4.781 | 23.535 | 1.00 | 23.51 | O |
| ATOM | 1056 | CE | LYS | A | 116 | 39.369 | 3.415 | 22.116 | 1.00 | 22.05 | C |
| ATOM | 1057 | CG | LYS | A | 116 | 39.053 | 3.032 | 23.524 | 1.00 | 55.38 | C |
| ATOM | 1058 | CD | LYS | A | 116 | 37.963 | 1.955 | 23.549 | 1.00 | 100.00 | C |
| ATOM | 1059 | CE | LYS | A | 116 | 37.120 | 1.953 | 24.835 | 1.00 | 100.00 | C |
| ATOM | 1060 | NZ | LYS | A | 116 | 35.767 | 1.310 | 24.630 | 1.00 | 100.00 | N |
| ATOM | 1061 | N | GLN | A | 117 | 40.021 | 6.208 | 23.856 | 1.00 | 18.23 | N |
| ATOM | 1062 | CA | GLN | A | 117 | 40.456 | 6.757 | 25.180 | 1.00 | 21.01 | C |
| ATOM | 1063 | C | GLN | A | 117 | 39.695 | 6.178 | 26.383 | 1.00 | 30.96 | C |
| ATOM | 1064 | O | GLN | A | 117 | 38.483 | 6.009 | 26.345 | 1.00 | 27.66 | O |
| ATOM | 1065 | CB | GLN | A | 117 | 40.215 | 8.263 | 25.179 | 1.00 | 11.32 | C |
| ATOM | 1066 | CG | GLN | A | 117 | 40.849 | 8.912 | 23.948 | 1.00 | 12.12 | C |
| ATOM | 1067 | CD | GLN | A | 117 | 42.404 | 8.823 | 23.954 | 1.00 | 24.10 | C |
| ATOM | 1068 | OE1 | GLN | A | 117 | 43.041 | 8.628 | 22.896 | 1.00 | 47.88 | O |
| ATOM | 1069 | NE2 | GLN | A | 117 | 43.001 | 8.953 | 25.131 | 1.00 | 14.24 | N |
| ATOM | 1070 | N | PRO | A | 118 | 40.374 | 5.992 | 27.499 | 1.00 | 30.02 | N |
| ATOM | 1071 | CA | PRO | A | 118 | 41.826 | 6.194 | 27.655 | 1.00 | 26.44 | C |
| ATOM | 1072 | C | PRO | A | 118 | 42.450 | 5.050 | 26.899 | 1.00 | 24.37 | C |
| ATOM | 1073 | O | PRO | A | 118 | 41.792 | 4.027 | 26.726 | 1.00 | 25.34 | O |
| ATOM | 1074 | CB | PRO | A | 118 | 42.055 | 5.994 | 29.167 | 1.00 | 23.89 | C |
| ATOM | 1075 | CG | PRO | A | 118 | 40.847 | 5.240 | 29.654 | 1.00 | 23.20 | C |
| ATOM | 1076 | CD | PRO | A | 118 | 39.695 | 5.519 | 28.709 | 1.00 | 15.79 | C |
| ATOM | 1077 | N | MET | A | 119 | 43.684 | 5.228 | 26.432 | 1.00 | 16.00 | N |
| ATOM | 1078 | CA | MET | A | 119 | 44.372 | 4.215 | 25.644 | 1.00 | 10.80 | C |
| ATOM | 1079 | C | MET | A | 119 | 45.062 | 3.083 | 26.444 | 1.00 | 23.61 | C |
| ATOM | 1080 | O | MET | A | 119 | 46.013 | 3.281 | 27.209 | 1.00 | 18.02 | O |
| ATOM | 1081 | CB | MET | A | 119 | 45.384 | 4.894 | 24.791 | 1.00 | 13.52 | C |
| ATOM | 1082 | CG | MET | A | 119 | 44.801 | 6.014 | 23.989 | 1.00 | 18.52 | C |
| ATOM | 1083 | SD | MET | A | 119 | 46.157 | 7.054 | 23.271 | 1.00 | 26.27 | S |
| ATOM | 1084 | CE | MET | A | 119 | 46.264 | 6.524 | 21.845 | 1.00 | 33.79 | C |
| ATOM | 1085 | N | ALA | A | 120 | 44.559 | 1.875 | 26.271 | 1.00 | 26.64 | N |
| ATOM | 1086 | CA | ALA | A | 120 | 45.177 | 0.712 | 26.884 | 1.00 | 29.17 | C |
| ATOM | 1087 | C | ALA | A | 120 | 46.356 | 0.308 | 25.984 | 1.00 | 23.21 | C |
| ATOM | 1088 | O | ALA | A | 120 | 46.439 | 0.759 | 24.833 | 1.00 | 20.19 | O |
| ATOM | 1089 | CB | ALA | A | 120 | 44.169 | −0.419 | 26.944 | 1.00 | 26.02 | C |
| ATOM | 1090 | N | GLU | A | 121 | 47.238 | −0.553 | 26.507 | 1.00 | 12.30 | N |
| ATOM | 1091 | CA | GLU | A | 121 | 48.427 | −1.009 | 25.788 | 1.00 | 9.45 | C |
| ATOM | 1092 | C | GLU | A | 121 | 48.070 | −1.697 | 24.450 | 1.00 | 11.68 | C |
| ATOM | 1093 | O | GLU | A | 121 | 48.828 | −1.670 | 23.450 | 1.00 | 14.84 | O |
| ATOM | 1094 | CB | GLU | A | 121 | 49.321 | −1.883 | 26.715 | 1.00 | 16.74 | C |
| ATOM | 1095 | CG | GLU | A | 121 | 50.132 | −1.122 | 27.763 | 1.00 | 18.14 | C |
| ATOM | 1096 | CD | GLU | A | 121 | 49.458 | −1.000 | 29.137 | 1.00 | 13.00 | C |
| ATOM | 1097 | OE1 | GLU | A | 121 | 48.252 | −1.294 | 29.276 | 1.00 | 20.79 | O |
| ATOM | 1098 | OE2 | GLU | A | 121 | 50.123 | −0.521 | 30.080 | 1.00 | 17.86 | O |
| ATOM | 1099 | N | SER | A | 122 | 46.887 | −2.273 | 24.409 | 1.00 | 11.79 | N |
| ATOM | 1100 | CA | SER | A | 122 | 46.427 | −2.977 | 23.218 | 1.00 | 12.16 | C |
| ATOM | 1101 | C | SER | A | 122 | 46.030 | −2.058 | 22.100 | 1.00 | 11.70 | C |
| ATOM | 1102 | O | SER | A | 122 | 45.717 | −2.529 | 21.010 | 1.00 | 13.91 | O |
| ATOM | 1103 | CB | SER | A | 122 | 45.186 | −3.781 | 23.568 | 1.00 | 21.50 | C |
| ATOM | 1104 | OG | SER | A | 122 | 44.143 | −2.908 | 23.976 | 1.00 | 28.52 | O |
| ATOM | 1105 | N | GLU | A | 123 | 46.041 | −0.754 | 22.341 | 1.00 | 14.65 | N |
| ATOM | 1106 | CA | GLU | A | 123 | 45.783 | 0.202 | 21.243 | 1.00 | 17.15 | C |
| ATOM | 1107 | C | GLU | A | 123 | 46.959 | 0.313 | 20.240 | 1.00 | 11.48 | C |
| ATOM | 1108 | O | GLU | A | 123 | 46.821 | 0.844 | 19.141 | 1.00 | 11.19 | O |
| ATOM | 1109 | CB | GLU | A | 123 | 45.481 | 1.600 | 21.805 | 1.00 | 21.66 | C |
| ATOM | 1110 | CG | GLU | A | 123 | 44.127 | 1.694 | 22.523 | 1.00 | 24.68 | C |
| ATOM | 1111 | CD | GLU | A | 123 | 42.984 | 1.374 | 21.585 | 1.00 | 35.56 | C |
| ATOM | 1112 | OE1 | GLU | A | 123 | 43.019 | 1.865 | 20.426 | 1.00 | 41.73 | O |
| ATOM | 1113 | OE2 | GLU | A | 123 | 42.158 | 0.497 | 21.940 | 1.00 | 100.00 | O |
| ATOM | 1114 | N | LEU | A | 124 | 48.134 | −0.185 | 20.618 | 1.00 | 14.02 | N |
| ATOM | 1115 | CA | LEU | A | 124 | 49.296 | −0.082 | 19.740 | 1.00 | 15.32 | C |
| ATOM | 1116 | C | LEU | A | 124 | 49.082 | −0.754 | 18.458 | 1.00 | 17.76 | C |
| ATOM | 1117 | O | LEU | A | 124 | 48.752 | −1.917 | 18.445 | 1.00 | 18.91 | O |
| ATOM | 1118 | CB | LEU | A | 124 | 50.564 | −0.680 | 20.362 | 1.00 | 18.07 | C |
| ATOM | 1119 | CG | LEU | A | 124 | 51.922 | −0.222 | 19.803 | 1.00 | 21.52 | C |
| ATOM | 1120 | CD1 | LEU | A | 124 | 52.080 | 1.258 | 20.117 | 1.00 | 20.35 | C |
| ATOM | 1121 | CD2 | LEU | A | 124 | 53.042 | −0.919 | 20.550 | 1.00 | 14.07 | C |
| ATOM | 1122 | N | LEU | A | 125 | 49.514 | −0.0711 | 7.409 | 1.00 | 18.44 | N |
| ATOM | 1123 | CA | LEU | A | 125 | 49.445 | −0.564 | 16.052 | 1.00 | 19.92 | C |
| ATOM | 1124 | C | LEU | A | 125 | 48.034 | −0.754 | 15.509 | 1.00 | 25.56 | C |
| ATOM | 1125 | O | LEU | A | 125 | 47.854 | −1.188 | 14.364 | 1.00 | 18.26 | O |
| ATOM | 1126 | CE | LEU | A | 125 | 50.355 | −1.800 | 15.840 | 1.00 | 20.79 | C |
| ATOM | 1127 | CG | LEU | A | 125 | 51.890 | −1.511 | 15.778 | 1.00 | 17.21 | C |
| ATOM | 1128 | CD1 | LEU | A | 125 | 52.744 | −2.649 | 16.316 | 1.00 | 19.95 | C |
| ATOM | 1129 | CD2 | LEU | A | 125 | 52.334 | −1.219 | 14.338 | 1.00 | 5.81 | C |
| ATOM | 1130 | N | GLN | A | 126 | 47.027 | −0.327 | 16.276 | 1.00 | 21.97 | N |
| ATOM | 1131 | CA | GLN | A | 126 | 45.652 | −0.504 | 15.790 | 1.00 | 19.97 | C |
| ATOM | 1132 | C | GLN | A | 126 | 45.213 | 0.447 | 14.724 | 1.00 | 28.31 | C |
| ATOM | 1133 | O | GLN | A | 126 | 44.076 | 0.391 | 14.293 | 1.00 | 47.49 | O |
| ATOM | 1134 | CE | GLN | A | 126 | 44.652 | −0.404 | 16.911 | 1.00 | 19.87 | C |
| ATOM | 1135 | CG | GLN | A | 126 | 44.949 | −1.312 | 18.048 | 1.00 | 18.39 | C |
| ATOM | 1136 | CD | GLN | A | 126 | 44.319 | −2.626 | 17.835 | 1.00 | 66.80 | C |
| ATOM | 1137 | OE1 | GLN | A | 126 | 44.064 | −3.376 | 18.792 | 1.00 | 40.75 | O |
| ATOM | 1138 | NE2 | GLN | A | 126 | 44.015 | −2.952 | 16.565 | 1.00 | 71.74 | N |
| ATOM | 1139 | N | GLY | A | 127 | 46.080 | 1.330 | 14.270 | 1.00 | 28.29 | N |
| ATOM | 1140 | CA | GLY | A | 127 | 45.627 | 2.260 | 13.252 | 1.00 | 23.31 | C |
| ATOM | 1141 | C | GLY | A | 127 | 46.662 | 3.315 | 12.953 | 1.00 | 22.90 | C |
| ATOM | 1142 | O | GLY | A | 127 | 47.755 | 3.254 | 13.474 | 1.00 | 25.30 | O |
| ATOM | 1143 | N | THR | A | 128 | 46.311 | 4.219 | 12.046 | 1.00 | 19.51 | N |
| ATOM | 1144 | CA | THR | A | 128 | 47.149 | 5.314 | 11.588 | 1.00 | 22.12 | C |
| ATOM | 1145 | C | THR | A | 128 | 47.705 | 6.219 | 12.695 | 1.00 | 22.60 | C |
| ATOM | 1146 | O | THR | A | 128 | 47.061 | 6.461 | 13.731 | 1.00 | 18.58 | O |
| ATOM | 1147 | CB | THR | A | 128 | 46.392 | 6.182 | 10.544 | 1.00 | 35.98 | C |
| ATOM | 1148 | OG1 | THR | A | 128 | 46.533 | 5.594 | 9.239 | 1.00 | 58.05 | O |
| ATOM | 1149 | CG2 | THR | A | 128 | 46.942 | 7.639 | 10.542 | 1.00 | 43.41 | C |
| ATOM | 1150 | N | LEU | A | 129 | 48.907 | 6.715 | 12.425 | 1.00 | 18.32 | N |
| ATOM | 1151 | CA | LEU | A | 129 | 49.674 | 7.534 | 13.356 | 1.00 | 16.76 | C |
| ATOM | 1152 | C | LEU | A | 129 | 49.504 | 8.959 | 12.967 | 1.00 | 4.89 | C |
| ATOM | 1153 | O | LEU | A | 129 | 49.232 | 9.260 | 11.814 | 1.00 | 16.14 | O |
| ATOM | 1154 | CB | LEU | A | 129 | 51.205 | 7.191 | 13.261 | 1.00 | 17.91 | C |
| ATOM | 1155 | CG | LEU | A | 129 | 51.769 | 5.804 | 13.752 | 1.00 | 18.21 | C |
| ATOM | 1156 | CD1 | LEU | A | 129 | 53.132 | 5.379 | 13.193 | 1.00 | 12.12 | C |
| ATOM | 1157 | CD2 | LEU | A | 129 | 51.683 | 5.532 | 15.251 | 1.00 | 3.89 | C |
| ATOM | 1158 | N | GLU | A | 130 | 49.816 | 9.827 | 13.917 | 1.00 | 10.23 | N |
| ATOM | 1159 | CA | GLU | A | 130 | 49.912 | 11.268 | 13.691 | 1.00 | 13.22 | C |
| ATOM | 1160 | C | GLU | A | 130 | 51.128 | 11.544 | 12.775 | 1.00 | 23.44 | C |
| ATOM | 1161 | O | GLU | A | 130 | 52.249 | 11.162 | 13.090 | 1.00 | 21.23 | O |
| ATOM | 1162 | CB | GLU | A | 130 | 50.150 | 11.979 | 15.035 | 1.00 | 18.48 | C |
| ATOM | 1163 | CG | GLU | A | 130 | 50.754 | 13.376 | 14.886 | 1.00 | 77.44 | C |
| ATOM | 1164 | CD | GLU | A | 130 | 49.833 | 14.328 | 14.121 | 1.00 | 100.00 | C |
| ATOM | 1165 | OR1 | GLU | A | 130 | 48.588 | 14.205 | 14.340 | 1.00 | 36.19 | O |
| ATOM | 1166 | OE2 | GLU | A | 130 | 50.347 | 15.161 | 13.295 | 1.00 | 21.03 | O |
| ATOM | 1167 | N | PRO | A | 131 | 50.920 | 12.219 | 11.648 | 1.00 | 21.35 | N |
| ATOM | 1168 | CA | PRO | A | 131 | 52.023 | 12.409 | 10.731 | 1.00 | 14.78 | C |
| ATOM | 1169 | C | PRO | A | 131 | 53.201 | 13.132 | 11.265 | 1.00 | 14.98 | C |
| ATOM | 1170 | O | PRO | A | 131 | 54.325 | 12.847 | 10.853 | 1.00 | 20.99 | O |
| ATOM | 1171 | CB | PRO | A | 131 | 51.413 | 13.154 | 9.552 | 1.00 | 14.76 | C |
| ATOM | 1172 | CG | PRO | A | 131 | 50.071 | 13.485 | 9.949 | 1.00 | 20.99 | C |
| ATOM | 1173 | CD | PRO | A | 131 | 49.641 | 12.626 | 11.047 | 1.00 | 17.25 | C |
| ATOM | 1174 | N | THR | A | 132 | 52.986 | 14.095 | 12.159 | 1.00 | 18.77 | N |
| ATOM | 1175 | CA | THR | A | 132 | 54.131 | 14.838 | 12.689 | 1.00 | 16.44 | C |
| ATOM | 1176 | C | THR | A | 132 | 55.102 | 13.951 | 13.408 | 1.00 | 21.91 | C |
| ATOM | 1177 | O | THR | A | 132 | 56.317 | 14.088 | 13.234 | 1.00 | 24.17 | O |
| ATOM | 1178 | CB | THR | A | 132 | 53.716 | 15.907 | 13.606 | 1.00 | 23.45 | C |
| ATOM | 1179 | OG1 | THR | A | 132 | 52.976 | 16.883 | 12.850 | 1.00 | 31.15 | O |
| ATOM | 1180 | CG2 | THR | A | 132 | 54.969 | 16.519 | 14.341 | 1.00 | 9.28 | C |
| ATOM | 1181 | N | ASN | A | 133 | 54.551 | 12.970 | 14.122 | 1.00 | 28.59 | N |
| ATOM | 1182 | CA | ASN | A | 133 | 55.359 | 12.007 | 14.875 | 1.00 | 26.38 | C |
| ATOM | 1183 | C | ASN | A | 133 | 55.666 | 10.682 | 14.207 | 1.00 | 14.85 | C |
| ATOM | 1184 | O | ASN | A | 133 | 56.446 | 9.884 | 14.755 | 1.00 | 18.67 | O |
| ATOM | 1185 | CE | ASN | A | 133 | 54.661 | 11.699 | 16.168 | 1.00 | 23.70 | C |
| ATOM | 1186 | CG | ASN | A | 133 | 54.480 | 12.894 | 16.968 | 1.00 | 50.55 | C |
| ATOM | 1187 | OD1 | ASN | A | 133 | 53.354 | 13.272 | 17.252 | 1.00 | 40.07 | O |
| ATOM | 1188 | ND2 | ASN | A | 133 | 55.568 | 13.638 | 17.163 | 1.00 | 40.36 | N |
| ATOM | 1189 | N | GLU | A | 134 | 55.100 | 10.469 | 13.022 | 1.00 | 9.98 | N |
| ATOM | 1190 | CA | GLU | A | 134 | 55.237 | 9.210 | 12.365 | 1.00 | 9.66 | C |
| ATOM | 1191 | C | GLU | A | 134 | 56.648 | 8.530 | 12.274 | 1.00 | 13.86 | C |
| ATOM | 1192 | O | GLU | A | 134 | 56.814 | 7.388 | 12.706 | 1.00 | 22.89 | O |
| ATOM | 1193 | CB | GLU | A | 134 | 54.448 | 9.200 | 11.070 | 1.00 | 17.55 | C |
| ATOM | 1194 | CG | GLU | A | 134 | 54.750 | 7.930 | 10.227 | 1.00 | 20.89 | C |
| ATOM | 1195 | CD | GLU | A | 134 | 53.926 | 7.868 | 8.970 | 1.00 | 13.59 | C |
| ATOM | 1196 | OE1 | GLU | A | 134 | 52.678 | 7.738 | 9.085 | 1.00 | 35.28 | O |
| ATOM | 1197 | OE2 | GLU | A | 134 | 54.497 | 8.048 | 7.869 | 1.00 | 13.44 | O |
| ATOM | 1198 | N | PRO | A | 135 | 57.680 | 9.222 | 11.789 | 1.00 | 15.72 | N |
| ATOM | 1199 | CA | PRO | A | 135 | 59.014 | 8.600 | 11.699 | 1.00 | 18.91 | C |
| ATOM | 1200 | C | PRO | A | 135 | 59.544 | 8.174 | 13.073 | 1.00 | 18.68 | C |
| ATOM | 1201 | O | PRO | A | 135 | 60.072 | 7.069 | 13.271 | 1.00 | 15.69 | O |
| ATOM | 1202 | CB | PRO | A | 135 | 59.896 | 9.755 | 11.169 | 1.00 | 13.84 | C |
| ATOM | 1203 | CG | PRO | A | 135 | 59.036 | 10.514 | 10.350 | 1.00 | 9.78 | C |
| ATOM | 1204 | CD | PRO | A | 135 | 57.594 | 10.395 | 10.908 | 1.00 | 14.43 | C |
| ATOM | 1205 | N | TYR | A | 136 | 59.449 | 9.117 | 13.994 | 1.00 | 8.64 | N |
| ATOM | 1206 | CA | TYR | A | 136 | 59.873 | 8.915 | 15.324 | 1.00 | 13.27 | C |
| ATOM | 1207 | C | TYR | A | 136 | 59.056 | 7.728 | 15.907 | 1.00 | 16.84 | C |
| ATOM | 1208 | O | TYR | A | 136 | 59.578 | 6.903 | 16.658 | 1.00 | 12.90 | O |
| ATOM | 1209 | CB | TYR | A | 136 | 59.604 | 10.234 | 16.100 | 1.00 | 15.51 | C |
| ATOM | 1210 | CG | TYR | A | 136 | 59.912 | 10.168 | 17.614 | 1.00 | 18.26 | C |
| ATOM | 1211 | CD1 | TYR | A | 136 | 61.200 | 10.062 | 18.072 | 1.00 | 20.53 | C |
| ATOM | 1212 | CD2 | TYR | A | 136 | 58.904 | 10.150 | 18.568 | 1.00 | 17.38 | C |
| ATOM | 1213 | CE1 | TYR | A | 136 | 61.484 | 9.959 | 19.440 | 1.00 | 30.44 | C |
| ATOM | 1214 | CE2 | TYR | A | 136 | 59.184 | 10.084 | 19.953 | 1.00 | 9.85 | C |
| ATOM | 1215 | CZ | TYR | A | 136 | 60.476 | 9.949 | 20.377 | 1.00 | 20.65 | C |
| ATOM | 1216 | OH | TYR | A | 136 | 60.792 | 9.873 | 21.734 | 1.00 | 24.41 | O |
| ATOM | 1217 | N | ALA | A | 137 | 57.760 | 7.687 | 15.638 | 1.00 | 7.19 | N |
| ATOM | 1218 | CA | ALA | A | 137 | 56.923 | 6.633 | 16.227 | 1.00 | 12.68 | C |
| ATOM | 1219 | C | ALA | A | 137 | 57.345 | 5.265 | 15.737 | 1.00 | 15.21 | C |
| ATOM | 1220 | O | ALA | A | 137 | 57.425 | 4.272 | 16.488 | 1.00 | 14.58 | O |
| ATOM | 1221 | CB | ALA | A | 137 | 55.517 | 6.849 | 15.871 | 1.00 | 11.40 | C |
| ATOM | 1222 | N | ILE | A | 138 | 57.567 | 5.213 | 14.447 | 1.00 | 8.93 | N |
| ATOM | 1223 | CA | ILE | A | 138 | 57.954 | 3.971 | 13.831 | 1.00 | 11.77 | C |
| ATOM | 1224 | C | ILE | A | 138 | 59.246 | 3.494 | 14.492 | 1.00 | 16.20 | C |
| ATOM | 1225 | O | ILE | A | 138 | 59.307 | 2.377 | 14.970 | 1.00 | 13.79 | O |
| ATOM | 1226 | CB | ILE | A | 138 | 58.064 | 4.172 | 12.316 | 1.00 | 17.85 | C |
| ATOM | 1227 | CG1 | ILE | A | 138 | 56.680 | 4.473 | 11.757 | 1.00 | 28.21 | C |
| ATOM | 1228 | CG2 | ILE | A | 138 | 58.674 | 2.986 | 11.602 | 1.00 | 9.81 | C |
| ATOM | 1229 | CD1 | ILE | A | 138 | 55.695 | 3.376 | 11.970 | 1.00 | 18.17 | C |
| ATOM | 1230 | N | ALA | A | 139 | 60.243 | 4.361 | 14.625 | 1.00 | 11.54 | N |
| ATOM | 1231 | CA | ALA | A | 139 | 61.494 | 3.937 | 15.288 | 1.00 | 13.22 | C |
| ATOM | 1232 | C | ALA | A | 139 | 61.256 | 3.364 | 16.675 | 1.00 | 18.73 | C |
| ATOM | 1233 | O | ALA | A | 139 | 61.791 | 2.318 | 17.031 | 1.00 | 20.44 | O |
| ATOM | 1234 | CB | ALA | A | 139 | 62.434 | 5.073 | 15.390 | 1.00 | 13.62 | C |
| ATOM | 1235 | N | LYS | A | 140 | 60.397 | 4.033 | 17.448 | 1.00 | 16.36 | N |
| ATOM | 1236 | CA | LYS | A | 140 | 60.083 | 3.600 | 18.815 | 1.00 | 15.14 | C |
| ATOM | 1237 | C | LYS | A | 140 | 59.392 | 2.262 | 18.824 | 1.00 | 15.18 | C |
| ATOM | 1238 | O | LYS | A | 140 | 59.824 | 1.346 | 19.475 | 1.00 | 21.42 | O |
| ATOM | 1239 | CB | LYS | A | 140 | 59.193 | 4.606 | 19.525 | 1.00 | 17.86 | C |
| ATOM | 1240 | CG | LYS | A | 140 | 59.925 | 5.806 | 20.152 | 1.00 | 21.11 | C |
| ATOM | 1241 | CD | LYS | A | 140 | 61.208 | 5.478 | 20.958 | 1.00 | 16.75 | C |
| ATOM | 1242 | CE | LYS | A | 140 | 61.664 | 6.735 | 21.835 | 1.00 | 10.06 | C |
| ATOM | 1243 | NZ | LYS | A | 140 | 62.688 | 6.496 | 22.921 | 1.00 | 14.40 | N |
| ATOM | 1244 | N | ILE | A | 141 | 58.356 | 2.116 | 18.027 | 1.00 | 11.49 | N |
| ATOM | 1245 | CA | ILE | A | 141 | 57.703 | 0.828 | 17.977 | 1.00 | 17.92 | C |
| ATOM | 1246 | C | ILE | A | 141 | 58.729 | −0.282 | 17.577 | 1.00 | 13.46 | C |
| ATOM | 1247 | O | ILE | A | 141 | 58.730 | −1.374 | 18.148 | 1.00 | 13.92 | O |
| ATOM | 1248 | CB | ILE | A | 141 | 56.497 | 0.925 | 17.019 | 1.00 | 22.59 | C |
| ATOM | 1249 | CG1 | ILE | A | 141 | 55.466 | 1.906 | 17.557 | 1.00 | 17.61 | C |
| ATOM | 1250 | CG2 | ILE | A | 141 | 55.863 | −0.411 | 16.700 | 1.00 | 10.49 | C |
| ATOM | 1251 | CD1 | ILE | A | 141 | 54.530 | 2.327 | 16.449 | 1.00 | 13.43 | C |
| ATOM | 1252 | N | ALA | A | 142 | 59.637 | 0.028 | 16.650 | 1.00 | 10.29 | N |
| ATOM | 1253 | CA | ALA | A | 142 | 60.657 | −0.931 | 16.228 | 1.00 | 7.15 | C |
| ATOM | 1254 | C | ALA | A | 142 | 61.456 | −1.301 | 17.456 | 1.00 | 16.58 | C |
| ATOM | 1255 | O | ALA | A | 142 | 61.839 | −2.454 | 17.621 | 1.00 | 13.04 | O |
| ATOM | 1256 | CB | ALA | A | 142 | 61.604 | −0.288 | 15.130 | 1.00 | 4.44 | C |
| ATOM | 1257 | N | GLY | A | 143 | 61.703 | −0.307 | 18.316 | 1.00 | 9.56 | N |
| ATOM | 1258 | CA | GLY | A | 143 | 62.448 | −0.525 | 19.527 | 1.00 | 5.15 | C |
| ATOM | 1259 | C | GLY | A | 143 | 61.770 | −1.555 | 20.430 | 1.00 | 16.36 | C |
| ATOM | 1260 | O | GLY | A | 143 | 62.392 | −2.482 | 20.967 | 1.00 | 14.11 | O |
| ATOM | 1261 | N | ILE | A | 144 | 60.476 | −1.418 | 20.564 | 1.00 | 20.33 | N |
| ATOM | 1262 | CA | ILE | A | 144 | 59.725 | −2.314 | 21.407 | 1.00 | 15.35 | C |
| ATOM | 1263 | C | ILE | A | 144 | 59.706 | −3.732 | 20.859 | 1.00 | 19.84 | C |
| ATOM | 1264 | O | ILE | A | 144 | 59.836 | −4.700 | 21.608 | 1.00 | 17.93 | O |
| ATOM | 1265 | CE | ILE | A | 144 | 58.317 | −1.819 | 21.559 | 1.00 | 10.60 | C |
| ATOM | 1266 | CG1 | ILE | A | 144 | 58.311 | −0.610 | 22.516 | 1.00 | 9.80 | C |
| ATOM | 1267 | CG2 | ILE | A | 144 | 57.410 | −2.928 | 22.122 | 1.00 | 9.60 | C |
| ATOM | 1268 | CD1 | ILE | A | 144 | 57.022 | 0.076 | 22.517 | 1.00 | 18.32 | C |
| ATOM | 1269 | N | LYS | A | 145 | 59.520 | −3.841 | 19.556 | 1.00 | 7.20 | N |
| ATOM | 1270 | CA | LYS | A | 145 | 59.459 | −5.139 | 18.926 | 1.00 | 7.64 | C |
| ATOM | 1271 | C | LYS | A | 145 | 60.840 | −5.788 | 18.931 | 1.00 | 15.32 | C |
| ATOM | 1272 | O | LYS | A | 145 | 60.923 | −6.989 | 18.981 | 1.00 | 14.76 | O |
| ATOM | 1273 | CB | LYS | A | 145 | 58.891 | −5.001 | 17.516 | 1.00 | 11.25 | C |
| ATOM | 1274 | CG | LYS | A | 145 | 57.414 | −4.581 | 17.489 | 1.00 | 12.13 | C |
| ATOM | 1275 | CD | LYS | A | 145 | 56.642 | −5.434 | 18.495 | 1.00 | 25.23 | C |
| ATOM | 1276 | CE | LYS | A | 145 | 55.189 | −4.995 | 18.692 | 1.00 | 13.64 | C |
| ATOM | 1277 | NZ | LYS | A | 145 | 54.441 | −6.111 | 19.392 | 1.00 | 11.94 | N |
| ATOM | 1278 | N | LEU | A | 146 | 61.934 | −5.011 | 18.986 | 1.00 | 26.98 | N |
| ATOM | 1279 | CA | LEU | A | 146 | 63.261 | −5.642 | 19.167 | 1.00 | 19.72 | C |
| ATOM | 1280 | C | LEU | A | 146 | 63.262 | −6.316 | 20.542 | 1.00 | 18.20 | C |
| ATOM | 1281 | O | LEU | A | 146 | 63.590 | −7.511 | 20.703 | 1.00 | 19.86 | O |
| ATOM | 1282 | CE | LEU | A | 146 | 64.398 | −4.618 | 19.150 | 1.00 | 13.56 | C |
| ATOM | 1283 | CG | LEU | A | 146 | 64.895 | −4.258 | 17.759 | 1.00 | 21.84 | C |
| ATOM | 1284 | CD1 | LEU | A | 146 | 65.672 | −2.945 | 17.817 | 1.00 | 17.94 | C |
| ATOM | 1285 | CD2 | LEU | A | 146 | 65.745 | −5.397 | 17.102 | 1.00 | 16.10 | C |
| ATOM | 1286 | N | CYS | A | 147 | 62.931 | −5.523 | 21.548 | 1.00 | 7.91 | N |
| ATOM | 1287 | CA | CYS | A | 147 | 62.875 | −6.064 | 22.893 | 1.00 | 9.14 | C |
| ATOM | 1288 | C | CYS | A | 147 | 62.072 | −7.378 | 22.945 | 1.00 | 22.72 | C |
| ATOM | 1289 | O | CYS | A | 147 | 62.568 | −8.401 | 23.383 | 1.00 | 16.90 | O |
| ATOM | 1290 | CE | CYS | A | 147 | 62.232 | −5.058 | 23.809 | 1.00 | 12.63 | C |
| ATOM | 1291 | SG | CYS | A | 147 | 63.411 | −3.823 | 24.316 | 1.00 | 15.02 | S |
| ATOM | 1292 | N | GLU | A | 148 | 60.823 | −7.352 | 22.508 | 1.00 | 20.03 | N |
| ATOM | 1293 | CA | GLU | A | 148 | 60.016 | −8.555 | 22.567 | 1.00 | 16.09 | C |
| ATOM | 1294 | C | GLU | A | 148 | 60.685 | −9.715 | 21.802 | 1.00 | 22.61 | C |
| ATOM | 1295 | O | GLU | A | 148 | 60.651 | −10.888 | 22.226 | 1.00 | 12.05 | O |
| ATOM | 1296 | CE | GLU | A | 148 | 58.597 | −8.268 | 22.046 | 1.00 | 14.66 | C |
| ATOM | 1297 | CG | GLU | A | 148 | 57.864 | −7.189 | 22.840 | 1.00 | 11.45 | C |
| ATOM | 1298 | CD | GLU | A | 148 | 56.471 | −6.821 | 22.277 | 1.00 | 11.75 | C |
| ATOM | 1299 | OE1 | GLU | A | 148 | 56.117 | −7.055 | 21.080 | 1.00 | 11.65 | O |
| ATOM | 1300 | OE2 | GLU | A | 148 | 55.728 | −6.231 | 23.081 | 1.00 | 22.56 | O |
| ATOM | 1301 | N | SER | A | 149 | 61.368 | −9.377 | 20.715 | 1.00 | 15.57 | N |
| ATOM | 1302 | CA | SER | A | 149 | 61.938 | −10.428 | 19.887 | 1.00 | 10.21 | C |
| ATOM | 1303 | C | SER | A | 149 | 63.040 | −11.245 | 20.502 | 1.00 | 15.83 | C |
| ATOM | 1304 | O | SER | A | 149 | 63.102 | −12.458 | 20.291 | 1.00 | 12.72 | O |
| ATOM | 1305 | CB | SER | A | 149 | 62.270 | −9.936 | 18.488 | 1.00 | 9.44 | C |
| ATOM | 1306 | OG | SER | A | 149 | 61.053 | −9.650 | 17.782 | 1.00 | 15.91 | O |
| ATOM | 1307 | N | TYR | A | 150 | 63.910 | −10.546 | 21.224 | 1.00 | 18.44 | N |
| ATOM | 1308 | CA | TYR | A | 150 | 65.065 | −11.100 | 21.948 | 1.00 | 20.50 | C |
| ATOM | 1309 | C | TYR | A | 150 | 64.514 | −11.848 | 23.158 | 1.00 | 21.87 | C |
| ATOM | 1310 | O | TYR | A | 150 | 64.939 | −12.949 | 23.486 | 1.00 | 31.39 | O |
| ATOM | 1311 | CB | TYR | A | 150 | 66.005 | −9.950 | 22.425 | 1.00 | 13.71 | C |
| ATOM | 1312 | CG | TYR | A | 150 | 66.994 | −9.509 | 21.365 | 1.00 | 14.13 | C |
| ATOM | 1313 | CD1 | TYR | A | 150 | 66.611 | −8.673 | 20.317 | 1.00 | 14.64 | C |
| ATOM | 1314 | CD2 | TYR | A | 150 | 68.288 | −10.000 | 21.360 | 1.00 | 18.32 | C |
| ATOM | 1315 | CE1 | TYR | A | 150 | 67.487 | −8.390 | 19.278 | 1.00 | 11.91 | C |
| ATOM | 1316 | CE2 | TYR | A | 150 | 69.198 | −9.682 | 20.345 | 1.00 | 11.10 | C |
| ATOM | 1317 | CZ | TYR | A | 150 | 68.804 | −8.900 | 19.326 | 1.00 | 20.95 | C |
| ATOM | 1318 | OH | TYR | A | 150 | 69.739 | −8.685 | 18.333 | 1.00 | 27.73 | O |
| ATOM | 1319 | N | ASN | A | 151 | 63.536 | −11.249 | 23.801 | 1.00 | 14.83 | N |
| ATOM | 1320 | CA | ASN | A | 151 | 62.903 | −11.889 | 24.937 | 1.00 | 23.62 | C |
| ATOM | 1321 | C | ASN | A | 151 | 62.417 | −13.244 | 24.410 | 1.00 | 28.53 | C |
| ATOM | 1322 | O | ASN | A | 151 | 62.630 | −14.248 | 25.072 | 1.00 | 25.89 | O |
| ATOM | 1323 | CB | ASN | A | 151 | 61.655 | −11.113 | 25.439 | 1.00 | 20.95 | C |
| ATOM | 1324 | CG | ASN | A | 151 | 61.988 | −9.867 | 26.284 | 1.00 | 15.07 | C |
| ATOM | 1325 | OD1 | ASN | A | 151 | 61.126 | −9.020 | 26.466 | 1.00 | 26.72 | O |
| ATOM | 1326 | ND2 | ASN | A | 151 | 63.231 | −9.709 | 26.700 | 1.00 | 6.31 | N |
| ATOM | 1327 | N | ARG | A | 152 | 61.731 | −13.249 | 23.259 | 1.00 | 19.91 | N |
| ATOM | 1328 | CA | ARG | A | 152 | 61.129 | −14.465 | 22.687 | 1.00 | 17.62 | C |
| ATOM | 1329 | C | ARG | A | 152 | 62.090 | −15.523 | 22.188 | 1.00 | 21.34 | C |
| ATOM | 1330 | O | ARG | A | 152 | 61.959 | −16.687 | 22.542 | 1.00 | 15.44 | O |
| ATOM | 1331 | CB | ARG | A | 152 | 60.086 | −14.148 | 21.610 | 1.00 | 15.30 | C |
| ATOM | 1332 | CG | ARG | A | 152 | 58.672 | −13.754 | 22.157 | 1.00 | 17.22 | C |
| ATOM | 1333 | CD | ARG | A | 152 | 57.652 | −13.297 | 21.049 | 1.00 | 9.11 | C |
| ATOM | 1334 | NE | ARG | A | 152 | 57.161 | −14.419 | 20.241 | 1.00 | 21.05 | N |
| ATOM | 1335 | CZ | ARG | A | 152 | 57.159 | −14.447 | 18.912 | 1.00 | 28.61 | C |
| ATOM | 1336 | NH1 | ARG | A | 152 | 57.590 | −13.387 | 18.221 | 1.00 | 21.98 | N |
| ATOM | 1337 | NH2 | ARG | A | 152 | 56.717 | −15.528 | 18.262 | 1.00 | 26.11 | N |
| ATOM | 1338 | N | GLN | A | 153 | 63.098 | −15.104 | 21.434 | 1.00 | 16.54 | N |
| ATOM | 1339 | CA | GLN | A | 153 | 64.044 | −16.036 | 20.842 | 1.00 | 9.74 | C |
| ATOM | 1340 | C | GLN | A | 153 | 65.082 | −16.443 | 21.807 | 1.00 | 16.70 | C |
| ATOM | 1341 | O | GLN | A | 153 | 65.529 | −17.545 | 21.763 | 1.00 | 24.35 | O |
| ATOM | 1342 | CB | GLN | A | 153 | 64.789 | −15.372 | 19.714 | 1.00 | 8.99 | C |
| ATOM | 1343 | CG | GLN | A | 153 | 65.935 | −16.225 | 19.116 | 1.00 | 4.63 | C |
| ATOM | 1344 | CD | GLN | A | 153 | 66.315 | −15.637 | 17.762 | 1.00 | 14.17 | C |
| ATOM | 1345 | OE1 | GLN | A | 153 | 65.611 | −14.763 | 17.254 | 1.00 | 12.53 | O |
| ATOM | 1346 | NE2 | GLN | A | 153 | 67.466 | −16.024 | 17.228 | 1.00 | 13.38 | N |
| ATOM | 1347 | N | TYR | A | 154 | 65.566 | −15.518 | 22.608 | 1.00 | 14.35 | N |
| ATOM | 1348 | CA | TYR | A | 154 | 66.677 | −15.839 | 23.483 | 1.00 | 12.16 | C |
| ATOM | 1349 | C | TYR | A | 154 | 66.323 | −15.930 | 24.954 | 1.00 | 19.06 | C |
| ATOM | 1350 | O | TYR | A | 154 | 67.185 | −16.207 | 25.777 | 1.00 | 25.59 | O |
| ATOM | 1351 | CB | TYR | A | 154 | 67.829 | −14.816 | 23.326 | 1.00 | 16.89 | C |
| ATOM | 1352 | CG | TYR | A | 154 | 68.418 | −14.733 | 21.943 | 1.00 | 17.53 | C |
| ATOM | 1353 | CD1 | TYR | A | 154 | 69.259 | −15.726 | 21.467 | 1.00 | 18.91 | C |
| ATOM | 1354 | CD2 | TYR | A | 154 | 68.080 | −13.712 | 21.091 | 1.00 | 13.97 | C |
| ATOM | 1355 | CE1 | TYR | A | 154 | 69.782 | −15.686 | 20.190 | 1.00 | 10.98 | C |
| ATOM | 1356 | CE2 | TYR | A | 154 | 68.621 | −13.639 | 19.806 | 1.00 | 23.81 | C |
| ATOM | 1357 | CZ | TYR | A | 154 | 69.488 | −14.634 | 19.380 | 1.00 | 23.08 | C |
| ATOM | 1358 | OH | TYR | A | 154 | 70.002 | −14.619 | 18.118 | 1.00 | 23.87 | O |
| ATOM | 1359 | N | GLY | A | 155 | 65.080 | −15.686 | 25.313 | 1.00 | 12.08 | N |
| ATOM | 1360 | CA | GLY | A | 155 | 64.747 | −15.702 | 26.731 | 1.00 | 15.80 | C |
| ATOM | 1361 | C | GLY | A | 155 | 65.323 | −14.498 | 27.580 | 1.00 | 33.97 | C |
| ATOM | 1362 | O | GLY | A | 155 | 65.491 | −14.640 | 28.789 | 1.00 | 25.76 | O |
| ATOM | 1363 | N | ARG | A | 156 | 65.564 | −13.318 | 26.981 | 1.00 | 25.91 | N |
| ATOM | 1364 | CA | ARG | A | 156 | 66.066 | −12.146 | 27.734 | 1.00 | 14.13 | C |
| ATOM | 1365 | C | ARG | A | 156 | 64.971 | −11.486 | 28.581 | 1.00 | 16.23 | C |
| ATOM | 1366 | O | ARG | A | 156 | 63.802 | −11.919 | 28.583 | 1.00 | 22.61 | O |
| ATOM | 1367 | CB | ARG | A | 156 | 66.601 | −11.124 | 26.750 | 1.00 | 13.16 | C |
| ATOM | 1368 | CG | ARG | A | 156 | 67.875 | −11.570 | 26.099 | 1.00 | 15.18 | C |
| ATOM | 1369 | CD | ARG | A | 156 | 68.930 | −11.418 | 27.121 | 1.00 | 26.42 | C |
| ATOM | 1370 | NE | ARG | A | 156 | 70.200 | −11.912 | 26.633 | 1.00 | 21.25 | N |
| ATOM | 1371 | CZ | ARG | A | 156 | 71.092 | −12.555 | 27.386 | 1.00 | 42.25 | C |
| ATOM | 1372 | NH1 | ARG | A | 156 | 70.870 | −12.795 | 28.679 | 1.00 | 20.02 | N |
| ATOM | 1373 | NH2 | ARG | A | 156 | 72.221 | −12.966 | 26.843 | 1.00 | 20.88 | N |
| ATOM | 1374 | N | ASP | A | 157 | 65.343 | −10.446 | 29.321 | 1.00 | 16.00 | N |
| ATOM | 1375 | CA | ASP | A | 157 | 64.370 | −9.749 | 30.166 | 1.00 | 16.20 | C |
| ATOM | 1376 | C | ASP | A | 157 | 64.444 | −8.245 | 29.841 | 1.00 | 19.20 | C |
| ATOM | 1377 | O | ASP | A | 157 | 64.865 | −7.429 | 30.650 | 1.00 | 10.71 | O |
| ATOM | 1378 | CE | ASP | A | 157 | 64.609 | −10.061 | 31.652 | 1.00 | 16.50 | C |
| ATOM | 1379 | CG | ASP | A | 157 | 63.489 | −9.560 | 32.566 1.00 | 26.45 | C | |
| ATOM | 1380 | OD1 | ASP | A | 157 | 62.433 | −9.060 | 32.108 | 1.00 | 26.82 | O |
| ATOM | 1381 | OD2 | ASP | A | 157 | 63.673 | −9.653 | 33.784 | 1.00 | 21.88 | O |
| ATOM | 1382 | N | TYR | A | 158 | 64.038 | −7.921 | 28.620 | 1.00 | 19.41 | N |
| ATOM | 1383 | CA | TYR | A | 158 | 64.099 | −6.564 | 28.083 | 1.00 | 18.96 | C |
| ATOM | 1384 | C | TYR | A | 158 | 62.688 | −5.977 | 28.127 | 1.00 | 22.62 | C |
| ATOM | 1385 | O | TYR | A | 158 | 61.854 | −6.296 | 27.282 | 1.00 | 10.12 | O |
| ATOM | 1386 | CB | TYR | A | 158 | 64.562 | −6.661 | 26.631 | 1.00 | 16.34 | C |
| ATOM | 1387 | CG | TYR | A | 158 | 65.982 | −7.166 | 26.484 | 1.00 | 12.04 | C |
| ATOM | 1388 | CD1 | TYR | A | 158 | 66.789 | −7.415 | 27.621 | 1.00 | 13.76 | C |
| ATOM | 1389 | CD2 | TYR | A | 158 | 66.544 | −7.349 | 25.218 | 1.00 | 16.35 | C |
| ATOM | 1390 | CE1 | TYR | A | 158 | 68.135 | −7.786 | 27.482 | 1.00 | 8.18 | C |
| ATOM | 1391 | CE2 | TYR | A | 158 | 67.886 | −7.732 | 25.060 | 1.00 | 13.73 | C |
| ATOM | 1392 | CZ | TYR | A | 158 | 68.676 | −7.942 | 26.186 | 1.00 | 24.45 | C |
| ATOM | 1393 | OH | TYR | A | 158 | 69.993 | −8.338 | 25.997 | 1.00 | 14.36 | O |
| ATOM | 1394 | N | ARG | A | 159 | 62.423 | −5.200 | 29.175 | 1.00 | 23.53 | N |
| ATOM | 1395 | CA | ARG | A | 159 | 61.105 | −4.603 | 29.483 | 1.00 | 21.15 | C |
| ATOM | 1396 | C | ARG | A | 159 | 60.930 | −3.172 | 28.878 | 1.00 | 23.55 | C |
| ATOM | 1397 | O | ARG | A | 159 | 61.911 | −2.566 | 28.424 | 1.00 | 18.12 | O |
| ATOM | 1398 | CB | ARG | A | 159 | 60.891 | −4.608 | 31.034 | 1.00 | 21.68 | C |
| ATOM | 1399 | CG | ARG | A | 159 | 60.986 | −6.029 | 31.722 | 1.00 | 16.41 | C |
| ATOM | 1400 | CD | ARG | A | 159 | 61.135 | −6.052 | 33.233 | 1.00 | 18.10 | C |
| ATOM | 1401 | NE | ARG | A | 159 | 61.305 | −7.402 | 33.772 | 1.00 | 19.25 | N |
| ATOM | 1402 | CZ | ARG | A | 159 | 61.164 | −7.720 | 35.058 | 1.00 | 36.67 | C |
| ATOM | 1403 | NH1 | ARG | A | 159 | 60.886 | −6.776 | 35.962 | 1.00 | 15.32 | N |
| ATOM | 1404 | NH2 | ARG | A | 159 | 61.309 | −8.986 | 35.448 | 1.00 | 11.79 | N |
| ATOM | 1405 | N | SER | A | 160 | 59.689 | −2.661 | 28.859 | 1.00 | 24.44 | N |
| ATOM | 1406 | CA | SER | A | 160 | 59.312 | −1.393 | 28.200 | 1.00 | 21.59 | C |
| ATOM | 1407 | C | SER | A | 160 | 58.242 | −0.577 | 28.950 | 1.00 | 25.07 | C |
| ATOM | 1408 | O | SER | A | 160 | 57.257 | −1.127 | 29.454 | 1.00 | 17.02 | O |
| ATOM | 1409 | CB | SER | A | 160 | 58.719 | −1.747 | 26.797 | 1.00 | 13.05 | C |
| ATOM | 1410 | OG | SER | A | 160 | 59.782 | −1.897 | 25.885 | 1.00 | 37.57 | O |
| ATOM | 1411 | N | VAL | A | 161 | 58.378 | 0.742 | 28.927 | 1.00 | 21.01 | N |
| ATOM | 1412 | CA | VAL | A | 161 | 57.369 | 1.644 | 29.509 | 1.00 | 9.70 | C |
| ATOM | 1413 | C | VAL | A | 161 | 57.068 | 2.747 | 28.504 | 1.00 | 16.77 | C |
| ATOM | 1414 | O | VAL | A | 161 | 57.955 | 3.149 | 27.729 | 1.00 | 16.33 | O |
| ATOM | 1415 | CB | VAL | A | 161 | 57.806 | 2.248 | 30.862 | 1.00 | 17.94 | C |
| ATOM | 1416 | CG1 | VAL | A | 161 | 57.873 | 1.185 | 31.984 | 1.00 | 16.16 | C |
| ATOM | 1417 | CG2 | VAL | A | 161 | 59.137 | 2.992 | 30.750 | 1.00 | 21.10 | C |
| ATOM | 1418 | N | MET | A | 162 | 55.794 | 3.147 | 28.443 | 1.00 | 22.46 | N |
| ATOM | 1419 | CA | MET | A | 162 | 55.296 | 4.185 | 27.513 | 1.00 | 19.23 | C |
| ATOM | 1420 | C | MET | A | 162 | 54.880 | 5.312 | 28.397 | 1.00 | 25.19 | C |
| ATOM | 1421 | O | MET | A | 162 | 53.788 | 5.269 | 28.961 | 1.00 | 18.35 | O |
| ATOM | 1422 | CB | MET | A | 162 | 53.979 | 3.796 | 26.850 | 1.00 | 15.55 | C |
| ATOM | 1423 | CG | MET | A | 162 | 54.013 | 2.630 | 25.949 | 1.00 | 37.79 | C |
| ATOM | 1424 | SD | MET | A | 162 | 54.354 | 3.100 | 24.235 | 1.00 | 52.07 | S |
| ATOM | 1425 | CE | MET | A | 162 | 56.193 | 3.134 | 24.410 | 1.00 | 36.30 | C |
| ATOM | 1426 | N | PRO | A | 163 | 55.730 | 6.313 | 28.521 | 1.00 | 18.43 | N |
| ATOM | 1427 | CA | PRO | A | 163 | 55.390 | 7.472 | 29.337 | 1.00 | 17.76 | C |
| ATOM | 1428 | C | PRO | A | 163 | 54.300 | 8.384 | 28.667 | 1.00 | 21.23 | C |
| ATOM | 1429 | O | PRO | A | 163 | 54.208 | 8.448 | 27.433 | 1.00 | 15.20 | O |
| ATOM | 1430 | CB | PRO | A | 163 | 56.727 | 8.196 | 29.423 | 1.00 | 11.43 | C |
| ATOM | 1431 | CG | PRO | A | 163 | 57.352 | 7.874 | 28.031 | 1.00 | 13.99 | C |
| ATOM | 1432 | CD | PRO | A | 163 | 57.086 | 6.401 | 27.949 | 1.00 | 12.24 | C |
| ATOM | 1433 | N | THR | A | 164 | 53.478 | 9.060 | 29.478 | 1.00 | 13.95 | N |
| ATOM | 1434 | CA | THR | A | 164 | 52.581 | 10.121 | 28.963 | 1.00 | 25.82 | C |
| ATOM | 1435 | C | THR | A | 164 | 53.406 | 11.441 | 28.781 | 1.00 | 19.67 | C |
| ATOM | 1436 | O | THR | A | 164 | 54.633 | 11.393 | 28.868 | 1.00 | 13.97 | O |
| ATOM | 1437 | CB | THR | A | 164 | 51.373 | 10.391 | 29.903 | 1.00 | 25.51 | C |
| ATOM | 1438 | OG1 | THR | A | 164 | 50.470 | 11.321 | 29.267 | 1.00 | 14.77 | O |
| ATOM | 1439 | CG2 | THR | A | 164 | 51.818 | 10.886 | 31.298 | 1.00 | 9.06 | C |
| ATOM | 1440 | N | ASN | A | 165 | 52.751 | 12.589 | 28.556 | 1.00 | 14.99 | N |
| ATOM | 1441 | CA | ASN | A | 165 | 53.448 | 13.901 | 28.481 | 1.00 | 7.83 | C |
| ATOM | 1442 | C | ASN | A | 165 | 54.167 | 14.064 | 29.824 | 1.00 | 11.21 | C |
| ATOM | 1443 | O | ASN | A | 165 | 53.554 | 13.929 | 30.894 | 1.00 | 17.66 | O |
| ATOM | 1444 | CE | ASN | A | 165 | 52.434 | 15.061 | 28.416 | 1.00 | 14.48 | C |
| ATOM | 1445 | CG | ASN | A | 165 | 51.492 | 14.941 | 27.262 | 1.00 | 23.70 | C |
| ATOM | 1446 | OD1 | ASN | A | 165 | 51.939 | 14.800 | 26.129 | 1.00 | 22.37 | O |
| ATOM | 1447 | ND2 | ASN | A | 165 | 50.173 | 14.925 | 27.539 | 1.00 | 27.22 | N |
| ATOM | 1448 | N | LEU | A | 166 | 55.418 | 14.490 | 29.777 | 1.00 | 8.23 | N |
| ATOM | 1449 | CA | LEU | A | 166 | 56.187 | 14.604 | 30.994 | 1.00 | 14.40 | C |
| ATOM | 1450 | C | LEU | A | 166 | 56.629 | 16.017 | 31.120 | 1.00 | 25.05 | C |
| ATOM | 1451 | O | LEU | A | 166 | 56.624 | 16.718 | 30.125 | 1.00 | 25.09 | O |
| ATOM | 1452 | CE | LEU | A | 166 | 57.460 | 13.743 | 30.870 | 1.00 | 17.48 | C |
| ATOM | 1453 | CG | LEU | A | 166 | 57.423 | 12.218 | 30.652 | 1.00 | 16.63 | C |
| ATOM | 1454 | CD1 | LEU | A | 166 | 58.837 | 11.639 | 31.000 | 1.00 | 22.52 | C |
| ATOM | 1455 | CD2 | LEU | A | 166 | 56.336 | 11.539 | 31.514 | 1.00 | 7.46 | C |
| ATOM | 1456 | N | TYR | A | 167 | 57.146 | 16.391 | 32.300 | 1.00 | 19.78 | N |
| ATOM | 1457 | CA | TYR | A | 167 | 57.678 | 17.760 | 32.511 | 1.00 | 18.58 | C |
| ATOM | 1458 | C | TYR | A | 167 | 58.534 | 17.763 | 33.767 | 1.00 | 15.53 | C |
| ATOM | 1459 | O | TYR | A | 167 | 58.474 | 16.852 | 34.575 | 1.00 | 16.71 | O |
| ATOM | 1460 | CB | TYR | A | 167 | 56.509 | 18.778 | 32.665 | 1.00 | 18.33 | C |
| ATOM | 1461 | CG | TYR | A | 167 | 55.671 | 18.561 | 33.931 | 1.00 | 14.23 | C |
| ATOM | 1462 | CD1 | TYR | A | 167 | 54.624 | 17.618 | 33.977 | 1.00 | 13.35 | C |
| ATOM | 1463 | CD2 | TYR | A | 167 | 55.984 | 19.258 | 35.106 | 1.00 | 16.52 | C |
| ATOM | 1464 | CE1 | TYR | A | 167 | 53.889 | 17.446 | 35.146 | 1.00 | 21.17 | C |
| ATOM | 1465 | CE2 | TYR | A | 167 | 55.302 | 19.084 | 36.264 | 1.00 | 8.26 | C |
| ATOM | 1466 | CZ | TYR | A | 167 | 54.228 | 18.203 | 36.296 | 1.00 | 23.56 | C |
| ATOM | 1467 | OH | TYR | A | 167 | 53.526 | 18.078 | 37.504 | 1.00 | 22.81 | O |
| ATOM | 1468 | N | GLY | A | 168 | 59.334 | 18.797 | 33.952 | 1.00 | 16.59 | N |
| ATOM | 1469 | CA | GLY | A | 168 | 60.158 | 18.817 | 35.152 | 1.00 | 18.21 | C |
| ATOM | 1470 | C | GLY | A | 168 | 61.534 | 19.428 | 34.880 | 1.00 | 13.69 | C |
| ATOM | 1471 | O | GLY | A | 168 | 61.746 | 20.028 | 33.837 | 1.00 | 16.52 | O |
| ATOM | 1472 | N | PRO | A | 169 | 62.473 | 19.263 | 35.817 | 1.00 | 20.33 | N |
| ATOM | 1473 | CA | PRO | A | 169 | 63.801 | 19.822 | 35.656 | 1.00 | 16.07 | C |
| ATOM | 1474 | C | PRO | A | 169 | 64.430 | 19.353 | 34.387 | 1.00 | 27.18 | C |
| ATOM | 1475 | O | PRO | A | 169 | 64.305 | 18.186 | 33.981 | 1.00 | 21.23 | O |
| ATOM | 1476 | CB | PRO | A | 169 | 64.595 | 19.206 | 36.805 | 1.00 | 17.28 | C |
| ATOM | 1477 | CG | PRO | A | 169 | 63.649 | 18.919 | 37.830 | 1.00 | 19.89 | C |
| ATOM | 1478 | CD | PRO | A | 169 | 62.263 | 18.772 | 37.189 | 1.00 | 22.47 | C |
| ATOM | 1479 | N | HIS | A | 170 | 65.226 | 20.235 | 33.829 | 1.00 | 19.48 | N |
| ATOM | 1480 | CA | HIS | A | 170 | 65.952 | 19.877 | 32.638 | 1.00 | 25.56 | C |
| ATOM | 1481 | C | HIS | A | 170 | 65.096 | 19.707 | 31.428 | 1.00 | 29.15 | C |
| ATOM | 1482 | O | HIS | A | 170 | 65.553 | 19.091 | 30.479 | 1.00 | 29.71 | O |
| ATOM | 1483 | CB | HIS | A | 170 | 66.783 | 18.600 | 32.845 | 1.00 | 28.94 | C |
| ATOM | 1484 | CG | HIS | A | 170 | 67.703 | 18.671 | 34.034 | 1.00 | 33.88 | C |
| ATOM | 1485 | ND1 | HIS | A | 170 | 68.975 | 19.203 | 33.969 | 1.00 | 25.46 | N |
| ATOM | 1486 | CD2 | HIS | A | 170 | 67.518 | 18.298 | 35.326 | 1.00 | 34.77 | C |
| ATOM | 1487 | CE1 | HIS | A | 170 | 69.531 | 19.151 | 35.166 | 1.00 | 25.63 | C |
| ATOM | 1488 | NE2 | HIS | A | 170 | 68.673 | 18.603 | 36.008 | 1.00 | 31.72 | N |
| ATOM | 1489 | N | ASP | A | 171 | 63.881 | 20.245 | 31.440 | 1.00 | 21.52 | N |
| ATOM | 1490 | CA | ASP | A | 171 | 63.041 | 20.267 | 30.218 | 1.00 | 28.63 | C |
| ATOM | 1491 | C | ASP | A | 171 | 63.630 | 21.459 | 29.359 | 1.00 | 41.94 | C |
| ATOM | 1492 | O | ASP | A | 171 | 64.534 | 22.171 | 29.835 | 1.00 | 29.69 | O |
| ATOM | 1493 | CB | ASP | A | 171 | 61.552 | 20.558 | 30.602 | 1.00 | 26.40 | C |
| ATOM | 1494 | CG | ASP | A | 171 | 60.552 | 20.097 | 29.540 | 1.00 | 22.32 | C |
| ATOM | 1495 | OD1 | ASP | A | 171 | 60.890 | 20.067 | 28.325 | 1.00 | 32.03 | O |
| ATOM | 1496 | OD2 | ASP | A | 171 | 59.427 | 19.719 | 29.916 | 1.00 | 42.13 | O |
| ATOM | 1497 | N | ASN | A | 172 | 63.141 | 21.712 | 28.137 | 1.00 | 42.08 | N |
| ATOM | 1498 | CA | ASN | A | 172 | 63.616 | 22.893 | 27.388 | 1.00 | 35.95 | C |
| ATOM | 1499 | C | ASN | A | 172 | 62.665 | 24.056 | 27.674 | 1.00 | 33.71 | C |
| ATOM | 1500 | O | ASN | A | 172 | 61.586 | 24.102 | 27.104 | 1.00 | 32.69 | O |
| ATOM | 1501 | CB | ASN | A | 172 | 63.632 | 22.667 | 25.869 | 1.00 | 41.60 | C |
| ATOM | 1502 | CG | ASN | A | 172 | 63.807 | 23.987 | 25.086 | 1.00 | 39.09 | C |
| ATOM | 1503 | OD1 | ASN | A | 172 | 62.973 | 24.347 | 24.259 | 1.00 | 83.94 | O |
| ATOM | 1504 | ND2 | ASN | A | 172 | 64.855 | 24.740 | 25.418 | 1.00 | 65.07 | N |
| ATOM | 1505 | N | PHE | A | 173 | 63.021 | 24.953 | 28.583 | 1.00 | 31.93 | N |
| ATOM | 1506 | CA | PHE | A | 173 | 62.082 | 26.030 | 28.944 | 1.00 | 48.24 | C |
| ATOM | 1507 | C | PHE | A | 173 | 61.989 | 27.260 | 28.045 | 1.00 | 69.01 | C |
| ATOM | 1508 | O | PHE | A | 173 | 62.278 | 28.395 | 28.465 | 1.00 | 58.79 | O |
| ATOM | 1509 | CB | PHE | A | 173 | 62.225 | 26.459 | 30.390 | 1.00 | 43.43 | C |
| ATOM | 1510 | CG | PHE | A | 173 | 61.867 | 25.399 | 31.356 | 1.00 | 34.19 | C |
| ATOM | 1511 | CD1 | PHE | A | 173 | 62.810 | 24.488 | 31.751 | 1.00 | 24.68 | C |
| ATOM | 1512 | CD2 | PHE | A | 173 | 60.621 | 25.354 | 31.925 | 1.00 | 24.84 | C |
| ATOM | 1513 | CE1 | PHE | A | 173 | 62.524 | 23.548 | 32.682 | 1.00 | 23.64 | C |
| ATOM | 1514 | CE2 | PHE | A | 173 | 60.305 | 24.366 | 32.804 | 1.00 | 31.32 | C |
| ATOM | 1515 | CZ | PHE | A | 173 | 61.263 | 23.457 | 33.192 | 1.00 | 24.30 | C |
| ATOM | 1516 | N | HIS | A | 174 | 61.510 | 27.036 | 26.831 | 1.00 | 68.16 | N |
| ATOM | 1517 | CA | HIS | A | 174 | 61.401 | 28.109 | 25.871 | 1.00 | 64.53 | C |
| ATOM | 1518 | C | HIS | A | 174 | 59.973 | 28.221 | 25.400 | 1.00 | 71.58 | C |
| ATOM | 1519 | O | HIS | A | 174 | 59.309 | 27.186 | 25.249 | 1.00 | 73.20 | O |
| ATOM | 1520 | CB | HIS | A | 174 | 62.418 | 27.870 | 24.736 | 1.00 | 71.71 | C |
| ATOM | 1521 | CG | HIS | A | 174 | 63.835 | 27.868 | 25.229 | 1.00 | 92.29 | C |
| ATOM | 1522 | ND1 | HIS | A | 174 | 64.921 | 27.539 | 24.440 | 1.00 | 100.00 | N |
| ATOM | 1523 | CD2 | HIS | A | 174 | 64.338 | 28.133 | 26.463 | 1.00 | 100.00 | C |
| ATOM | 1524 | CE1 | HIS | A | 174 | 66.032 | 27.628 | 25.160 | 1.00 | 100.00 | C |
| ATOM | 1525 | NE2 | HIS | A | 174 | 65.705 | 27.981 | 26.393 | 1.00 | 100.00 | N |
| ATOM | 1526 | N | PRO | A | 175 | 59.469 | 29.461 | 25.262 | 1.00 | 65.71 | N |
| ATOM | 1527 | CA | PRO | A | 175 | 58.109 | 29.658 | 24.770 | 1.00 | 55.72 | C |
| ATOM | 1528 | C | PRO | A | 175 | 58.233 | 29.297 | 23.267 | 1.00 | 75.83 | C |
| ATOM | 1529 | O | PRO | A | 175 | 57.224 | 29.226 | 22.554 | 1.00 | 69.59 | O |
| ATOM | 1530 | CE | PRO | A | 175 | 57.866 | 31.142 | 25.026 | 1.00 | 49.14 | C |
| ATOM | 1531 | CG | PRO | A | 175 | 59.258 | 31.790 | 24.901 | 1.00 | 42.23 | C |
| ATOM | 1532 | CD | PRO | A | 175 | 60.286 | 30.695 | 25.109 | 1.00 | 49.59 | C |
| ATOM | 1533 | N | SER | A | 176 | 59.480 | 28.954 | 22.879 | 1.00 | 85.09 | N |
| ATOM | 1534 | CA | SER | A | 176 | 59.954 | 28.474 | 21.548 | 1.00 | 81.18 | C |
| ATOM | 1535 | C | SER | A | 176 | 59.660 | 26.965 | 21.343 | 1.00 | 73.90 | C |
| ATOM | 1536 | O | SER | A | 176 | 59.617 | 26.458 | 20.213 | 1.00 | 57.03 | O |
| ATOM | 1537 | CB | SER | A | 176 | 61.493 | 28.666 | 21.447 | 1.00 | 71.32 | C |
| ATOM | 1538 | OG | SER | A | 176 | 62.048 | 29.349 | 22.578 | 1.00 | 51.93 | O |
| ATOM | 1539 | N | ASN | A | 177 | 59.520 | 26.276 | 22.480 | 1.00 | 66.23 | N |
| ATOM | 1540 | CA | ASN | A | 177 | 59.274 | 24.847 | 22.619 | 1.00 | 56.41 | C |
| ATOM | 1541 | C | ASN | A | 177 | 57.810 | 24.497 | 22.353 | 1.00 | 60.91 | C |
| ATOM | 1542 | O | ASN | A | 177 | 56.914 | 25.215 | 22.811 | 1.00 | 55.58 | O |
| ATOM | 1543 | CB | ASN | A | 177 | 59.619 | 24.469 | 24.065 | 1.00 | 50.45 | C |
| ATOM | 1544 | CG | ASN | A | 177 | 59.562 | 22.970 | 24.319 | 1.00 | 66.57 | C |
| ATOM | 1545 | OD1 | ASN | A | 177 | 59.095 | 22.216 | 23.476 | 1.00 | 100.00 | O |
| ATOM | 1546 | ND2 | ASN | A | 177 | 60.099 | 22.546 | 25.464 | 1.00 | 35.61 | N |
| ATOM | 1547 | N | SER | A | 178 | 57.583 | 23.387 | 21.627 | 1.00 | 57.10 | N |
| ATOM | 1548 | CA | SER | A | 178 | 56.234 | 22.853 | 21.279 | 1.00 | 50.50 | C |
| ATOM | 1549 | C | SER | A | 178 | 55.557 | 22.159 | 22.491 | 1.00 | 76.24 | C |
| ATOM | 1550 | O | SER | A | 178 | 54.575 | 21.400 | 22.304 | 1.00 | 99.63 | O |
| ATOM | 1551 | CB | SER | A | 178 | 56.316 | 21.800 | 20.118 | 1.00 | 10.17 | C |
| ATOM | 1552 | OG | SER | A | 178 | 57.397 | 22.112 | 19.217 | 1.00 | 71.69 | O |
| ATOM | 1553 | N | HIS | A | 179 | 56.134 | 22.284 | 23.694 | 1.00 | 37.39 | N |
| ATOM | 1554 | CA | HIS | A | 179 | 55.569 | 21.587 | 24.855 | 1.00 | 30.96 | C |
| ATOM | 1555 | C | HIS | A | 179 | 54.961 | 22.616 | 25.767 | 1.00 | 21.93 | C |
| ATOM | 1556 | O | HIS | A | 179 | 55.641 | 23.598 | 26.138 | 1.00 | 25.17 | O |
| ATOM | 1557 | CB | HIS | A | 179 | 56.634 | 20.683 | 25.575 | 1.00 | 36.20 | C |
| ATOM | 1558 | CG | HIS | A | 179 | 56.973 | 19.419 | 24.835 | 1.00 | 42.90 | C |
| ATOM | 1559 | ND1 | HIS | A | 179 | 56.973 | 19.335 | 23.457 | 1.00 | 49.52 | N |
| ATOM | 1560 | CD2 | HIS | A | 179 | 57.323 | 18.190 | 25.278 | 1.00 | 52.42 | C |
| ATOM | 1561 | CE1 | HIS | A | 179 | 57.283 | 18.109 | 23.084 | 1.00 | 44.78 | C |
| ATOM | 1562 | NE2 | HIS | A | 179 | 57.500 | 17.393 | 24.168 | 1.00 | 50.49 | N |
| ATOM | 1563 | N | VAL | A | 160 | 53.661 | 22.454 | 26.038 | 1.00 | 19.14 | N |
| ATOM | 1564 | CA | VAL | A | 180 | 52.886 | 23.449 | 26.789 | 1.00 | 29.03 | C |
| ATOM | 1565 | C | VAL | A | 180 | 53.373 | 23.890 | 28.142 | 1.00 | 31.29 | C |
| ATOM | 1566 | O | VAL | A | 180 | 53.348 | 25.075 | 28.447 | 1.00 | 19.55 | O |
| ATOM | 1567 | CB | VAL | A | 180 | 51.403 | 23.115 | 26.914 | 1.00 | 35.47 | C |
| ATOM | 1568 | CG1 | VAL | A | 180 | 50.630 | 24.399 | 27.217 | 1.00 | 35.84 | C |
| ATOM | 1569 | CG2 | VAL | A | 180 | 50.923 | 22.550 | 25.663 | 1.00 | 36.11 | C |
| ATOM | 1570 | N | ILE | A | 181 | 53.684 | 22.935 | 29.005 | 1.00 | 26.57 | N |
| ATOM | 1571 | CA | ILE | A | 181 | 54.138 | 23.285 | 30.360 | 1.00 | 24.49 | C |
| ATOM | 1572 | C | ILE | A | 181 | 55.371 | 24.213 | 30.361 | 1.00 | 16.51 | C |
| ATOM | 1573 | O | ILE | A | 181 | 55.326 | 25.315 | 30.909 | 1.00 | 24.42 | O |
| ATOM | 1574 | CB | ILE | A | 181 | 54.285 | 22.018 | 31.264 | 1.00 | 20.20 | C |
| ATOM | 1575 | CG1 | ILE | A | 181 | 52.878 | 21.428 | 31.528 | 1.00 | 18.22 | C |
| ATOM | 1576 | CG2 | ILE | A | 181 | 55.014 | 22.315 | 32.581 | 1.00 | 13.37 | C |
| ATOM | 1577 | CD1 | ILE | A | 181 | 52.867 | 20.086 | 32.286 | 1.00 | 8.03 | C |
| ATOM | 1578 | N | PRO | A | 182 | 56.452 | 23.779 | 29.718 | 1.00 | 22.21 | N |
| ATOM | 1579 | CA | PRO | A | 182 | 57.664 | 24.605 | 29.640 | 1.00 | 22.07 | C |
| ATOM | 1580 | C | PRO | A | 182 | 57.379 | 25.852 | 28.828 | 1.00 | 24.18 | C |
| ATOM | 1581 | O | PRO | A | 182 | 57.811 | 26.949 | 29.210 | 1.00 | 18.35 | O |
| ATOM | 1582 | CB | PRO | A | 182 | 58.682 | 23.725 | 28.890 | 1.00 | 24.97 | C |
| ATOM | 1583 | CG | PRO | A | 182 | 57.925 | 22.473 | 28.471 | 1.00 | 25.77 | C |
| ATOM | 1584 | CD | PRO | A | 182 | 56.727 | 22.359 | 29.401 | 1.00 | 18.23 | C |
| ATOM | 1585 | N | ALA | A | 183 | 56.628 | 25.707 | 27.729 | 1.00 | 21.45 | N |
| ATOM | 1586 | CA | ALA | A | 183 | 56.261 | 26.896 | 26.943 | 1.00 | 21.66 | C |
| ATOM | 1587 | C | ALA | A | 183 | 55.464 | 27.900 | 27.811 | 1.00 | 26.10 | C |
| ATOM | 1588 | O | ALA | A | 183 | 55.773 | 29.091 | 27.856 | 1.00 | 19.50 | O |
| ATOM | 1589 | CB | ALA | A | 183 | 55.473 | 26.513 | 25.703 | 1.00 | 13.26 | C |
| ATOM | 1590 | N | LEU | A | 184 | 54.472 | 27.389 | 28.543 | 1.00 | 23.34 | N |
| ATOM | 1591 | CA | LEU | A | 184 | 53.642 | 28.215 | 29.401 | 1.00 | 19.05 | C |
| ATOM | 1592 | C | LEU | A | 184 | 54.312 | 28.693 | 30.655 | 1.00 | 21.91 | C |
| ATOM | 1593 | O | LEU | A | 184 | 54.017 | 29.771 | 31.158 | 1.00 | 19.71 | O |
| ATOM | 1594 | CB | LEU | A | 184 | 52.309 | 27.553 | 29.715 | 1.00 | 14.41 | C |
| ATOM | 1595 | CG | LEU | A | 184 | 51.342 | 27.595 | 28.525 | 1.00 | 23.42 | C |
| ATOM | 1596 | CD1 | LEU | A | 184 | 49.918 | 27.244 | 28.928 | 1.00 | 31.06 | C |
| ATOM | 1597 | CD2 | LEU | A | 184 | 51.380 | 28.896 | 27.690 | 1.00 | 21.73 | C |
| ATOM | 1598 | N | LEU | A | 185 | 55.178 | 27.879 | 31.213 | 1.00 | 18.39 | N |
| ATOM | 1599 | CA | LEU | A | 185 | 55.833 | 28.332 | 32.417 | 1.00 | 16.39 | C |
| ATOM | 1600 | C | LEU | A | 185 | 56.669 | 29.528 | 31.985 | 1.00 | 23.67 | C |
| ATOM | 1601 | O | LEU | A | 185 | 56.681 | 30.590 | 32.644 | 1.00 | 29.38 | O |
| ATOM | 1602 | CB | LEU | A | 185 | 56.723 | 27.233 | 33.015 | 1.00 | 15.05 | C |
| ATOM | 1603 | CG | LEU | A | 185 | 56.021 | 26.348 | 34.041 | 1.00 | 15.56 | C |
| ATOM | 1604 | CD1 | LEU | A | 185 | 56.819 | 25.022 | 34.301 | 1.00 | 21.06 | C |
| ATOM | 1605 | CD2 | LEU | A | 185 | 55.722 | 27.113 | 35.321 | 1.00 | 11.02 | C |
| ATOM | 1606 | N | ARG | A | 186 | 57.337 | 29.397 | 30.852 | 1.00 | 17.09 | N |
| ATOM | 1607 | CA | ARG | A | 186 | 58.137 | 30.523 | 30.429 | 1.00 | 18.82 | C |
| ATOM | 1608 | C | ARG | A | 186 | 57.308 | 31.752 | 30.069 | 1.00 | 29.00 | C |
| ATOM | 1609 | O | ARG | A | 186 | 57.629 | 32.880 | 30.476 | 1.00 | 23.91 | O |
| ATOM | 1610 | CB | ARG | A | 186 | 59.026 | 30.146 | 29.281 | 1.00 | 22.06 | C |
| ATOM | 1611 | CG | ARG | A | 186 | 59.653 | 31.365 | 28.652 | 1.00 | 38.46 | C |
| ATOM | 1612 | CD | ARG | A | 186 | 60.825 | 31.804 | 29.462 | 1.00 | 83.66 | C |
| ATOM | 1613 | NE | ARG | A | 186 | 62.012 | 31.861 | 28.631 | 1.00 | 70.77 | N |
| ATOM | 1614 | CZ | ARG | A | 186 | 63.058 | 32.622 | 28.904 | 1.00 | 91.68 | C |
| ATOM | 1615 | NH1 | ARG | A | 186 | 63.053 | 33.386 | 29.995 | 1.00 | 56.56 | N |
| ATOM | 1616 | NH2 | ARG | A | 186 | 64.098 | 32.639 | 28.082 | 1.00 | 100.00 | N |
| ATOM | 1617 | N | ARG | A | 187 | 56.234 | 31.544 | 29.310 | 1.00 | 20.96 | N |
| ATOM | 1618 | CA | ARG | A | 187 | 55.361 | 32.662 | 28.941 | 1.00 | 19.32 | C |
| ATOM | 1619 | C | ARG | A | 187 | 54.765 | 33.453 | 30.142 | 1.00 | 28.41 | C |
| ATOM | 1620 | O | ARG | A | 187 | 54.823 | 34.700 | 30.193 | 1.00 | 17.23 | O |
| ATOM | 1621 | CB | ARG | A | 187 | 54.270 | 32.223 | 27.957 | 1.00 | 17.05 | C |
| ATOM | 1622 | CG | ARG | A | 187 | 54.813 | 31.546 | 26.720 | 1.00 | 61.42 | C |
| ATOM | 1623 | CD | ARG | A | 187 | 53.696 | 31.244 | 25.757 | 1.00 | 44.57 | C |
| ATOM | 1624 | NE | ARG | A | 187 | 53.033 | 32.472 | 25.354 | 1.00 | 29.47 | N |
| ATOM | 1625 | CZ | ARG | A | 187 | 51.831 | 32.534 | 24.790 | 1.00 | 17.82 | C |
| ATOM | 1626 | NH1 | ARG | A | 187 | 51.136 | 31.427 | 24.544 | 1.00 | 24.95 | N |
| ATOM | 1627 | NH2 | ARG | A | 187 | 51.341 | 33.716 | 24.447 | 1.00 | 37.77 | N |
| ATOM | 1628 | N | PHE | A | 188 | 54.192 | 32.734 | 31.101 | 1.00 | 23.48 | N |
| ATOM | 1629 | CA | PHE | A | 188 | 53.604 | 33.399 | 32.259 | 1.00 | 21.24 | C |
| ATOM | 1630 | C | PHE | A | 188 | 54.638 | 34.080 | 33.095 | 1.00 | 21.39 | C |
| ATOM | 1631 | O | PHE | A | 188 | 54.394 | 35.126 | 33.626 | 1.00 | 23.90 | O |
| ATOM | 1632 | CB | PHE | A | 188 | 52.723 | 32.466 | 33.077 | 1.00 | 19.95 | C |
| ATOM | 1633 | CG | PHE | A | 188 | 51.389 | 32.215 | 32.435 | 1.00 | 22.28 | C |
| ATOM | 1634 | CD1 | PHE | A | 188 | 50.440 | 33.229 | 32.375 | 1.00 | 19.42 | C |
| ATOM | 1635 | CD2 | PHE | A | 188 | 51.144 | 31.038 | 31.734 | 1.00 | 23.82 | C |
| ATOM | 1636 | CE1 | PHE | A | 188 | 49.191 | 33.026 | 31.742 | 1.00 | 24.77 | C |
| ATOM | 1637 | CE2 | PHE | A | 188 | 49.936 | 30.826 | 31.057 | 1.00 | 20.17 | C |
| ATOM | 1638 | CZ | PHE | A | 188 | 48.945 | 31.815 | 31.068 | 1.00 | 23.14 | C |
| ATOM | 1639 | N | HIS | A | 189 | 55.831 | 33.513 | 33.118 | 1.00 | 24.15 | N |
| ATOM | 1640 | CA | HIS | A | 189 | 56.933 | 34.122 | 33.837 | 1.00 | 28.79 | C |
| ATOM | 1641 | C | HIS | A | 189 | 57.303 | 35.506 | 33.315 | 1.00 | 28.58 | C |
| ATOM | 1642 | O | HIS | A | 189 | 57.480 | 36.463 | 34.083 | 1.00 | 20.07 | O |
| ATOM | 1643 | CB | HIS | A | 189 | 58.148 | 33.268 | 33.641 | 1.00 | 31.38 | C |
| ATOM | 1644 | CG | HIS | A | 189 | 59.364 | 33.844 | 34.290 | 1.00 | 29.98 | C |
| ATOM | 1645 | ND1 | HIS | A | 189 | 59.548 | 33.833 | 35.658 | 1.00 | 31.00 | N |
| ATOM | 1646 | CD2 | HIS | A | 189 | 60.449 | 34.464 | 33.766 | 1.00 | 21.79 | C |
| ATOM | 1647 | CE1 | HIS | A | 189 | 60.722 | 34.371 | 35.945 | 1.00 | 24.04 | C |
| ATOM | 1648 | NE2 | HIS | A | 189 | 61.257 | 34.815 | 34.821 | 1.00 | 19.53 | N |
| ATOM | 1649 | N | GLU | A | 190 | 57.539 | 35.561 | 32.006 | 1.00 | 28.43 | N |
| ATOM | 1650 | CA | GLU | A | 190 | 57.876 | 36.816 | 31.324 | 1.00 | 27.72 | C |
| ATOM | 1651 | C | GLU | A | 190 | 56.725 | 37.829 | 31.437 | 1.00 | 32.56 | C |
| ATOM | 1652 | O | GLU | A | 190 | 56.949 | 38.995 | 31.717 | 1.00 | 27.06 | O |
| ATOM | 1653 | CB | GLU | A | 190 | 58.122 | 36.529 | 29.849 | 1.00 | 28.55 | C |
| ATOM | 1654 | CG | GLU | A | 190 | 59.150 | 35.461 | 29.614 | 1.00 | 35.29 | C |
| ATOM | 1655 | CD | GLU | A | 190 | 60.553 | 35.941 | 29.892 | 1.00 | 99.81 | C |
| ATOM | 1656 | OH1 | GLU | A | 190 | 60.913 | 36.037 | 31.085 | 1.00 | 86.56 | O |
| ATOM | 1657 | OE2 | GLU | A | 190 | 61.293 | 36.167 | 28.910 | 1.00 | 100.00 | O |
| ATOM | 1658 | N | ALA | A | 191 | 55.493 | 37.391 | 31.196 | 1.00 | 32.67 | N |
| ATOM | 1659 | CA | ALA | A | 191 | 54.349 | 38.286 | 31.311 | 1.00 | 25.30 | C |
| ATOM | 1660 | C | ALA | A | 191 | 54.287 | 38.795 | 32.742 | 1.00 | 36.20 | C |
| ATOM | 1661 | O | ALA | A | 191 | 53.920 | 39.924 | 33.014 | 1.00 | 27.52 | O |
| ATOM | 1662 | CB | ALA | A | 191 | 53.055 | 37.563 | 31.000 | 1.00 | 16.48 | C |
| ATOM | 1663 | N | THR | A | 192 | 54.549 | 37.927 | 33.693 | 1.00 | 29.39 | N |
| ATOM | 1664 | CA | THR | A | 192 | 54.395 | 38.386 | 35.041 | 1.00 | 19.08 | C |
| ATOM | 1665 | C | THR | A | 192 | 55.420 | 39.494 | 35.298 | 1.00 | 44.78 | C |
| ATOM | 1666 | O | THR | A | 192 | 55.094 | 40.550 | 35.839 | 1.00 | 40.58 | O |
| ATOM | 1667 | CB | THR | A | 192 | 54.515 | 37.235 | 35.983 | 1.00 | 18.99 | C |
| ATOM | 1668 | OG1 | THR | A | 192 | 53.410 | 36.348 | 35.755 | 1.00 | 34.36 | O |
| ATOM | 1669 | CG2 | THR | A | 192 | 54.461 | 37.738 | 37.425 | 1.00 | 21.15 | C |
| ATOM | 1670 | N | ALA | A | 193 | 56.617 | 39.312 | 34.757 | 1.00 | 48.58 | N |
| ATOM | 1671 | CA | ALA | A | 193 | 57.705 | 40.286 | 34.905 | 1.00 | 50.59 | C |
| ATOM | 1672 | C | ALA | A | 193 | 57.496 | 41.613 | 34.145 | 1.00 | 54.42 | C |
| ATOM | 1673 | O | ALA | A | 193 | 57.952 | 42.698 | 34.553 | 1.00 | 48.28 | O |
| ATOM | 1674 | CB | ALA | A | 193 | 59.0473 | 9.640 | 34.496 | 1.00 | 51.78 | C |
| ATOM | 1675 | N | GLN | A | 194 | 56.810 | 41.530 | 33.022 | 1.00 | 43.16 | N |
| ATOM | 1676 | CA | GLN | A | 194 | 56.586 | 42.722 | 32.242 | 1.00 | 38.03 | C |
| ATOM | 1677 | C | GLN | A | 194 | 55.264 | 43.389 | 32.576 | 1.00 | 40.85 | C |
| ATOM | 1678 | O | GLN | A | 194 | 54.830 | 44.284 | 31.845 | 1.00 | 51.20 | O |
| ATOM | 1679 | CB | GLN | A | 194 | 56.599 | 42.358 | 30.750 | 1.00 | 35.96 | C |
| ATOM | 1680 | CG | GLN | A | 194 | 57.910 | 41.692 | 30.290 | 1.00 | 100.00 | C |
| ATOM | 1681 | CD | GLN | A | 194 | 57.715 | 40.661 | 29.158 | 1.00 | 100.00 | C |
| ATOM | 1682 | OE1 | GLN | A | 194 | 56.619 | 40.546 | 28.579 | 1.00 | 100.00 | O |
| ATOM | 1683 | NE2 | GLN | A | 194 | 58.782 | 39.904 | 28.848 | 1.00 | 100.00 | N |
| ATOM | 1684 | N | GLY | A | 195 | 54.583 | 42.949 | 33.630 | 1.00 | 32.29 | N |
| ATOM | 1685 | CA | GLY | A | 195 | 53.236 | 43.464 | 33.864 | 1.00 | 36.26 | C |
| ATOM | 1686 | C | GLY | A | 195 | 52.299 | 43.332 | 32.593 | 1.00 | 45.33 | C |
| ATOM | 1687 | O | GLY | A | 195 | 51.515 | 44.242 | 32.346 | 1.00 | 45.16 | O |
| ATOM | 1688 | N | GLY | A | 196 | 52.405 | 42.245 | 31.788 | 1.00 | 36.33 | N |
| ATOM | 1689 | CA | GLY | A | 196 | 51.515 | 41.965 | 30.608 | 1.00 | 19.06 | C |
| ATOM | 1690 | C | GLY | A | 196 | 50.037 | 41.958 | 31.117 | 1.00 | 22.49 | C |
| ATOM | 1691 | O | GLY | A | 196 | 49.724 | 41.479 | 32.223 | 1.00 | 33.09 | O |
| ATOM | 1692 | N | PRO | A | 197 | 49.144 | 42.657 | 30.431 | 1.00 | 29.22 | N |
| ATOM | 1693 | CA | PRO | A | 197 | 47.790 | 42.732 | 30.953 | 1.00 | 25.29 | C |
| ATOM | 1694 | C | PRO | A | 197 | 47.091 | 41.413 | 30.674 | 1.00 | 24.64 | C |
| ATOM | 1695 | O | PRO | A | 197 | 46.192 | 40.991 | 31.411 | 1.00 | 24.75 | O |
| ATOM | 1696 | CB | PRO | A | 197 | 47.162 | 43.911 | 30.176 | 1.00 | 26.31 | C |
| ATOM | 1697 | CG | PRO | A | 197 | 48.188 | 44.407 | 29.252 | 1.00 | 26.56 | C |
| ATOM | 1698 | CD | PRO | A | 197 | 49.307 | 43.454 | 29.203 | 1.00 | 30.25 | C |
| ATOM | 1699 | N | ASP | A | 198 | 47.572 | 40.723 | 29.658 | 1.00 | 16.88 | N |
| ATOM | 1700 | CA | ASP | A | 198 | 47.067 | 39.418 | 29.405 | 1.00 | 21.65 | C |
| ATOM | 1701 | C | ASP | A | 198 | 48.046 | 38.522 | 28.677 | 1.00 | 31.28 | C |
| ATOM | 1702 | O | ASP | A | 198 | 49.062 | 38.978 | 28.172 | 1.00 | 34.57 | O |
| ATOM | 1703 | CB | ASP | A | 198 | 45.739 | 39.507 | 28.669 | 1.00 | 32.80 | C |
| ATOM | 1704 | CG | ASP | A | 198 | 45.868 | 40.055 | 27.256 | 1.00 | 46.13 | C |
| ATOM | 1705 | OD1 | ASP | A | 198 | 46.982 | 40.230 | 26.725 | 1.00 | 57.45 | O |
| ATOM | 1706 | OD2 | ASP | A | 198 | 44.817 | 40.271 | 26.640 | 1.00 | 67.61 | O |
| ATOM | 1707 | N | VAL | A | 199 | 47.713 | 37.234 | 28.614 | 1.00 | 38.67 | N |
| ATOM | 1708 | CA | VAL | A | 199 | 48.499 | 36.226 | 27.901 | 1.00 | 27.79 | C |
| ATOM | 1709 | C | VAL | A | 199 | 47.462 | 35.469 | 27.065 | 1.00 | 25.88 | C |
| ATOM | 1710 | O | VAL | A | 199 | 46.460 | 35.023 | 27.598 | 1.00 | 24.22 | O |
| ATOM | 1711 | CB | VAL | A | 199 | 49.163 | 35.229 | 28.905 | 1.00 | 24.37 | C |
| ATOM | 1712 | CG1 | VAL | A | 199 | 49.874 | 34.047 | 28.160 | 1.00 | 20.28 | C |
| ATOM | 1713 | CG2 | VAL | A | 199 | 50.121 | 35.942 | 29.835 | 1.00 | 22.25 | C |
| ATOM | 1714 | N | VAL | A | 200 | 47.661 | 35.386 | 25.757 | 1.00 | 23.72 | N |
| ATOM | 1715 | CA | VAL | A | 200 | 46.701 | 34.694 | 24.903 | 1.00 | 23.99 | C |
| ATOM | 1716 | C | VAL | A | 200 | 47.167 | 33.286 | 24.499 | 1.00 | 22.85 | C |
| ATOM | 1717 | O | VAL | A | 200 | 48.321 | 33.108 | 24.188 | 1.00 | 29.77 | O |
| ATOM | 1718 | CB | VAL | A | 200 | 46.358 | 35.548 | 23.680 | 1.00 | 23.11 | C |
| ATOM | 1719 | CG1 | VAL | A | 200 | 45.561 | 34.737 | 22.598 | 1.00 | 16.25 | C |
| ATOM | 1720 | CG2 | VAL | A | 200 | 45.652 | 36.823 | 24.130 | 1.00 | 27.86 | C |
| ATOM | 1721 | N | VAL | A | 201 | 46.296 | 32.278 | 24.632 | 1.00 | 27.39 | N |
| ATOM | 1722 | CA | VAL | A | 201 | 46.588 | 30.893 | 24.265 | 1.00 | 9.63 | C |
| ATOM | 1723 | C | VAL | A | 201 | 45.653 | 30.529 | 23.165 | 1.00 | 19.63 | C |
| ATOM | 1724 | O | VAL | A | 201 | 44.452 | 30.755 | 23.312 | 1.00 | 17.61 | O |
| ATOM | 1725 | CB | VAL | A | 201 | 46.306 | 29.952 | 25.426 | 1.00 | 19.95 | C |
| ATOM | 1726 | CG1 | VAL | A | 201 | 46.703 | 28.519 | 25.054 | 1.00 | 20.85 | C |
| ATOM | 1727 | CG2 | VAL | A | 201 | 47.086 | 30.439 | 26.661 | 1.00 | 16.73 | C |
| ATOM | 1728 | N | TRP | A | 202 | 46.210 | 30.080 | 22.030 | 1.00 | 14.36 | N |
| ATOM | 1729 | CA | TRP | A | 202 | 45.422 | 29.693 | 20.865 | 1.00 | 18.97 | C |
| ATOM | 1730 | C | TRP | A | 202 | 44.495 | 28.572 | 21.313 | 1.00 | 36.22 | C |
| ATOM | 1731 | O | TRP | A | 202 | 44.934 | 27.694 | 22.057 | 1.00 | 31.46 | O |
| ATOM | 1732 | CB | TRP | A | 202 | 46.292 | 29.055 | 19.823 | 1.00 | 19.14 | C |
| ATOM | 1733 | CG | TRP | A | 202 | 47.243 | 29.894 | 19.066 | 1.00 | 33.65 | C |
| ATOM | 1734 | CD1 | TRP | A | 202 | 48.391 | 29.463 | 18.429 | 1.00 | 35.28 | C |
| ATOM | 1735 | CD2 | TRP | A | 202 | 47.126 | 31.282 | 18.772 | 1.00 | 39.90 | C |
| ATOM | 1736 | NE1 | TRP | A | 202 | 48.941 | 30.481 | 17.693 | 1.00 | 37.86 | N |
| ATOM | 1737 | CE2 | TRP | A | 202 | 48.228 | 31.624 | 17.922 | 1.00 | 38.35 | C |
| ATOM | 1738 | CE3 | TRP | A | 202 | 46.206 | 32.281 | 19.138 | 1.00 | 39.39 | C |
| ATOM | 1739 | CZ2 | TRP | A | 202 | 48.380 | 32.884 | 17.367 | 1.00 | 36.15 | C |
| ATOM | 1740 | CZ3 | TRP | A | 202 | 46.356 | 33.542 | 18.578 | 1.00 | 39.60 | C |
| ATOM | 1741 | CH2 | TRP | A | 202 | 47.428 | 33.828 | 17.684 | 1.00 | 40.99 | C |
| ATOM | 1742 | N | GLY | A | 203 | 43.245 | 28.564 | 20.842 | 1.00 | 25.59 | N |
| ATOM | 1743 | CA | GLY | A | 203 | 42.332 | 27.483 | 21.169 | 1.00 | 13.09 | C |
| ATOM | 1744 | C | GLY | A | 203 | 41.260 | 27.813 | 22.193 | 1.00 | 21.12 | C |
| ATOM | 1745 | O | GLY | A | 203 | 41.340 | 28.815 | 22.886 | 1.00 | 22.86 | O |
| ATOM | 1746 | N | SER | A | 204 | 40.270 | 26.919 | 22.262 | 1.00 | 16.88 | N |
| ATOM | 1747 | CA | SER | A | 204 | 39.163 | 26.979 | 23.192 | 1.00 | 18.36 | C |
| ATOM | 1748 | C | SER | A | 204 | 39.561 | 26.664 | 24.659 | 1.00 | 22.07 | C |
| ATOM | 1749 | O | SER | A | 204 | 38.888 | 27.096 | 25.604 | 1.00 | 34.39 | O |
| ATOM | 1750 | CB | SER | A | 204 | 38.053 | 25.998 | 22.740 | 1.00 | 9.99 | C |
| ATOM | 1751 | OG | SER | A | 204 | 38.237 | 24.695 | 23.291 | 1.00 | 16.37 | O |
| ATOM | 1752 | N | GLY | A | 205 | 40.562 | 25.813 | 24.854 | 1.00 | 12.42 | N |
| ATOM | 1753 | CA | GLY | A | 205 | 40.963 | 25.411 | 26.208 | 1.00 | 11.64 | C |
| ATOM | 1754 | C | GLY | A | 205 | 40.208 | 24.178 | 26.711 | 1.00 | 19.49 | C |
| ATOM | 1755 | O | GLY | A | 205 | 40.422 | 23.723 | 27.838 | 1.00 | 13.59 | O |
| ATOM | 1756 | N | THR | A | 206 | 39.292 | 23.683 | 25.881 | 1.00 | 15.38 | N |
| ATOM | 1757 | CA | THR | A | 206 | 38.432 | 22.594 | 26.281 | 1.00 | 10.80 | C |
| ATOM | 1758 | C | THR | A | 206 | 39.056 | 21.221 | 26.154 | 1.00 | 26.39 | C |
| ATOM | 1759 | O | THR | A | 206 | 38.564 | 20.267 | 26.737 | 1.00 | 23.28 | O |
| ATOM | 1760 | CB | THR | A | 206 | 37.124 | 22.562 | 25.460 | 1.00 | 12.86 | C |
| ATOM | 1761 | OG1 | THR | A | 206 | 37.438 | 22.395 | 24.082 | 1.00 | 13.12 | O |
| ATOM | 1762 | CG2 | THR | A | 206 | 36.348 | 23.840 | 25.620 | 1.00 | 10.62 | C |
| ATOM | 1763 | N | PRO | A | 207 | 40.101 | 21.083 | 25.354 | 1.00 | 21.10 | N |
| ATOM | 1764 | CA | PRO | A | 207 | 40.658 | 19.743 | 25.175 | 1.00 | 18.15 | C |
| ATOM | 1765 | C | PRO | A | 207 | 41.316 | 19.181 | 26.423 | 1.00 | 21.75 | C |
| ATOM | 1766 | O | PRO | A | 207 | 41.951 | 19.925 | 27.215 | 1.00 | 20.65 | O |
| ATOM | 1767 | CB | PRO | A | 207 | 41.638 | 19.909 | 24.013 | 1.00 | 17.51 | C |
| ATOM | 1768 | CG | PRO | A | 207 | 41.146 | 21.213 | 23.307 | 1.00 | 21.45 | C |
| ATOM | 1769 | CD | PRO | A | 207 | 40.698 | 22.062 | 24.431 | 1.00 | 23.44 | C |
| ATOM | 1770 | N | MET | A | 208 | 41.112 | 17.876 | 26.624 | 1.00 | 15.60 | N |
| ATOM | 1771 | CA | MET | A | 208 | 41.694 | 17.167 | 27.775 | 1.00 | 22.94 | C |
| ATOM | 1772 | C | MET | A | 208 | 43.058 | 16.427 | 27.579 | 1.00 | 21.90 | C |
| ATOM | 1773 | O | MET | A | 208 | 43.248 | 15.677 | 26.633 | 1.00 | 23.16 | O |
| ATOM | 1774 | CB | MET | A | 208 | 40.645 | 16.273 | 28.386 | 1.00 | 32.86 | C |
| ATOM | 1775 | CG | MET | A | 208 | 39.630 | 17.057 | 29.223 | 1.00 | 46.17 | C |
| ATOM | 1776 | SD | MET | A | 208 | 38.301 | 15.990 | 29.826 | 1.00 | 57.85 | S |
| ATOM | 1777 | CE | MET | A | 208 | 37.999 | 15.028 | 28.343 | 1.00 | 58.23 | C |
| ATOM | 1778 | N | ARG | A | 209 | 44.022 | 16.681 | 28.456 | 1.00 | 17.75 | N |
| ATOM | 1779 | CA | ARG | A | 209 | 45.318 | 16.042 | 28.324 | 1.00 | 19.88 | C |
| ATOM | 1780 | C | ARG | A | 209 | 45.871 | 15.534 | 29.639 | 1.00 | 16.92 | C |
| ATOM | 1781 | O | ARG | A | 209 | 45.433 | 15.946 | 30.697 | 1.00 | 16.58 | O |
| ATOM | 1782 | CB | ARG | A | 209 | 46.340 | 16.963 | 27.658 | 1.00 | 21.07 | C |
| ATOM | 1783 | CG | ARG | A | 209 | 45.980 | 17.478 | 26.275 | 1.00 | 22.57 | C |
| ATOM | 1784 | CD | ARG | A | 209 | 45.833 | 16.357 | 25.282 | 1.00 | 28.26 | C |
| ATOM | 1785 | NE | ARG | A | 209 | 45.586 | 16.819 | 23.906 | 1.00 | 23.15 | N |
| ATOM | 1786 | CZ | ARG | A | 209 | 44.420 | 16.742 | 23.267 | 1.00 | 34.52 | C |
| ATOM | 1787 | NH1 | ARG | A | 209 | 43.336 | 16.267 | 23.890 | 1.00 | 18.03 | N |
| ATOM | 1788 | NE2 | ARG | A | 209 | 44.339 | 17.175 | 22.012 | 1.00 | 29.78 | N |
| ATOM | 1789 | N | GLU | A | 210 | 46.878 | 14.675 | 29.547 | 1.00 | 20.87 | N |
| ATOM | 1790 | CA | GLU | A | 210 | 47.530 | 14.079 | 30.720 | 1.00 | 17.37 | C |
| ATOM | 1791 | C | GLU | A | 210 | 49.031 | 14.490 | 30.851 | 1.00 | 20.96 | C |
| ATOM | 1792 | O | GLU | A | 210 | 49.748 | 14.622 | 29.841 | 1.00 | 22.44 | O |
| ATOM | 1793 | CB | GLU | A | 210 | 47.400 | 12.562 | 30.571 | 1.00 | 16.26 | C |
| ATOM | 1794 | CG | GLU | A | 210 | 47.807 | 11.785 | 31.809 | 1.00 | 19.91 | C |
| ATOM | 1795 | CD | GLU | A | 210 | 48.057 | 10.304 | 31.531 | 1.00 | 27.81 | C |
| ATOM | 1796 | OE1 | GLU | A | 210 | 48.111 | 9.919 | 30.343 | 1.00 | 17.29 | O |
| ATOM | 1797 | OE2 | GLU | A | 210 | 48.268 | 9.540 | 32.494 | 1.00 | 21.63 | O |
| ATOM | 1798 | N | PHE | A | 211 | 49.504 | 14.712 | 32.084 | 1.00 | 14.02 | N |
| ATOM | 1799 | CA | PHE | A | 211 | 50.887 | 15.159 | 32.353 | 1.00 | 17.48 | C |
| ATOM | 1800 | C | PHE | A | 211 | 51.458 | 14.414 | 33.531 | 1.00 | 33.62 | C |
| ATOM | 1801 | O | PHE | A | 211 | 50.716 | 14.031 | 34.443 | 1.00 | 27.96 | O |
| ATOM | 1802 | CB | PHE | A | 211 | 50.933 | 16.677 | 32.644 | 1.00 | 17.78 | C |
| ATOM | 1803 | CG | PHE | A | 211 | 50.303 | 17.490 | 31.541 | 1.00 | 21.49 | C |
| ATOM | 1804 | CD1 | PHE | A | 211 | 51.009 | 17.676 | 30.320 | 1.00 | 17.36 | C |
| ATOM | 1805 | CD2 | PHE | A | 211 | 48.933 | 17.844 | 31.618 | 1.00 | 15.09 | C |
| ATOM | 1806 | CE1 | PHE | A | 211 | 50.399 | 18.334 | 29.237 | 1.00 | 16.37 | C |
| ATOM | 1807 | CE2 | PHE | A | 211 | 48.288 | 18.491 | 30.533 | 1.00 | 9.61 | C |
| ATOM | 1808 | CZ | PHE | A | 211 | 49.053 | 18.756 | 29.344 | 1.00 | 12.71 | C |
| ATOM | 1809 | N | LEU | A | 212 | 52.761 | 14.161 | 33.495 | 1.00 | 23.76 | N |
| ATOM | 1810 | CA | LEU | A | 212 | 53.405 | 13.448 | 34.603 | 1.00 | 21.24 | C |
| ATOM | 1811 | C | LEU | A | 212 | 54.772 | 14.053 | 34.898 | 1.00 | 14.00 | C |
| ATOM | 1812 | O | LEU | A | 212 | 55.519 | 14.398 | 33.985 | 1.00 | 13.99 | O |
| ATOM | 1813 | CB | LEU | A | 212 | 53.548 | 11.954 | 34.294 | 1.00 | 21.52 | C |
| ATOM | 1814 | CG | LEU | A | 212 | 54.033 | 11.039 | 35.406 | 1.00 | 21.09 | C |
| ATOM | 1815 | CD1 | LEU | A | 212 | 52.866 | 10.634 | 36.280 | 1.00 | 20.84 | C |
| ATOM | 1816 | CD2 | LEU | A | 212 | 54.768 | 9.829 | 34.832 | 1.00 | 13.18 | C |
| ATOM | 1817 | N | HIS | A | 213 | 55.023 | 14.302 | 36.175 | 1.00 | 9.60 | N |
| ATOM | 1818 | CA | HIS | A | 213 | 56.290 | 14.864 | 36.555 | 1.00 | 13.66 | C |
| ATOM | 1819 | C | HIS | A | 213 | 57.380 | 13.828 | 36.293 | 1.00 | 20.37 | C |
| ATOM | 1820 | O | HIS | A | 213 | 57.238 | 12.614 | 36.542 | 1.00 | 16.08 | O |
| ATOM | 1821 | CB | HIS | A | 213 | 56.280 | 15.250 | 38.002 | 1.00 | 18.72 | C |
| ATOM | 1822 | CG | HIS | A | 213 | 57.491 | 16.017 | 38.408 | 1.00 | 21.22 | C |
| ATOM | 1823 | ND1 | HIS | A | 213 | 58.703 | 15.406 | 38.656 | 1.00 | 24.29 | N |
| ATOM | 1824 | CD2 | HIS | A | 213 | 57.716 | 17.353 | 38.499 | 1.00 | 23.67 | C |
| ATOM | 1825 | CE1 | HIS | A | 213 | 59.615 | 16.331 | 38.917 | 1.00 | 19.13 | C |
| ATOM | 1826 | NE2 | HIS | A | 213 | 59.041 | 17.523 | 38.847 | 1.00 | 21.99 | N |
| ATOM | 1827 | N | VAL | A | 214 | 58.459 | 14.295 | 35.698 | 1.00 | 21.07 | N |
| ATOM | 1828 | CA | VAL | A | 214 | 59.532 | 13.383 | 35.361 | 1.00 | 19.23 | C |
| ATOM | 1829 | C | VAL | A | 214 | 60.067 | 12.523 | 36.551 | 1.00 | 27.20 | C |
| ATOM | 1830 | O | VAL | A | 214 | 60.604 | 11.444 | 36.359 | 1.00 | 22.23 | O |
| ATOM | 1831 | CE | VAL | A | 214 | 60.625 | 14.125 | 34.566 | 1.00 | 11.84 | C |
| ATOM | 1832 | CG1 | VAL | A | 214 | 61.390 | 15.199 | 35.485 | 1.00 | 8.52 | C |
| ATOM | 1833 | CG2 | VAL | A | 214 | 61.560 | 13.097 | 33.902 | 1.00 | 12.39 | C |
| ATOM | 1834 | N | ASP | A | 215 | 59.893 | 12.984 | 37.790 | 1.00 | 25.29 | N |
| ATOM | 1835 | CA | ASP | A | 215 | 60.406 | 12.228 | 38.936 | 1.00 | 18.19 | C |
| ATOM | 1836 | C | ASP | A | 215 | 59.530 | 11.023 | 39.230 | 1.00 | 13.85 | C |
| ATOM | 1837 | O | ASP | A | 215 | 59.988 | 9.981 | 39.666 | 1.00 | 17.44 | O |
| ATOM | 1838 | CB | ASP | A | 215 | 60.575 | 13.129 | 40.155 | 1.00 | 16.27 | C |
| ATOM | 1839 | CG | ASP | A | 215 | 61.859 | 13.979 | 40.068 | 1.00 | 30.73 | C |
| ATOM | 1840 | OD1 | ASP | A | 215 | 62.782 | 13.614 | 39.308 | 1.00 | 23.02 | O |
| ATOM | 1841 | OD2 | ASP | A | 215 | 61.957 | 15.029 | 40.730 | 1.00 | 26.00 | O |
| ATOM | 1842 | N | ASP | A | 216 | 58.276 | 11.136 | 38.863 | 1.00 | 20.08 | N |
| ATOM | 1843 | CA | ASP | A | 216 | 57.378 | 10.017 | 39.016 | 1.00 | 18.78 | C |
| ATOM | 1844 | C | ASP | A | 216 | 57.761 | 9.083 | 37.894 | 1.00 | 23.56 | C |
| ATOM | 1845 | O | ASP | A | 216 | 57.715 | 7.880 | 38.026 | 1.00 | 20.79 | O |
| ATOM | 1846 | CB | ASP | A | 216 | 55.912 | 10.457 | 38.821 | 1.00 | 17.18 | C |
| ATOM | 1847 | CG | ASP | A | 216 | 55.193 | 10.757 | 40.162 | 1.00 | 38.03 | C |
| ATOM | 1848 | OD1 | ASP | A | 216 | 55.503 | 10.119 | 41.223 | 1.00 | 26.02 | O |
| ATOM | 1849 | OD2 | ASP | A | 216 | 54.249 | 11.587 | 40.124 | 1.00 | 25.41 | O |
| ATOM | 1850 | N | MET | A | 217 | 58.092 | 9.653 | 36.755 | 1.00 | 18.11 | N |
| ATOM | 1851 | CA | MET | A | 217 | 58.394 | 8.785 | 35.636 | 1.00 | 22.41 | C |
| ATOM | 1852 | C | MET | A | 217 | 59.572 | 7.942 | 35.992 | 1.00 | 27.54 | C |
| ATOM | 1853 | O | MET | A | 217 | 59.579 | 6.752 | 35.710 | 1.00 | 20.86 | O |
| ATOM | 1854 | CB | MET | A | 217 | 58.637 | 9.592 | 34.345 | 1.00 | 21.24 | C |
| ATOM | 1855 | CG | MET | A | 217 | 59.478 | 8.918 | 33.287 | 1.00 | 16.37 | C |
| ATOM | 1856 | SD | MET | A | 217 | 58.962 | 7.412 | 32.473 | 1.00 | 30.51 | S |
| ATOM | 1857 | CE | MET | A | 217 | 57.465 | 7.608 | 32.391 | 1.00 | 19.57 | C |
| ATOM | 1858 | N | ALA | A | 218 | 60.561 | 8.562 | 36.623 | 1.00 | 19.09 | N |
| ATOM | 1859 | CA | ALA | A | 218 | 61.774 | 7.841 | 37.002 | 1.00 | 13.65 | C |
| ATOM | 1860 | C | ALA | A | 218 | 61.436 | 6.778 | 38.028 | 1.00 | 22.61 | C |
| ATOM | 1861 | O | ALA | A | 218 | 61.934 | 5.670 | 37.967 | 1.00 | 19.36 | O |
| ATOM | 1862 | CB | ALA | A | 218 | 62.809 | 8.780 | 37.579 | 1.00 | 12.23 | C |
| ATOM | 1863 | N | ALA | A | 219 | 60.605 | 7.109 | 39.000 | 1.00 | 19.34 | N |
| ATOM | 1864 | CA | ALA | A | 219 | 60.310 | 6.105 | 40.023 | 1.00 | 18.01 | C |
| ATOM | 1865 | C | ALA | A | 219 | 59.630 | 4.901 | 39.413 | 1.00 | 23.57 | C |
| ATOM | 1866 | O | ALA | A | 219 | 59.781 | 3.777 | 39.898 | 1.00 | 22.71 | O |
| ATOM | 1867 | CB | ALA | A | 219 | 59.387 | 6.678 | 41.083 | 1.00 | 10.11 | C |
| ATOM | 1868 | N | ALA | A | 220 | 58.753 | 5.174 | 38.454 | 1.00 | 18.99 | N |
| ATOM | 1869 | CA | ALA | A | 220 | 57.905 | 4.158 | 37.855 | 1.00 | 14.12 | C |
| ATOM | 1870 | C | ALA | A | 220 | 58.753 | 3.213 | 37.034 | 1.00 | 25.33 | C |
| ATOM | 1871 | O | ALA | A | 220 | 58.584 | 2.006 | 37.114 | 1.00 | 20.63 | O |
| ATOM | 1872 | CB | ALA | A | 220 | 56.796 | 4.798 | 37.023 | 1.00 | 8.53 | C |
| ATOM | 1873 | N | SER | A | 221 | 59.770 | 3.772 | 36.379 | 1.00 | 23.92 | N |
| ATOM | 1874 | CA | SER | A | 221 | 60.702 | 3.011 | 35.556 | 1.00 | 18.38 | C |
| ATOM | 1875 | C | SER | A | 221 | 61.537 | 1.989 | 36.353 | 1.00 | 20.90 | C |
| ATOM | 1876 | O | SER | A | 221 | 61.683 | 0.799 | 35.983 | 1.00 | 19.84 | O |
| ATOM | 1877 | CB | SER | A | 221 | 61.604 | 3.985 | 34.804 | 1.00 | 10.67 | C |
| ATOM | 1878 | OG | SER | A | 221 | 60.847 | 4.744 | 33.867 | 1.00 | 15.61 | O |
| ATOM | 1879 | N | ILE | A | 222 | 62.083 | 2.476 | 37.463 | 1.00 | 18.12 | N |
| ATOM | 1880 | CA | ILE | A | 222 | 62.866 | 1.644 | 38.381 | 1.00 | 21.56 | C |
| ATOM | 1881 | C | ILE | A | 222 | 62.020 | 0.554 | 39.068 | 1.00 | 29.10 | C |
| ATOM | 1882 | O | ILE | A | 222 | 62.504 | −0.566 | 39.307 | 1.00 | 19.03 | O |
| ATOM | 1883 | CB | ILE | A | 222 | 63.467 | 2.516 | 39.432 | 1.00 | 24.56 | C |
| ATOM | 1884 | CG1 | ILE | A | 222 | 64.465 | 3.473 | 38.765 | 1.00 | 32.13 | C |
| ATOM | 1885 | CG2 | ILE | A | 222 | 64.129 | 1.671 | 40.500 | 1.00 | 28.26 | C |
| ATOM | 1886 | CD1 | ILE | A | 222 | 64.973 | 4.585 | 39.649 | 1.00 | 15.61 | C |
| ATOM | 1887 | N | HIS | A | 223 | 60.772 | 0.907 | 39.384 | 1.00 | 19.34 | N |
| ATOM | 1888 | CA | HIS | A | 223 | 59.829 | −0.031 | 39.996 | 1.00 | 20.46 | C |
| ATOM | 1889 | C | HIS | A | 223 | 59.599 | −1.097 | 38.964 | 1.00 | 24.82 | C |
| ATOM | 1890 | O | HIS | A | 223 | 59.723 | −2.283 | 39.270 | 1.00 | 24.66 | O |
| ATOM | 1891 | CB | HIS | A | 223 | 58.465 | 0.637 | 40.359 | 1.00 | 19.53 | C |
| ATOM | 1892 | CG | HIS | A | 223 | 57.373 | −0.333 | 40.759 | 1.00 | 28.64 | C |
| ATOM | 1893 | ND1 | HIS | A | 223 | 57.021 | −0.564 | 42.082 | 1.00 | 24.16 | N |
| ATOM | 1894 | CD2 | HIS | A | 223 | 56.497 | −1.062 | 40.004 | 1.00 | 30.39 | C |
| ATOM | 1895 | CE1 | HIS | A | 223 | 55.983 | −1.399 | 42.112 | 1.00 | 30.39 | C |
| ATOM | 1896 | NE2 | HIS | A | 223 | 55.652 | −1.727 | 40.869 | 1.00 | 28.13 | N |
| ATOM | 1897 | N | VAL | A | 224 | 59.354 | −0.684 | 37.725 | 1.00 | 22.06 | N |
| ATOM | 1898 | CA | VAL | A | 224 | 59.111 | −1.657 | 36.652 | 1.00 | 19.15 | C |
| ATOM | 1899 | C | VAL | A | 224 | 60.350 | −2.490 | 36.333 | 1.00 | 25.89 | C |
| ATOM | 1900 | O | VAL | A | 224 | 60.282 | −3.709 | 36.250 | 1.00 | 22.37 | O |
| ATOM | 1901 | CB | VAL | A | 224 | 58.559 | −1.022 | 35.377 | 1.00 | 22.59 | C |
| ATOM | 1902 | CG1 | VAL | A | 224 | 58.512 | −2.050 | 34.231 | 1.00 | 22.61 | C |
| ATOM | 1903 | CG2 | VAL | A | 224 | 57.161 | −0.491 | 35.650 | 1.00 | 23.44 | C |
| ATOM | 1904 | N | MET | A | 225 | 61.499 | −1.838 | 36.255 | 1.00 | 27.83 | N |
| ATOM | 1905 | CA | MET | A | 225 | 62.710 | −2.577 | 36.004 | 1.00 | 23.69 | C |
| ATOM | 1906 | C | MET | A | 225 | 62.896 | −3.678 | 37.071 | 1.00 | 31.95 | C |
| ATOM | 1907 | O | MET | A | 225 | 63.290 | −4.805 | 36.785 | 1.00 | 24.33 | O |
| ATOM | 1908 | CB | MET | A | 225 | 63.902 | −1.604 | 36.056 | 1.00 | 21.34 | C |
| ATOM | 1909 | CG | MET | A | 225 | 65.295 | −2.296 | 35.999 | 1.00 | 17.83 | C |
| ATOM | 1910 | SD | MET | A | 225 | 65.750 | −2.958 | 34.306 | 1.00 | 23.33 | S |
| ATOM | 1911 | CE | MET | A | 225 | 67.080 | −1.896 | 33.785 | 1.00 | 16.46 | C |
| ATOM | 1912 | N | GLU | A | 226 | 62.644 | −3.319 | 38.316 | 1.00 | 19.54 | N |
| ATOM | 1913 | CA | GLU | A | 226 | 62.988 | −4.161 | 39.428 | 1.00 | 21.58 | C |
| ATOM | 1914 | C | GLU | A | 226 | 61.999 | −5.200 | 39.918 | 1.00 | 30.77 | C |
| ATOM | 1915 | O | GLU | A | 226 | 62.308 | −6.012 | 40.780 | 1.00 | 29.39 | O |
| ATOM | 1916 | CB | GLU | A | 226 | 63.613 | −3.323 | 40.547 | 1.00 | 20.47 | C |
| ATOM | 1917 | CG | GLU | A | 226 | 64.937 | −2.673 | 40.122 | 1.00 | 23.03 | C |
| ATOM | 1918 | CD | GLU | A | 226 | 65.504 | −1.809 | 41.208 | 1.00 | 32.62 | C |
| ATOM | 1919 | OE1 | GLU | A | 226 | 64.721 | −1.455 | 42.122 | 1.00 | 26.12 | O |
| ATOM | 1920 | OE2 | GLU | A | 226 | 66.711 | −1.479 | 41.152 | 1.00 | 17.67 | O |
| ATOM | 1921 | N | LEU | A | 227 | 60.837 | −5.248 | 39.295 | 1.00 | 34.11 | N |
| ATOM | 1922 | CA | LEU | A | 227 | 59.883 | −6.296 | 39.642 | 1.00 | 35.26 | C |
| ATOM | 1923 | C | LEU | A | 227 | 60.537 | −7.644 | 39.320 | 1.00 | 27.91 | C |
| ATOM | 1924 | O | LEU | A | 227 | 61.291 | −7.766 | 38.340 | 1.00 | 19.89 | O |
| ATOM | 1925 | CB | LEU | A | 227 | 58.693 | −6.236 | 38.678 | 1.00 | 36.48 | C |
| ATOM | 1926 | CG | LEU | A | 227 | 57.381 | −5.569 | 38.955 | 1.00 | 40.30 | C |
| ATOM | 1927 | CD1 | LEU | A | 227 | 57.697 | −4.194 | 39.382 | 1.00 | 42.04 | C |
| ATOM | 1928 | CD2 | LEU | A | 227 | 56.610 | −5.577 | 37.647 | 1.00 | 46.21 | C |
| ATOM | 1929 | N | ALA | A | 228 | 60.026 | −8.688 | 39.955 | 1.00 | 27.15 | N |
| ATOM | 1930 | CA | ALA | A | 228 | 60.425 | −10.051 | 39.616 | 1.00 | 25.26 | C |
| ATOM | 1931 | C | ALA | A | 228 | 59.801 | −10.435 | 38.279 | 1.00 | 27.93 | C |
| ATOM | 1932 | O | ALA | A | 228 | 58.624 | −10.093 | 37.934 | 1.00 | 31.26 | O |
| ATOM | 1933 | CB | ALA | A | 228 | 60.003 | −11.052 | 40.703 | 1.00 | 22.05 | C |
| ATOM | 1934 | N | HIS | A | 229 | 60.624 | −11.160 | 37.539 | 1.00 | 27.05 | N |
| ATOM | 1935 | CA | HIS | A | 229 | 60.275 | −11.605 | 36.222 | 1.00 | 24.42 | C |
| ATOM | 1936 | C | HIS | A | 229 | 58.905 | −12.260 | 36.184 | 1.00 | 21.74 | C |
| ATOM | 1937 | O | HIS | A | 229 | 58.015 | −11.851 | 35.398 | 1.00 | 22.22 | O |
| ATOM | 1938 | CB | HIS | A | 229 | 61.351 | −12.520 | 35.698 | 1.00 | 17.71 | C |
| ATOM | 1939 | CG | HIS | A | 229 | 61.284 | −12.701 | 34.220 | 1.00 | 27.24 | C |
| ATOM | 1940 | ND1 | HIS | A | 229 | 61.060 | −11.650 | 33.350 | 1.00 | 34.38 | N |
| ATOM | 1941 | CD2 | HIS | A | 229 | 61.292 | −13.821 | 33.465 | 1.00 | 31.45 | C |
| ATOM | 1942 | CB1 | HIS | A | 229 | 60.992 | −12.113 | 32.115 | 1.00 | 30.50 | C |
| ATOM | 1943 | NE2 | HIS | A | 229 | 61.124 | −13.427 | 32.159 | 1.00 | 35.23 | N |
| ATOM | 1944 | N | GLU | A | 230 | 58.681 | −13.161 | 37.140 | 1.00 | 20.24 | N |
| ATOM | 1945 | CA | GLU | A | 230 | 57.425 | −13.895 | 37.209 | 1.00 | 29.41 | C |
| ATOM | 1946 | C | GLU | A | 230 | 56.181 | −13.051 | 37.341 | 1.00 | 22.20 | C |
| ATOM | 1947 | O | GLU | A | 230 | 55.159 | −13.359 | 36.679 | 1.00 | 17.78 | O |
| ATOM | 1948 | CB | GLU | A | 230 | 57.464 | −14.997 | 38.274 | 1.00 | 38.51 | C |
| ATOM | 1949 | CG | GLU | A | 230 | 58.085 | −14.582 | 39.567 | 1.00 | 63.09 | C |
| ATOM | 1950 | CD | GLU | A | 230 | 57.036 | −14.473 | 40.661 | 1.00 | 100.00 | C |
| ATOM | 1951 | OE1 | GLU | A | 230 | 55.859 | −14.872 | 40.400 | 1.00 | 100.00 | O |
| ATOM | 1952 | OE2 | GLU | A | 230 | 57.409 | −14.003 | 41.768 | 1.00 | 81.48 | O |
| ATOM | 1953 | N | VAL | A | 231 | 56.272 | −12.004 | 38.182 | 1.00 | 16.53 | N |
| ATOM | 1954 | CA | VAL | A | 231 | 55.202 | −11.029 | 38.356 | 1.00 | 20.23 | C |
| ATOM | 1955 | C | VAL | A | 231 | 55.009 | −10.164 | 37.102 | 1.00 | 24.45 | C |
| ATOM | 1956 | O | VAL | A | 231 | 53.864 | −9.834 | 36.705 | 1.00 | 2 1.00 | O |
| ATOM | 1957 | CB | VAL | A | 231 | 55.541 | −10.057 | 39.426 | 1.00 | 28.61 | C |
| ATOM | 1958 | CG1 | VAL | A | 231 | 54.362 | −9.098 | 39.610 | 1.00 | 29.78 | C |
| ATOM | 1959 | CG2 | VAL | A | 231 | 55.881 | −10.757 | 40.677 | 1.00 | 28.96 | C |
| ATOM | 1960 | N | TRP | A | 232 | 56.133 | −9.798 | 36.486 | 1.00 | 17.17 | N |
| ATOM | 1961 | CA | TRP | A | 232 | 56.052 | −9.044 | 35.262 | 1.00 | 21.52 | C |
| ATOM | 1962 | C | TRP | A | 232 | 55.388 | −9.844 | 34.156 | 1.00 | 20.53 | C |
| ATOM | 1963 | O | TRP | A | 232 | 54.588 | −9.306 | 33.380 | 1.00 | 24.31 | O |
| ATOM | 1964 | CB | TRP | A | 232 | 57.438 | −8.644 | 34.801 | 1.00 | 29.88 | C |
| ATOM | 1965 | CG | TRP | A | 232 | 57.430 | −7.843 | 33.500 | 1.00 | 27.65 | C |
| ATOM | 1966 | CD1 | TRP | A | 232 | 57.184 | −6.464 | 33.356 | 1.00 | 25.42 | C |
| ATOM | 1967 | CD2 | TRP | A | 232 | 57.714 | −8.336 | 32.169 | 1.00 | 27.75 | C |
| ATOM | 1968 | NE1 | TRP | A | 232 | 57.325 | −6.095 | 32.033 | 1.00 | 22.53 | N |
| ATOM | 1969 | CB2 | TRP | A | 232 | 57.655 | −7.203 | 31.279 | 1.00 | 25.11 | C |
| ATOM | 1970 | CB3 | TRP | A | 232 | 58.037 | −9.603 | 31.640 | 1.00 | 22.72 | C |
| ATOM | 1971 | CZ2 | TRP | A | 232 | 57.917 | −7.316 | 29.879 | 1.00 | 17.23 | C |
| ATOM | 1972 | CZ3 | TRP | A | 232 | 58.238 | −9.720 | 30.223 | 1.00 | 25.97 | C |
| ATOM | 1973 | CH2 | TRP | A | 232 | 58.154 | −8.581 | 29.368 | 1.00 | 22.07 | C |
| ATOM | 1974 | N | LEU | A | 233 | 55.749 | −11.121 | 34.018 | 1.00 | 23.80 | N |
| ATOM | 1975 | CA | LEU | A | 233 | 55.141 | −11.949 | 32.937 | 1.00 | 24.78 | C |
| ATOM | 1976 | C | LEU | A | 233 | 53.652 | −12.118 | 33.122 | 1.00 | 24.51 | C |
| ATOM | 1977 | O | LEU | A | 233 | 52.865 | −12.075 | 32.163 | 1.00 | 28.50 | O |
| ATOM | 1978 | CB | LEU | A | 233 | 55.765 | −13.348 | 32.820 | 1.00 | 26.20 | C |
| ATOM | 1979 | CG | LEU | A | 233 | 57.250 | −13.505 | 32.503 | 1.00 | 19.39 | C |
| ATOM | 1980 | CD1 | LEU | A | 233 | 57.745 | −14.850 | 33.023 | 1.00 | 19.90 | C |
| ATOM | 1981 | CD2 | LEU | A | 233 | 57.561 | −13.287 | 31.017 | 1.00 | 16.01 | C |
| ATOM | 1982 | N | GLU | A | 234 | 53.298 | −12.343 | 34.372 | 1.00 | 25.45 | N |
| ATOM | 1983 | CA | GLU | A | 234 | 51.929 | −12.523 | 34.822 | 1.00 | 30.04 | C |
| ATOM | 1984 | C | GLU | A | 234 | 51.128 | −11.319 | 34.367 | 1.00 | 35.69 | C |
| ATOM | 1985 | O | GLU | A | 234 | 49.926 | −11.390 | 34.052 | 1.00 | 28.25 | O |
| ATOM | 1986 | CB | GLU | A | 234 | 52.007 | −12.468 | 36.344 | 1.00 | 37.30 | C |
| ATOM | 1987 | CG | GLU | A | 234 | 50.908 | −13.133 | 37.118 | 1.00 | 45.39 | C |
| ATOM | 1988 | CD | GLU | A | 234 | 51.112 | −12.881 | 38.601 | 1.00 | 100.00 | C |
| ATOM | 1989 | OE1 | GLU | A | 234 | 52.240 | −13.137 | 39.104 | 1.00 | 99.09 | O |
| ATOM | 1990 | OE2 | GLU | A | 234 | 50.211 | −12.257 | 39.211 | 1.00 | 100.00 | O |
| ATOM | 1991 | N | ASN | A | 235 | 51.802 | −10.184 | 34.364 | 1.00 | 25.04 | N |
| ATOM | 1992 | CA | ASN | A | 235 | 51.109 | −8.986 | 33.992 | 1.00 | 26.17 | C |
| ATOM | 1993 | C | ASN | A | 235 | 51.280 | −8.494 | 32.571 | 1.00 | 30.46 | C |
| ATOM | 1994 | O | ASN | A | 235 | 50.824 | −7.393 | 32.259 | 1.00 | 22.90 | O |
| ATOM | 1995 | CB | ASN | A | 235 | 51.427 | −7.895 | 34.981 | 1.00 | 29.23 | C |
| ATOM | 1996 | CG | ASN | A | 235 | 50.878 | −8.197 | 36.342 | 1.00 | 39.27 | C |
| ATOM | 1997 | OD1 | ASN | A | 235 | 49.722 | −7.882 | 36.628 | 1.00 | 29.06 | O |
| ATOM | 1998 | ND2 | ASN | A | 235 | 51.653 | −8.934 | 37.140 | 1.00 | 40.22 | N |
| ATOM | 1999 | N | THR | A | 236 | 51.935 | −9.268 | 31.708 | 1.00 | 20.97 | N |
| ATOM | 2000 | CA | THR | A | 236 | 52.108 | −8.795 | 30.344 | 1.00 | 22.30 | C |
| ATOM | 2001 | C | THR | A | 236 | 51.867 | −9.943 | 29.419 | 1.00 | 29.74 | C |
| ATOM | 2002 | O | THR | A | 236 | 51.551 | −11.033 | 29.895 | 1.00 | 21.23 | O |
| ATOM | 2003 | CB | THR | A | 236 | 53.545 | −8.306 | 30.161 | 1.00 | 22.73 | C |
| ATOM | 2004 | OG1 | THR | A | 236 | 54.422 | −9.325 | 30.636 | 1.00 | 21.23 | O |
| ATOM | 2005 | CG2 | THR | A | 236 | 53.801 | −7.048 | 31.041 | 1.00 | 19.69 | C |
| ATOM | 2006 | N | GLN | A | 237 | 52.003 | −9.699 | 28.109 | 1.00 | 22.23 | N |
| ATOM | 2007 | CA | GLN | A | 237 | 52.097 | −10.783 | 27.122 | 1.00 | 16.69 | C |
| ATOM | 2008 | C | GLN | A | 237 | 53.335 | −10.507 | 26.331 | 1.00 | 21.02 | C |
| ATOM | 2009 | O | GLN | A | 237 | 53.729 | −9.362 | 26.204 | 1.00 | 22.19 | O |
| ATOM | 2010 | CB | GLN | A | 237 | 50.913 | −10.999 | 26.189 | 1.00 | 8.23 | C |
| ATOM | 2011 | CG | GLN | A | 237 | 49.639 | −11.096 | 26.904 | 1.00 | 21.04 | C |
| ATOM | 2012 | CD | GLN | A | 237 | 48.907 | −9.862 | 26.606 | 1.00 | 62.07 | C |
| ATOM | 2013 | OE1 | GLN | A | 237 | 48.437 | ‘9.712 | 25.460 | 1.00 | 59.32 | O |
| ATOM | 2014 | NE2 | GLN | A | 237 | 49.220 | −8.847 | 27.388 | 1.00 | 37.82 | N |
| ATOM | 2015 | N | PRO | A | 238 | 54.002 | ‘11.579 | 25.917 | 1.00 | 28.76 | N |
| ATOM | 2016 | CA | PRO | A | 238 | 55.275 | −11.438 | 25.246 | 1.00 | 30.28 | C |
| ATOM | 2017 | C | PRO | A | 238 | 55.194 | −10.643 | 23.958 | 1.00 | 29.08 | C |
| ATOM | 2018 | O | PRO | A | 238 | 56.181 | −10.029 | 23.600 | 1.00 | 15.95 | O |
| ATOM | 2019 | CB | PRO | A | 238 | 55.733 | −12.879 | 25.011 | 1.00 | 22.54 | C |
| ATOM | 2020 | CG | PRO | A | 238 | 54.898 | −13.710 | 25.886 | 1.00 | 18.92 | C |
| ATOM | 2021 | CD | PRO | A | 238 | 53.626 | −12.998 | 26.068 | 1.00 | 11.75 | C |
| ATOM | 2022 | N | MET | A | 239 | 54.041 | −10.635 | 23.286 | 1.00 | 17.26 | N |
| ATOM | 2023 | CA | MET | A | 239 | 53.924 | −9.807 | 22.104 | 1.00 | 17.85 | C |
| ATOM | 2024 | C | MET | A | 239 | 53.109 | −8.509 | 22.362 | 1.00 | 18.63 | C |
| ATOM | 2025 | O | MET | A | 239 | 52.792 | −7.741 | 21.419 | 1.00 | 16.82 | O |
| ATOM | 2026 | CB | MET | A | 239 | 53.460 | −10.588 | 20.881 | 1.00 | 15.22 | C |
| ATOM | 2027 | CG | MET | A | 239 | 54.536 | −11.534 | 20.261 | 1.00 | 12.90 | C |
| ATOM | 2028 | SD | MET | A | 239 | 53.994 | −12.534 | 18.808 | 1.00 | 17.49 | S |
| ATOM | 2029 | CE | MET | A | 239 | 54.350 | −11.357 | 17.422 | 1.00 | 13.12 | C |
| ATOM | 2030 | N | LEU | A | 240 | 52.847 | −8.252 | 23.646 | 1.00 | 18.55 | N |
| ATOM | 2031 | CA | LEU | A | 240 | 52.159 | −7.037 | 24.131 | 1.00 | 16.68 | C |
| ATOM | 2032 | C | LEU | A | 240 | 52.774 | −6.733 | 25.493 | 1.00 | 11.82 | C |
| ATOM | 2033 | O | LEU | A | 240 | 52.124 | −6.803 | 26.549 | 1.00 | 13.84 | O |
| ATOM | 2034 | CB | LEU | A | 240 | 50.645 | −7.249 | 24.240 | 1.00 | 16.91 | C |
| ATOM | 2035 | CG | LEU | A | 240 | 49.646 | −6.120 | 23.852 | 1.00 | 22.29 | C |
| ATOM | 2036 | CD1 | LEU | A | 240 | 48.968 | −5.488 | 25.033 | 1.00 | 25.51 | C |
| ATOM | 2037 | CD2 | LEU | A | 240 | 50.070 | −5.059 | 22.815 | 1.00 | 28.07 | C |
| ATOM | 2038 | N | SER | A | 241 | 54.076 | −6.467 | 25.456 | 1.00 | 13.09 | N |
| ATOM | 2039 | CA | SER | A | 241 | 54.842 | −6.315 | 26.682 | 1.00 | 24.20 | C |
| ATOM | 2040 | C | SER | A | 241 | 54.947 | −4.938 | 27.377 | 1.00 | 30.52 | C |
| ATOM | 2041 | O | SER | A | 241 | 55.363 | −4.854 | 28.547 | 1.00 | 17.02 | O |
| ATOM | 2042 | CB | SER | A | 241 | 56.247 | −6.900 | 26.495 | 1.00 | 14.04 | C |
| ATOM | 2043 | OG | SER | A | 241 | 57.062 | −6.144 | 25.598 | 1.00 | 13.95 | O |
| ATOM | 2044 | N | HIS | A | 242 | 54.661 | −3.861 | 26.659 | 1.00 | 17.87 | N |
| ATOM | 2045 | CA | HIS | A | 242 | 54.894 | −2.548 | 27.221 | 1.00 | 13.55 | C |
| ATOM | 2046 | C | HIS | A | 242 | 53.990 | −2.254 | 28.373 | 1.00 | 13.70 | C |
| ATOM | 2047 | O | HIS | A | 242 | 52.974 | −2.885 | 28.539 | 1.00 | 13.29 | O |
| ATOM | 2048 | CB | HIS | A | 242 | 54.826 | −1.430 | 26.130 | 1.00 | 16.05 | C |
| ATOM | 2049 | CG | HIS | A | 242 | 53.595 | −1.504 | 25.272 | 1.00 | 18.88 | C |
| ATOM | 2050 | ND1 | HIS | A | 242 | 52.591 | −0.553 | 25.326 | 1.00 | 23.24 | N |
| ATOM | 2051 | CD2 | HIS | A | 242 | 53.165 | −2.461 | 24.413 | 1.00 | 13.19 | C |
| ATOM | 2052 | CE1 | HIS | A | 242 | 51.629 | −0.887 | 24.483 | 1.00 | 17.44 | C |
| ATOM | 2053 | NE2 | HIS | A | 242 | 51.962 | −2.031 | 23.901 | 1.00 | 19.54 | N |
| ATOM | 2054 | N | ILE | A | 243 | 54.310 | −1.203 | 29.095 | 1.00 | 15.84 | N |
| ATOM | 2055 | CA | ILE | A | 243 | 53.492 | −0.809 | 30.192 | 1.00 | 19.10 | C |
| ATOM | 2056 | C | ILE | A | 243 | 53.336 | 0.714 | 30.191 | 1.00 | 23.23 | C |
| ATOM | 2057 | O | ILE | A | 243 | 54.312 | 1.406 | 30.385 | 1.00 | 12.10 | O |
| ATOM | 2058 | CB | ILE | A | 243 | 54.166 | −1.273 | 31.482 | 1.00 | 24.62 | C |
| ATOM | 2059 | CG1 | ILE | A | 243 | 54.014 | −2.783 | 31.576 | 1.00 | 25.60 | C |
| ATOM | 2060 | CG2 | ILE | A | 243 | 53.497 | −0.665 | 32.735 | 1.00 | 17.37 | C |
| ATOM | 2061 | CD1 | ILE | A | 243 | 54.725 | −3.365 | 32.714 | 1.00 | 14.62 | C |
| ATOM | 2062 | N | ASN | A | 244 | 52.112 | 1.217 | 30.013 | 1.00 | 16.43 | N |
| ATOM | 2063 | CA | ASN | A | 244 | 51.824 | 2.689 | 30.038 | 1.00 | 18.99 | C |
| ATOM | 2064 | C | ASN | A | 244 | 52.252 | 3.292 | 31.348 | 1.00 | 18.83 | C |
| ATOM | 2065 | O | ASN | A | 244 | 51.965 | 2.727 | 32.405 | 1.00 | 19.58 | O |
| ATOM | 2066 | CB | ASN | A | 244 | 50.304 | 2.987 | 29.910 | 1.00 | 15.67 | C |
| ATOM | 2067 | CG | ASN | A | 244 | 49.768 | 2.702 | 28.517 | 1.00 | 14.57 | C |
| ATOM | 2068 | OD1 | ASN | A | 244 | 50.546 | 2.583 | 27.580 | 1.00 | 13.64 | O |
| ATOM | 2069 | ND2 | ASN | A | 244 | 48.443 | 2.491 | 28.393 | 1.00 | 10.16 | N |
| ATOM | 2070 | N | VAL | A | 245 | 52.800 | 4.499 | 31.326 | 1.00 | 13.50 | N |
| ATOM | 2071 | CA | VAL | A | 245 | 53.159 | 5.134 | 32.602 | 1.00 | 13.49 | C |
| ATOM | 2072 | C | VAL | A | 245 | 52.528 | 6.566 | 32.644 | 1.00 | 16.25 | C |
| ATOM | 2073 | O | VAL | A | 245 | 52.786 | 7.405 | 31.770 | 1.00 | 15.20 | O |
| ATOM | 2074 | CB | VAL | A | 245 | 54.754 | 5.163 | 32.810 | 1.00 | 21.07 | C |
| ATOM | 2075 | CG1 | VAL | A | 245 | 55.154 | 6.085 | 33.937 | 1.00 | 15.08 | C |
| ATOM | 2076 | CG2 | VAL | A | 245 | 55.280 | 3.817 | 33.143 | 1.00 | 15.82 | C |
| ATOM | 2077 | N | GLY | A | 246 | 51.696 | 6.843 | 33.649 | 1.00 | 14.03 | N |
| ATOM | 2078 | CA | GLY | A | 246 | 51.027 | 8.136 | 33.707 | 1.00 | 16.87 | C |
| ATOM | 2079 | C | GLY | A | 246 | 50.146 | 8.203 | 34.939 | 1.00 | 26.95 | C |
| ATOM | 2080 | O | GLY | A | 246 | 50.323 | 7.401 | 35.850 | 1.00 | 23.04 | O |
| ATOM | 2081 | N | THR | A | 247 | 49.207 | 9.161 | 34.963 | 1.00 | 21.44 | N |
| ATOM | 2082 | CA | THR | A | 247 | 48.232 | 9.276 | 36.063 | 1.00 | 21.39 | C |
| ATOM | 2083 | C | THR | A | 247 | 46.868 | 8.677 | 35.673 | 1.00 | 24.08 | C |
| ATOM | 2084 | O | THR | A | 247 | 46.069 | 8.306 | 36.508 | 1.00 | 21.03 | O |
| ATOM | 2085 | CB | THR | A | 247 | 47.988 | 10.730 | 36.404 | 1.00 | 22.24 | C |
| ATOM | 2086 | OG1 | THR | A | 247 | 47.409 | 11.389 | 35.265 | 1.00 | 18.62 | O |
| ATOM | 2087 | CG2 | THR | A | 247 | 49.275 | 11.378 | 36.724 | 1.00 | 18.99 | C |
| ATOM | 2088 | N | GLY | A | 248 | 46.583 | 8.651 | 34.384 | 1.00 | 24.95 | N |
| ATOM | 2089 | CA | GLY | A | 248 | 45.319 | 8.143 | 33.924 | 1.00 | 22.61 | C |
| ATOM | 2090 | C | GLY | A | 248 | 44.223 | 9.160 | 34.226 | 1.00 | 21.42 | C |
| ATOM | 2091 | O | GLY | A | 248 | 43.059 | 8.866 | 34.137 | 1.00 | 25.70 | O |
| ATOM | 2092 | N | VAL | A | 249 | 44.615 | 10.386 | 34.521 | 1.00 | 30.72 | N |
| ATOM | 2093 | CA | VAL | A | 249 | 43.673 | 11.464 | 34.827 | 1.00 | 26.09 | C |
| ATOM | 2094 | C | VAL | A | 249 | 43.747 | 12.596 | 33.786 | 1.00 | 32.70 | C |
| ATOM | 2095 | O | VAL | A | 249 | 44.853 | 13.006 | 33.387 | 1.00 | 26.92 | O |
| ATOM | 2096 | CB | VAL | A | 249 | 44.020 | 12.085 | 36.214 | 1.00 | 38.59 | C |
| ATOM | 2097 | CG1 | VAL | A | 249 | 43.225 | 13.324 | 36.470 | 1.00 | 36.11 | C |
| ATOM | 2098 | CG2 | VAL | A | 249 | 43.782 | 11.083 | 37.306 | 1.00 | 41.30 | C |
| ATOM | 2099 | N | ASP | A | 250 | 42.581 | 13.125 | 33.397 | 1.00 | 27.95 | N |
| ATOM | 2100 | CA | ASP | A | 250 | 42.488 | 14.232 | 32.439 | 1.00 | 20.64 | C |
| ATOM | 2101 | C | ASP | A | 250 | 42.611 | 15.581 | 33.155 | 1.00 | 27.63 | C |
| ATOM | 2102 | O | ASP | A | 250 | 42.188 | 15.783 | 34.308 | 1.00 | 26.23 | O |
| ATOM | 2103 | CB | ASP | A | 250 | 41.075 | 14.302 | 31.827 | 1.00 | 23.89 | C |
| ATOM | 2104 | CG | ASP | A | 250 | 40.768 | 13.180 | 30.850 | 1.00 | 39.52 | C |
| ATOM | 2105 | OD1 | ASP | A | 250 | 41.283 | 13.184 | 29.688 | 1.00 | 39.96 | O |
| ATOM | 2106 | OD2 | ASP | A | 250 | 39.767 | 12.501 | 31.153 | 1.00 | 45.34 | O |
| ATOM | 2107 | N | CYS | A | 251 | 43.0291 | 6.566 | 32.388 | 1.00 | 20.12 | N |
| ATOM | 2108 | CA | CYS | A | 251 | 42.962 | 17.906 | 32.851 | 1.00 | 27.20 | C |
| ATOM | 2109 | C | CYS | A | 251 | 42.918 | 18.779 | 31.577 | 1.00 | 26.47 | C |
| ATOM | 2110 | O | CYS | A | 251 | 43.699 | 18.560 | 30.633 | 1.00 | 19.45 | O |
| ATOM | 2111 | CB | CYS | A | 251 | 44.148 | 18.157 | 33.778 | 1.00 | 34.86 | C |
| ATOM | 2112 | SG | CYS | A | 251 | 45.129 | 19.619 | 33.453 | 1.00 | 29.47 | S |
| ATOM | 2113 | N | THR | A | 252 | 41.932 | 19.673 | 31.494 | 1.00 | 14.85 | N |
| ATOM | 2114 | CA | THR | A | 252 | 41.834 | 20.588 | 30.335 | 1.00 | 21.21 | C |
| ATOM | 2115 | C | THR | A | 252 | 42.999 | 21.592 | 30.236 | 1.00 | 20.53 | C |
| ATOM | 2116 | O | THR | A | 252 | 43.657 | 21.926 | 31.249 | 1.00 | 15.24 | O |
| ATOM | 2117 | CB | THR | A | 252 | 40.506 | 21.407 | 30.329 | 1.00 | 32.08 | C |
| ATOM | 2118 | OG1 | THR | A | 252 | 40.460 | 22.304 | 31.447 | 1.00 | 19.26 | O |
| ATOM | 2119 | CG2 | THR | A | 252 | 39.309 | 20.495 | 30.372 | 1.00 | 13.91 | C |
| ATOM | 2120 | N | ILE | A | 253 | 43.228 | 22.095 | 29.024 | 1.00 | 14.81 | N |
| ATOM | 2121 | CA | ILE | A | 253 | 44.264 | 23.118 | 28.812 | 1.00 | 16.90 | C |
| ATOM | 2122 | C | ILE | A | 253 | 43.934 | 24.383 | 29.627 | 1.00 | 23.41 | C |
| ATOM | 2123 | O | ILE | A | 253 | 44.834 | 25.012 | 30.247 | 1.00 | 15.27 | O |
| ATOM | 2124 | CB | ILE | A | 253 | 44.404 | 23.452 | 27.302 | 1.00 | 24.05 | C |
| ATOM | 2125 | CG1 | ILE | A | 253 | 44.862 | 22.200 | 26.561 | 1.00 | 27.33 | C |
| ATOM | 2126 | CG2 | ILE | A | 253 | 45.473 | 24.479 | 27.077 | 1.00 | 9.22 | C |
| ATOM | 2127 | CD1 | ILE | A | 253 | 45.662 | 21.276 | 27.452 | 1.00 | 49.56 | C |
| ATOM | 2128 | N | ARG | A | 254 | 42.637 | 24.709 | 29.707 | 1.00 | 19.56 | N |
| ATOM | 2129 | CA | ARG | A | 254 | 42.228 | 25.865 | 30.522 | 1.00 | 19.41 | C |
| ATOM | 2130 | C | ARG | A | 254 | 42.712 | 25.713 | 31.970 | 1.00 | 18.10 | C |
| ATOM | 2131 | O | ARG | A | 254 | 43.311 | 26.616 | 32.515 | 1.00 | 13.89 | O |
| ATOM | 2132 | CB | ARG | A | 254 | 40.704 | 26.101 | 30.480 | 1.00 | 15.98 | C |
| ATOM | 2133 | CG | ARG | A | 254 | 40.282 | 27.378 | 31.255 | 1.00 | 9.96 | C |
| ATOM | 2134 | CD | ARG | A | 254 | 38.809 | 27.702 | 31.218 | 1.00 | 24.79 | C |
| ATOM | 2135 | NE | ARG | A | 254 | 38.498 | 28.414 | 29.997 | 1.00 | 29.42 | N |
| ATOM | 2136 | CZ | ARG | A | 254 | 38.693 | 29.723 | 29.794 | 1.00 | 59.85 | C |
| ATOM | 2137 | NH1 | ARG | A | 254 | 39.194 | 30.527 | 30.732 | 1.00 | 42.58 | N |
| ATOM | 2138 | NH2 | ARG | A | 254 | 38.377 | 30.245 | 28.620 | 1.00 | 18.44 | N |
| ATOM | 2139 | N | ASP | A | 255 | 42.406 | 24.564 | 32.586 | 1.00 | 20.22 | N |
| ATOM | 2140 | CA | ASP | A | 255 | 42.795 | 24.205 | 33.974 | 1.00 | 16.48 | C |
| ATOM | 2141 | C | ASP | A | 255 | 44.321 | 24.372 | 34.069 | 1.00 | 22.43 | C |
| ATOM | 2142 | O | ASP | A | 255 | 44.868 | 24.897 | 35.060 | 1.00 | 18.53 | O |
| ATOM | 2143 | CB | ASP | A | 255 | 42.478 | 22.686 | 34.157 | 1.00 | 19.17 | C |
| ATOM | 2144 | CG | ASP | A | 255 | 42.144 | 22.246 | 35.610 | 1.00 | 47.08 | C |
| ATOM | 2145 | OD1 | ASP | A | 255 | 41.780 | 23.090 | 36.429 | 1.00 | 49.66 | O |
| ATOM | 2146 | OD2 | ASP | A | 255 | 42.020 | 21.016 | 35.880 | 1.00 | 48.12 | O |
| ATOM | 2147 | N | LEU | A | 256 | 45.014 | 23.809 | 33.078 | 1.00 | 15.98 | N |
| ATOM | 2148 | CA | LEU | A | 256 | 46.465 | 23.844 | 33.069 | 1.00 | 21.76 | C |
| ATOM | 2149 | C | LEU | A | 256 | 47.020 | 25.275 | 33.076 | 1.00 | 16.79 | C |
| ATOM | 2150 | O | LEU | A | 256 | 47.825 | 25.697 | 33.946 | 1.00 | 15.24 | O |
| ATOM | 2151 | CB | LEU | A | 256 | 46.967 | 23.056 | 31.859 | 1.00 | 23.33 | C |
| ATOM | 2152 | CG | LEU | A | 256 | 48.491 | 23.100 | 31.765 | 1.00 | 26.80 | C |
| ATOM | 2153 | CD1 | LEU | A | 256 | 49.171 | 22.334 | 32.984 | 1.00 | 17.13 | C |
| ATOM | 2154 | CD2 | LEU | A | 256 | 49.040 | 22.724 | 30.346 | 1.00 | 15.42 | C |
| ATOM | 2155 | N | ALA | A | 257 | 46.520 | 26.048 | 32.140 | 1.00 | 13.77 | N |
| ATOM | 2156 | CA | ALA | A | 257 | 46.938 | 27.436 | 32.025 | 1.00 | 12.70 | C |
| ATOM | 2157 | C | ALA | A | 257 | 46.656 | 28.237 | 33.267 | 1.00 | 10.73 | C |
| ATOM | 2158 | O | ALA | A | 257 | 47.451 | 29.073 | 33.672 | 1.00 | 20.33 | O |
| ATOM | 2159 | CB | ALA | A | 257 | 46.208 | 28.073 | 30.834 | 1.00 | 13.34 | C |
| ATOM | 2160 | N | GLN | A | 258 | 45.470 | 28.080 | 33.835 | 1.00 | 12.40 | N |
| ATOM | 2161 | CA | GLN | A | 258 | 45.102 | 28.911 | 34.981 | 1.00 | 8.39 | C |
| ATOM | 2162 | C | GLN | A | 258 | 45.879 | 28.480 | 36.166 | 1.00 | 13.48 | C |
| ATOM | 2163 | O | GLN | A | 258 | 46.178 | 29.281 | 37.029 | 1.00 | 22.96 | O |
| ATOM | 2164 | CB | GLN | A | 258 | 43.614 | 28.761 | 35.305 | 1.00 | 16.12 | C |
| ATOM | 2165 | CG | GLN | A | 258 | 42.674 | 29.096 | 34.130 | 1.00 | 30.19 | C |
| ATOM | 2166 | CD | GLN | A | 258 | 42.574 | 30.585 | 33.781 | 1.00 | 37.29 | C |
| ATOM | 2167 | OE1 | GLN | A | 258 | 42.911 | 31.471 | 34.610 | 1.00 | 21.24 | O |
| ATOM | 2168 | NE2 | GLN | A | 258 | 42.021 | 30.876 | 32.572 | 1.00 | 15.94 | N |
| ATOM | 2169 | N | THR | A | 259 | 46.179 | 27.182 | 36.232 | 1.00 | 16.21 | N |
| ATOM | 2170 | CA | THR | A | 259 | 46.982 | 26.678 | 37.336 | 1.00 | 16.85 | C |
| ATOM | 2171 | C | THR | A | 259 | 48.410 | 27.186 | 37.233 | 1.00 | 20.56 | C |
| ATOM | 2172 | O | THR | A | 259 | 49.002 | 27.621 | 38.214 | 1.00 | 21.44 | O |
| ATOM | 2173 | CB | THR | A | 259 | 47.066 | 25.192 | 37.361 | 1.00 | 27.56 | C |
| ATOM | 2174 | OG1 | THR | A | 259 | 45.752 | 24.620 | 37.509 | 1.00 | 20.92 | O |
| ATOM | 2175 | CG2 | THR | A | 259 | 47.936 | 24.796 | 38.545 | 1.00 | 12.85 | C |
| ATOM | 2176 | N | ILE | A | 260 | 48.952 | 27.170 | 36.028 | 1.00 | 19.96 | N |
| ATOM | 2177 | CA | ILE | A | 260 | 50.292 | 27.704 | 35.839 | 1.00 | 23.01 | C |
| ATOM | 2178 | C | ILE | A | 260 | 50.313 | 29.180 | 36.225 | 1.00 | 31.73 | C |
| ATOM | 2179 | O | ILE | A | 260 | 51.211 | 29.627 | 36.993 | 1.00 | 25.90 | O |
| ATOM | 2180 | CB | ILE | A | 260 | 50.835 | 27.456 | 34.390 | 1.00 | 22.46 | C |
| ATOM | 2181 | CG1 | ILE | A | 260 | 51.153 | 25.940 | 34.232 | 1.00 | 24.12 | C |
| ATOM | 2182 | CG2 | ILE | A | 260 | 52.099 | 28.361 | 34.106 | 1.00 | 13.47 | C |
| ATOM | 2183 | CD1 | ILE | A | 260 | 51.501 | 25.443 | 32.810 | 1.00 | 12.58 | C |
| ATOM | 2184 | N | ALA | A | 261 | 49.28 | 029.910 | 35.764 | 1.00 | 15.35 | N |
| ATOM | 2185 | CA | ALA | A | 261 | 49.177 | 31.355 | 36.048 | 1.00 | 16.00 | C |
| ATOM | 2186 | C | ALA | A | 261 | 49.316 | 31.604 | 37.550 | 1.00 | 20.58 | C |
| ATOM | 2187 | O | ALA | A | 261 | 50.104 | 32.443 | 37.987 | 1.00 | 16.09 | O |
| ATOM | 2188 | CB | ALA | A | 261 | 47.832 | 31.958 | 35.487 | 1.00 | 13.65 | C |
| ATOM | 2189 | N | LYS | A | 262 | 48.551 | 30.843 | 38.323 | 1.00 | 11.50 | N |
| ATOM | 2190 | CA | LYS | A | 262 | 48.578 | 30.905 | 39.770 | 1.00 | 10.13 | C |
| ATOM | 2191 | C | LYS | A | 262 | 49.968 | 30.460 | 40.296 | 1.00 | 28.08 | C |
| ATOM | 2192 | O | LYS | A | 262 | 50.503 | 31.084 | 41.205 | 1.00 | 29.37 | O |
| ATOM | 2193 | CB | LYS | A | 262 | 47.453 | 30.032 | 40.335 | 1.00 | 12.50 | C |
| ATOM | 2194 | CG | LYS | A | 262 | 47.332 | 29.962 | 41.888 | 1.00 | 16.51 | C |
| ATOM | 2195 | CD | LYS | A | 262 | 46.092 | 29.092 | 42.371 | 1.00 | 46.61 | C |
| ATOM | 2196 | CE | LYS | A | 262 | 46.344 | 27.555 | 42.661 | 1.00 | 99.70 | C |
| ATOM | 2197 | NZ | LYS | A | 262 | 45.157 | 26.703 | 43.200 | 1.00 | 36.59 | N |
| ATOM | 2198 | N | VAL | A | 263 | 50.589 | 29.443 | 39.705 | 1.00 | 17.44 | N |
| ATOM | 2199 | CA | VAL | A | 263 | 51.915 | 29.039 | 40.171 | 1.00 | 16.72 | C |
| ATOM | 2200 | C | VAL | A | 263 | 52.997 | 30.170 | 39.997 | 1.00 | 32.12 | C |
| ATOM | 2201 | O | VAL | A | 263 | 53.871 | 30.412 | 40.834 | 1.00 | 21.18 | O |
| ATOM | 2202 | CS | VAL | A | 263 | 52.389 | 27.709 | 39.476 | 1.00 | 16.35 | C |
| ATOM | 2203 | CG1 | VAL | A | 263 | 53.920 | 27.518 | 39.647 | 1.00 | 11.83 | C |
| ATOM | 2204 | CG2 | VAL | A | 263 | 51.646 | 26.522 | 40.093 | 1.00 | 14.99 | C |
| ATOM | 2205 | N | VAL | A | 264 | 52.913 | 30.899 | 38.909 | 1.00 | 21.75 | N |
| ATOM | 2206 | CA | VAL | A | 264 | 53.917 | 31.877 | 38.653 | 1.00 | 19.81 | C |
| ATOM | 2207 | C | VAL | A | 264 | 53.719 | 33.208 | 39.377 | 1.00 | 35.79 | C |
| ATOM | 2208 | O | VAL | A | 264 | 54.632 | 34.032 | 39.482 | 1.00 | 28.99 | O |
| ATOM | 2209 | CS | VAL | A | 264 | 54.059 | 32.014 | 37.175 | 1.00 | 24.27 | C |
| ATOM | 2210 | CG1 | VAL | A | 264 | 54.728 | 33.269 | 36.822 | 1.00 | 33.58 | C |
| ATOM | 2211 | CG2 | VAL | A | 264 | 54.840 | 30.808 | 36.674 | 1.00 | 23.01 | C |
| ATOM | 2212 | N | GLY | A | 265 | 52.550 | 33.378 | 39.969 | 1.00 | 25.30 | N |
| ATOM | 2213 | CA | GLY | A | 265 | 52.241 | 34.620 | 40.636 | 1.00 | 24.14 | C |
| ATOM | 2214 | C | GLY | A | 265 | 51.730 | 35.694 | 39.632 | 1.00 | 35.03 | C |
| ATOM | 2215 | O | GLY | A | 265 | 51.773 | 36.911 | 39.962 | 1.00 | 33.71 | O |
| ATOM | 2216 | N | TYR | A | 266 | 51.294 | 35.257 | 38.428 | 1.00 | 26.25 | N |
| ATOM | 2217 | CA | TYR | A | 266 | 50.698 | 36.151 | 37.373 | 1.00 | 26.55 | C |
| ATOM | 2218 | C | TYR | A | 266 | 49.364 | 36.745 | 37.818 | 1.00 | 31.01 | C |
| ATOM | 2219 | O | TYR | A | 266 | 48.532 | 36.067 | 38.456 | 1.00 | 27.99 | O |
| ATOM | 2220 | CB | TYR | A | 266 | 50.501 | 35.463 | 36.008 | 1.00 | 24.31 | C |
| ATOM | 2221 | CG | TYR | A | 266 | 49.994 | 36.381 | 34.884 | 1.00 | 28.64 | C |
| ATOM | 2222 | CD1 | TYR | A | 266 | 50.670 | 37.582 | 34.542 | 1.00 | 35.05 | C |
| ATOM | 2223 | CD2 | TYR | A | 266 | 48.860 | 36.038 | 34.118 | 1.00 | 22.60 | C |
| ATOM | 2224 | CE1 | TYR | A | 266 | 50.212 | 38.434 | 33.472 | 1.00 | 20.73 | C |
| ATOM | 2225 | CE2 | TYR | A | 266 | 48.428 | 36.859 | 33.012 | 1.00 | 20.91 | C |
| ATOM | 2226 | CZ | TYR | A | 266 | 49.088 | 38.062 | 32.735 | 1.00 | 23.85 | C |
| ATOM | 2227 | OH | TYR | A | 266 | 48.622 | 38.851 | 31.710 | 1.00 | 33.40 | O |
| ATOM | 2228 | N | LYS | A | 267 | 49.217 | 38.043 | 37.604 | 1.00 | 25.72 | N |
| ATOM | 2229 | CA | LYS | A | 267 | 47.988 | 38.697 | 38.009 | 1.00 | 30.77 | C |
| ATOM | 2230 | C | LYS | A | 267 | 47.217 | 39.280 | 36.798 | 1.00 | 28.85 | C |
| ATOM | 2231 | O | LYS | A | 267 | 46.179 | 39.894 | 36.949 | 1.00 | 31.17 | O |
| ATOM | 2232 | CB | LYS | A | 267 | 48.279 | 39.741 | 39.092 | 1.00 | 27.13 | C |
| ATOM | 2233 | CG | LYS | A | 267 | 48.728 | 39.128 | 40.403 | 1.00 | 23.18 | C |
| ATOM | 2234 | CD | LYS | A | 267 | 48.420 | 40.096 | 41.562 | 1.00 | 30.98 | C |
| ATOM | 2235 | CE | LYS | A | 267 | 47.933 | 39.358 | 42.820 | 1.00 | 48.52 | C |
| ATOM | 2236 | NZ | LYS | A | 267 | 47.005 | 38.208 | 42.505 | 1.00 | 100.00 | N |
| ATOM | 2237 | N | GLY | A | 268 | 47.716 | 39.054 | 35.594 | 1.00 | 22.67 | N |
| ATOM | 2238 | CA | GLY | A | 268 | 47.019 | 39.518 | 34.394 | 1.00 | 21.38 | C |
| ATOM | 2239 | C | GLY | A | 268 | 45.856 | 38.568 | 34.085 | 1.00 | 31.03 | C |
| ATOM | 2240 | O | GLY | A | 268 | 45.455 | 37.728 | 34.911 | 1.00 | 19.71 | O |
| ATOM | 2241 | N | ARG | A | 269 | 45.387 | 38.645 | 32.849 | 1.00 | 30.40 | N |
| ATOM | 2242 | CA | ARG | A | 269 | 44.263 | 37.846 | 32.399 | 1.00 | 26.47 | C |
| ATOM | 2243 | C | ARG | A | 269 | 44.680 | 36.705 | 31.489 | 1.00 | 22.35 | C |
| ATOM | 2244 | O | ARG | A | 269 | 45.378 | 36.926 | 30.524 | 1.00 | 22.75 | O |
| ATOM | 2245 | CB | ARG | A | 269 | 43.297 | 38.753 | 31.626 | 1.00 | 22.65 | C |
| ATOM | 2246 | CG | ARG | A | 269 | 42.201 | 39.390 | 32.463 | 1.00 | 24.21 | C |
| ATOM | 2247 | CD | ARG | A | 269 | 40.936 | 39.465 | 31.568 | 1.00 | 83.45 | C |
| ATOM | 2248 | NE | ARG | A | 269 | 40.113 | 40.676 | 31.762 | 1.00 | 100.00 | N |
| ATOM | 2249 | CZ | ARG | A | 269 | 38.808 | 40.751 | 31.431 | 1.00 | 100.00 | C |
| ATOM | 2250 | NH1 | ARG | A | 269 | 38.201 | 39.691 | 30.921 | 1.00 | 99.93 | N |
| ATOM | 2251 | NH2 | ARG | A | 269 | 38.094 | 41.865 | 31.663 | 1.00 | 100.00 | N |
| ATOM | 2252 | N | VAL | A | 270 | 44.195 | 35.494 | 31.758 | 1.00 | 19.87 | N |
| ATOM | 2253 | CA | VAL | A | 270 | 44.468 | 34.389 | 30.856 | 1.00 | 24.82 | C |
| ATOM | 2254 | C | VAL | A | 270 | 43.319 | 34.456 | 29.824 | 1.00 | 22.51 | C |
| ATOM | 2255 | O | VAL | A | 270 | 42.145 | 34.501 | 30.181 | 1.00 | 25.79 | O |
| ATOM | 2256 | CB | VAL | A | 270 | 44.436 | 32.979 | 31.571 | 1.00 | 24.03 | C |
| ATOM | 2257 | CG1 | VAL | A | 270 | 44.576 | 31.861 | 30.533 | 1.00 | 20.72 | C |
| ATOM | 2258 | CG2 | VAL | A | 270 | 45.506 | 32.849 | 32.639 | 1.00 | 11.27 | C |
| ATOM | 2259 | N | VAL | A | 271 | 43.660 | 34.409 | 28.554 | 1.00 | 25.18 | N |
| ATOM | 2260 | CA | VAL | A | 271 | 42.666 | 34.492 | 27.487 | 1.00 | 28.32 | C |
| ATOM | 2261 | C | VAL | A | 271 | 42.819 | 33.370 | 26.442 | 1.00 | 24.89 | C |
| ATOM | 2262 | O | VAL | A | 271 | 43.923 | 33.115 | 25.980 | 1.00 | 21.98 | O |
| ATOM | 2263 | CB | VAL | A | 271 | 42.901 | 35.813 | 26.736 | 1.00 | 29.25 | C |
| ATOM | 2264 | CG1 | VAL | A | 271 | 42.256 | 35.773 | 25.370 | 1.00 | 31.91 | C |
| ATOM | 2265 | CG2 | VAL | A | 271 | 42.421 | 36.989 | 27.565 | 1.00 | 18.72 | C |
| ATOM | 2266 | N | PHE | A | 272 | 41.716 | 32.758 | 26.019 | 1.00 | 26.14 | N |
| ATOM | 2267 | CA | PHE | A | 272 | 41.752 | 31.747 | 24.963 | 1.00 | 24.34 | C |
| ATOM | 2268 | C | PHE | A | 272 | 41.236 | 32.266 | 23.623 | 1.00 | 28.95 | C |
| ATOM | 2269 | O | PHE | A | 272 | 40.155 | 32.826 | 23.582 | 1.00 | 22.01 | O |
| ATOM | 2270 | CB | PHE | A | 272 | 40.960 | 30.506 | 25.391 | 1.00 | 20.97 | C |
| ATOM | 2271 | CG | PHE | A | 272 | 41.764 | 29.570 | 26.243 | 1.00 | 21.77 | C |
| ATOM | 2272 | CD1 | PHE | A | 272 | 41.940 | 29.842 | 27.610 | 1.00 | 14.60 | C |
| ATOM | 2273 | CD2 | PHE | A | 272 | 42.504 | 28.550 | 25.656 | 1.00 | 22.19 | C |
| ATOM | 2274 | CE1 | PHE | A | 272 | 42.763 | 29.041 | 28.434 | 1.00 | 17.89 | C |
| ATOM | 2275 | CE2 | PHE | A | 272 | 43.336 | 27.726 | 26.454 | 1.00 | 27.64 | C |
| ATOM | 2276 | CZ | PHE | A | 272 | 43.478 | 27.979 | 27.851 | 1.00 | 25.14 | C |
| ATOM | 2277 | N | ASP | A | 273 | 42.012 | 32.114 | 22.542 | 1.00 | 29.45 | N |
| ATOM | 2278 | CA | ASP | A | 273 | 41.557 | 32.536 | 21.214 | 1.00 | 22.33 | C |
| ATOM | 2279 | C | ASP | A | 273 | 40.896 | 31.365 | 20.493 | 1.00 | 25.67 | C |
| ATOM | 2280 | O | ASP | A | 273 | 41.539 | 30.570 | 19.793 | 1.00 | 17.81 | O |
| ATOM | 2281 | CB | ASP | A | 273 | 42.672 | 33.114 | 20.343 | 1.00 | 21.45 | C |
| ATOM | 2282 | CG | ASP | A | 273 | 42.131 | 33.626 | 18.990 | 1.00 | 26.89 | C |
| ATOM | 2283 | OD1 | ASP | A | 273 | 40.975 | 33.249 | 18.598 | 1.00 | 27.76 | O |
| ATOM | 2284 | OD2 | ASP | A | 273 | 42.838 | 34.421 | 18.327 | 1.00 | 30.06 | O |
| ATOM | 2285 | N | ALA | A | 274 | 39.589 | 31.284 | 20.649 | 1.00 | 15.59 | N |
| ATOM | 2286 | CA | ALA | A | 274 | 38.932 | 30.128 | 20.128 | 1.00 | 23.75 | C |
| ATOM | 2287 | C | ALA | A | 274 | 38.853 | 30.168 | 18.653 | 1.00 | 32.30 | C |
| ATOM | 2288 | O | ALA | A | 274 | 38.284 | 29.256 | 18.029 | 1.00 | 29.37 | O |
| ATOM | 2289 | CB | ALA | A | 274 | 37.567 | 29.905 | 20.777 | 1.00 | 18.87 | C |
| ATOM | 2290 | N | SER | A | 275 | 39.372 | 31.243 | 18.081 | 1.00 | 21.10 | N |
| ATOM | 2291 | CA | SER | A | 275 | 39.343 | 31.288 | 16.631 | 1.00 | 26.90 | C |
| ATOM | 2292 | C | SER | A | 275 | 40.390 | 30.300 | 16.116 | 1.00 | 43.37 | C |
| ATOM | 2293 | O | SER | A | 275 | 40.421 | 29.949 | 14.927 | 1.00 | 46.32 | O |
| ATOM | 2294 | CB | SER | A | 275 | 39.547 | 32.683 | 16.074 | 1.00 | 15.19 | C |
| ATOM | 2295 | OG | SER | A | 275 | 40.904 | 33.070 | 16.078 | 1.00 | 28.71 | O |
| ATOM | 2296 | N | LYS | A | 276 | 41.192 | 29.780 | 17.037 | 1.00 | 22.98 | N |
| ATOM | 2297 | CA | LYS | A | 276 | 42.178 | 28.791 | 16.638 | 1.00 | 23.28 | C |
| ATOM | 2298 | C | LYS | A | 276 | 41.645 | 27.405 | 16.976 | 1.00 | 29.73 | C |
| ATOM | 2299 | O | LYS | A | 276 | 40.992 | 27.206 | 18.010 | 1.00 | 25.10 | O |
| ATOM | 2300 | CS | LYS | A | 276 | 43.544 | 29.051 | 17.275 | 1.00 | 19.19 | C |
| ATOM | 2301 | CG | LYS | A | 276 | 43.957 | 30.496 | 17.218 | 1.00 | 32.11 | C |
| ATOM | 2302 | CD | LYS | A | 276 | 44.062 | 30.852 | 15.798 | 1.00 | 22.43 | C |
| ATOM | 2303 | CE | LYS | A | 276 | 44.930 | 32.067 | 15.570 | 1.00 | 23.18 | C |
| ATOM | 2304 | NZ | LYS | A | 276 | 45.454 | 32.117 | 14.152 | 1.00 | 29.42 | N |
| ATOM | 2305 | N | PRO | A | 277 | 41.892 | 26.476 | 16.055 | 1.00 | 36.04 | N |
| ATOM | 2306 | CA | PRO | A | 277 | 41.446 | 25.087 | 16.170 | 1.00 | 35.93 | C |
| ATOM | 2307 | C | PRO | A | 277 | 42.022 | 24.332 | 17.363 | 1.00 | 29.30 | C |
| ATOM | 2308 | O | PRO | A | 277 | 43.103 | 24.650 | 17.885 | 1.00 | 30.54 | O |
| ATOM | 2309 | CB | PRO | A | 277 | 41.975 | 24.453 | 14.878 | 1.00 | 39.65 | C |
| ATOM | 2310 | CG | PRO | A | 277 | 43.249 | 25.261 | 14.566 | 1.00 | 42.90 | C |
| ATOM | 2311 | CD | PRO | A | 277 | 42.787 | 26.670 | 14.892 | 1.00 | 37.84 | C |
| ATOM | 2312 | N | ASP | A | 278 | 41.273 | 23.339 | 17.809 | 1.00 | 22.35 | N |
| ATOM | 2313 | CA | ASP | A | 278 | 41.745 | 22.501 | 18.903 | 1.00 | 22.16 | C |
| ATOM | 2314 | C | ASP | A | 278 | 42.184 | 21.189 | 18.272 | 1.00 | 19.66 | C |
| ATOM | 2315 | O | ASP | A | 278 | 41.905 | 20.917 | 17.117 | 1.00 | 23.49 | O |
| ATOM | 2316 | CB | ASP | A | 278 | 40.636 | 22.241 | 19.971 | 1.00 | 15.09 | C |
| ATOM | 2317 | CG | ASP | A | 278 | 40.216 | 23.503 | 20.702 | 1.00 | 22.86 | C |
| ATOM | 2318 | OD1 | ASP | A | 278 | 41.113 | 24.254 | 21.096 | 1.00 | 25.18 | O |
| ATOM | 2319 | OD2 | ASP | A | 278 | 38.999 | 23.787 | 20.812 | 1.00 | 39.55 | O |
| ATOM | 2320 | N | GLY | A | 279 | 42.846 | 20.355 | 19.044 | 1.00 | 30.65 | N |
| ATOM | 2321 | CA | GLY | A | 279 | 43.229 | 19.034 | 18.546 | 1.00 | 33.78 | C |
| ATOM | 2322 | C | GLY | A | 279 | 42.115 | 18.099 | 18.944 | 1.00 | 38.10 | C |
| ATOM | 2323 | O | GLY | A | 279 | 40.963 | 18.517 | 19.068 | 1.00 | 47.52 | O |
| ATOM | 2324 | N | THR | A | 280 | 42.419 | 16.839 | 19.177 | 1.00 | 29.44 | N |
| ATOM | 2325 | CA | THR | A | 280 | 41.328 | 15.990 | 19.587 | 1.00 | 26.68 | C |
| ATOM | 2326 | C | THR | A | 280 | 40.889 | 16.439 | 20.972 | 1.00 | 23.52 | C |
| ATOM | 2327 | O | THR | A | 280 | 41.670 | 17.067 | 21.713 | 1.00 | 23.62 | O |
| ATOM | 2328 | CB | THR | A | 280 | 41.695 | 14.492 | 19.540 | 1.00 | 40.78 | C |
| ATOM | 2329 | OG1 | THR | A | 280 | 42.889 | 14.272 | 20.296 | 1.00 | 25.56 | O |
| ATOM | 2330 | CG2 | THR | A | 280 | 41.893 | 14.054 | 18.095 | 1.00 | 37.71 | C |
| ATOM | 2331 | N | PRO | A | 281 | 39.672 | 16.063 | 21.346 | 1.00 | 25.54 | N |
| ATOM | 2332 | CA | PRO | A | 281 | 39.129 | 16.454 | 22.628 | 1.00 | 25.72 | C |
| ATOM | 2333 | C | PRO | A | 281 | 39.776 | 15.778 | 23.800 | 1.00 | 26.02 | C |
| ATOM | 2334 | O | PRO | A | 281 | 39.752 | 16.314 | 24.915 | 1.00 | 22.68 | O |
| ATOM | 2335 | CB | PRO | A | 281 | 37.650 | 15.990 | 22.559 | 1.00 | 28.89 | C |
| ATOM | 2336 | CG | PRO | A | 281 | 37.417 | 15.540 | 21.201 | 1.00 | 29.39 | C |
| ATOM | 2337 | CD | PRO | A | 281 | 38.761 | 15.138 | 20.646 | 1.00 | 26.82 | C |
| ATOM | 2338 | N | ARG | A | 282 | 40.281 | 14.567 | 23.587 | 1.00 | 27.88 | N |
| ATOM | 2339 | CA | ARG | A | 282 | 40.806 | 13.817 | 24.720 | 1.00 | 34.08 | C |
| ATOM | 2340 | C | ARG | A | 282 | 41.977 | 12.918 | 24.384 | 1.00 | 27.62 | C |
| ATOM | 2341 | O | ARG | A | 282 | 41.913 | 12.182 | 23.425 | 1.00 | 23.83 | O |
| ATOM | 2342 | CB | ARG | A | 282 | 39.676 | 13.017 | 25.405 | 1.00 | 20.89 | C |
| ATOM | 2343 | CG | ARG | A | 282 | 40.035 | 12.467 | 26.775 | 1.00 | 22.81 | C |
| ATOM | 2344 | CD | ARG | A | 282 | 38.762 | 11.925 | 27.442 | 1.00 | 26.77 | C |
| ATOM | 2345 | NE | ARG | A | 282 | 38.963 | 11.345 | 28.781 | 1.00 | 36.48 | N |
| ATOM | 2346 | CZ | ARG | A | 282 | 38.518 | 10.139 | 29.164 | 1.00 | 37.74 | C |
| ATOM | 2347 | NH1 | ARG | A | 282 | 37.813 | 9.360 | 28.346 | 1.00 | 28.45 | N |
| ATOM | 2348 | NH2 | ARG | A | 282 | 38.754 | 9.700 | 30.384 | 1.00 | 27.25 | N |
| ATOM | 2349 | N | LYS | A | 283 | 43.016 | 12.963 | 25.223 | 1.00 | 28.91 | N |
| ATOM | 2350 | CA | LYS | A | 283 | 44.217 | 12.171 | 25.051 | 1.00 | 24.32 | C |
| ATOM | 2351 | C | LYS | A | 283 | 44.796 | 11.766 | 26.404 | 1.00 | 29.57 | C |
| ATOM | 2352 | O | LYS | A | 283 | 45.262 | 12.626 | 27.138 | 1.00 | 33.16 | O |
| ATOM | 2353 | CB | LYS | A | 283 | 45.226 | 13.008 | 24.287 | 1.00 | 21.93 | C |
| ATOM | 2354 | CG | LYS | A | 283 | 46.111 | 12.251 | 23.316 | 1.00 | 32.38 | C |
| ATOM | 2355 | CD | LYS | A | 283 | 46.526 | 13.171 | 22.143 | 1.00 | 95.77 | C |
| ATOM | 2356 | CE | LYS | A | 283 | 45.710 | 12.937 | 20.836 | 1.00 | 100.00 | C |
| ATOM | 2357 | NZ | LYS | A | 283 | 46.418 | 13.332 | 19.535 | 1.00 | 100.00 | N |
| ATOM | 2358 | N | LEU | A | 284 | 44.747 | 10.467 | 26.734 | 1.00 | 23.37 | N |
| ATOM | 2359 | CA | LEU | A | 284 | 45.327 | 9.905 | 27.997 | 1.00 | 16.08 | C |
| ATOM | 2360 | C | LEU | A | 284 | 45.463 | 8.386 | 28.047 | 1.00 | 20.46 | C |
| ATOM | 2361 | O | LEU | A | 284 | 44.679 | 7.655 | 27.446 | 1.00 | 25.45 | O |
| ATOM | 2362 | CB | LEU | A | 284 | 44.641 | 10.387 | 29.284 | 1.00 | 16.30 | C |
| ATOM | 2363 | CG | LEU | A | 284 | 43.334 | 9.700 | 29.714 | 1.00 | 25.97 | C |
| ATOM | 2364 | CD1 | LEU | A | 284 | 42.881 | 10.089 | 31.152 | 1.00 | 22.11 | C |
| ATOM | 2365 | CD2 | LEU | A | 284 | 42.203 | 9.953 | 28.693 | 1.00 | 23.92 | C |
| ATOM | 2366 | N | LEU | A | 285 | 46.453 | 7.939 | 28.820 | 1.00 | 18.51 | N |
| ATOM | 2367 | CA | LEU | A | 285 | 46.792 | 6.527 | 29.003 | 1.00 | 16.77 | C |
| ATOM | 2368 | C | LEU | A | 285 | 45.880 | 5.865 | 30.006 | 1.00 | 30.75 | C |
| ATOM | 2369 | O | LEU | A | 285 | 45.576 | 6.439 | 31.058 | 1.00 | 22.02 | O |
| ATOM | 2370 | CB | LEU | A | 285 | 48.229 | 6.389 | 29.585 | 1.00 | 15.85 | C |
| ATOM | 2371 | CG | LEU | A | 285 | 49.307 | 6.970 | 28.672 | 1.00 | 21.51 | C |
| ATOM | 2372 | CD1 | LEU | A | 285 | 50.703 | 6.705 | 29.122 | 1.00 | 15.15 | C |
| ATOM | 2373 | CD2 | LEU | A | 285 | 49.051 | 6.368 | 27.330 | 1.00 | 16.94 | C |
| ATOM | 2374 | N | ASP | A | 286 | 45.565 | 4.599 | 29.734 | 1.00 | 26.62 | N |
| ATOM | 2375 | CA | ASP | A | 286 | 44.945 | 3.726 | 30.698 | 1.00 | 10.90 | C |
| ATOM | 2376 | C | ASP | A | 286 | 46.128 | 3.055 | 31.498 | 1.00 | 20.54 | C |
| ATOM | 2377 | O | ASP | A | 286 | 46.991 | 2.372 | 30.938 | 1.00 | 23.38 | O |
| ATOM | 2378 | CB | ASP | A | 286 | 44.073 | 2.702 | 29.970 | 1.00 | 14.65 | C |
| ATOM | 2379 | CG | ASP | A | 286 | 43.409 | 1.699 | 30.943 | 1.00 | 24.60 | C |
| ATOM | 2380 | OD1 | ASP | A | 286 | 43.932 | 1.437 | 32.083 | 1.00 | 24.60 | O |
| ATOM | 2381 | OD2 | ASP | A | 286 | 42.316 | 1.231 | 30.583 | 1.00 | 26.03 | O |
| ATOM | 2382 | N | VAL | A | 287 | 46.230 | 3.317 | 32.791 | 1.00 | 15.44 | N |
| ATOM | 2383 | CA | VAL | A | 287 | 47.354 | 2.816 | 33.556 | 1.00 | 15.58 | C |
| ATOM | 2384 | C | VAL | A | 287 | 46.973 | 1.695 | 34.521 | 1.00 | 16.48 | C |
| ATOM | 2385 | O | VAL | A | 287 | 47.613 | 1.473 | 35.572 | 1.00 | 16.63 | O |
| ATOM | 2386 | CB | VAL | A | 287 | 48.101 | 4.006 | 34.260 | 1.00 | 29.84 | C |
| ATOM | 2387 | CG1 | VAL | A | 287 | 48.534 | 5.085 | 33.224 | 1.00 | 18.39 | C |
| ATOM | 2388 | CG2 | VAL | A | 287 | 47.173 | 4.670 | 35.258 | 1.00 | 37.79 | C |
| ATOM | 2389 | N | THR | A | 288 | 45.904 | 0.992 | 34.152 | 1.00 | 22.27 | N |
| ATOM | 2390 | CA | THR | A | 288 | 45.428 | −0.152 | 34.956 | 1.00 | 19.34 | C |
| ATOM | 2391 | C | THR | A | 288 | 46.561 | −1.177 | 35.227 | 1.00 | 27.47 | C |
| ATOM | 2392 | O | THR | A | 288 | 46.778 | −1.586 | 36.365 | 1.00 | 24.87 | O |
| ATOM | 2393 | CB | THR | A | 288 | 44.288 | −0.909 | 34.244 | 1.00 | 22.86 | C |
| ATOM | 2394 | OG1 | THR | A | 288 | 43.120 | −0.096 | 34.106 | 1.00 | 24.84 | O |
| ATOM | 2395 | CG2 | THR | A | 288 | 43.916 | −2.113 | 35.024 | 1.00 | 25.08 | C |
| ATOM | 2396 | N | ARG | A | 289 | 47.290 | −1.585 | 34.179 | 1.00 | 26.08 | N |
| ATOM | 2397 | CA | ARG | A | 289 | 48.428 | −2.506 | 34.319 | 1.00 | 16.92 | C |
| ATOM | 2398 | C | ARG | A | 289 | 49.405 | −2.037 | 35.408 | 1.00 | 22.96 | C |
| ATOM | 2399 | O | ARG | A | 289 | 49.847 | −2.790 | 36.275 | 1.00 | 23.03 | O |
| ATOM | 2400 | CB | ARG | A | 289 | 49.208 | −2.607 | 32.976 | 1.00 | 12.43 | C |
| ATOM | 2401 | CG | ARG | A | 289 | 48.934 | −3.804 | 32.103 | 1.00 | 29.39 | C |
| ATOM | 2402 | CD | ARG | A | 289 | 50.016 | −4.102 | 31.037 | 1.00 | 25.88 | C |
| ATOM | 2403 | NE | ARG | A | 289 | 49.441 | −4.996 | 30.020 | 1.00 | 17.26 | N |
| ATOM | 2404 | CZ | ARG | A | 289 | 50.053 | −5.459 | 28.930 | 1.00 | 38.82 | C |
| ATOM | 2405 | NH1 | ARG | A | 289 | 51.306 | −5.153 | 28.660 | 1.00 | 13.51 | N |
| ATOM | 2406 | NH2 | ARG | A | 289 | 49.400 | −6.262 | 28.096 | 1.00 | 37.68 | N |
| ATOM | 2407 | N | LEU | A | 290 | 49.815 | −0.786 | 35.306 | 1.00 | 26.60 | N |
| ATOM | 2408 | CA | LEU | A | 290 | 50.809 | −0.254 | 36.219 | 1.00 | 25.42 | C |
| ATOM | 2409 | C | LEU | A | 290 | 50.324 | −0.376 | 37.656 | 1.00 | 24.17 | C |
| ATOM | 2410 | O | LEU | A | 290 | 51.072 | −0.759 | 38.574 | 1.00 | 19.94 | O |
| ATOM | 2411 | CB | LEU | A | 290 | 51.000 | 1.219 | 35.876 | 1.00 | 24.66 | C |
| ATOM | 2412 | CG | LEU | A | 290 | 52.281 | 2.019 | 36.066 | 1.00 | 24.67 | C |
| ATOM | 2413 | CD1 | LEU | A | 290 | 51.992 | 3.479 | 36.504 | 1.00 | 29.25 | C |
| ATOM | 2414 | CD2 | LEU | A | 290 | 53.450 | 1.335 | 36.788 | 1.00 | 15.82 | C |
| ATOM | 2415 | N | HIS | A | 291 | 49.093 | 0.075 | 37.868 | 1.00 | 30.10 | N |
| ATOM | 2416 | CA | HIS | A | 291 | 48.513 | 0.074 | 39.212 | 1.00 | 34.17 | C |
| ATOM | 2417 | C | HIS | A | 291 | 48.411 | −1.367 | 39.730 | 1.00 | 43.41 | C |
| ATOM | 2418 | O | HIS | A | 291 | 48.621 | −1.654 | 40.929 | 1.00 | 38.81 | O |
| ATOM | 2419 | CB | HIS | A | 291 | 47.113 | 0.674 | 39.143 | 1.00 | 28.01 | C |
| ATOM | 2420 | CG | HIS | A | 291 | 47.097 | 2.153 | 38.984 | 1.00 | 29.68 | C |
| ATOM | 2421 | ND1 | HIS | A | 291 | 48.242 | 2.921 | 39.015 | 1.00 | 35.63 | N |
| ATOM | 2422 | CD2 | HIS | A | 291 | 46.068 | 3.024 | 38.855 | 1.00 | 31.18 | C |
| ATOM | 2423 | CE1 | HIS | A | 291 | 47.926 | 4.197 | 38.845 | 1.00 | 24.20 | C |
| ATOM | 2424 | NE2 | HIS | A | 291 | 46.612 | 4.289 | 38.747 | 1.00 | 21.92 | N |
| ATOM | 2425 | N | GLN | A | 292 | 48.048 | −2.260 | 38.821 | 1.00 | 30.71 | N |
| ATOM | 2426 | CA | GLN | A | 292 | 47.950 | −3.654 | 39.181 | 1.00 | 34.82 | C |
| ATOM | 2427 | C | GLN | A | 292 | 49.287 | −4.197 | 39.622 | 1.00 | 36.93 | C |
| ATOM | 2428 | O | GLN | A | 292 | 49.323 | −5.040 | 40.510 | 1.00 | 27.56 | O |
| ATOM | 2429 | CB | GLN | A | 292 | 47.322 | −4.487 | 38.069 | 1.00 | 28.23 | C |
| ATOM | 2430 | CG | GLN | A | 292 | 45.798 | −4.405 | 38.171 | 1.00 | 81.15 | C |
| ATOM | 2431 | CD | GLN | A | 292 | 45.023 | ‘4.954 | 36.963 | 1.00 | 100.00 | C |
| ATOM | 2432 | OE1 | GLN | A | 292 | 45.597 | −5.410 | 35.951 | 1.00 | 99.65 | O |
| ATOM | 2433 | NE2 | GLN | A | 292 | 43.687 | −4.895 | 37.073 | 1.00 | 40.86 | N |
| ATOM | 2434 | N | LEU | A | 293 | 50.375 | −3.658 | 39.058 | 1.00 | 31.75 | N |
| ATOM | 2435 | CA | LEU | A | 293 | 51.750 | −4.072 | 39.383 | 1.00 | 22.67 | C |
| ATOM | 2436 | C | LEU | A | 293 | 52.238 | −3.323 | 40.613 | 1.00 | 28.64 | C |
| ATOM | 2437 | O | LEU | A | 293 | 53.420 | −3.377 | 41.017 | 1.00 | 22.27 | O |
| ATOM | 2438 | CB | LEU | A | 293 | 52.665 | −3.769 | 38.205 | 1.00 | 25.57 | C |
| ATOM | 2439 | CG | LEU | A | 293 | 52.497 | −4.703 | 37.016 | 1.00 | 35.11 | C |
| ATOM | 2440 | CD1 | LEU | A | 293 | 53.306 | −4.170 | 35.836 | 1.00 | 28.25 | C |
| ATOM | 2441 | CD2 | LEU | A | 293 | 52.965 | −6.110 | 37.439 | 1.00 | 47.81 | C |
| ATOM | 2442 | N | GLY | A | 294 | 51.316 | −2.510 | 41.111 | 1.00 | 33.08 | N |
| ATOM | 2443 | CA | GLY | A | 294 | 51.488 | −1.793 | 42.347 | 1.00 | 24.90 | C |
| ATOM | 2444 | C | GLY | A | 294 | 52.272 | −0.512 | 42.326 | 1.00 | 29.31 | C |
| ATOM | 2445 | O | GLY | A | 294 | 53.070 | −0.249 | 43.223 | 1.00 | 25.25 | O |
| ATOM | 2446 | N | TRP | A | 295 | 52.000 | 0.347 | 41.368 | 1.00 | 27.83 | N |
| ATOM | 2447 | CA | TRP | A | 295 | 52.687 | 1.623 | 41.385 | 1.00 | 19.45 | C |
| ATOM | 2448 | C | TRP | A | 295 | 51.684 | 2.731 | 41.081 | 1.00 | 25.79 | C |
| ATOM | 2449 | O | TRP | A | 295 | 50.765 | 2.527 | 40.297 | 1.00 | 20.43 | O |
| ATOM | 2450 | CB | TRP | A | 295 | 53.961 | 1.614 | 40.524 | 1.00 | 12.85 | C |
| ATOM | 2451 | CG | TRP | A | 295 | 54.750 | 2.911 | 40.618 | 1.00 | 23.04 | C |
| ATOM | 2452 | CD1 | TRP | A | 295 | 55.897 | 3.161 | 41.368 | 1.00 | 23.68 | C |
| ATOM | 2453 | CD2 | TRP | A | 295 | 54.415 | 4.159 | 39.979 | 1.00 | 20.72 | C |
| ATOM | 2454 | NE1 | TRP | A | 295 | 56.258 | 4.493 | 41.244 | 1.00 | 18.67 | N |
| ATOM | 2455 | CE2 | TRP | A | 295 | 55.389 | 5.113 | 40.373 | 1.00 | 20.95 | C |
| ATOM | 2456 | CE3 | TRP | A | 295 | 53.406 | 4.550 | 39.102 | 1.00 | 21.47 | C |
| ATOM | 2457 | CZ2 | TRP | A | 295 | 55.338 | 6.439 | 39.958 | 1.00 | 17.58 | C |
| ATOM | 2456 | CZ3 | TRP | A | 295 | 53.403 | 5.873 | 38.632 | 1.00 | 21.57 | C |
| ATOM | 2459 | CH2 | TRP | A | 295 | 54.368 | 6.787 | 39.058 | 1.00 | 19.45 | C |
| ATOM | 2460 | N | TYR | A | 296 | 51.709 | 3.797 | 41.884 | 1.00 | 25.17 | N |
| ATOM | 2461 | CA | TYR | A | 296 | 50.720 | 4.883 | 41.731 | 1.00 | 24.90 | C |
| ATOM | 2462 | C | TYR | A | 296 | 51.517 | 6.178 | 41.857 | 1.00 | 30.85 | C |
| ATOM | 2463 | O | TYR | A | 296 | 52.363 | 6.272 | 42.745 | 1.00 | 21.27 | O |
| ATOM | 2464 | CB | TYR | A | 296 | 49.654 | 4.813 | 42.840 | 1.00 | 25.16 | C |
| ATOM | 2465 | CG | TYR | A | 296 | 48.685 | 3.651 | 42.744 | 1.00 | 23.04 | C |
| ATOM | 2466 | CD1 | TYR | A | 296 | 49.078 | 2.343 | 43.088 | 1.00 | 31.62 | C |
| ATOM | 2467 | CD2 | TYR | A | 296 | 47.380 | 3.853 | 42.289 | 1.00 | 26.02 | C |
| ATOM | 2468 | CE1 | TYR | A | 296 | 48.203 | 1.268 | 42.935 | 1.00 | 24.42 | C |
| ATOM | 2469 | CE2 | TYR | A | 296 | 46.493 | 2.770 | 42.127 | 1.00 | 24.81 | C |
| ATOM | 2470 | CZ | TYR | A | 296 | 46.902 | 1.483 | 42.464 | 1.00 | 39.41 | C |
| ATOM | 2471 | OH | TYR | A | 296 | 45.984 | 0.434 | 42.337 | 1.00 | 66.19 | O |
| ATOM | 2472 | N | HIS | A | 297 | 51.324 | 7.123 | 40.924 | 1.00 | 20.95 | N |
| ATOM | 2473 | CA | HIS | A | 297 | 52.130 | 8.343 | 40.938 | 1.00 | 26.86 | C |
| ATOM | 2474 | C | HIS | A | 297 | 51.947 | 9.175 | 42.210 | 1.00 | 35.01 | C |
| ATOM | 2475 | O | HIS | A | 297 | 50.885 | 9.132 | 42.874 | 1.00 | 26.92 | O |
| ATOM | 2476 | CB | HIS | A | 297 | 51.819 | 9.192 | 39.733 | 1.00 | 25.77 | C |
| ATOM | 2477 | CG | HIS | A | 297 | 50.489 | 9.842 | 39.803 | 1.00 | 31.16 | C |
| ATOM | 2478 | ND1 | HIS | A | 297 | 49.314 | 9.145 | 39.633 | 1.00 | 34.21 | N |
| ATOM | 2479 | CD2 | HIS | A | 297 | 50.135 | 11.094 | 40.167 | 1.00 | 25.83 | C |
| ATOM | 2480 | CE1 | HIS | A | 297 | 48.290 | 9.972 | 39.776 | 1.00 | 24.14 | C |
| ATOM | 2481 | NE2 | HIS | A | 297 | 48.761 | 11.164 | 40.087 | 1.00 | 23.35 | N |
| ATOM | 2482 | N | GLU | A | 298 | 52.983 | 9.926 | 42.554 | 1.00 | 24.98 | N |
| ATOM | 2483 | CA | GLU | A | 298 | 52.957 | 10.683 | 43.798 | 1.00 | 27.65 | C |
| ATOM | 2484 | C | GLU | A | 298 | 52.831 | 12.187 | 43.741 | 1.00 | 36.86 | C |
| ATOM | 2485 | O | GLU | A | 298 | 52.433 | 12.792 | 44.718 | 1.00 | 43.61 | O |
| ATOM | 2486 | CB | GLU | A | 298 | 54.153 | 10.319 | 44.686 | 1.00 | 22.02 | C |
| ATOM | 2487 | CG | GLU | A | 298 | 54.004 | 8.943 | 45.285 | 1.00 | 36.42 | C |
| ATOM | 2488 | CD | GLU | A | 298 | 54.999 | 8.664 | 46.406 | 1.00 | 100.00 | C |
| ATOM | 2489 | OE1 | GLU | A | 298 | 56.223 | 8.561 | 46.152 | 1.00 | 44.79 | O |
| ATOM | 2490 | OE2 | GLU | A | 298 | 54.526 | 8.470 | 47.547 | 1.00 | 100.00 | O |
| ATOM | 2491 | N | ILE | A | 299 | 53.232 | 12.800 | 42.639 | 1.00 | 23.49 | N |
| ATOM | 2492 | CA | ILE | A | 299 | 53.268 | 14.244 | 42.562 | 1.00 | 13.25 | C |
| ATOM | 2493 | C | ILE | A | 299 | 52.016 | 14.848 | 41.906 | 1.00 | 27.05 | C |
| ATOM | 2494 | O | ILE | A | 299 | 51.681 | 14.530 | 40.757 | 1.00 | 26.73 | O |
| ATOM | 2495 | CB | ILE | A | 299 | 54.586 | 14.711 | 41.862 | 1.00 | 15.93 | C |
| ATOM | 2496 | CG1 | ILE | A | 299 | 55.836 | 14.183 | 42.606 | 1.00 | 23.83 | C |
| ATOM | 2497 | CG2 | ILE | A | 299 | 54.596 | 16.213 | 41.541 | 1.00 | 17.37 | C |
| ATOM | 2498 | CD1 | ILE | A | 299 | 57.232 | 14.221 | 41.787 | 1.00 | 21.32 | C |
| ATOM | 2499 | N | SER | A | 300 | 51.323 | 15.716 | 42.648 | 1.00 | 18.55 | N |
| ATOM | 2500 | CA | SER | A | 300 | 50.177 | 16.449 | 42.091 | 1.00 | 19.58 | C |
| ATOM | 2501 | C | SER | A | 300 | 50.714 | 17.415 | 41.042 | 1.00 | 17.29 | C |
| ATOM | 2502 | O | SER | A | 300 | 51.824 | 17.941 | 41.178 | 1.00 | 21.06 | O |
| ATOM | 2503 | CB | SER | A | 300 | 49.542 | 17.307 | 43.181 | 1.00 | 16.78 | C |
| ATOM | 2504 | OG | SER | A | 300 | 50.548 | 17.969 | 43.923 | 1.00 | 75.80 | O |
| ATOM | 2505 | N | LEU | A | 301 | 49.870 | 17.755 | 40.075 | 1.00 | 16.13 | N |
| ATOM | 2506 | CA | LEU | A | 301 | 50.246 | 18.675 | 39.014 | 1.00 | 17.70 | C |
| ATOM | 2507 | C | LEU | A | 301 | 50.689 | 19.964 | 39.646 | 1.00 | 20.11 | C |
| ATOM | 2508 | O | LEU | A | 301 | 51.714 | 20.568 | 39.303 | 1.00 | 20.46 | O |
| ATOM | 2509 | CB | LEU | A | 301 | 48.990 | 18.981 | 38.197 | 1.00 | 17.92 | C |
| ATOM | 2510 | CG | LEU | A | 301 | 49.182 | 20.030 | 37.112 | 1.00 | 25.15 | C |
| ATOM | 2511 | CD1 | LEU | A | 301 | 50.233 | 19.552 | 36.086 | 1.00 | 18.82 | C |
| ATOM | 2512 | CD2 | LEU | A | 301 | 47.854 | 20.177 | 36.436 | 1.00 | 25.88 | C |
| ATOM | 2513 | N | GLU | A | 302 | 49.845 | 20.398 | 40.554 | 1.00 | 27.01 | N |
| ATOM | 2514 | CA | GLU | A | 302 | 50.053 | 21.636 | 41.280 | 1.00 | 37.72 | C |
| ATOM | 2515 | C | GLU | A | 302 | 51.410 | 21.618 | 41.996 | 1.00 | 29.99 | C |
| ATOM | 2516 | O | GLU | A | 302 | 52.245 | 22.514 | 41.798 | 1.00 | 27.15 | O |
| ATOM | 2517 | CB | GLU | A | 302 | 48.899 | 21.841 | 42.275 | 1.00 | 43.10 | C |
| ATOM | 2518 | CG | GLU | A | 302 | 49.061 | 23.061 | 43.174 | 1.00 | 90.85 | C |
| ATOM | 2519 | CD | GLU | A | 302 | 48.451 | 24.324 | 42.580 | 1.00 | 100.00 | C |
| ATOM | 2520 | OE1 | GLU | A | 302 | 47.566 | 24.209 | 41.706 | 1.00 | 100.00 | O |
| ATOM | 2521 | OE2 | GLU | A | 302 | 48.808 | 25.432 | 43.036 | 1.00 | 64.50 | O |
| ATOM | 2522 | N | ALA | A | 303 | 51.646 | 20.591 | 42.801 | 1.00 | 8.72 | N |
| ATOM | 2523 | CA | ALA | A | 303 | 52.937 | 20.455 | 43.459 | 1.00 | 15.03 | C |
| ATOM | 2524 | C | ALA | A | 303 | 54.102 | 20.355 | 42.450 | 1.00 | 19.85 | C |
| ATOM | 2525 | O | ALA | A | 303 | 55.104 | 21.090 | 42.553 | 1.00 | 22.24 | O |
| ATOM | 2526 | CB | ALA | A | 303 | 52.938 | 19.258 | 44.410 | 1.00 | 18.97 | C |
| ATOM | 2527 | N | GLY | A | 304 | 53.953 | 19.472 | 41.467 | 1.00 | 13.05 | N |
| ATOM | 2528 | CA | GLY | A | 304 | 54.970 | 19.321 | 40.448 | 1.00 | 8.94 | C |
| ATOM | 2529 | C | GLY | A | 304 | 55.239 | 20.621 | 39.695 | 1.00 | 20.31 | C |
| ATOM | 2530 | O | GLY | A | 304 | 56.394 | 20.900 | 39.322 | 1.00 | 14.30 | O |
| ATOM | 2531 | N | LEU | A | 305 | 54.191 | 21.383 | 39.361 | 1.00 | 10.76 | N |
| ATOM | 2532 | CA | LEU | A | 305 | 54.483 | 22.622 | 38.611 | 1.00 | 20.29 | C |
| ATOM | 2533 | C | LEU | A | 305 | 55.281 | 23.669 | 39.456 | 1.00 | 28.92 | C |
| ATOM | 2534 | O | LEU | A | 305 | 56.194 | 24.385 | 38.974 | 1.00 | 17.69 | O |
| ATOM | 2535 | CB | LEU | A | 305 | 53.202 | 23.245 | 38.033 | 1.00 | 24.03 | C |
| ATOM | 2536 | CG | LEU | A | 305 | 52.357 | 22.647 | 36.880 | 1.00 | 27.66 | C |
| ATOM | 2537 | CD1 | LEU | A | 305 | 50.975 | 23.384 | 36.789 | 1.00 | 13.44 | C |
| ATOM | 2538 | CD2 | LEU | A | 305 | 53.079 | 22.724 | 35.543 | 1.00 | 18.39 | C |
| ATOM | 2539 | N | ALA | A | 306 | 54.904 | 23.757 | 40.724 | 1.00 | 19.94 | N |
| ATOM | 2540 | CA | ALA | A | 306 | 55.544 | 24.660 | 41.655 | 1.00 | 24.79 | C |
| ATOM | 2541 | C | ALA | A | 306 | 57.035 | 24.380 | 41.743 | 1.00 | 27.51 | C |
| ATOM | 2542 | O | ALA | A | 306 | 57.852 | 25.280 | 41.662 | 1.00 | 29.68 | O |
| ATOM | 2543 | CB | ALA | A | 306 | 54.937 | 24.471 | 43.002 | 1.00 | 17.87 | C |
| ATOM | 2544 | N | SER | A | 307 | 57.378 | 23.137 | 42.011 | 1.00 | 18.46 | N |
| ATOM | 2545 | CA | SER | A | 307 | 58.793 | 22.756 | 42.162 | 1.00 | 16.31 | C |
| ATOM | 2546 | C | SER | A | 307 | 59.547 | 22.885 | 40.832 | 1.00 | 22.66 | C |
| ATOM | 2547 | O | SER | A | 307 | 60.742 | 23.212 | 40.786 | 1.00 | 28.47 | O |
| ATOM | 2548 | CB | SER | A | 307 | 58.851 | 21.304 | 42.622 | 1.00 | 20.47 | C |
| ATOM | 2549 | OG | SER | A | 307 | 58.517 | 20.454 | 41.526 | 1.00 | 29.03 | O |
| ATOM | 2550 | N | THR | A | 308 | 58.849 | 22.631 | 39.735 | 1.00 | 27.31 | N |
| ATOM | 2551 | CA | THR | A | 308 | 59.458 | 22.738 | 38.413 | 1.00 | 22.89 | C |
| ATOM | 2552 | C | THR | A | 308 | 59.757 | 24.216 | 38.107 | 1.00 | 26.06 | C |
| ATOM | 2553 | O | THR | A | 308 | 60.819 | 24.546 | 37.591 | 1.00 | 29.89 | O |
| ATOM | 2554 | CB | THR | A | 308 | 58.536 | 22.115 | 37.318 | 1.00 | 18.72 | C |
| ATOM | 2555 | OG1 | THR | A | 308 | 58.356 | 20.714 | 37.545 | 1.00 | 20.17 | O |
| ATOM | 2556 | CG2 | THR | A | 308 | 59.094 | 22.330 | 35.923 | 1.00 | 12.37 | C |
| ATOM | 2557 | N | TYR | A | 309 | 58.846 | 25.118 | 38.453 | 1.00 | 28.20 | N |
| ATOM | 2558 | CA | TYR | A | 309 | 59.110 | 26.549 | 38.241 | 1.00 | 31.09 | C |
| ATOM | 2559 | C | TYR | A | 309 | 60.383 | 27.059 | 39.045 | 1.00 | 16.31 | C |
| ATOM | 2560 | O | TYR | A | 309 | 61.179 | 27.858 | 38.577 | 1.00 | 16.91 | O |
| ATOM | 2561 | CB | TYR | A | 309 | 57.819 | 27.373 | 38.533 | 1.00 | 31.19 | C |
| ATOM | 2562 | CG | TYR | A | 309 | 57.944 | 28.895 | 38.392 | 1.00 | 14.57 | C |
| ATOM | 2563 | CD1 | TYR | A | 309 | 58.397 | 29.457 | 37.224 | 1.00 | 17.51 | C |
| ATOM | 2564 | CD2 | TYR | A | 309 | 57.575 | 29.757 | 39.442 | 1.00 | 24.99 | C |
| ATOM | 2565 | CE1 | TYR | A | 309 | 58.527 | 30.801 | 37.100 | 1.00 | 18.41 | C |
| ATOM | 2566 | CE2 | TYR | A | 309 | 57.744 | 31.129 | 39.351 | 1.00 | 19.04 | C |
| ATOM | 2567 | CZ | TYR | A | 309 | 58.212 | 31.641 | 38.164 | 1.00 | 29.13 | C |
| ATOM | 2568 | OH | TYR | A | 309 | 58.300 | 33.004 | 37.966 | 1.00 | 28.22 | O |
| ATOM | 2569 | N | GLN | A | 310 | 60.560 | 26.579 | 40.260 | 1.00 | 15.41 | N |
| ATOM | 2570 | CA | GLN | A | 310 | 61.705 | 26.964 | 41.087 | 1.00 | 22.35 | C |
| ATOM | 2571 | C | GLN | A | 310 | 63.001 | 26.492 | 40.446 | 1.00 | 31.46 | C |
| ATOM | 2572 | O | GLN | A | 310 | 64.009 | 27.191 | 40.442 | 1.00 | 33.42 | O |
| ATOM | 2573 | CB | GLN | A | 310 | 61.587 | 26.335 | 42.482 | 1.00 | 17.67 | C |
| ATOM | 2574 | CG | GLN | A | 310 | 62.579 | 26.921 | 43.461 | 1.00 | 57.58 | C |
| ATOM | 2575 | CD | GLN | A | 310 | 62.287 | 28.370 | 43.782 | 1.00 | 65.14 | C |
| ATOM | 2576 | OE1 | GLN | A | 310 | 61.134 | 28.754 | 44.000 | 1.00 | 41.94 | O |
| ATOM | 2577 | NE2 | GLN | A | 310 | 63.330 | 29.194 | 43.801 | 1.00 | 99.09 | N |
| ATOM | 2578 | N | TRP | A | 311 | 62.957 | 25.321 | 39.830 | 1.00 | 28.76 | N |
| ATOM | 2579 | CA | TRP | A | 311 | 64.146 | 24.822 | 39.163 | 1.00 | 26.29 | C |
| ATOM | 2580 | C | TRP | A | 311 | 64.474 | 25.769 | 38.040 | 1.00 | 17.91 | C |
| ATOM | 2581 | O | TRP | A | 311 | 65.599 | 26.193 | 37.880 | 1.00 | 22.89 | O |
| ATOM | 2582 | CB | TRP | A | 311 | 63.938 | 23.383 | 38.643 | 1.00 | 27.53 | C |
| ATOM | 2583 | CG | TRP | A | 311 | 65.176 | 22.784 | 38.119 | 1.00 | 17.82 | C |
| ATOM | 2584 | CD1 | TRP | A | 311 | 66.132 | 22.090 | 38.826 | 1.00 | 20.21 | C |
| ATOM | 2585 | CD2 | TRP | A | 311 | 65.652 | 22.881 | 36.784 | 1.00 | 17.99 | C |
| ATOM | 2586 | NE1 | TRP | A | 311 | 67.197 | 21.776 | 37.992 | 1.00 | 20.39 | N |
| ATOM | 2587 | CE2 | TRP | A | 311 | 66.933 | 22.284 | 36.746 | 1.00 | 19.57 | C |
| ATOM | 2588 | CE3 | TRP | A | 311 | 65.141 | 23.461 | 35.621 | 1.00 | 20.26 | C |
| ATOM | 2589 | CZ2 | TRP | A | 311 | 67.686 | 22.236 | 35.599 | 1.00 | 14.25 | C |
| ATOM | 2590 | CZ3 | TRP | A | 311 | 65.901 | 23.446 | 34.501 | 1.00 | 18.59 | C |
| ATOM | 2591 | CH2 | TRP | A | 311 | 67.169 | 22.831 | 34.494 | 1.00 | 16.86 | C |
| ATOM | 2592 | N | PHE | A | 312 | 63.469 | 26.109 | 37.256 | 1.00 | 17.47 | N |
| ATOM | 2593 | CA | PHE | A | 312 | 63.665 | 27.064 | 36.179 | 1.00 | 20.14 | C |
| ATOM | 2594 | C | PHE | A | 312 | 64.224 | 28.371 | 36.733 | 1.00 | 18.33 | C |
| ATOM | 2595 | O | PHE | A | 312 | 65.080 | 29.024 | 36.104 | 1.00 | 24.76 | O |
| ATOM | 2596 | CB | PHE | A | 312 | 62.328 | 27.318 | 35.458 | 1.00 | 29.51 | C |
| ATOM | 2597 | CG | PHE | A | 312 | 62.328 | 28.544 | 34.603 | 1.00 | 28.52 | C |
| ATOM | 2598 | CD1 | PHE | A | 312 | 62.883 | 28.508 | 33.338 | 1.00 | 30.53 | C |
| ATOM | 2599 | CD2 | PHE | A | 312 | 61.825 | 29.758 | 35.104 | 1.00 | 29.31 | C |
| ATOM | 2600 | CE1 | PHE | A | 312 | 62.936 | 29.660 | 32.554 | 1.00 | 34.73 | C |
| ATOM | 2601 | CE2 | PHE | A | 312 | 61.900 | 30.904 | 34.362 | 1.00 | 38.40 | C |
| ATOM | 2602 | CZ | PHE | A | 312 | 62.432 | 30.860 | 33.063 | 1.00 | 40.73 | C |
| ATOM | 2603 | N | LEU | A | 313 | 63.697 | 28.787 | 37.876 | 1.00 | 22.46 | N |
| ATOM | 2604 | CA | LEU | A | 313 | 64.170 | 30.025 | 38.516 | 1.00 | 28.47 | C |
| ATOM | 2605 | C | LEU | A | 313 | 65.627 | 29.827 | 38.898 | 1.00 | 37.53 | C |
| ATOM | 2606 | O | LEU | A | 313 | 66.452 | 30.693 | 38.629 | 1.00 | 34.20 | O |
| ATOM | 2607 | CB | LEU | A | 313 | 63.375 | 30.410 | 39.783 | 1.00 | 20.44 | C |
| ATOM | 2608 | CG | LEU | A | 313 | 61.955 | 30.897 | 39.555 | 1.00 | 16.29 | C |
| ATOM | 2609 | CD1 | LEU | A | 313 | 61.499 | 31.399 | 40.871 | 1.00 | 15.94 | C |
| ATOM | 2610 | CD2 | LEU | A | 313 | 61.959 | 31.961 | 38.524 | 1.00 | 14.44 | C |
| ATOM | 2611 | N | GLU | A | 314 | 65.953 | 28.685 | 39.508 | 1.00 | 30.70 | N |
| ATOM | 2612 | CA | GLU | A | 314 | 67.353 | 28.432 | 39.875 | 1.00 | 24.15 | C |
| ATOM | 2613 | C | GLU | A | 314 | 68.291 | 28.149 | 38.703 | 1.00 | 36.34 | C |
| ATOM | 2614 | O | GLU | A | 314 | 69.485 | 28.047 | 38.890 | 1.00 | 43.10 | O |
| ATOM | 2615 | CB | GLU | A | 314 | 67.459 | 27.366 | 40.947 | 1.00 | 19.90 | C |
| ATOM | 2616 | CG | GLU | A | 314 | 66.634 | 27.754 | 42.141 | 1.00 | 27.37 | C |
| ATOM | 2617 | CD | GLU | A | 314 | 66.450 | 26.666 | 43.182 | 1.00 | 31.09 | C |
| ATOM | 2618 | OE1 | GLU | A | 314 | 67.157 | 25.648 | 43.085 | 1.00 | 59.60 | O |
| ATOM | 2619 | OE2 | GLU | A | 314 | 65.634 | 26.872 | 44.125 | 1.00 | 46.20 | O |
| ATOM | 2620 | N | ASN | A | 315 | 67.778 | 28.114 | 37.479 | 1.00 | 40.17 | N |
| ATOM | 2621 | CA | ASN | A | 315 | 68.637 | 27.802 | 36.343 | 1.00 | 37.76 | C |
| ATOM | 2622 | C | ASN | A | 315 | 68.383 | 28.578 | 35.112 | 1.00 | 43.75 | C |
| ATOM | 2623 | O | ASN | A | 315 | 68.591 | 28.001 | 34.047 | 1.00 | 39.15 | O |
| ATOM | 2624 | CB | ASN | A | 315 | 68.425 | 26.360 | 35.884 | 1.00 | 33.74 | C |
| ATOM | 2625 | CG | ASN | A | 315 | 69.028 | 25.383 | 36.801 | 1.00 | 53.18 | C |
| ATOM | 2626 | OD1 | ASN | A | 315 | 68.456 | 25.087 | 37.835 | 1.00 | 49.13 | O |
| ATOM | 2627 | ND2 | ASN | A | 315 | 70.239 | 24.926 | 36.479 | 1.00 | 97.72 | N |
| ATOM | 2628 | N | GLN | A | 316 | 67.852 | 29.803 | 35.197 | 1.00 | 49.87 | N |
| ATOM | 2629 | CA | GLN | A | 316 | 67.627 | 30.550 | 33.957 | 1.00 | 77.90 | C |
| ATOM | 2630 | C | GLN | A | 316 | 68.797 | 31.448 | 33.525 | 1.00 | 100.00 | C |
| ATOM | 2631 | O | GLN | A | 316 | 69.272 | 31.387 | 32.375 | 1.00 | 51.33 | O |
| ATOM | 2632 | CB | GLN | A | 316 | 66.280 | 31.276 | 33.902 | 1.00 | 75.89 | C |
| ATOM | 2633 | CG | GLN | A | 316 | 65.683 | 31.589 | 35.231 | 1.00 | 80.97 | C |
| ATOM | 2634 | CD | GLN | A | 316 | 65.233 | 33.036 | 35.350 | 1.00 | 54.58 | C |
| ATOM | 2635 | OE1 | GLN | A | 316 | 64.881 | 33.699 | 34.367 | 1.00 | 46.46 | O |
| ATOM | 2636 | NE2 | GLN | A | 316 | 65.257 | 33.538 | 36.566 | 1.00 | 33.46 | N |
| TER | 2637 | GLN | A | 316 | | | | | | | |
| CONECT | 110 | 111 | | | | | | | | | |
| CONECT | 111 | 110 | 112 | | | | | | | | |
| CONECT | 112 | 111 | 113 | 114 | | | | | | | |
| CONECT | 113 | 112 | 118 | | | | | | | | |
| CONECT | 114 | 112 | 115 | 116 | | | | | | | |
| CONECT | 115 | 114 | | | | | | | | | |
| CONECT | 116 | 114 | 117 | 118 | | | | | | | |
| CONECT | 117 | 116 | 129 | | | | | | | | |
| CONECT | 118 | 113 | 116 | | | | | | | | |
| CONECT | 120 | 121 | | | | | | | | | |
| CONECT | 121 | 120 | 122 | | | | | | | | |
| CONECT | 122 | 121 | 123 | 124 | | | | | | | |
| CONECT | 123 | 122 | 128 | | | | | | | | |
| CONECT | 124 | 122 | 125 | 126 | | | | | | | |
| CONECT | 125 | 124 | | | | | | | | | |
| CONECT | 126 | 124 | 127 | 128 | | | | | | | |
| CONECT | 127 | 126 | | | | | | | | | |
| CONECT | 128 | 123 | 126 | | | | | | | | |
| CONECT | 129 | 117 | 130 | 131 | 132 | | | | | | |
| CONECT | 130 | 129 | | | | | | | | | |
| CONECT | 131 | 129 | | | | | | | | | |
| CONECT | 132 | 129 | | | | | | | | | |

| | | | | | | | | | | | | |
|---|---|---|---|---|---|---|---|---|---|---|---|---|
| MASTER | 208 | O | 1 | 13 | 10 | O | 3 | 6 | 2636 | 1 | 22 | 25 |
| END | | | | | | | | | | | | |

While various embodiments of the present invention have been described in detail, it is apparent that modifications and adaptations of those embodiments will occur to those skilled in the art. However, it is to be expressly understood that such modifications and adaptations are within the spirit and scope of the present invention, as set forth in the following claims.

What is claimed:

1. A method for producing ascorbic acid or esters thereof in a microorganism, comprising culturing a microorganism having a genetic modification to increase the action of an enzyme selected from the group consisting of hexokinase, glucose phosphate isomerase, phosphomannose isomerase, phosphomannomutase, GDP-D-mannose pyrophosphorylase, GDP-D-mannose:GDP-L-galactose epimerase, GDP-L-galactose phosphorylase, L-galactose-1-P-phosphatase, L-galactose dehydrogenase, and L-galactono-γ-lactone dehydrogenase; and recovering said ascorbic acid or esters thereof.

2. A method, as claimed in claim 1, wherein said genetic modification is a genetic modification to increase the action of an enzyme selected from the group consisting of GDP-D-mannose:GDP-L-galactose epimerase, GDP-L-galactose phosphorylase, L-galactose-1-P-phosphatase, L-galactose dehydrogenase, and L-galactono-γ-lactone dehydrogenase.

3. A method, as claimed in claim 1, wherein said genetic modification is a genetic modification to increase the action of an epimerase that catalyzes conversion of GDP-D-mannose to GDP-L-galactose.

4. A method, as claimed in claim 3, wherein said genetic modification is a genetic modification to increase the action of GDP-D-mannose:GDP-L-galactose epimerase.

5. The method of claim 3, wherein said genetic modification comprises transformation of said microorganism with a recombinant nucleic acid molecule that expresses said epimerase.

6. The method of claim 5, wherein said epimerase has a tertiary structure that substantially conforms to the tertiary structure of a GDP-4-keto-6-deoxy-D-mannose epimerase/reductase represented by atomic coordinates having Brookhaven Protein Data Bank Accession Code 1bws.

7. The method of claim 5, wherein said epimerase has a structure having an average root mean square deviation of less than about 2.5 Å over at least about 25% of Cα positions of the tertiary structure of a GDP-4-keto-6-deoxy-D-mannose epimerase/reductase represented by atomic coordinates having Brookhaven Protein Data Bank Accession Code 1bws.

8. The method of claim 5, wherein said epimerase has a tertiary structure having an average root mean square deviation of less than about 1 Å over at least about 25% of Cα positions of the tertiary structure of a GDP-4-keto-6-deoxy-D-mannose epimerase/reductase represented by atomic coordinates having Brookhaven Protein Data Bank Accession Code 1bws.

9. The method of claim 5, wherein said epimerase comprises a substrate binding site having a tertiary structure that substantially conforms to the tertiary structure of the substrate binding site of a GDP-4-keto-6-deoxy-D-mannose epimerase/reductase represented by atomic coordinates having Brookhaven Protein Data Bank Accession Code 1bws.

10. The method of claim 9, wherein said substrate binding site has a tertiary structure with an average root mean square deviation of less than about 2.5 Å over at least about 25% of Cα positions of the tertiary structure of a substrate binding site of a GDP-4-keto-6-deoxy-D-mannose epimerase/reductase represented by atomic coordinates having Brookhaven Protein Data Bank Accession Code 1bws.

11. The method of claim 5, wherein said epimerase comprises a catalytic site having a tertiary structure that substantially conforms to the tertiary structure of the catalytic site of a GDP-4-keto-6-deoxy-D-mannose epimerase/reductase represented by atomic coordinates having Brookhaven Protein Data Bank Accession Code 1bws.

12. The method of claim 11, wherein said catalytic site has a tertiary structure with an average root mean square deviation of less than about 2.5 Å over at least about 25% of Cα positions of the tertiary structure of a catalytic site of a GDP-4-keto-6-deoxy-D-mannose epimerase/reductase represented by atomic coordinates having Brookhaven Protein Data Bank Accession Code 1bws.

13. The method of claim 11, wherein said catalytic site comprises the amino acid residues serine, tyrosine and lysine.

14. The method of claim 13, wherein tertiary structure positions of said amino acid residues serine, tyrosine and lysine substantially conform to tertiary structure positions of residues Ser107, Tyr136 and Lys140, respectively, as represented by atomic coordinates in Brookhaven Protein Data Bank Accession Code 1bws.

15. The method of claim 5, wherein said epimerase binds NADPH.

16. The method of claim 5, wherein said epimerase comprises an amino acid sequence that aligns with SEQ ID NO:11 using a CLUSTAL alignment program, wherein amino acid residues in said amino acid sequence align with 100% identity with at least about 50% of non-Xaa residues in SEQ ID NO:11.

17. The method of claim 5, wherein said epimerase comprises an amino acid sequence that aligns with SEQ ID NO:11 using a CLUSTAL alignment program, wherein amino acid residues in said amino acid sequence align with 100% identity with at least about 75% of non-Xaa residues in SEQ ID NO:11.

18. The method of claim 5, wherein said epimerase comprises an amino acid sequence that aligns with SEQ ID NO:11 using a CLUSTAL alignment program, wherein amino acid residues in said amino acid sequence align with 100% identity with at least about 90% of non-Xaa residues in SEQ ID NO:11.

19. The method of claim 5, wherein said epimerase comprises an amino acid sequence having at least 4 contiguous amino acid residues that are 100% identical to at least 4 contiguous amino acid residues of an amino acid sequence selected from the group consisting of SEQ ID NO:2, SEQ ID NO:4, SEQ ID NO:6, SEQ ID NO:8 and SEQ ID NO:10.

20. The method of claim 5, wherein said recombinant nucleic acid molecule comprises a nucleic acid sequence comprising at least about 12 contiguous nucleotides having 100% identity with at least about 12 contiguous nucleotides of a nucleic acid sequence selected from the group consisting of SEQ ID NO:1, SEQ ID NO:3, SEQ ID NO:5, SEQ ID NO:7 and SEQ ID NO:9.

21. The method of claim 5, wherein said epimerase comprises an amino acid sequence having a motif: Gly-Xaa-Xaa-Gly-Xaa-Xaa-Gly.

22. The method of claim 5, wherein said recombinant nucleic acid molecule comprises a nucleic acid sequence that is at least about 15% identical to a nucleic acid sequence selected from the group consisting of SEQ ID NO:1, SEQ ID NO:3, SEQ ID NO:5, SEQ ID NO:7 and SEQ ID NO:9, as determined using a Lipman-Pearson method with Lipman-Pearson standard default parameters.

23. The method of claim 5, wherein said recombinant nucleic acid molecule comprises a nucleic acid sequence that is at least about 20% identical to a nucleic acid sequence selected from the group consisting of SEQ ID NO:1, SEQ ID 5 NO:3, SEQ ID NO:5, SEQ ID NO:7 and SEQ ID NO:9, as determined using a Lipman-Pearson method with Lipman-Pearson standard default parameters.

24. The method of claim 5, wherein said recombinant nucleic acid molecule comprises a nucleic acid sequence that is at least about 25% identical to a nucleic acid sequence selected from the group consisting of SEQ ID NO:1, SEQ ID 5 NO:3, SEQ ID NO:5, SEQ ID NO:7 and SEQ ID NO:9, as determined using a Lipman-Pearson method with Lipman-Pearson standard default parameters.

25. The method of claim 5, wherein said recombinant nucleic acid molecule comprises a nucleic acid sequence that hybridizes under stringent hybridization conditions to a nucleic acid sequence encoding a GDP-4-keto-6-deoxy-D-mannose epimerase/reductase.

26. The method of claim 25, wherein said nucleic acid sequence encoding said GDP-4-keto-6-deoxy-D-mannose epimerase/reductase is selected from the group consisting of SEQ ID NO:1, SEQ ID NO:3 and SEQ ID NO:5.

27. The method of claim 25, wherein said GDP-4-keto-6-deoxy-D-mannose epimerase/reductase comprises an amino acid sequence selected from the group consisting of SEQ ID NO:2, SEQ ID NO:4 and SEQ ID NO:6.

28. A method, as claimed in claim 1, wherein said microorganism is selected from the group consisting of bacteria, fungi and microalgae.

29. A method, as claimed in claim 1, wherein said microorganism is acid-tolerant.

30. A method, as claimed in claim 1, wherein said microorganism is a bacterium.

31. A method, as claimed in claim 30, wherein said bacterium is selected from the group consisting of Azotobacter and Pseudomonas.

32. A method, as claimed in claim 1, wherein said microorganism is a fungus.

33. A method, as claimed in claim 32, wherein said microorganism is a yeast.

34. A method, as claimed in claim 33, wherein said yeast is selected from the group consisting of Saccharomyces yeast.

35. A method, as claimed in claim 1, wherein said microorganism is a microalga.

36. A method, as claimed in claim 35, wherein said microalga is selected from the group consisting of microalgae of the genera Prototheca and Chlorella.

37. A method, as claimed in claim 36, wherein said microalga is selected from the genus Prototheca.

38. A method, as claimed in claim 1, wherein said microorganism further comprises a genetic modification to decrease the action of an enzyme having GDP-D-mannose as a substrate, other than GDP-D-mannose:GDP-L-galactose epimerase.

39. A method, as claimed in claim 38, wherein said genetic modification to decrease the action of an enzyme having GDP-D-mannose as a substrate, other than GDP-D-mannose:GDP-L-galactose epimerase is a genetic modification to decrease the action of GDP-D-mannose-dehydrogenase.

40. A method, as claimed in claim 1, wherein said microorganism is acid-tolerant and said step of culturing is conducted at a pH of less than about 6.0.

41. A method, as claimed in claim 1, wherein said microorganism is acid-tolerant and said step of culturing is conducted at a pH of less than about 5.5.

42. A method, as claimed in claim 1, wherein said microorganism is acid-tolerant and said step of culturing is conducted at a pH of less than about 5.0.

43. A method, as claimed in claim 1, wherein said step of culturing is conducted in a fermentation medium that is magnesium (Mg) limited.

44. A method, as claimed in claim 1, wherein said step of culturing is conducted in a fermentation medium that is Mg limited during a cell growth phase.

45. A method, as claimed in claim 1, wherein said step of culturing is conducted in a fermentation medium that comprises less than about 0.5 g/L of Mg during a cell growth phase.

46. A method, as claimed in claim 1, wherein said step of culturing is conducted in a fermentation medium that comprises less than about 0.2 g/L of Mg during a cell growth phase.

47. A method, as claimed in claim 1, wherein said step of culturing is conducted in a fermentation medium that comprises less than about 0.1 g/L of Mg during a cell growth phase.

48. A method, as claimed in claim 1, wherein said step of culturing is conducted in a fermentation medium that comprises a carbon source other than D-mannose.

49. A method, as claimed in claim 1, wherein said step of culturing is conducted in a fermentation medium that comprises glucose as a carbon source.

50. A microorganism for producing ascorbic acid or esters thereof, wherein said microorganism has a genetic modification to increase the action of an enzyme selected from the group consisting of hexokinase, glucose phosphate isomerase, phosphomannose isomerase, phosphomannomutase, GDP-D-mannose pyrophosphorylase, GDP-D-mannose:GDP-L-galactose epimerase, GDP-L-galactose phosphorylase, L-galactose-1-P-phosphatase, L-galactose dehydrogenase, and L-galactono-γ-lactone dehydrogenase.

51. A microorganism, as claimed in claim 50, wherein said genetic modification is a genetic modification to increase the action of an enzyme selected from the group consisting of GDP-D-mannose:GDP-L-galactose epimerase, GDP-L-galactose phosphorylase, L-galactose-1-P-phosphatase, L-galactose dehydrogenase, and L-galactono-γ-lactone dehydrogenase.

52. A microorganism, as claimed in claim 50, wherein said genetic modification is a genetic modification to increase the action of GDP-D-mannose:GDP-L-galactose epimerase.

53. A microorganism, as claimed in claim 50, wherein said microorganism has been genetically modified to express a recombinant nucleic acid molecule encoding an epimerase that catalyzes conversion of GDP-D-mannose to GDP-L-galactose, wherein said epimerase has a tertiary structure having an average root mean square deviation of less than about 2.5 Å over at least about 25% of Ca positions of the tertiary structure of a GDP-4-keto-6-deoxy-D-mannose epimerase/reductase represented by atomic coordinates having Brookhaven Protein Data Bank Accession Code 1bws.

54. A microorganism, as claimed in claim 50, wherein said microorganism is selected from the group consisting of bacteria, fungi and microalgae.

55. A microorganism, as claimed in claim 50, wherein said microorganism is a bacterium.

56. A microorganism, as claimed in claim 55, wherein said bacterium is selected from the group consisting of Azotobacter and Pseudomonas.

57. A microorganism, as claimed in claim 50, wherein said microorganism is a fungus.

58. A microorganism, as claimed in claim 57, wherein said microorganism is a yeast.

59. A microorganism, as claimed in claim 58, wherein said yeast is selected from the group consisting of Saccharomyces yeast.

60. A plant for producing ascorbic acid or esters thereof, wherein said plant has a genetic modification to increase the action of an enzyme selected from the group consisting of hexokinase, glucose phosphate isomerase, phosphomannose isomerase, phosphomannomutase, GDP-D-mannose pyrophosphorylase, GDP-D-mannose:GDP-L-galactose epimerase, GDP-L-galactose phosphorylase, L-galactose-1-P-phosphatase, L-galactose dehydrogenase, and L-galactono-γ-lactone dehydrogenase.

61. A plant, as claimed in claim 60, wherein said genetic modification is a genetic modification to increase the action of an enzyme selected from the group consisting of GDP-D-mannose:GDP-L-galactose epimerase, GDP-L-galactose phosphorylase, L-galactose-1-P-phosphatase, L-galactose dehydrogenase, and L-galactono-γ-lactone dehydrogenase.

62. A plant, as claimed in claim 60, wherein said genetic modification is a genetic modification to increase the action of GDP-D-mannose:GDP-L-galactose epimerase.

63. A plant, as claimed in claim 60, wherein said plant has been genetically modified to express a recombinant nucleic acid molecule encoding an epimerase that catalyzes conversion of GDP-D-mannose to GDP-L-galactose, wherein said epimerase has a tertiary structure having an average root mean square deviation of less than about 2.5 Å over at least about 25% of Cα positions of the tertiary structure of a GDP-4-keto-6-deoxy-D-mannose epimerase/reductase represented by atomic coordinates having Brookhaven Protein Data Bank Accession Code 1bws.

64. A plant, as claimed in claim 60, wherein said plant further comprises a genetic modification to decrease the action of an enzyme having GDP-D-mannose as a substrate other than GDP-D-mannose:GDP-L-galactose epimerase.

65. A plant, as claimed in claim 60, wherein said genetic modification to decrease the action of an enzyme having GDP-D-mannose as a substrate other than GDP-D-mannose:GDP-L-galactose epimerase is a genetic modification to decrease the action of GDP-D-mannose-dehydrogenase.

66. A plant, as claimed in claim 60, wherein said plant is a microalga.

67. A plant, as claimed in claim 66, wherein said plant is selected from the group consisting of microalgae of the genera Prototheca and Chlorella.

68. A plant, as claimed in claim 66, wherein said microalga is selected from the genus Prototheca.

69. A plant, as claimed in claim 60, wherein said plant is a higher plant.

70. A plant, as claimed in claim 60, wherein said plant is a consumable higher plant.

71. A microorganism for producing ascorbic acid or esters thereof, wherein said microorganism has been genetically modified to express a recombinant nucleic acid molecule encoding an epimerase that catalyzes conversion of GDP-D-mannose to GDP-L-galactose, wherein said epimerase comprises an amino acid sequence that aligns with SEQ ID NO:11 using a CLUSTAL alignment program, wherein amino acid residues in said amino acid sequence align with 100% identity with at least about 50% of non-Xaa residues in SEQ ID NO:11.

72. A plant for producing ascorbic acid or esters thereof, wherein said plant has been genetically modified to express a recombinant nucleic acid molecule encoding an epimerase that catalyzes conversion of GDP-D-mannose to GDP-L-galactose, wherein said epimerase comprises an amino acid sequence that aligns with SEQ ID NO:11 using a CLUSTAL alignment program, wherein amino acid residues in said amino acid sequence align with 100% identity with at least about 50% of non-Xaa residues in SEQ ID NO:11.


SEQUENCE LISTING (15 sequences)

SEQ ID NO: 1 - DNA, 1583 bp, Arabidopsis thaliana
  CDS (49)..(990)
tagtctttaatttcgcagcgtttttataattgtgcagaggtttcgtccatgtctgacaaatctgccaaaatcttcgtcgcgggtcatcgtggtttggttggatctgccattgtccgcaagcttcaggaacaaggtttcaccaatctcgttcttaaaacacacgccgagcttgatctcactcgtcaagccgatgttgaatccttcttttctcaagagaagccagtttatgtaatcctagcagcagctaaagttggtggtattcacgctaacaacacctatcctgctgatttcattggtgtcaatctccagattcagaccaatgtgatccactctgcatatgagcacggtgtgaagaagcttctcttccttggatcatcctgcatttaccctaaatttgctcctcagccaattcctgagtctgctttgttaacagcatcgcttgaaccaactaatgagtggtatgctattgctaagatcgctgggattaagacttgtcaggcttataggattcagcacggatgggatgcaatctctggcatgcctactaatctctatggtcctaatgacaatttccacccggagtctcatgtgcttcctgctcttatgaggaggttccacgaggcgaaagtgaattggagcggaggaagttgtggtgtggggtacaaggtagtcccgttggaagggaagttcttgcatgttgatgatttggctgatgcttgtgttttcttgctggatcggatacagcgggggttggagcatgttaacattggaagtggtcaagaagtgactattagagagttggctgagttggtgaaagaggttgttggttttgaagggaagcttggatgggattgcactaagccagatggcacaccgaggaaacttatggacagctcaaagctcgcgtctttgggttggacacctaaggtttctcttagagatggtctgagccaaacttatgattggtatttgaagaatgtttgcaaccgataagttaatggtttctcttctcatatatacacaactattgagtctcaggtaaatcagcttatcaccacattgtgatttaaacctttctttgagattcgagaattgcttttttttttatcaaaattgattcatttagagataagacttgcttctttatacaacattgtctgaggaattttaattttggatctccgagtatggtctattattagctctcttctatacaaattatcaaaacagttgtaagaagtttcaagaaaaacatttgatatctcactaatttggctatccttgcaagttgcaacgctaaaatgacaaataatgaattctcggcccaatgggcttacacaagccttgttaaagatagcgtgaacaaaacgcggctcactagccctaacctgtctctctttcgcttaccttcttcttcgtcttcgttggctcagtcacttgacttcacggcccgctcaagctctgacacgaaactcatttcaaattaatttaataaaaccttaatcacaaaaggggcaaaagcaatcgccggcgattatgccttctcctccggtgccggagacggttgtgagccaacccgttcg

SEQ ID NO: 2 - protein, 314 aa, Arabidopsis thaliana
MSDKSAKIFVAGHRGLVGSAIVRKLQEQGFTNLVLKTHAELDLTRQADVESFFSQEKPVYVILAAAKVGGIHANNTYPADFIGVNLQIQTNVIHSAYEHGVKKLLFLGSSCIYPKFAPQPIPESALLTASLEPTNEWYAIAKIAGIKTCQAYRIQHGWDAISGMPTNLYGPNDNFHPESHVLPALMRRFHEAKVNWSGGSCGVGYKVVPLEGKFLHVDDLADACVFLLDRIQRGLEHVNIGSGQEVTIRELAELVKEVVGFEGKLGWDCTKPDGTPRKLMDSSKLASLGWTPKVSLRDGLSQTYDWYLKNVCNR

SEQ ID NO: 3 - DNA, 966 bp, Escherichia coli
  CDS (1)..(966)
atgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgacgtggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaa

SEQ ID NO: 4 - protein, 321 aa, Escherichia coli
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRDVAQTIAKVVGYKGRVVFDASKPDGTPRKLLDVTRLHQLGWYHEISLEAGLASTYQWFLENQDRFRG

SEQ ID NO: 5 - DNA, 1340 bp, Homo sapiens
  CDS (75)..(1040)
ctagaattcagcggccgctgaattctagctagaattcagcggccgctgaattctagaacccaggtgcaactgacatgggtgaaccccagggatccatgcggattctagtgacagggggctctgggctggtaggcaaagccatccagaaggtggtagcagatggagctggacttcctggagaggactgggtgtttgtctcctctaaagacgccgatctcacggatacagcacagacccgcgccctgtttgagaaggtccaacccacacacgtcatccatcttgctgcaatggtggggggcctgttccggaatatcaaatacaatttggacttctggaggaaaaacgtgcacatgaacgacaacgtcctgcactcggcctttgaggtgggggcccgcaaggtggtgtcctgcctgtccacctgtatcttccctgacaagacgacctacccgatagatgagaccatgatccacaatgggcctccccacaacagcaattttgggtactcgtatgccaagaggatgatcgacgtgcagaacagggcctacttccagcagtacggctgcaccttcaccgctgtcatccccaccaacgttttcgggccccacgacaacttcaacatcgaggatggccacgtgctgcctggcctcatccacaaggtgcacctggccaagagcagcggctcggccctgacggtgtggggtacagggaatccgcggaggcagttcatatactcgctggacctggcccagctctttatctgggtcctgcgggagtacaatgaagtggagcccatcatcctctccgtgggcgaggaagatgaggtctccatcaaggaggcagccgaggcggtggtggaggccatggacttccatggggaagtcacctttgatacaaccaagtcggatgggcagtttaagaagacagccagtaacagcaagctgaggacctacctgcccgacttccggttcacacccttcaagcaggcggtgaaggagacctgtgcttggttcactgacaactacgagcaggcccggaagtgaagctggaagacaggatcaggtgccagcggaccatcggctggcagagcccagcggccaccacccgtcaaccctgccaggagctgagggcaccacccagcaacctgggcctgcattccatccgctctgcagccccaagcatctttccagtggggcccccattcacgttggtcctcagggaaaccagggtccggggcaggcccggcgctttgctccccacaccagccccctgcgcgtgtccactctgatcctgcatcccactccctgggagccaataaagtgcattttcacagaaaaaaaaaa

SEQ ID NO: 6 - protein, 321 aa, Homo sapiens
MGEPQGSMRILVTGGSGLVGKAIQKVVADGAGLPGEDWVFVSSKDADLTDTAQTRALFEKVQPTHVIHLAAMVGGLFRNIKYNLDFWRKNVHMNDNVLHSAFEVGARKVVSCLSTCIFPDKTTYPIDETMIHNGPPHNSNFGYSYAKRMIDVQNRAYFQQYGCTFTAVIPTNVFGPHDNFNIEDGHVLPGLIHKVHLAKSSGSALTVWGTGNPRRQFIYSLDLAQLFIWVLREYNEVEPIILSVGEEDEVSIKEAAEAVVEAMDFHGEVTFDTTKSDGQFKKTASNSKLRTYLPDFRFTPFKQAVKETCAWFTDNYEQARK

SEQ ID NO: 7 - DNA, 1017 bp, Escherichia coli
  CDS (1)..(1017)
atgagagttctggttaccggtggtagcggttacattggaagtcatacctgtgtgcaattactgcaaaacggtcatgatgtcatcattcttgataacctctgtaacagtaagcgcagcgtactgcctgttatcgagcgtttaggcggcaaacatccaacgtttgttgaaggcgatattcgtaacgaagcgttgatgaccgagatcctgcacgatcacgctatcgacaccgtgatccacttcgccgggctgaaagccgtgggcgaatcggtacaaaaaccgctggaatattacgacaacaatgtcaacggcactctgcgcctgattagcgccatgcgcgccgctaacgtcaaaaactttatttttagctcctccgccaccgtttatggcgatcagcccaaaattccatacgttgaaagcttcccgaccggcacaccgcaaagcccttacggcaaaagcaagctgatggtggaacagatcctcaccgatctgcaaaaagcccagccggactggagcattgccctgctgcgctacttcaacccggttggcgcgcatccgtcgggcgatatgggcgaagatccgcaaggcattccgaataacctgatgccatacatcgcccaggttgctgtaggccgtcgcgactcgctggcgatttttggtaacgattatccgaccgaagatggtactggcgtacgcgattacatccacgtaatggatctggcggacggtcacgtcgtggcgatggaaaaactggcgaacaagccaggcgtacacatctacaacctcggcgctggcgtaggcaacagcgtgctggacgtggttaatgccttcagcaaagcctgcggcaaaccggttaattatcattttgcaccgcgtcgcgagggcgaccttccggcctactgggcggacgccagcaaagccgaccgtgaactgaactggcgcgtaacgcgcacactcgatgaaatggcgcaggacacctggcactggcagtcacgccatccacagggatatcccgattaa

SEQ ID NO: 8 - protein, 338 aa, Escherichia coli
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 9 - DNA, 1047 bp, Homo sapiens
  CDS (1)..(1047)
atggcagagaaggtgctggtaacaggtggggctggctacattggcagccacacggtgctggagctgctggaggctggctacttgcctgtggtcatcgataacttccataatgccttccgtggagggggctccctgcctgagagcctgcggcgggtccaggagctgacaggccgctctgtggagtttgaggagatggacattttggaccagggagccctacagcgtctcttcaaaaagtacagctttatggcggtcatccactttgcggggctcaaggccgtgggcgagtcggtgcagaagcctctggattattacagagttaacctgaccgggaccatccagcttctggagatcatgaaggcccacggggtgaagaacctggtgttcagcagctcagccactgtgtacgggaacccccagtacctgccccttgatgaggcccaccccacgggtggttgtaccaacccttacggcaagtccaagttcttcatcgaggaaatgatccgggacctgtgccaggcagacaagacttggaacgtagtgctgctgcgctatttcaaccccacaggtgcccatgcctctggctgcattggtgaggatccccagggcatacccaacaacctcatgccttatgtctcccaggtggcgatcgggcgacgggaggccctgaatgtctttggcaatgactatgacacagaggatggcacaggtgtccgggattacatccatgtcgtggatctggccaagggccacattgcagccttaaggaagctgaaagaacagtgtggctgccggatctacaacctgggcacgggcacaggctattcagtgctgcagatggtccaggctatggagaaggcctctgggaagaagatcccgtacaaggtggtggcacggcgggaaggtgatgtggcagcctgttacgccaaccccagcctggcccaagaggagctggggtggacagcagccttagggctggacaggatgtgtgaggatctctggcgctggcagaagcagaatccttcaggctttggcacgcaagcctga

SEQ ID NO: 10 - protein, 348 aa, Homo sapiens
MAEKVLVTGGAGYIGSHTVLELLEAGYLPVVIDNFHNAFRGGGSLPESLRRVQELTGRSVEFEEMDILDQGALQRLFKKYSFMAVIHFAGLKAVGESVQKPLDYYRVNLTGTIQLLEIMKAHGVKNLVFSSSATVYGNPQYLPLDEAHPTGGCTNPYGKSKFFIEEMIRDLCQADKTWNVVLLRYFNPTGAHASGCIGEDPQGIPNNLMPYVSQVAIGRREALNVFGNDYDTEDGTGVRDYIHVVDLAKGHIAALRKLKEQCGCRIYNLGTGTGYSVLQMVQAMEKASGKKIPYKVVARREGDVAACYANPSLAQEELGWTAALGLDRMCEDLWRWQKQNPSGFGTQA

SEQ ID NO: 11 - protein, 317 aa, Artificial Sequence
  Description of Artificial SequenceCONSENSUS
XXRXXXXGXXGXXGXXXXXXXXXXXXXXXXXXXXXXXXXXXXXXXXXXXXXXXXXXVXXXAXXXXXXXXXXXXXXXXXXXNXXXXXXXXXXXXXXXXXXXXXXXSXXXXXXXXXXPXXEXXXXXGXXXXXXXXYXXXKXXXXXXXXXXXXXXXXXXXXXXXXNXXGXHXXXXXXXXXXXPXXXXXXXXXXXXXXXXXXXXGXGXXXRXXXXXXDXAXXXXXXXXXXXXXXXXXXXXXXXXXXXGXXXXXXXXXXXXXXXXXXXXXXXXXFXXXXXXXXXXXXXXXXXXXXLXXXXXXXXXXXXXXTXXWXXXXXXXX

SEQ ID NO: 12 - DNA, 34 bp, Escherichia coli
tagaattcagtaaacaacgagtttttattgctgg

SEQ ID NO: 13 - DNA, 32 bp, Escherichia coli
aactcgagttacccccaaagcggtcttgattc

SEQ ID NO: 14 - DNA, 30 bp, Escherichia coli
ctggagtcgaattcatgagtaaacaacgag

SEQ ID NO: 15 - DNA, 33 bp, Escherichia coli
aactgcagttacccccgaaagcggtcttgattc